SEQUENCE LISTING (216 sequences)

SEQ ID NO: 1 - protein, 571 aa, synthetic construct
  source 1..571: note = BE-830 heavy chain AA
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSS

SEQ ID NO: 2 - DNA, 1716 bp, synthetic construct
  source 1..1716: mol_type = other DNA; note = BE-830 heavy chain DNA
caggtgcagctggtgcagtctggagcagaggtgaagaagccaggagccagcgtgaaggtgtcctgcaaggcctctggctataccttcacagactacgagatgcactgggtgcggcaggcaccaggacagggactggagtggatgggcgccctggaccctaagaccggcgatacagcctatagccagaagtttaagggcagagtgaccctgacagccgacaagtccacctctacagcctacatggagctgagctccctgaccagcgaggatacagccgtgtactattgtacccggttctactcctatacatactggggccagggcaccctggtgacagtgtctagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcgtga

SEQ ID NO: 3 - protein, 219 aa, synthetic construct
  source 1..219: note = BE-830 light chain AA
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - DNA, 657 bp, synthetic construct
  source 1..657: mol_type = other DNA; note = BE-830 light chain DNA
gacgtggtcatgacacagtcccctctgtctctgccagtgaccccaggagagcctgcctctatcagctgcaggagctcccagagcctggtgcactccaaccgcaatacatacctgcactggtatctgcagaagccaggccagtccccccagctgctgatctacaaggtgtctaaccggttcagcggcgtgcccgacagattttccggctctggcagcggcaccgatttcacactgaagatctcccgggtggaggcagaggatgtgggcgtgtactattgttctcagaatacccacgtgccccctacatttggccagggcaccaagctggagatcaagcgtacggtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 5 - protein, 115 aa, synthetic construct
  source 1..115: note = GPC3 VH in BE-830 AA
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 6 - DNA, 345 bp, synthetic construct
  source 1..345: mol_type = other DNA; note = GPC3 VH in BE-830 DNA
caggtgcagctggtgcagtctggagcagaggtgaagaagccaggagccagcgtgaaggtgtcctgcaaggcctctggctataccttcacagactacgagatgcactgggtgcggcaggcaccaggacagggactggagtggatgggcgccctggaccctaagaccggcgatacagcctatagccagaagtttaagggcagagtgaccctgacagccgacaagtccacctctacagcctacatggagctgagctccctgaccagcgaggatacagccgtgtactattgtacccggttctactcctatacatactggggccagggcaccctggtgacagtgtctagc

SEQ ID NO: 7 - protein, 112 aa, synthetic construct
  source 1..112: note = GPC3 VL in BE-830 AA
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 8 - DNA, 336 bp, synthetic construct
  source 1..336: mol_type = other DNA; note = "GPC3 VL in BE-830 DNA"
gacgtggtcatgacacagtcccctctgtctctgccagtgaccccaggagagcctgcctctatcagctgcaggagctcccagagcctggtgcactccaaccgcaatacatacctgcactggtatctgcagaagccaggccagtccccccagctgctgatctacaaggtgtctaaccggttcagcggcgtgcccgacagattttccggctctggcagcggcaccgatttcacactgaagatctcccgggtggaggcagaggatgtgggcgtgtactattgttctcagaatacccacgtgccccctacatttggccagggcaccaagctggagatcaag

SEQ ID NO: 9 - protein, 231 aa, synthetic construct
  source 1..231: note = Fc in BE-830 AA
EPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 5 aa, synthetic construct
  source 1..5: note = Anti-GPC3 antibody HCDR1 (Kabat)
DYEMH

SEQ ID NO: 11 - protein, 17 aa, synthetic construct
  source 1..17: note = Anti-GPC3 antibody HCDR2 (Kabat)
ALDPKTGDTAYSQKFKG

SEQ ID NO: 12 - protein, 6 aa, synthetic construct
  source 1..6: note = Anti-GPC3 antibody HCDR3 (Kabat)
FYSYTY

SEQ ID NO: 13 - protein, 16 aa, synthetic construct
  source 1..16: note = Anti-GPC3 antibody LCDR1 (Kabat)
RSSQSLVHSNRNTYLH

SEQ ID NO: 14 - protein, 7 aa, synthetic construct
  source 1..7: note = Anti-GPC3 antibody LCDR2 (Kabat)
KVSNRFS

SEQ ID NO: 15 - protein, 9 aa, synthetic construct
  source 1..9: note = Anti-GPC3 antibody LCDR3 (Kabat)
SQNTHVPPT

SEQ ID NO: 16-20 - skipped (5 entries)

SEQ ID NO: 21 - protein, 4 aa, synthetic construct
  source 1..4: note = Linker
GGGS

SEQ ID NO: 22 - protein, 4 aa, synthetic construct
  source 1..4: note = Linker
SGGG

SEQ ID NO: 23 - protein, 5 aa, synthetic construct
  source 1..5: note = Linker
GGGGS

SEQ ID NO: 24 - protein, 7 aa, synthetic construct
  source 1..7: note = Linker
GGGGSGS

SEQ ID NO: 25 - protein, 7 aa, synthetic construct
  source 1..7: note = Linker
GGGGSGS

SEQ ID NO: 26 - protein, 8 aa, synthetic construct
  source 1..8: note = Linker
GGGGSGGS

SEQ ID NO: 27 - protein, 10 aa, synthetic construct
  source 1..10: note = Linker
GGGGSGGGGS

SEQ ID NO: 28 - protein, 15 aa, synthetic construct
  source 1..15: note = Linker
GGGGSGGGGSGGGGS

SEQ ID NO: 29 - protein, 16 aa, synthetic construct
  source 1..16: note = Linker
AKTTPKLEEGEFSEAR

SEQ ID NO: 30 - protein, 17 aa, synthetic construct
  source 1..17: note = Linker
AKTTPKLEEGEFSEARV

SEQ ID NO: 31 - protein, 9 aa, synthetic construct
  source 1..9: note = Linker
AKTTPKLGG

SEQ ID NO: 32 - protein, 10 aa, synthetic construct
  source 1..10: note = Linker
SAKTTPKLGG

SEQ ID NO: 33 - protein, 17 aa, synthetic construct
  source 1..17: note = Linker
AKTTPKLEEGEFSEARV

SEQ ID NO: 34 - protein, 6 aa, synthetic construct
  source 1..6: note = Linker
SAKTTP

SEQ ID NO: 35 - protein, 10 aa, synthetic construct
  source 1..10: note = Linker
SAKTTPKLGG

SEQ ID NO: 36 - protein, 6 aa, synthetic construct
  source 1..6: note = Linker
RADAAP

SEQ ID NO: 37 - protein, 9 aa, synthetic construct
  source 1..9: note = Linker
RADAAPTVS

SEQ ID NO: 38 - protein, 12 aa, synthetic construct
  source 1..12: note = Linker
RADAAAAGGPGS

SEQ ID NO: 39 - protein, 27 aa, synthetic construct
  source 1..27: note = Linker
RADAAAAGGGGSGGGGSGGGGSGGGGS

SEQ ID NO: 40 - protein, 6 aa, synthetic construct
  source 1..6: note = Linker
SAKTTP

SEQ ID NO: 41 - protein, 10 aa, synthetic construct
  source 1..10: note = Linker
SAKTTPKLGG

SEQ ID NO: 42 - protein, 18 aa, synthetic construct
  source 1..18: note = Linker
SAKTTPKLEEGEFSEARV

SEQ ID NO: 43 - protein, 5 aa, synthetic construct
  source 1..5: note = Linker
ADAAP

SEQ ID NO: 44 - protein, 12 aa, synthetic construct
  source 1..12: note = Linker
ADAAPTVSIFPP

SEQ ID NO: 45 - protein, 5 aa, synthetic construct
  source 1..5: note = Linker
TVAAP

SEQ ID NO: 46 - protein, 12 aa, synthetic construct
  source 1..12: note = Linker
TVAAPSVFIFPP

SEQ ID NO: 47 - protein, 6 aa, synthetic construct
  source 1..6: note = Linker
QPKAAP

SEQ ID NO: 48 - protein, 13 aa, synthetic construct
  source 1..13: note = Linker
QPKAAPSVTLFPP

SEQ ID NO: 49 - protein, 6 aa, synthetic construct
  source 1..6: note = Linker
AKTTPP

SEQ ID NO: 50 - protein, 13 aa, synthetic construct
  source 1..13: note = Linker
AKTTPPSVTPLAP

SEQ ID NO: 51 - protein, 6 aa, synthetic construct
  source 1..6: note = Linker
AKTTAP

SEQ ID NO: 52 - protein, 8 aa, synthetic construct
  source 1..8: note = HCDR1 (IMGT)
GFRLDTTE

SEQ ID NO: 53 - protein, 8 aa, Synthetic construct
  source 1..8: note = HCDR2 (IMGT)
IMGISGST

SEQ ID NO: 54 - protein, 16 aa, synthetic construct
  source 1..16: note = HCDR3 (IMGT)
ARVVDSLFDDSAVFDY

SEQ ID NO: 55 - protein, 5 aa, synthetic construct
  source 1..5: note = HCDR1 (Kabat)
TTEVG

SEQ ID NO: 56 - protein, 17 aa, synthetic construct
  source 1..17: note = HCDR2 (Kabat)
TIMGISGSTYYADSVKG

SEQ ID NO: 57 - protein, 14 aa, synthetic construct
  source 1..14: note = HCDR3 (Kabat)
VVDSLFDDSAVFDY

SEQ ID NO: 58 - protein, 7 aa, synthetic construct
  source 1..7: note = HCDR1 (Chothia)
GFRLDTT

SEQ ID NO: 59 - protein, 6 aa, synthetic construct
  source 1..6: note = HCDR2 (Chothia)
MGISGS

SEQ ID NO: 60 - protein, 123 aa, synthetic construct
  source 1..123: note = VH
EVQLLESGGGLVQPGGSLRLSCAASGFRLDTTEVGWVRQAPGKGLEWVSTIMGISGSTYYADSVKGRFTISRDNSKNTLYLQMNSLRAEDTAVYYCARVVDSLFDDSAVFDYWGQGTLVTVSS

SEQ ID NO: 61 - DNA, 369 bp, synthetic construct
  source 1..369: mol_type = other DNA; note = VH DNA
gaggtccagttacttgagagtggtggaggtctggtccaaccaggaggttcgctgcgtttatcctgcgccgcgtctggattcagattggacaccaccgaagtaggttgggtgcgtcaagcgccggggaaaggactggaatgggtctccaccatcatgggtattagtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtcgattccctctttgatgacagcgccgtttttgattactggggacagggcaccttagttacagtctcatcg

SEQ ID NO: 62 - protein, 8 aa, synthetic construct
  source 1..8: note = HCDR1 (IMGT)
GFMLSAED

SEQ ID NO: 63 - protein, 8 aa, synthetic construct
  source 1..8: note = HCDR2 (IMGT)
ILDFGGST

SEQ ID NO: 64 - protein, 15 aa, synthetic construct
  source 1..15: note = HCDR3 (IMGT)
ARVVYHAGGGVTFDY

SEQ ID NO: 65 - protein, 5 aa, synthetic construct
  source 1..5: note = HCDR1 (Kabat)
AEDVG

SEQ ID NO: 66 - protein, 17 aa, synthetic construct
  source 1..17: note = HCDR2 (Kabat)
AILDFGGSTYYADSVKG

SEQ ID NO: 67 - protein, 13 aa, synthetic construct
  source 1..13: note = HCDR3 (Kabat)
VVYHAGGGVTFDY

SEQ ID NO: 68 - protein, 7 aa, synthetic construct
  source 1..7: note = HCDR1 (Chothia)
GFMLSAE

SEQ ID NO: 69 - protein, 6 aa, synthetic construct
  source 1..6: note = HCDR2 (Chothia)
LDFGGS

SEQ ID NO: 70 - protein, 122 aa, synthetic construct
  source 1..122: note = VH
EVQLLESGGGLVQPGGSLRLSCAASGFMLSAEDVGWVRQAPGKGLEWVSAILDFGGSTYYADSVKGRFTISRDNSKNTLYLQMNSLRAEDTAVYYCARVVYHAGGGVTFDYWGQGTLVTVSS

SEQ ID NO: 71 - DNA, 366 bp, synthetic construct
  source 1..366: mol_type = other DNA; note = VH DNA
gaggtccagttacttgagagtggtggaggtctggtccaaccaggaggttcgctgcgtttatcctgcgccgcgtctggattcatgttgtccgccgaagacgtgggttgggtgcgtcaagcgccggggaaaggactggaatgggtctccgccatcttggattttggtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtctaccatgctggtggtggcgtcacctttgattactggggacagggcaccttagttacagtctcatcg

SEQ ID NO: 72 - protein, 8 aa, synthetic construct
  source 1..8: note = HCDR1 (IMGT)
GFTLSAED

SEQ ID NO: 73 - protein, 17 aa, synthetic construct
  source 1..17: note = HCDR2 (Kabat)
AILDFGGSTYYAESVKG

SEQ ID NO: 74 - protein, 7 aa, synthetic construct
  source 1..7: note = HCDR1 (Chothia)
GFTLSAE

SEQ ID NO: 75 - protein, 122 aa, synthetic construct
  source 1..122: note = VH
EVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYWGQGTLVTVSS

SEQ ID NO: 76 - DNA, 366 bp, synthetic construct
  source 1..366: mol_type = other DNA; note = VH DNA
gaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattactggggacagggcaccttagttacagtctcatcg

SEQ ID NO: 77 - protein, 8 aa, synthetic construct
  source 1..8: note = HCDR1 (IMGT)
GFTVSAED

SEQ ID NO: 78 - protein, 8 aa, synthetic construct
  source 1..8: note = HCDR2 (IMGT)
ILDKGGST

SEQ ID NO: 79 - protein, 15 aa, synthetic construct
  source 1..15: note = HCDR3 (IMGT)
ARIVYHAGGGVTFDT

SEQ ID NO: 80 - protein, 17 aa, synthetic construct
  source 1..17: note = HCDR2 (Kabat)
AILDKGGSTYYAESVKG

SEQ ID NO: 81 - protein, 13 aa, synthetic construct
  source 1..13: note = HCDR3 (Kabat)
IVYHAGGGVTFDT

SEQ ID NO: 82 - protein, 7 aa, synthetic construct
  source 1..7: note = HCDR1 (Chothia)
GFTVSAE

SEQ ID NO: 83 - protein, 6 aa, synthetic construct
  source 1..6: note = HCDR2 (Chothia)
LDKGGS

SEQ ID NO: 84 - protein, 122 aa, synthetic construct
  source 1..122: note = BGA-5623 AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSS

SEQ ID NO: 85 - DNA, 366 bp, synthetic construct
  source 1..366: mol_type = other DNA; note = BGA-5623 DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 86 - protein, 122 aa, synthetic construct
  source 1..122: note = BGA-7556
EVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYRGQGTQVTVSS

SEQ ID NO: 87 - DNA, 366 bp, synthetic construct
  source 1..366: mol_type = other DNA; note = VH DNA
gaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattaccggggacagggcacccaagttacagtctcatcg

SEQ ID NO: 88 - protein, 13 aa, synthetic construct
  source 1..13: note = Linker
AKTTAPSVYPLAP

SEQ ID NO: 89 - protein, 6 aa, Synthetic construct
  source 1..6: note = Linker
ASTKGP

SEQ ID NO: 90 - protein, 13 aa, synthetic construct
  source 1..13: note = Linker
ASTKGPSVFPLAP

SEQ ID NO: 91 - protein, 15 aa, synthetic construct
  source 1..15: note = Linker
GENKVEYAPALMALS

SEQ ID NO: 92 - protein, 15 aa, synthetic construct
  source 1..15: note = Linker
GPAKELTPLKEAKVS

SEQ ID NO: 93 - protein, 15 aa, synthetic construct
  source 1..15: note = Linker
GHEAAAVMQVQYPAS

SEQ ID NO: 94 - protein, 255 aa, Homo sapiens
  source 1..255: note = human CD137 FL AA
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 95 - DNA, 765 bp, Homo sapiens
  source 1..765: mol_type = other DNA; note = human CD137 FL DNA
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 96 - protein, 160 aa, Homo sapiens
  source 1..160: note = human CD137 AA 24-183
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGH

SEQ ID NO: 97 - DNA, 480 bp, Homo sapiens
  source 1..480: mol_type = other DNA; note = human CD137 24-183 DNA
ctgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacac

SEQ ID NO: 98 - protein, 183 aa, Homo sapiens
  source 1..183: note = human CD137 -his
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHHHHHHHGGGLNDIFEAQKIEWHE

SEQ ID NO: 99 - DNA, 549 bp, Homo sapiens
  source 1..549: mol_type = other DNA; note = human CD137 -his DNA
ctgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacaccatcaccatcaccatcacggaggcggtctgaacgacatcttcgaggctcagaaaatcgaatggcacgaa

SEQ ID NO: 100 - protein, 393 aa, Homo sapiens
  source 1..393: note = human CD137 ECD-mIgG2a AA
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 101 - DNA, 1179 bp, Homo sapiens
  source 1..1179: mol_type = other DNA; note = human CD137 ECD-mIgG2a DNA
ctgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacacgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 102 - protein, 233 aa, synthetic construct
  source 1..233: note = mIgG2a AA
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 103 - DNA, 699 bp, synthetic construct
  source 1..699: mol_type = other DNA; note = mIgG2a DNA
gagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 104 - protein, 254 aa, Homo sapiens
  source 1..254: note = human CD137 ligand AA
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 105 - DNA, 765 bp, Homo sapiens
  source 1..765: mol_type = other DNA; note = human CD137 ligand cDNA
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 106 - protein, 184 aa, Homo sapiens
  source 1..184: note = human CD137 ligand 71-254 AA
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 107 - DNA, 552 bp, Homo sapiens
  source 1..552: mol_type = other DNA; note = human CD137 ligand 71-254 DNA
cgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 108 - protein, 422 aa, Homo sapiens
  source 1..422: note = human CD137 ligand-mIgG2a AA
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 109 - DNA, 1266 bp, Homo sapiens
  source 1..1266: mol_type = other DNA; note = human CD137 ligand-mIgG2a DNA
gagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaggtggaggcggttcacgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 110 - protein, 254 aa, synthetic construct
  source 1..254: note = cyno CD137 FL AA
MGNSCYNIVATLLLVLNFERTRSLQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQIIFFLALTSTVVLFLLFFLVLRFSVVKRSRKKLLYIFKQPFMRPVQTTQEEDGCSCRFPEEEEGGCEL

SEQ ID NO: 111 - DNA, 765 bp, synthetic construct
  source 1..765: mol_type = other DNA; note = cyno CD137 FL DNA
atgggaaacagctgttacaacatagtagccactctgttgctggtcctgaactttgagaggacaagatccttgcaggatctgtgtagtaactgcccagctggtacattctgtgataataacaggagtcagatttgcagtccctgtcctccaaatagtttctccagcgcaggtggacaaaggacctgtgacatatgcaggcagtgtaaaggtgttttcaagaccaggaaggagtgttcctccaccagcaatgcagagtgtgactgcatttcaggatatcactgcctgggggcagaatgcagcatgtgtgaacaggattgtaaacaaggtcaagaattgacaaaaaaaggttgtaaagactgttgctttgggacatttaatgaccagaaacgtggcatctgtcgaccctggacaaactgttctttggatggaaagtctgtgcttgtgaatgggacgaaggagagggacgtggtctgcggaccatctccagccgacctctctccaggagcatcctctgcgaccccgcctgcccctgcgagagagccaggacactctccgcagatcatcttctttcttgcgctgacttcgactgtggtactcttcctgctgttcttcctcgtgctccgtttctctgttgttaaacggagcagaaagaaactcctgtatatattcaaacaaccatttatgagaccggtacaaaccactcaagaggaagatggatgtagctgccgatttccagaagaagaagaaggaggatgtgaactgtga

SEQ ID NO: 112 - protein, 163 aa, synthetic construct
  source 1..163: note = cyno CD137 ECD 24-186 AA
LQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQ

SEQ ID NO: 113 - DNA, 489 bp, synthetic construct
  source 1..489: mol_type = other DNA; note = cyno CD137 ECD 24-186 DNA
ctgcaggatctgtgtagtaactgcccagctggtacattctgtgataataacaggagtcagatttgcagtccctgtcctccaaatagtttctccagcgcaggtggacaaaggacctgtgacatatgcaggcagtgtaaaggtgttttcaagaccaggaaggagtgttcctccaccagcaatgcagagtgtgactgcatttcaggatatcactgcctgggggcagaatgcagcatgtgtgaacaggattgtaaacaaggtcaagaattgacaaaaaaaggttgtaaagactgttgctttgggacatttaatgaccagaaacgtggcatctgtcgaccctggacaaactgttctttggatggaaagtctgtgcttgtgaatgggacgaaggagagggacgtggtctgcggaccatctccagccgacctctctccaggagcatcctctgcgaccccgcctgcccctgcgagagagccaggacactctccgcag

SEQ ID NO: 114 - protein, 396 aa, synthetic construct
  source 1..396: note = cyno CD137 ECD-mIgG2a AA
LQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 115 - DNA, 1188 bp, synthetic construct
  source 1..1188: mol_type = other DNA; note = cyno CD137 ECD-mIgG2a DNA
ctgcaggatctgtgtagtaactgcccagctggtacattctgtgataataacaggagtcagatttgcagtccctgtcctccaaatagtttctccagcgcaggtggacaaaggacctgtgacatatgcaggcagtgtaaaggtgttttcaagaccaggaaggagtgttcctccaccagcaatgcagagtgtgactgcatttcaggatatcactgcctgggggcagaatgcagcatgtgtgaacaggattgtaaacaaggtcaagaattgacaaaaaaaggttgtaaagactgttgctttgggacatttaatgaccagaaacgtggcatctgtcgaccctggacaaactgttctttggatggaaagtctgtgcttgtgaatgggacgaaggagagggacgtggtctgcggaccatctccagccgacctctctccaggagcatcctctgcgaccccgcctgcccctgcgagagagccaggacactctccgcaggagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 116 - protein, 277 aa, Homo sapiens
  source 1..277: note = human CD40 AA
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 117 - DNA, 834 bp, Homo sapiens
  source 1..834: mol_type = other DNA; note = human CD40 DNA
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtga

SEQ ID NO: 118 - protein, 194 aa, Homo sapiens
  source 1..194: note = huCD40 ECD AA (1-194)
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRA

SEQ ID NO: 119 - DNA, 582 bp, Homo sapiens
  source 1..582: mol_type = other DNA; note = huCD40 ECD DNA (1-194)
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagcc

SEQ ID NO: 120 - protein, 428 aa, Homo sapiens
  source 1..428: note = human CD40 ECD-mIgG2a AA
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRASEPRGPTIKPCPPCKCPAPNLLGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 121 - DNA, 1284 bp, Homo sapiens
  source 1..1284: mol_type = other DNA; note = human CD40 ECD-mIgG2a DNA
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgagttcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagctagcgagcccagagggcccacaatcaagccctgtcctccatgcaaatgcccagcacctaacctcttgggtggaccatccgtcttcatcttccctccaaagatcaaggatgtactcatgatctccctgagccccatggtcacatgtgtggtggtggatgtgagcgaggatgacccagatgtccagatcagctggttcgtgaacaacgtggaagtactcacagctcagacacaaacccatagagaggattacaacagtactctccgggtggtcagtgccctccccatccagcaccaggactggatgagtggcaaggagttcaaatgcaaggtcaacaacaaagccctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgctcagtggtccacgagggtctgcacaatcaccacacgactaagagcttctcccggactccgggtaaa

SEQ ID NO: 122 - protein, 277 aa, Homo sapiens
  source 1..277: note = human OX40 AA
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 123 - DNA, 834 bp, Homo sapiens
  source 1..834: mol_type = other DNA; note = human OX40 DNA
atgtgcgtgggggctcggcggctgggccgcgggccgtgtgcggctctgctcctcctgggcctggggctgagcaccgtgacggggctccactgtgtcggggacacctaccccagcaacgaccggtgctgccacgagtgcaggccaggcaacgggatggtgagccgctgcagccgctcccagaacacggtgtgccgtccgtgcgggccgggcttctacaacgacgtggtcagctccaagccgtgcaagccctgcacgtggtgtaacctcagaagtgggagtgagcggaagcagctgtgcacggccacacaggacacagtctgccgctgccgggcgggcacccagcccctggacagctacaagcctggagttgactgtgccccctgccctccagggcacttctccccaggcgacaaccaggcctgcaagccctggaccaactgcaccttggctgggaagcacaccctgcagccggccagcaatagctcggacgcaatctgtgaggacagggaccccccagccacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctccggagggaccagaggctgccccccgatgcccacaagccccctgggggaggcagtttccggacccccatccaagaggagcaggccgacgcccactccaccctggccaagatctga

SEQ ID NO: 124 - protein, 216 aa, Homo sapiens
  source 1..216: note = human OX40 ECD (1-216) AA
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVA

SEQ ID NO: 125 - DNA, 648 bp, Homo sapiens
  source 1..648: mol_type = other DNA; note = human OX40 ECD (1-216) DNA
atgtgcgtgggggctcggcggctgggccgcgggccgtgtgcggctctgctcctcctgggcctggggctgagcaccgtgacggggctccactgtgtcggggacacctaccccagcaacgaccggtgctgccacgagtgcaggccaggcaacgggatggtgagccgctgcagccgctcccagaacacggtgtgccgtccgtgcgggccgggcttctacaacgacgtggtcagctccaagccgtgcaagccctgcacgtggtgtaacctcagaagtgggagtgagcggaagcagctgtgcacggccacacaggacacagtctgccgctgccgggcgggcacccagcccctggacagctacaagcctggagttgactgtgccccctgccctccagggcacttctccccaggcgacaaccaggcctgcaagccctggaccaactgcaccttggctgggaagcacaccctgcagccggccagcaatagctcggacgcaatctgtgaggacagggaccccccagccacgcagccccaggagacccagggccccccggccaggcccatcactgtccagcccactgaagcctggcccagaacctcacagggaccctccacccggcccgtggaggtccccgggggccgtgcggttgcc

SEQ ID NO: 126 - protein, 222 aa, Homo sapiens
  source 1..222: note = human OX40 ECD-His AA
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAHHHHHH

SEQ ID NO: 127 - DNA, 666 bp, Homo sapiens
  source 1..666: mol_type = other DNA; note = human OX40 ECD-His DNA
atgtgcgtgggggctcggcggctgggccgcgggccgtgtgcggctctgctcctcctgggcctggggctgagcaccgtgacggggctccactgtgtcggggacacctaccccagcaacgaccggtgctgccacgagtgcaggccaggcaacgggatggtgagccgctgcagccgctcccagaacacggtgtgccgtccgtgcgggccgggcttctacaacgacgtggtcagctccaagccgtgcaagccctgcacgtggtgtaacctcagaagtgggagtgagcggaagcagctgtgcacggccacacaggacacagtctgccgctgccgggcgggcacccagcccctggacagctacaagcctggagttgactgtgccccctgccctccagggcacttctccccaggcgacaaccaggcctgcaagccctggaccaactgcaccttggctgggaagcacaccctgcagccggccagcaatagctcggacgcaatctgtgaggacagggaccccccagccacgcagccccaggagacccagggccccccggccaggcccatcactgtccagcccactgaagcctggcccagaacctcacagggaccctccacccggcccgtggaggtccccgggggccgtgcggttgcccatcaccatcaccatcac

SEQ ID NO: 128 - protein, 98 aa, Synthetic construct
  source 1..98: note = 3-23 germline AA
EVQLLESGGGLVQPGGSLRLSCAASGFTFSSYAISWVRQAPGKGLEWVSAISGSGGSTYYADSVKGRFTISRDNSKNTLYLQMNSLRAEDTAVYYCAR

SEQ ID NO: 129 - DNA, 294 bp, synthetic construct
  source 1..294: mol_type = other DNA; note = 3-23 germline DNA
gaagtgcagctgctggaaagcggcggcggcctggtgcagccgggcggcagcctgcgcctgagctgcgcggcgagcggctttacctttagcagctatgcgattagctgggtgcgccaggcgccgggcaaaggcctggaatgggtgagcgcgattagcggcagcggcggtagcacctattatgcggatagcgtgaaaggccgctttaccattagccgcgataacagcaaaaacaccctgtatctgcagatgaacagcctgcgcgcggaagataccgccgtgtattattgcgcgcgt

SEQ ID NO: 130 - protein, 354 aa, synthetic construct
  source 1..354: note = VH-Fc
EVQLLESGGGLVQPGGSLRLSCAASGFRLDTTEVGWVRQAPGKGLEWVSTIMGISGSTYYADSVKGRFTISRDNSKNTLYLQMNSLRAEDTAVYYCARVVDSLFDDSAVFDYWGQGTLVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - DNA, 1062 bp, synthetic construct
  source 1..1062: mol_type = other DNA; note = VH-Fc DNA
gaggtccagttacttgagagtggtggaggtctggtccaaccaggaggttcgctgcgtttatcctgcgccgcgtctggattcagattggacaccaccgaagtaggttgggtgcgtcaagcgccggggaaaggactggaatgggtctccaccatcatgggtattagtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtcgattccctctttgatgacagcgccgtttttgattactggggacagggcaccttagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 132 - protein, 353 aa, synthetic construct
  source 1..353: note = VH-Fc
EVQLLESGGGLVQPGGSLRLSCAASGFMLSAEDVGWVRQAPGKGLEWVSAILDFGGSTYYADSVKGRFTISRDNSKNTLYLQMNSLRAEDTAVYYCARVVYHAGGGVTFDYWGQGTLVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - DNA, 1059 bp, synthetic construct
  source 1..1059: mol_type = other DNA; note = VH-Fc DNA
gaggtccagttacttgagagtggtggaggtctggtccaaccaggaggttcgctgcgtttatcctgcgccgcgtctggattcatgttgtccgccgaagacgtgggttgggtgcgtcaagcgccggggaaaggactggaatgggtctccgccatcttggattttggtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtctaccatgctggtggtggcgtcacctttgattactggggacagggcaccttagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 134 - protein, 226 aa, synthetic construct
  source 1..226: note = A Fc-null version IgG1
DKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - DNA, 678 bp, synthetic construct
  source 1..678: mol_type = other DNA; note = A Fc-null version IgG1 DNA
gacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 136 - protein, 353 aa, synthetic construct
  source 1..353: note = BGA-4712-M3-mutFc
EVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYWGQGTLVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - DNA, 1059 bp, synthetic construct
  source 1..1059: mol_type = other DNA; note = BGA-4712-M3-mutFc
gaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattactggggacagggcaccttagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 138 - protein, 353 aa, synthetic construct
  source 1..353: note = BGA-7556-mutFc
EVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - DNA, 1059 bp, synthetic construct
  source 1..1059: mol_type = other DNA; note = BGA-7556-mutFc
gaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattaccggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 140 - protein, 107 aa, synthetic construct
  source 1..107: note = CH3
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - DNA, 321 bp, synthetic construct
  source 1..321: mol_type = other DNA; note = CH3
ggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 142 - protein, 234 aa, synthetic construct
  source 1..234: note = CH3- BGA-4712-M3
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYWGQGTLVTVSS

SEQ ID NO: 143 - DNA, 702 bp, synthetic construct
  source 1..702: mol_type = other DNA; note = CH3- BGA-4712-M3
ggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattactggggacagggcaccttagttacagtctcatcg

SEQ ID NO: 144 - protein, 122 aa, synthetic construct
  source 1..122: note = BGA-2690 AA
EVQLLESGGGLVQPGGSLRLSCAASGFTLSREDVGWVRQAPGKGLEWVSAILDFGSSTYYAESVKGRFTISRDNAKNTLYLQMSSLRAEDTAVYYCARVVYHAGGGVTYDYRGQGTQVTVSS

SEQ ID NO: 145 - DNA, 366 bp, synthetic construct
  source 1..366: mol_type = other DNA; note = BGA-2690 DNA
gaggtccagttacttgagagtggtggaggtctggtccaaccaggaggttcgctgcgtttatcctgcgccgcgtctggattcacgctttccagagaagacgtgggttgggtgcgtcaagcgccggggaaaggactggaatgggtctccgccatcttggatttcggttcttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcg

SEQ ID NO: 146 - protein, 122 aa, synthetic construct
  source 1..122: note = BGA-3849 AA
EVQLLESGGGLVQPGGSLRLSCAASGFTLSHEDVGWVRQAPGKGLEWVSAILDSGGSTYYAESVKGRFTISRDNAKNTLYLQMSSLRAEDTAVYYCARYVYHAGGGVTFDTRGQGTQVTVSS

SEQ ID NO: 147 - DNA, 366 bp, synthetic construct
  source 1..366: mol_type = other DNA; note = BGA-3849 DNA
gaggtccagttacttgagagtggtggaggtctggtccaaccaggaggttcgctgcgtttatcctgcgccgcgtctggattcacgctttcccatgaagacgtgggttgggtgcgtcaagcgccggggaaaggactggaatgggtctccgccatcttggattccggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatactcggggacagggcacccaagttacagtctcatcg

SEQ ID NO: 148 - protein, 122 aa, synthetic construct
  source 1..122: note = BGA-7916 AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHAGGGVTYDYRGQGTQVTVSS

SEQ ID NO: 149 - DNA, 366 bp, synthetic construct
  source 1..366: mol_type = other DNA; note = BGA-7916 DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcg

SEQ ID NO: 150 - protein, 122 aa, synthetic construct
  source 1..122: note = BGA-4988 AA
EVQLLESGGGLVQPGGSLRLSCAASGFTVSAEDVGWVRQAPGKGLEWVSAILDRGGSTYYAESVKGRFTISRDNAKNTLYLQMSSLRAEDTAVYYCARVVYHAGGGVTYDYRGQGTQVTVSS

SEQ ID NO: 151 - DNA, 366 bp, synthetic construct
  source 1..366: mol_type = other DNA; note = BGA-4988 DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggatcggggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcg

SEQ ID NO: 152 - protein, 122 aa, synthetic construct
  source 1..122: note = "BGA-5164 AA"
EVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVVYHAGGGVTYDYRGQGTQVTVSS

SEQ ID NO: 153 - DNA, 366 bp, synthetic construct
  source 1..366: mol_type = other DNA; note = BGA-5164 DNA
gaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcg

SEQ ID NO: 154 - protein, 122 aa, synthetic construct
  source 1..122: note = BGA-3953 AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARIVYHAGGGVTFDTRGQGTQVTVSS

SEQ ID NO: 155 - DNA, 366 bp, synthetic construct
  source 1..366: mol_type = other DNA; note = BGA-3953 DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 156 - protein, 122 aa, synthetic construct
  source 1..122: note = BGA-5385 AA
EVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVIYHAGGGVTFDTRGQGTQVTVSS

SEQ ID NO: 157 - DNA, 366 bp, synthetic construct
  source 1..366: mol_type = other DNA; note = "BGA-5385 DNA"
gaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttatctaccatgctggtggtggcgtcacctttgatactcggggacagggcacccaagttacagtctcatcg

SEQ ID NO: 158 - protein, 122 aa, synthetic construct
  source 1..122: note = BGA-9468 AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHSGGGVTFDLRGQGTQVTVSS

SEQ ID NO: 159 - DNA, 366 bp, synthetic construct
  source 1..366: mol_type = other DNA; note = BGA-9468 DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccattctggtggtggcgtcacctttgatcttcggggacagggcacccaagttacagtctcatcg

SEQ ID NO: 160 - protein, 122 aa, synthetic construct
  source 1..122: note = BGA-3285 AA
EVQLLESGGGLVQPGGSLRLSCAASGFTISAEDVGWVRQAPGKGLEWVSAILDFGGSTYYAESVKGRFTISRDNAKNTLYLQMSSLRAEDTAVYYCARVVYHAGGGVTFDTRGQGTQVTVSS

SEQ ID NO: 161 - DNA, 366 bp, synthetic construct
  source 1..366: mol_type = other DNA; note = BGA-3285 DNA
gaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctttgatactcggggacagggcacccaagttacagtctcatcg

SEQ ID NO: 162 - protein, 122 aa, synthetic construct
  source 1..122: note = BGA-9442 AA
EVQLLESGGGLVQPGGSLRLSCAASGFTISAEDVGWVRQAPGKGLEWVSAILDSGGSTYYAESVKGRFTISRDNAKNTLYLQMSSLRAEDTAVYYCARYVYHAGGGVTFDLRGQGTQVTVSS

SEQ ID NO: 163 - DNA, 366 bp, synthetic construct
  source 1..366: mol_type = other DNA; note = BGA-9442 DNA
gaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattctggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatcttcggggacagggcacccaagttacagtctcatcg

SEQ ID NO: 164 - protein, 122 aa, synthetic construct
  source 1..122: note = BGA-7746 AA
EVQLLESGGGLVQPGGSLRLSCAASGFTVSAEDVGWVRQAPGKGLEWVSAILDFGGSTYYAESVKGRFTISRDNAKNTLYLQMSSLRAEDTAVYYCARVVYHAGGGVTFDTRGQGTQVTVSS

SEQ ID NO: 165 - DNA, 366 bp, synthetic construct
  source 1..366: mol_type = other DNA; note = BGA-7746 DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattttggtggttcgacatactatgcagaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgatacgcggggacagggcacccaagttacagtctcatcg

SEQ ID NO: 166 - protein, 122 aa, synthetic construct
  source 1..122: note = BGA-8425 AA
EVQLLESGGGLVQPGGSLRLSCAASGFTLSAEDVGWVRQAPGKGLEWVSAILDSGGSTYYAESVKGRFTISRDNAKNTLYLQMSSLRAEDTAVYYCARVVYHAGGGVTYDYRGQGTQVTVSS

SEQ ID NO: 167 - DNA, 366 bp, synthetic construct
  source 1..366: mol_type = other DNA; note = BGA-8425 DNA
gaggtccagttacttgagagtggtggaggtctggtccaaccaggaggttcgctgcgtttatcctgcgccgcgtctggattcacgctgtccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctatgattaccggggacagggcacccaagttacagtctcatcg

SEQ ID NO: 168 - protein, 122 aa, synthetic construct
  source 1..122: note = BGA-3386 AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTWGQGTLVTVSS

SEQ ID NO: 169 - DNA, 366 bp, synthetic construct
  source 1..366: mol_type = other DNA; note = BGA-3386 DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatacttggggacagggcaccttagttacagtctcatcg

SEQ ID NO: 170 - protein, 353 aa, synthetic construct
  source 1..353: note = BGA-5623-mutFc-AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - DNA, 1059 bp, synthetic construct
  source 1..1059: mol_type = other DNA; note = BGA-5623-mutFc DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 172 - protein, 353 aa, synthetic construct
  source 1..353: note = BGA-2690- mutFc AA
EVQLLESGGGLVQPGGSLRLSCAASGFTLSREDVGWVRQAPGKGLEWVSAILDFGSSTYYAESVKGRFTISRDNAKNTLYLQMSSLRAEDTAVYYCARVVYHAGGGVTYDYRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - DNA, 1059 bp, synthetic construct
  source 1..1059: mol_type = other DNA; note = BGA-2690 mutFc DNA
gaggtccagttacttgagagtggtggaggtctggtccaaccaggaggttcgctgcgtttatcctgcgccgcgtctggattcacgctttccagagaagacgtgggttgggtgcgtcaagcgccggggaaaggactggaatgggtctccgccatcttggatttcggttcttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 174 - protein, 353 aa, synthetic construct
  source 1..353: note = BGA-3849-mutFc AA
EVQLLESGGGLVQPGGSLRLSCAASGFTLSHEDVGWVRQAPGKGLEWVSAILDSGGSTYYAESVKGRFTISRDNAKNTLYLQMSSLRAEDTAVYYCARYVYHAGGGVTFDTRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - DNA, 1059 bp, synthetic construct
  source 1..1059: mol_type = other DNA; note = BGA-3849 mutFc DNA
gaggtccagttacttgagagtggtggaggtctggtccaaccaggaggttcgctgcgtttatcctgcgccgcgtctggattcacgctttcccatgaagacgtgggttgggtgcgtcaagcgccggggaaaggactggaatgggtctccgccatcttggattccggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatactcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 176 - protein, 353 aa, synthetic construct
  source 1..353: note = BGA-7916-mutFc AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHAGGGVTYDTRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - DNA, 1059 bp, synthetic construct
  source 1..1059: mol_type = other DNA; note = BGA-7916-mutFc DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 178 - protein, 353 aa, synthetic construct
  source 1..353: note = BGA-4988-mutFc AA
EVQLLESGGGLVQPGGSLRLSCAASGFTVSAEDVGWVRQAPGKGLEWVSAILDRGGSTYYAESVKGRFTISRDNAKNTLYLQMSSLRAEDTAVYYCARVVYHAGGGVTYDTRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - DNA, 1059 bp, synthetic construct
  source 1..1059: mol_type = other DNA; note = BGA-4988-mutFc DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggatcggggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 180 - protein, 353 aa, synthetic construct
  source 1..353: note = BGA-5164-mutFc AA
EVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVVYHAGGGVTYDYRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - DNA, 1059 bp, synthetic construct
  source 1..1059: mol_type = other DNA; note = BGA-5164-mutFc DNA
gaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 182 - protein, 353 aa, synthetic construct
  source 1..353: note = BGA-3953-mutFc AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARIVYHAGGGVTFDTRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - DNA, 1059 bp, synthetic construct
  source 1..1059: mol_type = other DNA; note = BGA-3953-mutFc DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 184 - protein, 353 aa, synthetic construct
  source 1..353: note = BGA-5385-mutFc AA
EVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVIYHAGGGVTYDYRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - DNA, 1059 bp, synthetic construct
  source 1..1059: mol_type = other DNA; note = BGA-5385-mutFc DNA
gaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttatctaccatgctggtggtggcgtcacctatgattatcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 186 - protein, 353 aa, synthetic construct
  source 1..353: note = BGA-9468-mutFc AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHSGGGVTFDLRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - DNA, 1059 bp, synthetic construct
  source 1..1059: mol_type = other DNA; note = BGA-9468-mutFc DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccattctggtggtggcgtcacctttgatcttcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 188 - protein, 353 aa, synthetic construct
  source 1..353: note = BGA-3285-mutFc AA
EVQLLESGGGLVQPGGSLRLSCAASGFTISAEDVGWVRQAPGKGLEWVSAILDFGGSTYYAESVKGRFTISRDNAKNTLYLQMSSLRAEDTAVYYCARVVYHAGGGVTFDTRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - DNA, 1059 bp, synthetic construct
  source 1..1059: mol_type = other DNA; note = BGA-3285-mutFc DNA
gaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctttgatactcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 190 - protein, 353 aa, synthetic construct
  source 1..353: note = BGA-9442-mutFc AA
EVQLLESGGGLVQPGGSLRLSCAASGFTISAEDVGWVRQAPGKGLEWVSAILDSGGSTYYAESVKGRFTISRDNAKNTLYLQMSSLRAEDTAVYYCARYVYHAGGGVTFDLRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - DNA, 1059 bp, synthetic construct
  source 1..1059: mol_type = other DNA; note = BGA-9442-mutFc DNA
gaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattctggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatcttcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 192 - protein, 353 aa, synthetic construct
  source 1..353: note = BGA-7746-mutFc AA
EVQLLESGGGLVQPGGSLRLSCAASGFTVSAEDVGWVRQAPGKGLEWVSAILDFGGSTYYAESVKGRFTISRDNAKNTLYLQMSSLRAEDTAVYYCARVVYHAGGGVTFDTRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - DNA, 1059 bp, synthetic construct
  source 1..1059: mol_type = other DNA; note = BGA-7746-mutFc DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattttggtggttcgacatactatgcagaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgatacgcggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 194 - protein, 353 aa, synthetic construct
  source 1..353: note = BGA-8425-mutFc AA
EVQLLESGGGLVQPGGSLRLSCAASGFTLSAEDVGWVRQAPGKGLEWVSAILDSGGSTYYAESVKGRFTISRDNAKNTLYLQMSSLRAEDTAVYYCARVVYHAGGGVTYDYRGQGTQVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - DNA, 1059 bp, synthetic construct
  source 1..1059: mol_type = other DNA; note = BGA-8425-mutFc DNA
gaggtccagttacttgagagtggtggaggtctggtccaaccaggaggttcgctgcgtttatcctgcgccgcgtctggattcacgctgtccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctatgattaccggggacagggcacccaagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 196 - protein, 353 aa, synthetic construct
  source 1..353: note = BGA-3386-mutFc AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTWGQGTLVTVSSGGGGSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - DNA, 1059 bp, synthetic construct
  source 1..1059: mol_type = other DNA; note = BGA-3386-mutFc DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatacttggggacagggcaccttagttacagtctcatcgggcggcggagggtctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 198 - protein, 354 aa, synthetic construct
  source 1..354: note = IgG1 AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - DNA, 1062 bp, synthetic construct
  source 1..1062: mol_type = other DNA; note = IgG1 DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcggagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 200 - protein, 350 aa, synthetic construct
  source 1..350: note = IgG4 AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 201 - DNA, 1050 bp, synthetic construct
  source 1..1050: mol_type = other DNA; note = IgG4 DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcggaatccaagtatggtccgccatgtccaccgtgcccggcacccgagtttttgggagggcctagtgtgtttttgtttcctcctaagccaaaggacacgcttatgattagtcgcacacccgaggttacctgtgtagtggtggacgtatcacaagaggatccggaagtccaatttaattggtacgtcgacggagtggaagtccacaatgcaaagaccaagcctagggaagaacagttcaattctacctatcgctggtctgtactcactgtactccaccaggattggctcaatggaaaggaatacaaatgcaaggtttccaacaaagggctcccctcttccattgagaaaactattagcaaggctaaaggccaacccagagaaccacaggtttatacccttccaccttcccaggaggagatgacgaagaatcaggttagtcttacgtgcctcgttaaaggattctacccttcagatatcgcagttgaatgggagtctaacgggcaacccgagaacaactacaagaccaccccgccggtgctggactcagatggtagtttctttctttacagtcggcttaccgtcgacaagtcacgctggcaggagggaaatgtattctcctgcagcgtaatgcacgaagctctgcacaatcactatacccagaagtcactgagcctttctctgggtaag

SEQ ID NO: 202 - protein, 447 aa, synthetic construct
  source 1..447: note = Heavy chain AA
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 203 - DNA, 1341 bp, synthetic construct
  source 1..1341: mol_type = other DNA; note = Heavy chain DNA
caagttcaacttcagcagtggggagcgggtctcttgaagcctagtgagacgctgagcctgacctgtgcagtctacgggggttcattttctgggtactactggtcctggatacgccaaagcccagagaaaggcctggaatggattggggagataaatcacgggggttatgtgacatataatccttctcttgagagccgggtcacgatatctgttgacacaagtaaaaatcagttctccctcaaactgagctccgttaccgcagcagacactgccgtttactactgtgcgcgggactatgggccaggtaattacgattggtacttcgacctgtggggtagaggaacgctggtcactgtaagcagtgcatctacaaagggccccagcgtgtttccccttgcaccatgttcacggagcactagtgaaagcactgccgcgctcgggtgtctcgttaaggattattttccggagcctgtcactgtctcttggaactctggtgcactgacatcaggcgttcacacgttccctgcagtactccagagctcaggattgtacagcctgtccagtgtagttacagtgccttcctcctcccttggaacaaagacctacacctgcaacgtggaccataagcccagcaatacgaaagtagacaaacgagtcgaatccaagtatggtccgccatgtccaccgtgcccggcacccgagtttttgggagggcctagtgtgtttttgtttcctcctaagccaaaggacacgcttatgattagtcgcacacccgaggttacctgtgtagtggtggacgtatcacaagaggatccggaagtccaatttaattggtacgtcgacggagtggaagtccacaatgcaaagaccaagcctagggaagaacagttcaattctacctatcgctggtctgtactcactgtactccaccaggattggctcaatggaaaggaatacaaatgcaaggtttccaacaaagggctcccctcttccattgagaaaactattagcaaggctaaaggccaacccagagaaccacaggtttatacccttccaccttcccaggaggagatgacgaagaatcaggttagtcttacgtgcctcgttaaaggattctacccttcagatatcgcagttgaatgggagtctaacgggcaacccgagaacaactacaagaccaccccgccggtgctggactcagatggtagtttctttctttacagtcggcttaccgtcgacaagtcacgctggcaggagggaaatgtattctcctgcagcgtaatgcacgaagctctgcacaatcactatacccagaagtcactgagcctttctctgggtaag

SEQ ID NO: 204 - protein, 216 aa, synthetic construct
  source 1..216: note = Light chain AA
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - DNA, 648 bp, synthetic construct
  source 1..648: mol_type = other DNA; note = Light chain DNA
gaaatcgtgctgacccaatcacctgcgaccctctcccttagccctggagaacgagcaactctcagttgcagagcaagccagagcgtcagctcatatctcgcatggtatcaacaaaaaccaggccaagctcctagactgttgatttatgatgcctctaaccgggcgactggcataccagcccgctttagcgggtctgggagtggcactgattttacgctcacgatttcatctttggagccagaagatttcgctgtatactactgtcaacaaaggtcaaactggccgcctgcacttacgttcggcgggggtaccaaggtggagattaagaggacagttgcggcgccatcagtttttatattcccaccctccgatgagcagttgaaaagtggcacggcatcagttgtctgtctgctgaacaatttctatccacgggaggcgaaagtccaatggaaagtggacaatgcgcttcagagcgggaactctcaggagagtgtcactgaacaggattcaaaggactccacgtactcactctcatccactctcaccctttcaaaggcggattacgaaaagcataaggtctacgcttgtgaggtaacacatcaggggctttccagtcctgtaaccaagagcttcaatcgcggcgaatgc

SEQ ID NO: 206 - protein, 442 aa, synthetic construct
  source 1..442: note = Heavy chain AA
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 207 - DNA, 1326 bp, synthetic construct
  source 1..1326: mol_type = other DNA; note = Heavy chain DNA
gaagttcaactggtacagtccggtgctgaagttaaaaaacccggagagagtctgagaatctcttgcaaggggtctgggtactcttttagtacatattggatatcttgggtgagacaaatgccggggaaaggcctcgaatggatgggcaagatttatcccggagacagctatacaaattactcacctagttttcaaggtcaggtaactatcagcgcggacaaatccataagtacggcctatctgcaatggtcttccttgaaggcatccgacacggcgatgtattactgcgcaagagggtatggtatattcgattactgggggcagggcactctcgttaccgtgagcagcgcttcaacaaaaggcccatctgtcttccctctcgcgccttgctcccgctctacttcagagtcaacagctgctctggggtgcttggtgaaggattactttcctgaaccggttactgtcagctggaattctggggctttgacgagcggagttcacactttcccagctgtactgcaaagttctggactttacagcttgagctccgtggtgacagtgccgagctcaaacttcggtacgcagacatatacatgcaatgtggatcataaacccagcaatacgaaagtagataagacagtcgaaaggaagtgttgtgtagagtgcccaccgtgccccgcccctccggttgcaggcccatcagtgtttctcttcccgcccaaacctaaggacacgctgatgatttcacgaacgcccgaggtcacctgtgtcgtagtcgatgtgtcccatgaggacccagaagttcagttcaattggtatgtagacggcgtggaggtacacaatgcaaaaacgaaaccacgcgaggaacagttcaatagtacgttccgggtcgtgagtgtgctcacggtggttcaccaagactggctgaacgggaaagaatataagtgcaaagtatccaataaaggcctgcccgcacccatcgaaaaaactataagtaaaactaaagggcaaccgcgagagcctcaggtctatactttgccaccgtcacgcgaagagatgaccaagaaccaagtaagtcttacttgcttggtcaaggggttctacccctcagatatagcggtggagtgggaatccaatggccaacccgagaacaattacaagacaacaccaccaatgctggactctgacgggagtttctttctgtatagtaaacttaccgtcgataaatcacgctggcagcaaggcaacgtattctcatgctcagtaatgcatgaagccttgcataatcactacactcagaaaagcctgtccctctctccaggtaag

SEQ ID NO: 208 - protein, 214 aa, synthetic construct
  source 1..214: note = Light chain AA
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 209 - DNA, 642 bp, synthetic construct
  source 1..642: mol_type = other DNA; note = Light chain DNA
agctacgagttgactcagccaccatccgtcagtgttagccccggacagacggcgtcaattacttgtagtggagacaacattggcgatcagtacgcccattggtatcagcaaaaaccgggtcaatcaccagttctggtgatatatcaggataagaatcgaccatcagggattccagagaggttctcaggtagtaactcaggtaacacagctactctgaccataagtggtacccaggctatggacgaagcagactactactgcgccacctacacaggcttcggatcacttgcggttttcgggggcggaaccaaactaaccgttcttgggcaacctaaggctgccccgtccgtgactctttttcctccgtcttccgaagagcttcaagccaataaagcaaccttggtatgtttgatttccgatttttaccctggagcggtgacggtggcatggaaagctgattcaagccctgtgaaagctggggtggaaactactacgccgtccaaacagagcaataataagtacgcggcctccagctatttgtcacttaccccggagcaatggaagtcacaccgaagttacagttgtcaagttactcacgaaggcagcactgttgaaaagaccgtagcacccaccgaatgctca

SEQ ID NO: 210 - protein, 118 aa, synthetic construct
  source 1..118: note = Tislelizumab VH
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 211 - protein, 107 aa, synthetic construct
  source 1..107: note = Tislelizumab VL
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 212 - protein, 580 aa, Homo sapiens
  source 1..580: note = human GPC3 FL AA
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 213 - DNA, 1743 bp, Homo sapiens
  source 1..1743: mol_type = other DNA; note = human GPC3 FL DNA
atggccgggaccgtgcgcaccgcgtgcttggtggtggcgatgctgctcagcttggacttcccgggacaggcgcagcccccgccgccgccgccggacgccacctgtcaccaagtccgctccttcttccagagactgcagcccggactcaagtgggtgccagaaactcccgtgccaggatcagatttgcaagtatgtctccctaagggcccaacatgctgctcaagaaagatggaagaaaaataccaactaacagcacgattgaacatggaacagctgcttcagtctgcaagtatggagctcaagttcttaattattcagaatgctgcggttttccaagaggcctttgaaattgttgttcgccatgccaagaactacaccaatgccatgttcaagaacaactacccaagcctgactccacaagcttttgagtttgtgggtgaatttttcacagatgtgtctctctacatcttgggttctgacatcaatgtagatgacatggtcaatgaattgtttgacagcctgtttccagtcatctatacccagctaatgaacccaggcctgcctgattcagccttggacatcaatgagtgcctccgaggagcaagacgtgacctgaaagtatttgggaatttccccaagcttattatgacccaggtttccaagtcactgcaagtcactaggatcttccttcaggctctgaatcttggaattgaagtgatcaacacaactgatcacctgaagttcagtaaggactgtggccgaatgctcaccagaatgtggtactgctcttactgccagggactgatgatggttaaaccctgtggcggttactgcaatgtggtcatgcaaggctgtatggcaggtgtggtggagattgacaagtactggagagaatacattctgtcccttgaagaacttgtgaatggcatgtacagaatctatgacatggagaacgtactgcttggtctcttttcaacaatccatgattctatccagtatgtccagaagaatgcaggaaagctgaccaccactattggcaagttatgtgcccattctcaacaacgccaatatagatctgcttattatcctgaagatctctttattgacaagaaagtattaaaagttgctcatgtagaacatgaagaaaccttatccagccgaagaagggaactaattcagaagttgaagtctttcatcagcttctatagtgctttgcctggctacatctgcagccatagccctgtggcggaaaacgacaccctttgctggaatggacaagaactcgtggagagatacagccaaaaggcagcaaggaatggaatgaaaaaccagttcaatctccatgagctgaaaatgaagggccctgagccagtggtcagtcaaattattgacaaactgaagcacattaaccagctcctgagaaccatgtctatgcccaaaggtagagttctggataaaaacctggatgaggaagggtttgaaagtggagactgcggtgatgatgaagatgagtgcattggaggctctggtgatggaatgataaaagtgaagaatcagctccgcttccttgcagaactggcctatgatctggatgtggatgatgcgcctggaaacagtcagcaggcaactccgaaggacaacgagataagcacctttcacaacctcgggaacgttcattccccgctgaagcttctcaccagcatggccatctcggtggtgtgcttcttcttcctggtgcactga

SEQ ID NO: 214 - protein, 27 aa, Homo sapiens
  source 1..27: note = human GPC3 AA 537-563 (GK28b)
GNSQQATPKDNEISTFHNLGNVHSPLK

SEQ ID NO: 215 - protein, 50 aa, Homo sapiens
  source 1..50: note = human GPC3 AA 511-560 (DS50b)
DGMIKVKNQLRFLAELAYDLDVDDAPGNSQQATPKDNEISTFHNLGNVHS

SEQ ID NO: 216 - protein, 535 aa, Homo sapiens
  source 1..535: note = human GPC3 AA 25-559
QPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVH

(19) United States
(12) Patent Application Publication
(10) Pub. No.: US 2024/0209113 A1
(43) Pub. Date: Jun. 27, 2024

(54) ANTI-GPC3 AND ANTI-CD137 MULTISPECIFIC ANTIBODIES AND METHODS OF USE

(71) Applicant: BeiGene Switzerland GmbH, Basel (CH)

(72) Inventors: Dan LI, Beijing (CN); Xi YUAN, Beijing (CN); Jie LI, Beijing (CN); Yuanyuan XIE, Beijing (CN); Zhuo LI, Beijing (CN); Liang QU, Beijing (CN); Tong ZHANG, Beijing (CN); Jian SUN, Beijing (CN); Xuehui LI, Beijing (CN); Jing SONG, Beijing (CN); Xiaomin SONG, Beijing (CN)

(73) Assignee: BeiGene Switzerland GmbH, Basel (CH)

(21) Appl. No.: 18/511,764

(22) Filed: Nov. 16, 2023

Related U.S. Application Data
(63) Continuation of application No. PCT/CN22/93567, filed on May 18, 2022.

(30) Foreign Application Priority Data
May 21, 2021 (WO) PCT/CN2021/095113

(51) Int. Cl. C07K 16/30; A61K 39/00; A61P 35/00; C07K 16/28

(52) U.S. Cl. CPC C07K 16/303; A61P 35/00; C07K 16/2878; A61K 39/00; A61K 2039/505; C07K 2317/21; C07K 2317/24; C07K 2317/31; C07K 2317/622

(57) ABSTRACT

Provided are multispecific antibodies and antigen-binding fragments thereof that bind to human GPC3 and CD137, a pharmaceutical composition comprising said antibody, and use of the multispecific antibody or the composition for treating a disease, such as cancer.

### CROSS-REFERENCE TO RELATED APPLICATIONS

This application is a continuation of International Patent Application No. PCT/CN2022/093567, filed May 18, 2022, which claims priority from International Patent Application No. PCT/CN2021/095113, filed May 21, 2021. The contents of these applications are incorporated herein by reference in their entirety.

### SEQUENCE LISTING

The instant application contains a Sequence Listing which has been submitted electronically in XML format and is hereby incorporated by reference in its entirety. Said xml file was created on Jan. 10, 2024, is named 138881_0289_Sequence_Listing.xml, and is 285 bytes in size.

### FIELD OF THE DISCLOSURE

Disclosed herein are multispecific antibodies or antigen-binding fragments thereof that bind to human GPC3 and human CD137, a composition comprising said antibody, as well as methods of use for the treatment of cancer.

### BACKGROUND

Glypican-3 (GPC3) belongs to the heparan sulfate proteoglycan (HSPG) family, including a 60-70 kD core protein, which is linked to the surface of the cell membrane by a glycosylphosphatidylinositol anchor (GPI), and the carboxy terminus is modified with a heparan sulfate side chain (Filmus J et al., J. Clin. Inv. 2001; 108: 497-501).

The specific expression of GPC3 in tumor cells has received widespread attention. GPC3 is expressed in hepatocellular carcinoma (HCC), the most common type of liver cancer. Notably, its expression is not detected in non-malignant tissues. The overexpression of GPC3 has also been reported in hepatoblastoma, lung squamous cell carcinoma (LSCC), and other cancers. It is indicated that GPC3 is suitable for targeted therapy as a tumor antigen. (Li N et al., Trends Cancer. 2018; 4: 741-54; Ho M, et al., Eur J Cancer. 2011; 47: 333-8; Moek et al., Am. J. Pathol. 2018; 188 (9): 1973-1981).

CD137 (also known asTNFRSF9/41BB) is a co-stimulatory molecule belonging to the TNFRSF family. It was discovered by T-cell-factor-screening on mouse helper and cytotoxic cells stimulated by concanavalin A and was identified in 1989 as an inducible gene that was expressed on antigen-primed T cells but not on resting ones (Kwon et al., Proc. Natl. Acad. Sci. USA. 1989; 86:1963-1967). It was discovered in the late 80s during T-cell-factor-screening on mouse helper and cytotoxic cells stimulated by concanavalin A. In addition, it is known to be expressed in dendritic (DCs), natural killer cells (NKs) (Vinay et al., Mol. Cancer Ther. 2012; 11:1062-1070), activated CD4+ and CD8+ T lymphocytes, eosinophils, natural killer T cells (NKTs), and mast cells (Kwon et al., 1989 supra; Vinay D., Int. J. Hematol. 2006; 83:23-28).

The anti-CD137 antibodies Urelumab (BMS-663513) which binds to CRD I of CD137 and Utomilumab (PF-05082566) which binds to CRDs III and IV of CD137 show potential as cancer therapeutics for their ability to activate cytotoxic T cells and to increase the production of interferon gamma (IFN-γ). The mechanisms underlying tumor regression by these antibodies are the effects on the immune cells response to cancer cells. Anti-CD137 antibody stimulates and activates effector T lymphocytes (e.g., stimulating CD8 T lymphocytes to produce INFγ), NKTs, and APCs (e.g., macrophages).

Urelumab demonstrated promising results in preclinical experiments and early clinical studies (Sznol et al., Clin. Oncol. 2008; 26(Suppl. 15)). However, in later studies, Urelumab demonstrated liver toxicity resulting in pausing development of the antibody until February 2012 (Segal et al., Clin. Cancer Res. 2017; 23:1929-1936). The liver toxicity was mostly due to S100A4 protein secreted by tumor and stromal cells, and studies that dose limited Urelumab to 8 mg or 0.1 mg/kg per patient for every 3 weeks has restored interest in this antibody (Segal et al., Clin. Cancer Res. 2017; 23:1929-1936).

In contrast with Urelumab, Utomilumab showed a better safety profile and initial studies show no liver toxicity or other dose limiting factors (Segal et al., J. Clin. Oncol. 2014; 32(Suppl. 15)). The reported outcomes from a phase I trial of Utomulumab as monotherapy indicated a good safety profile (Segal et al., Clin. Cancer Res. 2018; 24:1816-1823). The difference between the two antibodies has been speculated to be due to their different binding sites on the CD137 receptor.

Given the unique biology of both targets, anti-GPC3×CD137 multispecific antibodies that recruit immune cells to GPC3 expressing cancers would be useful in the treatment of cancer.

### SUMMARY OF THE DISCLOSURE

The present disclosure is directed to multispecific anti-GPC3×CD137 antibodies and antigen-binding fragments thereof. The present disclosure encompasses the following embodiments.

A multispecific antibody or antigen-binding fragment thereof, comprising a first antigen binding domain that specifically binds to human Glypican 3 (GPC3) and a second antigen binding domain that specifically binds to human CD137.

The multispecific antibody or antigen-binding fragment thereof, wherein the second antigen binding domain that specifically binds to human CD137 comprises:
(i) a HCDR1 (Heavy Chain Complementarity Determining Region 1), a HCDR2 and a HCDR3 from the heavy chain variable region (VH) set forth in SEQ ID NO: 84;
(ii) a HCDR1, a HCDR2 and a HCDR3 from the heavy chain variable region (VH) set forth in SEQ ID NO: 75;
(iii) a HCDR1, a HCDR2 and a HCDR3 from the heavy chain variable region (VH) set forth in SEQ ID NO: 70; or
(iv) a HCDR1, a HCDR2 and a HCDR3 from the heavy chain variable region (VH) set forth in SEQ ID NO: 60.

The multispecific antibody or antigen-binding fragment thereof, wherein the second antigen binding domain that specifically binds to human CD137 comprises:
(i) a heavy chain variable region (VH) that comprises (a) a HCDR1 of SEQ ID NO: 65, (b) a HCDR2 of SEQ ID NO: 80, (c) a HCDR3 of SEQ ID NO: 81;
(ii) a heavy chain variable region (VH) that comprises (a) a HCDR1 of SEQ ID NO: 65, (b) a HCDR2 of SEQ ID NO: 73, (c) a HCDR3 of SEQ ID NO: 67;
(iii) a heavy chain variable region (VH) that comprises (a) a HCDR1 of SEQ ID NO: 65, (b) a HCDR2 of SEQ ID NO: 66, (c) a HCDR3 of SEQ ID NO: 67; or
(iv) a heavy chain variable region (VH) that comprises (a) a HCDR1 of SEQ ID NO: 55, (b) a HCDR2 of SEQ ID NO: 56, (c) a HCDR3 of SEQ ID NO: 57, according to the Kabat numbering.

The multispecific antibody or antigen-binding fragment thereof, wherein the second antigen binding domain that specifically binds to human CD137 comprises:
(i) a heavy chain variable region (VH) comprising an amino acid sequence at least 90, 91, 92, 93, 94, 95, 96, 97, 98 or 99% identical to SEQ ID NO: 84;
(ii) a heavy chain variable region (VH) comprising an amino acid sequence at least 90, 91, 92, 93, 94, 95, 96, 97, 98 or 99% identical to SEQ ID NO: 86;
(iii) a heavy chain variable region (VH) comprising an amino acid sequence at least 90, 91, 92, 93, 94, 95, 96, 97, 98 or 99% identical to SEQ ID NO: 75;
(iv) a heavy chain variable region (VH) comprising an amino acid sequence at least 90, 91, 92, 93, 94, 95, 96, 97, 98 or 99% identical to SEQ ID NO: 70; or
(v) a heavy chain variable region (VH) comprising an amino acid sequence at least 90, 91, 92, 93, 94, 95, 96, 97, 98 or 99% identical to SEQ ID NO: 60.

The multispecific antibody or antigen-binding fragment thereof, wherein one, two, three, four, five, six, seven, eight, nine, or ten amino acids within SEQ ID NO: 84, SEQ ID NO: 86, SEQ ID NO: 75, SEQ ID NO: 70, or SEQ ID NO: 60 have been inserted, deleted or substituted.

The multispecific antibody or antigen-binding fragment thereof, wherein the second antigen binding domain that specifically binds to human CD137 comprises:
(i) a heavy chain variable region (VH) that comprises SEQ ID NO: 84;
(ii) a heavy chain variable region (VH) that comprises SEQ ID NO: 86;
(iii) a heavy chain variable region (VH) that comprises SEQ ID NO: 75;
(iv) a heavy chain variable region (VH) that comprises SEQ ID NO: 70; or
(v) a heavy chain variable region (VH) that comprises SEQ ID NO: 60.

The multispecific antibody or antigen-binding fragment thereof, wherein the second antigen binding domain that specifically binds to human CD137 binds to an epitope of human CD137 comprising amino acids F36, P47 and P49.

The multispecific antibody or antigen-binding fragment thereof, wherein the first antigen binding domain that specifically binds to human GPC3 comprises:
a HCDR1, a HCDR2 and a HCDR3 from the heavy chain variable region (VH) set forth in SEQ ID NO: 5; and
a LCDR1, a LCDR2 and a LCDR3 from the light chain variable region (VL) set forth in SEQ ID NO: 7.

The multispecific antibody or antigen-binding fragment thereof, wherein the first antigen binding domain that specifically binds to human GPC3 comprises:
a heavy chain variable region (VH) that comprises (a) a HCDR1 of SEQ ID NO: 10, (b) a HCDR2 of SEQ ID NO: 11, (c) a HCDR3 of SEQ ID NO: 12; and
a light chain variable region (VL) that comprises (d) a LCDR1 of SEQ ID NO: 13, (e) a LCDR2 of SEQ ID NO: 14, (f) a LCDR3 of SEQ ID NO: 15, according to the Kabat numbering.

The multispecific antibody or antigen-binding fragment thereof, wherein the first antigen binding domain that specifically binds to human GPC3 comprises:
a heavy chain variable region (VH) comprising an amino acid sequence at least 90, 91, 92, 93, 94, 95, 96, 97, 98 or 99% identical to SEQ ID NO: 5, and a light chain variable region (VL) comprising an amino acid sequence at least 90, 91, 92, 93, 94, 95, 96, 97, 98 or 99% identical to SEQ ID NO: 7.

The multispecific antibody or antigen-binding fragment thereof, wherein one, two, three, four, five, six, seven, eight, nine, or ten amino acids within SEQ ID NO: 5 or SEQ ID NO: 7 have been inserted, deleted or substituted.

The multispecific antibody or antigen-binding fragment thereof, wherein the first antigen binding domain that specifically binds to human GPC3 comprises:
a heavy chain variable region (VH) that comprises SEQ ID NO: 5, and a light chain variable region (VL) that comprises SEQ ID NO: 7.

The multispecific antibody or antigen-binding fragment thereof,
wherein the first antigen binding domain that specifically binds to human GPC3 comprises:
a heavy chain variable region (VH) that comprises (a) a HCDR1 of SEQ ID NO: 10, (b) a HCDR2 of SEQ ID NO: 11, (c) a HCDR3 of SEQ ID NO: 12; and
a light chain variable region (VL) that comprises (d) a LCDR1 of SEQ ID NO: 13, (e) a LCDR2 of SEQ ID NO: 14, (f) a LCDR3 of SEQ ID NO: 15,
according to the Kabat numbering; and
wherein the second antigen binding domain that specifically binds to human CD137 comprises:
(i) a heavy chain variable region (VH) that comprises (a) a HCDR1 of SEQ ID NO: 65, (b) a HCDR2 of SEQ ID NO: 80, (c) a HCDR3 of SEQ ID NO: 81;
(ii) a heavy chain variable region (VH) that comprises (a) a HCDR1 of SEQ ID NO: 65, (b) a HCDR2 of SEQ ID NO: 73, (c) a HCDR3 of SEQ ID NO: 67;
(iii) a heavy chain variable region (VH) that comprises (a) a HCDR1 of SEQ ID NO: 65, (b) a HCDR2 of SEQ ID NO: 66, (c) a HCDR3 of SEQ ID NO: 67; or
(iv) a heavy chain variable region (VH) that comprises (a) a HCDR1 of SEQ ID NO: 55, (b) a HCDR2 of SEQ ID NO: 56, (c) a HCDR3 of SEQ ID NO: 57,
according to the Kabat numbering.

The multispecific antibody or antigen-binding fragment thereof,
wherein the first antigen binding domain that specifically binds to human GPC3 comprises:
a heavy chain variable region (VH) that comprises SEQ ID NO: 5, and a light chain variable region (VL) that comprises SEQ ID NO: 7; and
wherein the second antigen binding domain that specifically binds to human CD137 comprises:
(i) a heavy chain variable region (VH) that comprises SEQ ID NO: 84;
(ii) a heavy chain variable region (VH) that comprises SEQ ID NO: 86;
(iii) a heavy chain variable region (VH) that comprises SEQ ID NO: 75;
(iv) a heavy chain variable region (VH) that comprises SEQ ID NO: 70; or
(v) a heavy chain variable region (VH) that comprises SEQ ID NO: 60.

The multispecific antibody or antigen-binding fragment thereof, wherein the multispecific antibody or antigen-binding fragment is a monoclonal antibody, a chimeric antibody, a humanized antibody, a human engineered antibody, a single chain antibody (scFv), a Fab fragment, a Fab′ fragment, or a F(ab′)₂ fragment.

The multispecific antibody or antigen-binding fragment thereof, wherein the first antigen binding domain that specifically binds to human GPC3 is a monoclonal antibody, a chimeric antibody, a humanized antibody, a human engineered antibody, a single chain antibody (scFv), a single domain antibody, a Fab fragment, a Fab′ fragment, or a F(ab′)₂ fragment, and the second antigen binding domain that specifically binds to human CD137 is a monoclonal antibody, a chimeric antibody, a humanized antibody, a human engineered antibody, a single chain antibody (scFv), a single domain antibody, a Fab fragment, a Fab′ fragment, or a F(ab′)₂ fragment.

The multispecific antibody or antigen-binding fragment thereof, wherein the multispecific antibody or antigen-binding fragment thereof is a bispecific antibody.

The multispecific antibody or antigen-binding fragment thereof, wherein the multiple specific antibody or antigen-binding fragment contains a linker from SEQ ID NO:16 to SEQ ID NO: 51 and SEQ ID NO: 88 to SEQ ID NO: 93.

The multispecific antibody or antigen-binding fragment thereof of, wherein the linker is SEQ ID NO: 23.

The multispecific antibody or antigen-binding fragment thereof, wherein the linker is SEQ ID NO: 28.

The multispecific antibody or antigen-binding fragment thereof, wherein the multispecific antibody or antigen-binding fragment comprises a heavy chain constant region of the subclass of IgG1, IgG2, IgG3, or IgG4, and/or a light chain constant region of the type of kappa or lambda, and wherein the heavy chain constant region comprises CH1 and/or Fc domain.

The multispecific antibody or antigen-binding fragment thereof, wherein the multispecific antibody or antigen-binding fragment thereof has antibody dependent cellular cytotoxicity (ADCC) or complement dependent cytotoxicity (CDC).

The multispecific antibody or antigen-binding fragment thereof, wherein the multispecific antibody or antigen-binding fragment thereof has reduced glycosylation or no glycosylation or is hypofucosylated.

The multispecific antibody or antigen-binding fragment thereof, wherein the multiple specific antibody or antigen-binding fragment thereof comprises increased bisecting GlcNac structures.

The multispecific antibody or antigen-binding fragment thereof, wherein the multispecific antibody or antigen-binding fragment thereof comprises a Fc domain, and wherein the Fc domain is an IgG1 with reduced effector function, optionally the Fc domain comprises an amino acid sequence of SEQ ID NO: 9.

The multispecific antibody or antigen-binding fragment thereof, wherein the multispecific antibody or antigen-binding fragment thereof comprises a Fc domain, and wherein the Fc domain is an IgG4.

The multispecific antibody or antigen-binding fragment thereof, which comprises:
a) a first polypeptide comprising from N terminal to C terminal:
a first heavy chain variable region, optionally one first heavy chain variable region;
a CH1 domain,
a Fc domain, and
a second heavy chain variable region, optionally one second heavy chain variable region;
optionally, C terminal of the Fc domain is linked to N terminal of the second heavy chain variable region via a linker; and
b) a second polypeptide comprising from N terminal to C terminal:
a first light chain variable region, optionally one first light chain variable region; and
a first light chain constant region;
wherein the first heavy chain variable region and the first light chain variable region form the first antigen binding domain that specifically binds to human GPC3, and the second heavy chain variable region forms the second antigen binding domain that specifically binds to human CD137.

The multispecific antibody or antigen-binding fragment thereof, wherein:
a) the heavy chain variable region (VH) of the first antigen binding domain that specifically binds to human GPC3, a CH1 domain, the Fc domain, and the heavy chain variable region (VH) of the second antigen binding domain that specifically binds to human CD137 are arranged in a first polypeptide in the direction of N terminal to C terminal;
optionally, C terminal of the Fc domain is linked to N terminal of the heavy chain variable region (VH) of the second antigen binding domain via a linker; and
b) the light chain variable region (VH) of the first antigen binding domain that specifically binds to human GPC3 and a first light chain constant region are arranged in a second polypeptide in the direction of N terminal to C terminal.

The multispecific antibody or antigen-binding fragment thereof, wherein the multispecific antibody or antigen-binding fragment is BE-830 comprising the first polypeptide of SEQ ID NO: 1 and the second polypeptide of SEQ ID NO: 3.

A pharmaceutical composition comprising the multispecific antibody or antigen-binding fragment thereof of the present disclosure and a pharmaceutically acceptable carrier.

A method of treating a cancer expressing GPC3 comprising administering to a patient in need an effective amount of the multispecific antibody or antigen-binding fragment thereof, or the pharmaceutical composition of the present disclosure.

The method, wherein the cancer is liver cancer, lung cancer, gastric cancer, germ cell tumors, thyroid cancer, pancreatic cancer, ovarian cancer, skin cancer, kidney cancer, atypical teratoid rhabdoid tumor of the brain, and undifferentiated synovial sarcoma.

The method, wherein the liver cancer is hepatoblastoma or hepatocellular carcinoma (HCC).

The method, wherein the lung cancer is non-small cell lung carcinoma (NSCLC) or small cell lung carcinomas (SCLC).

The method, wherein the non-small cell lung carcinoma is squamous non-small cell lung carcinoma.

The method, wherein the gastric cancer is AFP+ gastric cancer.

The method, wherein the kidney cancer is Wilms tumor.

The method, wherein the multispecific antibody or antigen-binding fragment thereof, or the pharmaceutical composition is administered in combination with another therapeutic agent.

The method, wherein the therapeutic agent is any one or more of paclitaxel or a paclitaxel agent, carboplatin, cisplatin, tislelizumab, bevacizumab, sorafenib, lenvatinib, afatinib, erlotinib, dacomitinib, gefitinib, osimertinib, ramucirumab, gemcitabine, trastuzumab, fluorouracil, capecitabine and oxaliplatin.

The method, wherein the therapeutic agent is a paclitaxel agent, carboplatin, cisplatin, bevacizumab, gemcitabine, fluorouracil, capecitabine or oxaliplatin.

The method, wherein the therapeutic agent an anti-PD1 or anti-PDL1 antibody. The method, wherein the anti-PD1 antibody is Tislelizumab.

An isolated nucleic acid that encodes the multispecific antibody or antigen-binding fragment thereof of the present disclosure.

A vector comprising the nucleic acid of the present disclosure.

A host cell comprising the nucleic acid or the vector of the present disclosure.

A process for producing a multispecific antibody or antigen-binding fragment thereof comprising cultivating the host cell of the present disclosure and recovering the antibody or antigen-binding fragment thereof from the culture.

The multispecific antibody or antigen-binding fragment thereof of the present disclosure has at least one or more of the following features:
(1) has specific binding to CD137, and does not bind to other TNF receptor family members;
(2) shows high affinity to both human CD137 and monkey CD137;
(3) has specific binding to GPC3, shows high affinity to both human GPC3 and monkey GPC3, and shows high affinity to wide range of GPC expression (low to high expression); (4) induces T cell activation including cytokine release (such as IFN-γ and IL-2) and T cell killing activity in a GPC3 dependent manner, and does not induce T cell activation or T cell killing activity in the absence of GPC3 expressing cells;
(5) induces T cell activation and potent T cell killing activity on wide range of GPC3 expressing (low, medium, high expression) cells, and the expression level of GPC3 does not significantly impact the potency;
(6) inhibits tumor growth effectively when it is administered alone;
(7) induces synergistic effects (e.g., tumor growth inhibition and/or tumor free ratio) when it is administered with anti-PD-1 antibody; and
(8) demonstrates good safety and minimal toxicity.

### BRIEF DESCRIPTION OF THE DRAWINGS

FIG. 1A is a summary of human anti-huCD137 VH domain antibodies identified from each sub-library. FIG. 1B is graphic phylogenetic trees of human anti-huCD137 VH domain antibodies from each sub-library. The VH sequences of candidate anti-huCD137 VH domain antibodies were aligned using DNASTAR's Megalign™ software. Sequence homology was displayed in phylogenetic trees.

FIG. 2A shows the schematic diagram of human Fc fusion VH antibody format (VH-Fc). VH domain antibodies were fused at the N terminal of an inert Fc (without FcγR-binding) with a GS4 linker in between. FIG. 2B shows a representative screening result using supernatants containing VH-Fc proteins, and FIG. 2C shows one of the clones, BGA-4712 was capable of stimulating IL-2 production in Hut78/huCD137 cells in a dose dependent manner.

FIG. 3A-3B is the binding profiles of a representative anti-huCD137 VH domain antibody BGA-4712. FIG. 3A depicts the determination of human anti-huCD137 VH domain antibody BGA-4712 binding by flow cytometry. FIG. 3B shows the blocking of human anti-huCD137 VH domain antibody BGA-4712 by huCD137 ligand (human CD137 ligand-ECD-mIgG2a fusion protein) interaction. The binding of purified human anti-huCD137 VH domain antibody BGA-4712 to CD137-expressing Hut78/huCD137 cells (Hut78/huCD137) was determined by flow cytometry.

FIG. 4 shows the sequence of CDR regions of BGA-4712-M3 after four rounds of selections.

FIG. 5 is a binding assay of anti-huCD137 VH domain antibody BGA-5623 by flow cytometry, demonstrating that binding to CD137 was improved after affinity maturation.

FIG. 6 demonstrates no off-target binding of BGA-5623 on other TNF Receptor family members by ELISA.

FIG. 7A-7B shows the epitope mapping of human anti-huCD137 VH domain antibody BGA-5623. FIG. 7A is a representative screening result in a cell based binding assay. Expression of huCD137 mutants was monitored by Urelumab analog. FIG. 7B shows BGA-5623 binding of purified huCD137 mutants.

FIG. 8 demonstrates CD137 ligand competes with human anti-huCD137 VH domain antibody BGA-5623 via ELISA.

FIG. 9 shows partially competitive binding of VH (BGA-5623) against CD137L for CD137. The crystal structure of VH (BGA-5623)/CD137 was superposed with CD137L/CD137 complex (PDB: 6MGP) via CD137. The CD137, CD137L and VH are colored in black, white and grey, respectively.

FIG. 10 shows CDR3 of VH (BGA-5623) undergoes dramatically conformation change upon CD137 binding. The CD137 bound VH (BGA-5623) in black was superposed with apoVH (BGA-5623) in white.

FIG. 11 shows the atomic interactions on the binding surface of VH (BGA-5623)/CD137 complex. The binding interface between VH (BGA-5623) and CD137 identifies certain key residues of BGA-5623 (paratope residues) and CD137 (epitope residues). The CRD1 and 2 domains of CD137 are shown in grey cartoon covered with white transparent surface. The paratope residues is colored in black.

FIG. 12 is a schematic diagram of designed tumor-targeted GPC3×CD137 multispecific antibody format.

FIG. 13 shows antigen binding ELISA of BE-830.

FIG. 14 shows BE-830 binding to human CD137 by FACS.

FIG. 15 shows BE-830 binding to human GPC3 by FACS.

FIG. 16A-E shows that BE-830 has no off target binding by FACS.

FIG. 17A-C shows sensorgrams of BE-830 binding to CD137 of human (FIG. 17A), monkey (FIG. 17B), and mouse (FIG. 17C).

FIG. 18 A-C shows sensorgrams of BE-830 Binding to GPC3 of human (FIG. 18A), monkey (FIG. 18B), and mouse (FIG. 18C).

FIG. 19 shows the analysis of the critical epitopes in CD137 required for BE-830 binding.

FIG. 20A-C shows the ELISA-based epitope analysis of GPC3 binding to BE-830.

FIG. 21 shows BE-830 enhances T Cell activation using a cell based bioluminescent assay.

FIG. 22A-G shows ELISA based FcγRs binding analysis of BE-830.

FIG. 23 shows BE-830 C1q-binding activity by ELISA assay.

FIG. 24A-C demonstrates GPC3×CD137 multispecific antibody BE-830 induces the IL-2 and IFN-γ release from human PBMCs. FIG. 24A is a schematic diagram of CD137 activation via co-stimulating huPBMCs with BE-830 and OS8-expressing hepatocellular carcinoma (HCC) cell lines. FIG. 24B-C shows BE-830 induced dose-dependent cytokine release in PBMC in a GPC3 expression dependent manner. Expression level of GPC3 on target cells did not significantly impact the potency of BE-830. PBMCs from three donors were tested and results were shown as mean±SD of triplicates.

FIG. 25A-C demonstrates GPC3×CD137 multispecific antibody BE-830 induces T-cell killing activity of human PBMCs. FIG. 25A is a schematic diagram of CD137 activation via co-stimulating huPBMCs with BE-830 in combination with an EpCAM/CD3 bispecific T-cell engager (BiTE) which provides a first signal for T cell activation. FIG. 25B-C shows BE-830 dose-dependently enhanced T cell killing activity to GPC3 expressing cells but not to GPC3 negative cells. PBMCs from three donors were tested and results were shown as mean±SD of triplicates.

FIG. 26A-C shows the effects of BE-830 and BGB-A317 (Tislelizumab) on human T cell activation. FIG. 26A is a schematic diagram of PBMC co-cultured with GPC3 and PD-L1 expressing target cells in the presence of BGB-A317 (Tislelizumab) and BE-830.

FIG. 26B-C shows the combination of BGB-A317 (Tislelizumab) and BE-830 further enhanced IFN-γ production in PBMC co-cultured with GPC3 and PD-L1 expressing cells.

FIG. 27 shows the efficacy of BE-830 monotherapy in Hepa1-6/hGPC3 model in humanized CD137 knock-in Mice. BE-830 (0.1, 0.5, and 3.0 mg/kg, once weekly) effectively inhibited tumor growth.

FIG. 28 shows the efficacy of the combination of BE-830 and an anti-PD-1 antibody in H22/hGPC3 model in humanized CD137 knock-in mice.

FIG. 29 shows that BE-830 does not have liver toxicity in vivo. High-dose Urelumab analog, but not BE-830, induced significantly increased alanine transaminase (ALT) and aspartate aminotransferase (AST) concentrations, and increased inflammatory cells infiltration in liver.

FIG. 30A-D is a schematic diagram of GPC3×CD137 multispecific antibody formats.

FIG. 31 is a schematic diagram of GPC3×CD137 multispecific antibody formats.

### DETAILED DESCRIPTION

The present disclosure provides for antibodies, antigen-binding fragments, and anti-GPC3×CD137 multispecific antibodies. Furthermore, the present disclosure provides antibodies that have desirable pharmacokinetic characteristics and other desirable attributes, and thus can be used for reducing the likelihood of or treating cancer. The present disclosure further provides pharmaceutical compositions comprising the antibodies and methods of making and using such pharmaceutical compositions for the prevention and treatment of cancer and associated disorders.

### I. Anti-GPC3 Antibodies

The present disclosure provides for antibodies or antigen-binding fragments thereof that specifically bind to GPC3. In one embodiment, the anti-GPC3 antibodies or antigen-binding fragments thereof specifically bind to GPC3 with a binding affinity (K_{D}) of from 1×10⁻⁶ M to 1×10⁻¹⁰ M. In another embodiment, the anti-GPC3 antibodies or antigen-binding fragments thereof bind to GCP3 with a binding affinity (K_{D}) of about 1×10⁻⁶ M, about 1×10⁻⁷ M, about 1×10⁻⁸ M, about 1×10⁻⁹ M or about 1×10⁻¹⁰ M.

In one embodiment, the anti-GPC3 antibodies or antigen-binding fragments thereof comprise: a heavy chain variable region (VH) that comprises (a) a HCDR1 of SEQ ID NO: 10, (b) a HCDR2 of SEQ ID NO: 11, (c) a HCDR3 of SEQ ID NO: 12; and a light chain variable region (VL) that comprises (d) a LCDR1 of SEQ ID NO: 13, (e) a LCDR2 of SEQ ID NO: 14, (f) a LCDR3 of SEQ ID NO: 15, according to the Kabat numbering.

In another embodiment, the anti-GPC3 antibodies or antigen-binding fragments thereof comprise: a HCDR1, a HCDR2 and a HCDR3 from the heavy chain variable region (VH) set forth in SEQ ID NO: 5; and a LCDR1, a LCDR2 and a LCDR3 from the light chain variable region (VL) set forth in SEQ ID NO: 7.

In another embodiment, the anti-GPC3 antibodies or antigen-binding fragments thereof further comprise no more than one, two, three, four or five amino acid deletions, insertions or substitutions in the CDR, preferably the amino acid substitutions are conservative amino acid substitutions, while maintaining binding specificity and affinity.

In another embodiment, the anti-GPC3 antibodies or antigen-binding fragments thereof comprise a heavy chain variable region (VH) comprising an amino acid sequence at least 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% identical to SEQ ID NO: 5, and a light chain variable region (VL) comprising an amino acid sequence at least 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% identical to SEQ ID NO: 7. In another embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids within SEQ ID NO: 5 or SEQ ID NO: 7 have been inserted, deleted or substituted (optionally conservative amino acid substitutions). In another embodiment, such variations are in the framework region of the variable region. In another embodiment, anti-GPC3 antibodies or antigen-binding fragments thereof having such variations maintains binding specificity and affinity.

In another embodiment, the anti-GPC3 antibodies or antigen-binding fragments thereof comprise a heavy chain variable region (VH) that comprises SEQ ID NO: 5, and a light chain variable region (VL) that comprises SEQ ID NO: 7.

In another embodiment, the anti-human GPC3 antibodies or antigen-binding fragments thereof show a cross-species binding activity to cynomolgus GPC3.

### II. Anti-CD137 Antibodies

TABLE 1
Sequences of CD137 sequences

| Antibody | SEQ ID NO | | SEQUENCE |
|---|---|---|---|
| BGA-7207 | SEQ ID NO: 52 | HCDR1 (IMGT) | GFRLDTTE |
| (IMGT) | SEQ ID NO: 53 | HCDR2 (IMGT) | IMGISGST |
| | SEQ ID NO: 54 | HCDR3 (IMGT) | ARVVDSLFDDSAVFDY |
| BGA-7207 | SEQ ID NO: 55 | HCDR1 (Kabat) | TTEVG |
| (Kabat) | SEQ ID NO: 56 | HCDR2 (Kabat) | TIMGISGSTYYADSVKG |
| | SEQ ID NO: 57 | HCDR3 (Kabat) | VVDSLFDDSAVFDY |
| BGA-7207 | SEQ ID NO: 58 | HCDR1 (Chothia) | GFRLDTT |
| (Chothia) | SEQ ID NO: 59 | HCDR2 (Chothia) | MGISGS |
| | SEQ ID NO: 57 | HCDR3 (Chothia) | VVDSLFDDSAVFDY |
| BGA-7207 | SEQ ID NO: 60 | VH | EVQLLESGGGLVQPGGSLRLSCAASGFR |
| | | | LDTTEVGWVRQAPGKGLEWVSTIMGIS |
| | | | GSTYYADSVKGRFTISRDNSKNTLYLQ |
| | | | MNSLRAEDTAVYYCARVVDSLFDDSAV |
| | | | FDYWGQGTLVTVSS |
| | SEQ ID NO: 61 | VH DNA | GAGGTCCAGTTACTTGAGAGTGGTGG |
| | | | AGGTCTGGTCCAACCAGGAGGTTCGC |
| | | | TGCGTTTATCCTGCGCCGCGTCTGGAT |
| | | | TCAGATTGGACACCACCGAAGTAGGT |
| | | | TGGGTGCGTCAAGCGCCGGGGAAAGG |
| | | | ACTGGAATGGGTCTCCACCATCATGGG |
| | | | TATTAGTGGTTCGACATACTATGCGGA |
| | | | CAGTGTCAAAGGGCGCTTTACGATCTC |
| | | | GCGCGATAACTCAAAAAATACTCTTTA |
| | | | CCTTCAAATGAATAGCCTTCGTGCTGA |
| | | | GGACACTGCGGTGTACTACTGCGCCC |
| | | | GCGTCGTCGATTCCCTCTTTGATGACA |
| | | | GCGCCGTTTTTGATTACTGGGGACAGG |
| | | | GCACCTTAGTTACAGTCTCATCG |
| BGA-4712 | SEQ ID NO: 62 | HCDR1 (IMGT) | GFMLSAED |
| (IMGT) | SEQ ID NO: 63 | HCDR2 (IMGT) | ILDFGGST |
| | SEQ ID NO: 64 | HCDR3 (IMGT) | ARVVYHAGGGVTFDY |
| BGA-4712 | SEQ ID NO: 65 | HCDR1 (Kabat) | AEDVG |
| (Kabat) | SEQ ID NO: 66 | HCDR2 (Kabat) | AILDFGGSTYYADSVKG |
| | SEQ ID NO: 67 | HCDR3 (Kabat) | VVYHAGGGVTFDY |
| BGA-4712 | SEQ ID NO: 68 | HCDR1 (Chothia) | GFMLSAE |
| (Chothia) | SEQ ID NO: 69 | HCDR2 (Chothia) | LDFGGS |
| | SEQ ID NO: 67 | HCDR3 (Chothia) | VVYHAGGGVTFDY |
| BGA-4712 | SEQ ID NO: 70 | VH | EVQLLESGGGLVQPGGSLRLSCAASGF |
| | | | MLSAEDVGWVRQAPGKGLEWVSAILD |
| | | | FGGSTYYADSVKGRFTISRDNSKNTLYL |
| | | | QMNSLRAEDTAVYYCARVVYHAGGGV |
| | | | TFDYWGQGTLVTVSS |
| | SEQ ID NO: 71 | VH DNA | GAGGTCCAGTTACTTGAGAGTGGTGG |
| | | | AGGTCTGGTCCAACCAGGAGGTTCGC |
| | | | TGCGTTTATCCTGCGCCGCGTCTGGAT |
| | | | TCATGTTGTCCGCCGAAGACGTGGGTT |
| | | | GGGTGCGTCAAGCGCCGGGGAAAGG |
| | | | ACTGGAATGGGTCTCCGCCATCTTGGA |
| | | | TTTTGGTGGTTCGACATACTATGCGGA |
| | | | CAGTGTCAAAGGGCGCTTTACGATCTC |
| | | | GCGCGATAACTCAAAAAATACTCTTTA |
| | | | CCTTCAAATGAATAGCCTTCGTGCTGA |
| | | | GGACACTGCGGTGTACTACTGCGCCC |
| | | | GCGTCGTCTACCATGCTGGTGGTGGCG |
| | | | TCACCTTTGATTACTGGGGACAGGGCA |
| | | | CCTTAGTTACAGTCTCATCG |
| BGA-4712-M3 | SEQ ID NO: 72 | HCDR1 (IMGT) | GFTLSAED |
| (IMGT) | SEQ ID NO: 63 | HCDR2 (IMGT) | ILDFGGST |
| | SEQ ID NO: 64 | HCDR3 (IMGT) | ARVVYHAGGGVTFDY |
| BGA-4712-M3 | SEQ ID NO: 65 | HCDR1 (Kabat) | AEDVG |
| (Kabat) | SEQ ID NO: 73 | HCDR2 (Kabat) | AILDFGGSTYYAESVKG |
| | SEQ ID NO: 67 | HCDR3 (Kabat) | VVYHAGGGVTFDY |
| BGA-4712-M3 | SEQ ID NO: 74 | HCDR1 (Chothia) | GFTLSAE |
| (Chothia) | SEQ ID NO: 69 | HCDR2 (Chothia) | LDFGGS |
| | SEQ ID NO: 67 | HCDR3 (Chothia) | VVYHAGGGVTFDY |
| BGA-4712-M3 | SEQ ID NO: 75 | VH | EVQLLESGGGLVQPGGSLRLSCAASGFT |
| | | | LSAEDVGWVRQAPGKGLEWVSAILDF |
| | | | GGSTYYAESVKGRFTISRDNAKNTLYLQ |
| | | | MSSLRAEDTAVYYCARVVYHAGGGVT |
| | | | FDYWGQGTLVTVSS |
| | SEQ ID NO: 76 | VH DNA | GAGGTCCAGTTACTTGAGAGTGGTGG |
| | | | AGGTCTGGTCCAACCAGGAGGTTCGC |
| | | | TGCGTTTATCCTGCGCCGCGTCTGGAT |
| | | | TCACGTTGTCCGCCGAAGACGTGGGT |
| | | | TGGGTGCGTCAAGCGCCGGGGAAAGG |
| | | | ACTGGAATGGGTCTCCGCCATCTTGGA |
| | | | TTTTGGTGGTTCGACATACTATGCGGA |
| | | | AAGTGTCAAAGGGCGCTTTACGATCTC |
| | | | GCGCGATAACGCAAAAAATACTCTTTA |
| | | | CCTTCAAATGTCTAGCCTTCGTGCTGA |
| | | | GGACACTGCGGTGTACTACTGCGCCC |
| | | | GCGTCGTCTACCATGCTGGTGGTGGCG |
| | | | TCACCTTTGATTACTGGGGACAGGGCA |
| | | | CCTTAGTTACAGTCTCATCG |
| BGA-5623 | SEQ ID NO: 77 | HCDR1 (IMGT) | GFTVSAED |
| (IMGT) | SEQ ID NO: 78 | HCDR2 (IMGT) | ILDKGGST |
| | SEQ ID NO: 79 | HCDR3 (IMGT) | ARIVYHAGGGVTFDT |
| BGA-5623 | SEQ ID NO: 65 | HCDR1 (Kabat) | AEDVG |
| (Kabat) | SEQ ID NO: 80 | HCDR2 (Kabat) | AILDKGGSTYYAESVKG |
| | SEQ ID NO: 81 | HCDR3 (Kabat) | IVYHAGGGVTFDT |
| BGA-5623 | SEQ ID NO: 82 | HCDR1 (Chothia) | GFTVSAE |
| (Chothia) | SEQ ID NO: 83 | HCDR2 (Chothia) | LDKGGS |
| | SEQ ID NO: 81 | HCDR3 (Chothia) | IVYHAGGGVTFDT |
| BGA-5623 | SEQ ID NO: 84 | VH | EVQLLESGGGLVQPGGSLRLSCAASGFT |
| | | | VSAEDVGWVRQAPGKGLEWVSAILDK |
| | | | GGSTYYAESVKGRFTISRDNAKNTLYLQ |
| | | | MSSLRAEDTAVYYCARIVYHAGGGVTF |
| | | | DTRGQGTQVTVSS |
| | SEQ ID NO: 85 | VH DNA | GAGGTCCAGTTACTTGAGAGTGGTGG |
| | | | AGGTCTGGTCCAACCAGGAGGTTCGC |
| | | | TGCGTTTATCCTGCGCCGCGTCTGGAT |
| | | | TCACGGTTTCCGCCGAAGACGTGGGT |
| | | | TGGGTGCGTCAAGCGCCGGGGAAAGG |
| | | | ACTGGAATGGGTCTCCGCCATCTTGGA |
| | | | TAAGGGTGGTTCGACATACTATGCGGA |
| | | | AAGTGTCAAAGGGCGCTTTACGATCTC |
| | | | GCGCGATAACGCAAAAAATACTCTTTA |
| | | | CCTTCAAATGTCTAGCCTTCGTGCTGA |
| | | | GGACACTGCGGTGTACTACTGCGCCC |
| | | | GCATTGTCTACCATGCTGGTGGTGGCG |
| | | | TCACCTTTGATACTCGGGGACAGGGC |
| | | | ACCCAAGTTACAGTCTCATCG |
| BGA-7556 | SEQ ID NO: 86 | VH | EVQLLESGGGLVQPGGSLRLSCAASGFT |
| | | | LSAEDVGWVRQAPGKGLEWVSAILDF |
| | | | GGSTYYAESVKGRFTISRDNAKNTLYLQ |
| | | | MSSLRAEDTAVYYCARVVYHAGGGVT |
| | | | FDYRGQGTQVTVSS |
| | SEQ ID NO: 87 | VH DNA | GAGGTCCAGTTACTTGAGAGTGGTGG |
| | | | AGGTCTGGTCCAACCAGGAGGTTCGC |
| | | | TGCGTTTATCCTGCGCCGCGTCTGGAT |
| | | | TCACGTTGTCCGCCGAAGACGTGGGT |
| | | | TGGGTGCGTCAAGCGCCGGGGAAAGG |
| | | | ACTGGAATGGGTCTCCGCCATCTTGGA |
| | | | TTTTGGTGGTTCGACATACTATGCGGA |
| | | | AAGTGTCAAAGGGCGCTTTACGATCTC |
| | | | GCGCGATAACGCAAAAAATACTCTTTA |
| | | | CCTTCAAATGTCTAGCCTTCGTGCTGA |
| | | | GGACACTGCGGTGTACTACTGCGCCC |
| | | | GCGTCGTCTACCATGCTGGTGGTGGCG |
| | | | TCACCTTTGATTACCGGGGACAGGGC |
| | | | ACCCAAGTTACAGTCTCATCG |

The present disclosure provides for antibodies or antigen-binding fragments thereof that specifically bind to CD137. Antibodies or antigen-binding fragments of the present disclosure include, but are not limited to, the antibodies or antigen-binding fragments thereof, generated as described below.

The present disclosure provides for antibodies or antigen-binding fragments that specifically bind to CD137, wherein said antibodies or antibody fragments (e.g., antigen-binding fragments) comprise a VH domain having an amino acid sequence of SEQ ID NO: 60, SEQ ID NO: 70, SEQ ID NO: 75, SEQ ID NO: 84 or SEQ ID NO: 86 (Table 1). The present disclosure also provides antibodies or antigen-binding fragments that specifically bind CD137, wherein said antibodies or antigen-binding fragments comprise a HCDR having an amino acid sequence of any one of the HCDRs listed in Table 1. In one aspect, the present disclosure provides antibodies or antigen-binding fragments that specifically bind to CD137, wherein said antibodies comprise (or alternatively, consist of) one, two, three, or more HCDRs having an amino acid sequence of any of the HCDRs listed in Table 1.

In one embodiment, the anti-CD137 antibodies or antigen-binding fragments thereof comprise: (i) a HCDR1 (Heavy Chain Complementarity Determining Region 1), a HCDR2 and a HCDR3 from the heavy chain variable region (VH) set forth in SEQ ID NO: 84; (ii) a HCDR1, a HCDR2 and a HCDR3 from the heavy chain variable region (VH) set forth in SEQ ID NO: 75; (iii) a HCDR1, a HCDR2 and a HCDR3 from the heavy chain variable region (VH) set forth in SEQ ID NO: 70; or (iv) a HCDR1, a HCDR2 and a HCDR3 from the heavy chain variable region (VH) set forth in SEQ ID NO: 60.

In one embodiment, the anti-CD137 antibodies or antigen-binding fragments thereof comprise: (i) a heavy chain variable region (VH) that comprises (a) a HCDR1 of SEQ ID NO: 65, (b) a HCDR2 of SEQ ID NO: 80, (c) a HCDR3 of SEQ ID NO: 81; (ii) a heavy chain variable region (VH) that comprises (a) a HCDR1 of SEQ ID NO: 65, (b) a HCDR2 of SEQ ID NO: 73, (c) a HCDR3 of SEQ ID NO: 67; (iii) a heavy chain variable region (VH) that comprises (a) a HCDR1 of SEQ ID NO: 65, (b) a HCDR2 of SEQ ID NO: 66, (c) a HCDR3 of SEQ ID NO: 67; or (iv) a heavy chain variable region (VH) that comprises (a) a HCDR1 of SEQ ID NO: 55, (b) a HCDR2 of SEQ ID NO: 56, (c) a HCDR3 of SEQ ID NO: 57, according to the Kabat numbering.

Other antibodies or antigen-binding fragments thereof of the present disclosure include amino acids that have been changed, yet have at least 60%, 70%, 80%, 90%, 91%, 92%, 93%, 94%, 95% 96%, 97%, 98%, or 99% percent identity in the CDR regions with the CDR regions disclosed in Table 1. In some aspects, it includes amino acid changes (insertion, deletion or substitution, optionally conservative amino acid substitutions) wherein no more than 1, 2, 3, 4 or 5 amino acids have been changed in the CDR regions when compared with the CDR regions depicted in the sequence described in Table 1, while maintaining binding specificity and affinity.

Other antibodies of the present disclosure include those where the amino acids or nucleic acids encoding the amino acids have been changed; yet have at least 60, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% percent identity to the sequences described in Table 1. In some aspects, it includes changes in the amino acid sequences wherein no more than 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids have been changed in the variable regions when compared with the variable regions depicted in the sequence described in Table 1, while retaining therapeutic activity/binding specificity/affinity.

In another embodiment, the present disclosure provides for anti-CD137 antibodies or antigen-binding fragments thereof specifically binds to an epitope of human CD137 comprising amino acids F36, P47 and P49, or comprising amino acids F36, P47, P49 and S52. In another embodiment, the present disclosure provides for anti-CD137 antibodies or antigen-binding fragments thereof specifically binds to human CD137 at amino acids 36 to 52 of SEQ ID NO: 94.

In another embodiment, the present disclosure provides for antibodies or antigen-binding fragments thereof that specifically bind to CD137 with a binding affinity (K_{D}) of from 1×10⁻⁶ M to 1×10⁻¹⁰ M. In another embodiment, the anti-CD137 antibodies or antigen-binding fragments thereof bind to CD137 with a binding affinity (K_{D}) of about 1×10⁻⁶ M, about 1×10⁻⁷ M, about 1×10⁻⁸ M, about 1×10⁻⁹ M or about 1×10⁻¹⁰ M.

The present disclosure also provides nucleic acid sequences that encode VH, VL, the full length heavy chain, and the full length light chain of the antibodies that specifically bind to CD137. Such nucleic acid sequences can be optimized for expression in mammalian cells.

The present disclosure provides for antibodies and antigen-binding fragments thereof that bind to an epitope of human CD137. In certain aspects, the antibodies and antigen-binding fragments can bind to the same epitope of CD137.

The present disclosure also provides for antibodies and antigen-binding fragments thereof that bind to the same epitope as do the anti-CD137 antibodies described in Table 1. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of the present disclosure to CD137 demonstrates that the test antibody can compete with that antibody or antigen-binding fragments thereof for binding to CD137. Such an antibody can, without being bound to any one theory, bind to the same or a related (e.g., a structurally similar or spatially proximal) epitope on CD137 as the antibody or antigen-binding fragments thereof with which it competes. In a certain aspect, the antibody that binds to the same epitope on CD137 as the antibodies or antigen-binding fragments thereof of the present disclosure is a human or humanized monoclonal antibody. Such human or humanized monoclonal antibodies can be prepared and isolated as described herein.

### III. Anti-GPC3×CD137 Multispecific Antibodies

In one embodiment, the anti-GPC3 and anti-CD137 antibodies as disclosed herein can be incorporated into an anti-GPC3×CD137 multispecific antibody. An antibody molecule is a multispecific antibody molecule, for example, it comprises a number of antigen binding domains, wherein at least one antigen binding domain sequence specifically binds GPC3 as a first epitope and a second antigen binding domain sequence specifically binds CD137 as a second epitope. In one embodiment, the multispecific antibody comprises a third, fourth or fifth antigen binding domain. In one embodiment, the multispecific antibody is a bispecific antibody, a trispecific antibody, or tetraspecific antibody. In each example, the multispecific antibody comprises at least one anti-GPC3 antigen binding domain and at least one anti-CD137 antigen binding domain.

In one embodiment, the multispecific antibody is a bispecific antibody. As used herein, a bispecific antibody specifically binds only two antigens. The bispecific antibody comprises a first antigen binding domain which specifically binds GPC3 and a second antigen binding domain that specifically binds CD137. This includes a bispecific antibody comprising a heavy chain variable domain and a light chain variable domain which specifically binds GPC3 as a first epitope and a heavy chain variable domain which specifically binds CD137 as a second epitope. In another embodiment, the bispecific antibody comprises an antigen binding fragment of an antibody that specifically binds GPC3 and an antigen binding fragment that specially binds CD137. The bispecific antibody that comprises antigen binding fragments, the antigen-binding fragment can be a Fab, F(ab′)₂, Fv, a single chain Fv (scFv), or a single domain antibody.

The present disclosure provides for a multispecific antibody or antigen-binding fragment thereof, comprising a first antigen binding domain that specifically binds to human Glypican 3 (GPC3) and a second antigen binding domain that specifically binds to human CD137.

The first antigen binding domain that specifically binds to human Glypican 3 (GPC3) includes the anti-GPC3 antibodies described in Section I. The second antigen binding domain that specifically binds to human CD137 include the anti-CD137 antibodies disclosed in Section II.

In one embodiment, the multispecific antibody of the present disclosure binds to GPC3 and/or CD137 with a binding affinity (K_{D}) of from 1×10⁻⁶ M to 1×10⁻¹⁰ M. In another embodiment, the multispecific antibody of the present disclosure binds to GPC3 and/or CD137 with a binding affinity (K_{D}) of about 1×10⁻⁶ M, about 1×10⁻⁷ M, about 1×10⁻⁸ M, about 1×10⁻⁹ M or about 1×10⁻¹⁰ M.

In one embodiment, the multispecific antibody of the present disclosure has specific binding to GPC3, and shows high affinity to both human GPC3 and monkey GPC3. In another embodiment, the multispecific antibody of the present disclosure has specific binding to CD137, and does not bind to other TNF receptor family members. In another embodiment, the multispecific antibody of the present disclosure shows high affinity to human CD137 and monkey CD137.

In one embodiment, the multispecific antibody or antigen-binding fragment thereof specifically binds to an epitope of human CD137 comprising amino acids F36, P47 and P49, or comprising amino acids F36, P47, P49 and S52. In another embodiment, the present disclosure provides for multispecific antibody or antigen-binding fragment thereof specifically binds to human CD137 at amino acids 36 to 52 of SEQ ID NO: 94.

In one embodiment, the present disclosure provides for a multispecific antibody or antigen-binding fragment thereof, wherein the first antigen binding domain that specifically binds to human GPC3 comprises: a heavy chain variable region (VH) that comprises (a) a HCDR1 of SEQ ID NO: 10, (b) a HCDR2 of SEQ ID NO: 11, (c) a HCDR3 of SEQ ID NO: 12; and a light chain variable region (VL) that comprises (d) a LCDR1 of SEQ ID NO: 13, (e) a LCDR2 of SEQ ID NO: 14, (f) a LCDR3 of SEQ ID NO: 15, according to the Kabat numbering; and wherein the second antigen binding domain that specifically binds to human CD137 comprises:
(i) a heavy chain variable region (VH) that comprises (a) a HCDR1 of SEQ ID NO: 65, (b) a HCDR2 of SEQ ID NO: 80, (c) a HCDR3 of SEQ ID NO: 81;
(ii) a heavy chain variable region (VH) that comprises (a) a HCDR1 of SEQ ID NO: 65, (b) a HCDR2 of SEQ ID NO: 73, (c) a HCDR3 of SEQ ID NO: 67;
(iii) a heavy chain variable region (VH) that comprises (a) a HCDR1 of SEQ ID NO: 65, (b) a HCDR2 of SEQ ID NO: 66, (c) a HCDR3 of SEQ ID NO: 67; or
(iv) a heavy chain variable region (VH) that comprises (a) a HCDR1 of SEQ ID NO: 55, (b) a HCDR2 of SEQ ID NO: 56, (c) a HCDR3 of SEQ ID NO: 57, according to the Kabat numbering.

In another embodiment, the present disclosure provides for a multispecific antibody or antigen-binding fragment thereof, wherein the first antigen binding domain that specifically binds to human GPC3 comprises: a heavy chain variable region (VH) that comprises SEQ ID NO: 5, and a light chain variable region (VL) that comprises SEQ ID NO: 7; and wherein the second antigen binding domain that specifically binds to human CD137 comprises: (i) a heavy chain variable region (VH) that comprises SEQ ID NO: 84; (ii) a heavy chain variable region (VH) that comprises SEQ ID NO: 86; (iii) a heavy chain variable region (VH) that comprises SEQ ID NO: 75; (iv) a heavy chain variable region (VH) that comprises SEQ ID NO: 70; or (v) a heavy chain variable region (VH) that comprises SEQ ID NO: 60.

In another embodiment, the present disclosure provides a multispecific antibody or antigen-binding fragment thereof, wherein the multispecific antibody or antigen-binding fragment is BE-830 comprising the first polypeptide of SEQ ID NO: 1 and the second polypeptide of SEQ ID NO: 3.

Other multispecific antibody or antigen-binding fragment thereof of the present disclosure include those where the amino acids or nucleic acids encoding the amino acids have been changed; yet have at least 60, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% percent identity to the sequences described herein. In some aspects, it includes changes in the amino acid sequences wherein no more than 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids have been changed in the variable regions when compared with the variable regions described herein, while retaining therapeutic activity/binding specificity/affinity.

Previous experimentation (Coloma and Morrison Nature Biotech. 15: 159-163 (1997)) described a tetravalent bispecific antibody which was engineered by fusing DNA encoding a single chain anti-dansyl antibody Fv (scFv) after the C terminus (CH3-scFv) or after the hinge (hinge-scFv) of an lgG3 anti-dansyl antibody. The present disclosure provides multivalent antibodies (e.g., tetravalent antibodies) with at least two antigen binding domains, which can be readily produced by recombinant expression of nucleic acid encoding the polypeptide chains of the antibody. The multivalent antibody herein comprises three to eight, but preferably four, antigen binding domains, which specifically bind at least two antigens.

### Format and Module Ratios

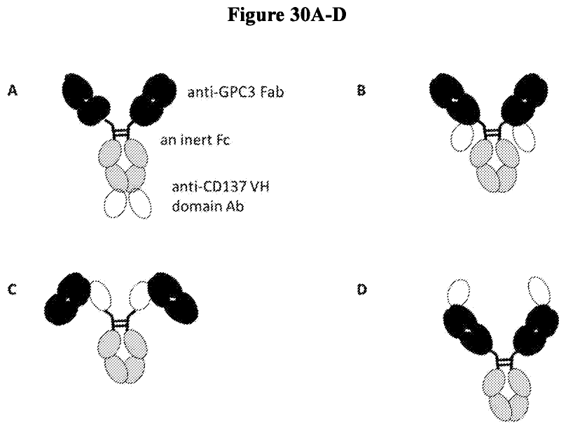

The multispecific antibodies of the disclosure could be in different formats. In one embodiment, the multispecific antibodies of the disclosure have the format as disclosed in FIG. 30, including: (1) the format A provides a symmetric IgG-like multispecific molecule with Fab×VH configuration. Anti-huCD137 VH domain antibody was fused to the c-termini of Fc (CH3 domain) of an anti-GPC3 antibody with a linker in between as shown; (2) the format B also provides a symmetric IgG-like multispecific molecule with Fab×VH configuration. Anti-huCD137 VH domain antibody was fused to the c-termini of light chain (CK) of an anti-GPC3 antibody with a linker in between; (3) the format C provides a symmetric VH antibody-like multispecific molecule with Fab×VH configuration. The Fab region of an anti-GPC3 antibody was fused to the N-termini of VH of anti-huCD137 VH domain Ab with a linker in between; and (4) the format D also provides a symmetric IgG-like multispecific molecule with Fab×VH configuration. Anti-huCD137 VH domain antibody was fused to the N-termini of heavy chain (VH) of an anti-GPC3 antibody with a linker in between. In one embodiment, the multispecific antibodies are in the format A.

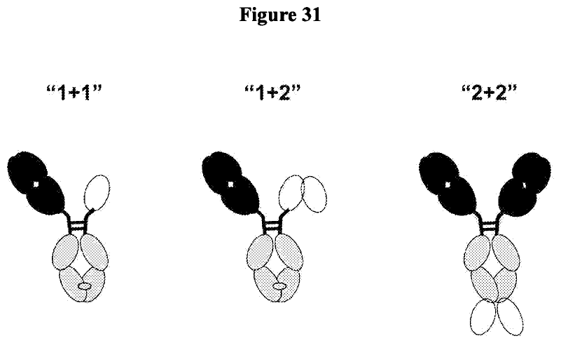

The multispecific antibodies of the disclosure can be constructed with different module ratios such as 1:1 and 1:2 such as shown in FIG. 31 which demonstrates the format of these configurations. In one embodiment, an inert Fc can be used for multispecific antibodies and the Azymetric™ Platform from Zymeworks can be utilized to assemble the Fab×VH configuration, in which ZW1 mutations (chain A: T350V/L351Y/F405A/Y407V; chain B: T350V/T366L/K392L/T394W) can be introduced in the CH3 domain of heavy chain to allow efficient heterodimer formation (Von Kreudenstein et al., (2013) Mabs 5(5):646-54). In one aspect, the specific ratios will activate CD137 in a GPC3 dependent manner, and without the activation of CD137 in the absence of GPC3.

In one embodiment, the multispecific antibody or antigen-binding fragment thereof comprises: a) a first polypeptide comprising from N terminal to C terminal: a first heavy chain variable region (such as one first heavy chain variable region); a CH1 domain, a Fc domain, and a second heavy chain variable region (such as one second heavy chain variable region); optionally, C terminal of the Fc domain is linked to N terminal of the second heavy chain variable region via a linker; and b) a second polypeptide comprising from N terminal to C terminal: a first light chain variable region (such as one first light chain variable region); and a first light chain constant region; wherein the first heavy chain variable region and the first light chain variable region form the first antigen binding domain that specifically binds to human GPC3, and the second heavy chain variable region forms the second antigen binding domain that specifically binds to human CD137. In another embodiment, the multispecific antibody or antigen-binding fragment thereof comprises two of the first polypeptides and two of the second polypeptides.

### Linkers

It is understood that whether there is a linker has minimal influence on the activity of the multispecific antibody of the present disclosure.

It is also understood that the domains and/or regions of the polypeptide chains of the bispecific antibody can be separated by linker regions of various lengths. In some embodiments, the antigen binding domains are separated from each other, a CL, CH1, hinge, CH2, CH3, or the entire Fc region by a linker region. For example, VL1-CL-(linker) VH2-CH1. Such linker region may comprise a random assortment of amino acids, or a restricted set of amino acids. Such linker regions can be flexible or rigid (see US2009/0155275).

Multispecific antibodies have been constructed by genetically fusing two single chain Fv (scFv) or Fab fragments with or without the use of flexible linkers (Mallender et al., J. Biol. Chem. 1994 269: 199-206; Mack et al., Proc. Natl. Acad. Sci. USA. 1995 92:7021-5; Zapata et al., Protein Eng. 1995 8.1057-62), via a dimerization device such as leucine Zipper (Kostelny et al., J. Immunol. 1992148: 1547-53; de Kruifetal J. Biol. Chem. 1996 271:7630-4) and Ig C/CH1 domains (Muller et al., FEBS Lett. 422:259-64); by diabody (Holliger et al., (1993) Proc. Nat. Acad. Sci. USA. 1998 90:6444-8; Zhu et al., Bio/Technology (NY) 1996 14:192-6); Fab-scFv fusion (Schoonjans et al., J. Immunol. 2000 165:7050-7); and mini antibody formats (Pack et al., Biochemistry 1992.31:1579-84; Pack et al., Bio/Technology 1993 11:1271-7).

The multispecific antibodies as disclosed herein comprise a linker region of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues between one or more of its antigen binding domains, CL domains, CH1 domains, Hinge region, CH2 domains, CH3 domains, or Fc regions. In some embodiments, the amino acids glycine and serine are comprised within the linker region. In another embodiment, the linker can be GS (SEQ ID NO: 16), GGS (SEQ ID NO: 17), GSG (SEQ ID NO: 18), SGG (SEQ ID NO: 19), GGG (SEQ ID NO: 20), GGGS (SEQ ID NO: 21), SGGG (SEQ ID NO: 22), GGGGS (SEQ ID NO: 23), GGGGSGS (SEQ ID NO: 24), GGGGSGS (SEQ ID NO: 25), GGGGSGGS (SEQ ID NO: 26), GGGGGGGGS (SEQ ID NO: 27), GGGGSGGGGSGGGGS (SEQ ID NO: 28), AKTTPKLEEGEFSEAR (SEQ ID NO: 29), AKTTPKLEEGEFSEARV (SEQ ID NO: 30), AKTTPKLGG (SEQ ID NO: 31), SAKTTPKLGG (SEQ ID NO: 32), AKTTPKLEEGEFSEARV (SEQ ID NO: 33), SAKTTP (SEQ ID NO: 34), SAKTTPKLGG (SEQ ID NO: 35), RADAAP (SEQ ID NO: 36), RADAAPTVS (SEQ ID NO: 37), RADAAAAGGPGS (SEQ ID NO: 38), RADAAAA(G₄S)₄ (SEQ ID NO: 39), SAKTTP (SEQ ID NO: 40), SAKTTPKLGG (SEQ ID NO: 41), SAKTTPKLEEGEFSEARV (SEQ ID NO: 42), ADAAP (SEQ ID NO: 43), ADAAPTVSIFPP (SEQ ID NO: 44), TVAAP (SEQ ID NO: 45), TVAAPSVFIFPP (SEQ ID NO: 46), QPKAAP (SEQ ID NO: 47), QPKAAPSVTLFPP (SEQ ID NO: 48), AKTTPP (SEQ ID NO: 49), AKTTPPSVTPLAP (SEQ ID NO: 50), AKTTAP (SEQ ID NO: 51), AKTTAPSVYPLAP (SEQ ID NO: 88), ASTKGP (SEQ ID NO: 89), ASTKGPSVFPLAP (SEQ ID NO: 90), GENKVEYAPALMALS (SEQ ID NO: 91), GPAKELTPLKEAKVS (SEQ ID NO: 92), and GHEAAAVMQVQYPAS (SEQ ID NO: 93) or any combination thereof (see WO2007/024715).

### Dimerization Specific Amino Acids

In one embodiment, the multivalent antibody comprises at least one dimerization specific amino acid change. The dimerization specific amino acid changes result in “knobs into holes” interactions, and increases the assembly of correct multivalent antibodies. The dimerization specific amino acids can be within the CH1 domain or the CL domain or combinations thereof. The dimerization specific amino acids used to pair CH1 domains with other CH1 domains (CH1-CH1) and CL domains with other CL domains (CL-CL) and can be found at least in the disclosures of WO2014082179, WO2015181805 family and WO2017059551. The dimerization specific amino acids can also be within the Fc domain and can be in combination with dimerization specific amino acids within the CH1 or CL domains. In one embodiment, the disclosure provides a bispecific antibody comprising at least one dimerization specific amino acid pair.

### Further Alteration of the Framework of Fc Region

The Fc region could be wild type Fc region of the subclass of IgG1, IgG2, IgG3, or IgG4.

In one embodiment, the multispecific antibody or antigen-binding fragment thereof comprises a Fc domain of IgG1 or IgG4 with reduced effector function. In another embodiment, the Fc domain comprises an amino acid sequence of SEQ ID NO: 9.

In another embodiment, antibodies of the present disclosure have strong Fc-mediated effector functions, and the antibodies mediate antibody-dependent cellular cytotoxicity (ADCC) against target cells expressing GPC3.

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al.

In yet another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcγRI, FcγRII, FcγRIII and FcRn have been mapped and variants with improved binding have been described (see Shields et al., J. Biol. Chem. 276:6591-6604, 2001).

In still another aspect, the glycosylation of the multispecific antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al., describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). WO99/54342 by Umana et al., describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another aspect, if a reduction of ADCC is desired, human antibody subclass IgG4 was shown in many previous reports to have only modest ADCC and almost no CDC effector function (Moore G L, et al., 2010 MAbs, 2:181-189). However, natural IgG4 was found less stable in stress conditions such as in acidic buffer or under increasing temperature (Angal, S. 1993 Mol Immunol, 30:105-108; Dall'Acqua, W. et al., 1998 Biochemistry, 37:9266-9273; Aalberse et al., 2002 Immunol, 105:9-19). Reduced ADCC can be achieved by operably linking the antibody to an IgG4 Fc engineered with combinations of alterations that reduce FcγR binding or C1q binding activities, thereby reducing or eliminating ADCC and CDC effector functions. Considering the physicochemical properties of antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., 2007 Science, 317:1554-157). The mutation of serine to proline at position 228 (EU numbering system) appeared inhibitory to the IgG4 heavy chain separation (Angal, S. 1993 Mol Immunol, 30:105-108; Aalberse et al., 2002 Immunol, 105:9-19). Some of the amino acid residues in the hinge and γFc region were reported to have impact on antibody interaction with Fcγ receptors (Chappel S M, et al., 1991 Proc. Natl. Acad. Sci. USA, 88:9036-9040; Mukherjee, J. et al., 1995 FASEB J, 9:115-119; Armour, K. L. et al., 1999 Eur J Immunol, 29:2613-2624; Clynes, R. A. et al, 2000 Nature Medicine, 6:443-446; Arnold J. N., 2007 Annu Rev immunol, 25:21-50). Furthermore, some rarely occurring IgG4 isoforms in human population can also elicit different physicochemical properties (Brusco, A. et al., 1998 Eur J Immunogenet, 25:349-55; Aalberse et al., 2002 Immunol, 105:9-19). To generate multispecific antibodies with low ADCC and CDC but with good stability, it is possible to modify the hinge and Fc region of human IgG4 and introduce a number of alterations. These modified IgG4 Fc molecules can be found in SEQ ID NOs: 83-88, U.S. Pat. No. 8,735,553 to Li et al.

In another embodiment, the antibody of the present disclosure comprises Fc domain of human IgG4 with S228P and/or R409K substitutions (according to EU numbering system).

### Antibody Production

Antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions or light chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NO: 61, SEQ ID NO: 71, SEQ ID NO: 76, SEQ ID NO: 85, SEQ ID NO: 87, SEQ ID NO: 2, SEQ ID NO: 4, SEQ ID NO: 6 or SEQ ID NO: 8.

The polynucleotides of the present disclosure can encode the variable region sequence of an anti-GPC3×CD137 antibody. They can also encode both a variable region and a constant region of the antibody. Some of the polynucleotide sequences encode a polypeptide that comprises variable regions of both the heavy chain and the light chain of the exemplified anti-GPC3×CD137 antibodies.

Also provided in the present disclosure are expression vectors and host cells for producing the anti-GPC3×CD137 antibodies. The choice of expression vector depends on the intended host cells in which the vector is to be expressed. Typically, the expression vectors contain a promoter and other regulatory sequences (e.g., enhancers) that are operably linked to the polynucleotides encoding an anti-GPC3×CD137 antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an anti-GPC3×CD137 antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-GPC3×CD137 antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-GPC3×CD137 antibodies. Insect cells in combination with baculovirus vectors can also be used. In other aspects, mammalian host cells are used to express and produce the anti-GPC3×CD137 antibodies of the present disclosure. For example, they can be either a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### Production of Bispecific Antibodies

The current standard for an engineered heterodimeric antibody Fc domain is the knobs-into-holes (KiH) design, which introduced mutations at the core CH3 domain interface. The resulted heterodimers have a reduced CH3 melting temperature (69° ° C. or less). On the contrary, the ZW heterodimeric Fc design has a thermal stability of 81.5° C., which is comparable to the wild-type CH3 domain.

### Methods of Detection and Diagnosis

The antibodies or antigen-binding fragments of the present disclosure are useful in a variety of applications including, but not limited to, methods for the detection of GPC3. In one aspect, the antibodies or antigen-binding fragments are useful for detecting the presence of GPC3 in a biological sample. The term “detecting” as used herein includes quantitative or qualitative detection. In certain aspects, a biological sample comprises a cell or tissue. In other aspects, such tissues include normal and/or cancerous tissues that express GPC3 at higher levels relative to other tissues.

In one aspect, the present disclosure provides a method of detecting the presence of GPC3 in a biological sample. In certain aspects, the method comprises contacting the biological sample with an anti-GPC3×CD137 antibody under conditions permissive for binding of the antibody to the antigen and detecting whether a complex is formed between the antibody and the antigen. The biological sample can include, without limitation, urine, tissue, sputum or blood samples.

Also included is a method of diagnosing a disorder associated with expression of GPC3. In certain aspects, the method comprises contacting a test cell with an anti-GPC3×CD137 antibody; determining the level of expression (either quantitatively or qualitatively) of GPC3 expressed by the test cell by detecting binding of the anti-GPC3×CD137 antibody to the GPC3 polypeptide; and comparing the level of expression by the test cell with the level of GPC3 expression in a control cell (e.g., a normal cell of the same tissue origin as the test cell or a non-GPC3 expressing cell), wherein a higher level of GPC3 expression in the test cell as compared to the control cell indicates the presence of a disorder associated with expression of GPC3.

### Methods of Treatment

The antibodies or antigen-binding fragments of the present disclosure are useful in a variety of applications including, but not limited to, methods for the treatment of a GPC3-associated disorder or disease. In one aspect, the GPC3-associated disorder or disease is a cancer.

In one aspect, the present disclosure provides a method of treating cancer. In certain aspects, the method comprises administering to a patient in need an effective amount of an anti-GPC3×CD137 antibody or antigen-binding fragment. In another aspect, the present disclosure provides the multispecific antibody or antigen-binding fragment thereof, or the pharmaceutical composition for use in the treatment of cancer expressing GPC3. In another aspect, the present disclosure provides the use of the multispecific antibody or antigen-binding fragment thereof, or the pharmaceutical composition in the manufacture of a medicament for the treatment of cancer expressing GPC3.

The cancer can include, without limitation, liver cancer, lung cancer, gastric cancer, germ cell tumors, thyroid cancer, pancreatic cancer, ovarian cancer, skin cancer, kidney cancer (e.g., Wilms tumor), atypical teratoid rhabdoid tumor of the brain, and undifferentiated synovial sarcoma. In one embodiment, the liver cancer is hepatoblastoma or hepatocellular carcinoma (HCC). In another embodiment, the lung cancer is non-small cell lung carcinoma (NSCLC) or small cell lung carcinomas (SCLC). In another embodiment, the non-small cell lung carcinoma is squamous non-small cell lung carcinoma. In another embodiment, the gastric cancer is alpha-fetoprotein positive (AFP+) gastric cancer. In another embodiment, the kidney cancer is Wilms tumor.

The antibody or antigen-binding fragment as disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or antigen-binding fragments of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above.

For the prevention or treatment of disease, the appropriate dosage of an antibody or antigen-binding fragment of the disclosure will depend on the type of disease to be treated, the type of antibody, the severity and course of the disease, whether the antibody is administered for preventive or therapeutic purposes, previous therapy, the patient's clinical history and response to the antibody, and the discretion of the attending physician.

### Combination Therapy

In one aspect, anti-GPC3×CD137 antibodies of the present disclosure can be used in combination with other therapeutic agents. Other therapeutic agents that can be used with the anti-GPC3×CD137 antibodies of the present disclosure include, but are not limited to, a chemotherapeutic agent (e.g., paclitaxel or a paclitaxel agent; (e.g. Abraxane®), docetaxel; carboplatin; topotecan; cisplatin; irinotecan, doxorubicin, lenalidomide, 5-azacytidine, ifosfamide, oxaliplatin, pemetrexed disodium, cyclophosphamide, etoposide, decitabine, fludarabine, vincristine, bendamustine, chlorambucil, busulfan, gemcitabine, melphalan, pentostatin, mitoxantrone, pemetrexed disodium), tyrosine kinase inhibitor (e.g., EGFR inhibitor (e.g., erlotinib), multikinase inhibitor (e.g., MGCD265, RGB-286638), CD-20 targeting agent (e.g., rituximab, ofatumumab, RO5072759, LFB-R603), CD52 targeting agent (e.g., alemtuzumab), prednisolone, darbepoetin alfa, lenalidomide, Bcl-2 inhibitor (e.g., oblimersen sodium), aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), EGEN-001, Polo-like kinase 1 inhibitor (e.g., BI 672). In another embodiment, other therapeutic agents include tislelizumab+bevacizumab, sorafenib, lenvatinib, or tislelizumab. In another embodiment, other therapeutic agents include carboplatin, cisplatin or paclitaxel as chemoradiation regimen. In another embodiment, other therapeutic agents include afatinib, erlotinib, dacomitinib, gefitinib, osimertinib, erlotinib+ramucirumab, or erlotinib+bevacizumab. In another embodiment, other therapeutic agents include tislelizumab/carboplatin/paclitaxel, tislelizumab/carboplatin/albumin-bound paclitaxel, carboplatin/albumin-bound paclitaxel, carboplatin/gemcitabine, or carboplatin/paclitaxel. In another embodiment, other therapeutic agents include trastuzumab, tislelizumab, fluorouracil, capecitabine, oxaliplatin or cisplatin.

In another embodiment, the other therapeutic agent is any one or more of paclitaxel or a paclitaxel agent, carboplatin, cisplatin, tislelizumab, bevacizumab, sorafenib, lenvatinib, afatinib, erlotinib, dacomitinib, gefitinib, osimertinib, ramucirumab, gemcitabine, trastuzumab, fluorouracil, capecitabine and oxaliplatin. In another embodiment, the other therapeutic agent is a paclitaxel agent, carboplatin, cisplatin, bevacizumab, gemcitabine, fluorouracil, capecitabine or oxaliplatin.

Anti-GPC3×CD137 antibodies of the present disclosure can be used in combination with other therapeutics, for example, other immune checkpoint antibodies. Such immune checkpoint antibodies can include anti-PD1 antibodies. Anti-PD1 antibodies can include, without limitation, Tislelizumab, Pembrolizumab or Nivolumab. Tislelizumab is disclosed in U.S. Pat. No. 8,735,553. Pembrolizumab (formerly MK-3475), is disclosed in U.S. Pat. Nos. 8,354,509 and 8,900,587 and is a humanized lgG4-K immunoglobulin which targets the PD1 receptor and inhibits binding of the PD1 receptor ligands PD-L1 and PD-L2. Pembrolizumab has been approved for the indications of metastatic melanoma and metastatic non-small cell lung cancer (NSCLC) and is under clinical investigation for the treatment of head and neck squamous cell carcinoma (HNSCC), and refractory Hodgkin's lymphoma (cHL). Nivolumab (as disclosed by Bristol-Meyers Squibb) is a fully human lgG4-K monoclonal antibody. Nivolumab (clone 5C4) is disclosed in U.S. Pat. No. 8,008,449 and WO 2006/121168. Nivolumab is approved for the treatment of melanoma, lung cancer, kidney cancer, and Hodgkin's lymphoma.

Other immune checkpoint antibodies for combination with anti-GPC3×CD137 antibodies can include anti-TIGIT antibodies. Such anti-TIGIT antibodies can include without limitation, anti-TIGIT antibodies as disclosed in WO2019/129261.

In one embodiment, the present disclosure provides a use of the combination of the multispecific antibody (anti-GPC3×CD137 antibody) and anti-PD-1 antibody (such as Tislelizumab or other anti-PD-1 antibody mentioned above) in the manufacture of a medicament for the treatment of cancer expressing GPC3. In another embodiment, the present disclosure provides the combination of the multispecific antibody (anti-GPC3×CD137 antibody) and anti-PD-1 antibody (such as Tislelizumab or other anti-PD-1 antibody mentioned above) for use in the treatment of cancer expressing GPC3.

### Pharmaceutical Compositions

Also provided are compositions, including pharmaceutical formulations, comprising an anti-GPC3×CD137 antibody or antigen-binding fragment thereof, or polynucleotides comprising sequences encoding an anti-GPC3×CD137 antibody or antigen-binding fragment. In certain embodiments, compositions comprise one or more anti-GPC3×CD137 antibodies or antigen-binding fragments, or one or more polynucleotides comprising sequences encoding one or more anti-GPC3×CD137 antibodies or antigen-binding fragments. These compositions can further comprise suitable carriers, such as pharmaceutically acceptable excipients including buffers, which are well known in the art.

Pharmaceutical formulations of an anti-GPC3×CD137 antibody or antigen-binding fragment as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility can be readily accomplished, e.g., by filtration through sterile filtration membranes.

### Definitions

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The term “CD137” or “TNFRSF9,” “ILA” or “41BB” refers to a co-stimulatory molecule belonging to the TNFRSF family. The amino acid sequence of human CD137 (SEQ ID NO: 94) can also be found at accession number Q07011 (TNR9_HUMAN) or U03397. The nucleic acid sequence of human CD137 is set forth in SEQ ID NO: 95.

The term “Glypican 3” (GPC3) is also known as DGSX, GTR2-2, MXR7, OCI-5, SDYS, SGB, SGBS, SGBS1. The amino acid sequence of human GPC3 (SEQ ID NO: 212) can also be found at NCBI Reference Sequence: NP_004475.1. The nucleic acid sequence of human GPC3 is set forth in SEQ ID NO: 213.

The terms “administration,” “administering,” “treating,” and “treatment” as used herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “subject” in the context of the present disclosure is a mammal, e.g., a primate, preferably a higher primate, e.g., a human (e.g., a patient comprising, or at risk of having, a disorder described herein).

The term “affinity” as used herein refers to the strength of interaction between antibody and antigen. Within the antigen, the variable regions of the antibody interacts through non-covalent forces with the antigen at numerous sites. In general, the more interactions, the stronger the affinity.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL or VK) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four framework regions (FRs) arranged from amino-terminus to carboxyl-terminus in the following order: FRI, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “antibody” includes, but is not limited to, monoclonal antibodies, human antibodies, humanized antibodies, chimeric antibodies, and anti-idiotypic (anti-Id) antibodies, a human engineered antibody, a single chain antibody (scFv), a single domain antibody, a Fab fragment, a Fab′ fragment, or a F(ab′)₂ fragment. The antibodies can be of any isotype/class (e.g., IgG, IgE, IgM, IgD, IgA and IgY), or subclass (e.g., IgG1, IgG2, IgG3, IgG4, IgA1 and IgA2). In addition, the antibody includes the derivative agents thereof, such as by linking to another agent (such as other drug) directly or indirectly or forming a complex with another agent.

In some embodiments, the anti-GPC3 antibodies comprise at least one antigen-binding site, at least a variable region. In some embodiments, the anti-GPC3 antibodies comprise an antigen-binding fragment from an GPC3 antibody described herein. In some embodiments, the anti-GPC3 antibody is isolated or recombinant.

In some embodiments, the anti-CD137 antibodies comprise at least one antigen-binding site, at least a variable region. In some embodiments, the anti-CD137 antibodies comprise an antigen-binding fragment from an CD137 antibody described herein. In some embodiments, the anti-CD137 antibody is isolated or recombinant.

The term “monoclonal antibody” or “mAb” or “Mab” herein means a population of substantially homogeneous antibodies, i.e., the antibody molecules comprised in the population are identical in amino acid sequence except for possible naturally occurring mutations that can be present in minor amounts. In contrast, conventional (polyclonal) antibody preparations typically include a multitude of different antibodies having different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art. See, for example Kohler et al., Nature 1975 256:495-497; U.S. Pat. No. 4,376,110; Ausubel et al., CURRENT PROTOCOLS IN MOLECULAR BIOLOGY 1992; Harlow et al., ANTIBODIES: A LABORATORY MANUAL, Cold spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain can define a constant region primarily responsible for effector function. Typically, human light chains are classified as kappa and lambda light chains. Furthermore, human heavy chains are typically classified as a, 8, &, Y, or u, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same in primary sequence.

Typically, the variable domains of both the heavy and light chains comprise three hypervariable regions, also called “complementarity determining regions (CDRs),” which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (or FR3), CDR-3 (CDR3), and FR-4 (or FR4). The positions of the CDRs and framework regions can be determined using various well known definitions in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997) ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203: 121-153 (1991); and Rees et al., In Sternberg M. J. E. (ed.), Protein Structure Prediction, Oxford University Press, Oxford, 141-172 (1996). For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGap Align.

The term “hypervariable region” means the amino acid residues of an antibody that are responsible for antigen-binding. The hypervariable region comprises amino acid residues from a “CDR” (e.g., LCDR1, LCDR2 and LCDR3 in the light chain variable domain and HCDR1, HCDR2 and HCDR3 in the heavy chain variable domain). See, Kabat et al., (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues means those variable domain residues other than the hypervariable region residues defined herein as CDR residues.

Unless otherwise indicated, an “antigen-binding fragment” means antigen-binding fragments of antibodies, i.e. antibody fragments that retain the ability to bind specifically to the antigen bound by the full-length antibody, e.g., fragments that retain one or more CDR regions. Examples of antigen-binding fragments include, but not limited to, Fab, Fab′, F(ab′)₂, and Fv fragments; diabodies; linear antibodies; single-chain antibody molecules, e.g., single chain Fv (ScFv); nanobodies and multispecific antibodies formed from antibody fragments.

As used herein, an antibody “specifically binds” to a target protein, meaning the antibody exhibits preferential binding to that target as compared to other proteins, but this specificity does not require absolute binding specificity. An antibody “specifically binds” or “selectively binds,” is used in the context of describing the interaction between an antigen (e.g., a protein) and an antibody, or antigen binding antibody fragment, refers to a binding reaction that is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times when compared to the background level and do not specifically bind in a significant amount to other antigens present in the sample. In one aspect, under designated immunoassay conditions, the antibody or antigen-binding fragment thereof, specifically bind to a particular antigen at least ten (10) times when compared to the background level of binding and does not specifically bind in a significant amount to other antigens present in the sample.

“Antigen-binding domain” as used herein, comprise at least six CDRs and specifically bind to an epitope (or three CDRs in terms of single domain antibody). An “antigen-binding domain” of a multispecific antibody (e.g., a bispecific antibody) comprises a first antigen binding domain that specifically binds to a first epitope and a second antigen binding domain specifically binds to a second epitope. Multispecific antibodies can be bispecific, trispecific, tetraspecific etc., with antigen binding domains directed to each specific epitope. Multispecific antibodies can be multivalent (e.g., a bispecific tetravalent antibody) that comprises multiple antigen binding domains, for example, 2, 3, 4 or more antigen binding domains that specifically bind to a first epitope and 2, 3, 4 or more antigen binding domains that specifically bind a second epitope.

The term “human antibody” herein means an antibody that comprises human immunoglobulin protein sequences only. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

The term “equilibrium dissociation constant (K_{D}), M)” refers to the dissociation rate constant (kd, time⁻¹) divided by the association rate constant (ka, time⁻¹, M⁻¹). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M or 10⁻¹³ M.

The terms “cancer” or “tumor” herein has the broadest meaning as understood in the art and refers to the physiological condition in mammals that is typically characterized by unregulated cell growth. In the context of the present disclosure, the cancer is not limited to certain type or location.

In the context of the present disclosure, when reference is made to an amino acid sequence, the term “conservative substitution” means substitution of the original amino acid by a new amino acid that does not substantially alter the chemical, physical and/or functional properties of the antibody or fragment, e.g., its binding affinity to GPC3 or to CD137. Specifically, common conservative substations of amino acids are well known in the art.

The term “knob-into-hole” technology as used herein refers to amino acids that direct the pairing of two polypeptides together either in vitro or in vivo by introducing a spatial protuberance (knob) into one polypeptide and a socket or cavity (hole) into the other polypeptide at an interface in which they interact. For example, knob-into-holes have been introduced in the Fc:Fc binding interfaces, C_{L}:C_{H}I interfaces or V_{H}/V_{L} interfaces of antibodies (see, e.g., US 2011/0287009, US2007/0178552, WO 96/027011, WO 98/050431, and Zhu et al., 1997, Protein Science 6:781-788). In some embodiments, knob-into-holes insure the correct pairing of two different heavy chains together during the manufacture of multispecific antibodies. For example, multispecific antibodies having knob-into-hole amino acids in their Fc regions can further comprise single variable domains linked to each Fc region, or further comprise different heavy chain variable domains that pair with similar or different light chain variable domains. Knob-into-hole technology can also be used in the VH or VL regions to also insure correct pairing.

The term “knob” as used herein in the context of “knob-into-hole” technology refers to an amino acid change that introduces a protuberance (knob) into a polypeptide at an interface in which the polypeptide interacts with another polypeptide. In some embodiments, the other polypeptide has a hole mutation.

The term “hole” as used herein in the context of “knob-into-hole” refers to an amino acid change that introduces a socket or cavity (hole) into a polypeptide at an interface in which the polypeptide interacts with another polypeptide. In some embodiments, the other polypeptide has a knob mutation.

Examples of algorithms that are suitable for determining percent sequence identity and sequence similarity are the BLAST algorithms, which are described in Altschul et al, Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89: 10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4: 11-17, (1988), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some aspects, the present disclosure provides compositions, e.g., pharmaceutically acceptable compositions, which include anti-GPC3×CD137 multispecific antibodies as described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g., by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

The term “therapeutically effective amount” as herein used, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The term “combination therapy” refers to the administration of two or more therapeutic agents to treat a therapeutic condition or disorder described in the present disclosure. Such administration encompasses co-administration of these therapeutic agents in a substantially simultaneous manner. Such administration also encompasses co-administration in multiple, or in separate containers (e.g., capsules, powders, and liquids) for each active ingredient. Powders and/or liquids can be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an anti-GPC3×CD137 multispecific antibody is administered to the subject at the same time as, just before, or just after administration of an additional therapeutic agent. In certain embodiments, an anti-GPC3×CD137 multispecific antibody is administered as a co-formulation with an additional therapeutic agent.

### Equivalent

It is to be understood that while the present invention has been described in conjunction with the detailed description thereof, the foregoing description is intended to illustrate and not limit the scope of the invention, which is defined by the scope of the appended claims. Other aspects, advantages, and modifications are within the scope of the following claims.

It is to be understood that one, some, any or all of the features of the various embodiments described herein may be combined to form further embodiments of the present disclosure. These and other aspects of the present disclosure will become apparent to those skilled in the art.

### EXAMPLES

### Example 1. Generation of Recombinant Proteins and Stable Cell Lines

### CD137 Recombinant Proteins for Phage Campaign and Binding Assays

To discover VH domain antibodies against CD137 with cross-binding of human and Macaca mulatta CD137, but without off-target binding with other human TNF receptor members, several recombinant proteins were designed and expressed for phage panning and screening (see Table 2). The cDNA coding regions for the full-length human CD137 (SEQ ID NO: 94) was ordered based on the CD137 GenBank sequence (Accession No: NM_001561.4, the gene is available from Sinobio, Cat.: HG10041-M). Human CD137 ligand (TNFSF9) (SEQ ID NO: 104) was ordered based on (Accession No: NM_003811.3, the gene is available from Sinobio, Cat.: HG15693-G). Monkey (Macaca mulatta) CD137 (SEQ ID NO: 110) was ordered based on (Accession No: NM_001266128.1, the gene is available from Genscript, Cat.: OMb00270). The full-length human CD40 (SEQ ID NO: 116) was ordered based on (Accession No: NM_001250.4, the gene is available from Sinobio, Cat.: HG10774-M). OX40 (SEQ ID NO: 122) was ordered based on (Accession No: NM_003327.2, the gene is available from Sinobio, Cat.: HG10481-UT). In brief, the coding region of extracellular domain (ECD) consisting of amino acid (AA) 24-183 of huCD137 (SEQ ID NO: 96), the coding region of ECD consisting of AA 71-254 of human CD137 ligand (SEQ ID NO: 106), the coding region of ECD consisting of AA 24-186 of cynoCD137 (SEQ ID NO: 112), and the coding region of ECD consisting of AA 1-194 of human CD40 (SEQ ID NO: 118) were PCR-amplified, respectively. The coding region of mIgG2a Fc (SEQ ID NO: 102) was PCR-amplified, and then conjugated with ECDs of human CD137, human CD137 ligand, monkey CD137 or human CD40 by overlap-PCR to make mIgG2a Fc-fusion proteins. PCR products were then cloned into a pcDNA3.1-based expression vector (Invitrogen, Carlsbad, CA, USA), which resulted in four recombinant mIgG2a Fc-fusion protein expression plasmids, human CD137 ECD-mIgG2a, human CD137 ligand-mIgG2a, cyno CD137 ECD-mIgG2a and human CD40 ECD-mIgG2a. Alternatively the coding regions of ECD consisting of AA 24-183 (SEQ ID NO: 96) of huCD137 (SEQ ID NO: 94) and the coding region of ECD consisting of AA 1-216 of human OX40 (SEQ ID NO: 124) were also cloned into a pcDNA3.1-based expression vector (Invitrogen, Carlsbad, CA, USA) with C-terminus fused with 6×His tags, which resulted in human CD137-his and human OX40-his, respectively. For the recombinant fusion protein production, plasmids were transiently transfected into a HEK293-based mammalian cell expression system (developed in house) and cultured for 5-7 days in a CO₂ incubator equipped with rotating shaker. The supernatants containing the recombinant proteins were collected and cleared by centrifugation. Recombinant proteins were purified using a Protein A column (Cat.: 17127901, GE Life Sciences) or a Ni-NTA agarose (Cat.: R90115, Invitrogen). All recombinant proteins were dialyzed against phosphate buffered saline (PBS) and stored in −80° C. freezer in small aliquots.

### Stable Expression Cell Lines

To establish stable cell lines that express full-length human CD137 (huCD137), huCD137 sequences were cloned into a retroviral vector pFB-Neo (Cat.: 217561, Agilent, USA). Dual-tropic retroviral vectors were generated according to a previous protocol (Zhang, et al., (2005) Blood, 106, 1544-1551). Vectors containing huCD137 were transduced into Hut78 cells (ATCC, TIB-161) or NK92-mi cells (ATCC, CRL-2408), to generate the huCD137 expressing cell lines, Hut78/huCD137 or NK92-mi/huCD137. huCD137 expressing cell lines were selected by culture in medium containing 10% FBS with G418, and then verified via FACS.

TABLE 2
Sequences for recombinant CD137 proteins

| CONSTRUCT | SEQ ID NO: | SEQUENCE |
|---|---|---|
| human CD137 FL | SEQ ID NO: 94 | MGNSCYNIVATLLLVLNFERTRSLQDPCSNC |
| AA | | PAGTFCDNNRNQICSPCPPNSFSSAGGQRTC |
| | | DICRQCKGVFRTRKECSSTSNAECDCTPGFH |
| | | CLGAGCSMCEQDCKQGQELTKKGCKDCCF |
| | | GTFNDQKRGICRPWTNCSLDGKSVLVNGTK |
| | | ERDVVCGPSPADLSPGASSVTPPAPAREPGHS |
| | | PQIISFFLALTSTALLFLLFFLTLRFSVVKRGR |
| | | KKLLYIFKQPFMRPVQTTQEEDGCSCRFPEE |
| | | EEGGCEL |
| human CD137 FL | SEQ ID NO: 95 | ATGGGAAACAGCTGTTACAACATAGTAGCC |
| DNA | | ACTCTGTTGCTGGTCCTCAACTTTGAGAGG |
| | | ACAAGATCATTGCAGGATCCTTGTAGTAAC |
| | | TGCCCAGCTGGTACATTCTGTGATAATAAC |
| | | AGGAATCAGATTTGCAGTCCCTGTCCTCCA |
| | | AATAGTTTCTCCAGCGCAGGTGGACAAAG |
| | | GACCTGTGACATATGCAGGCAGTGTAAAG |
| | | GTGTTTTCAGGACCAGGAAGGAGTGTTCC |
| | | TCCACCAGCAATGCAGAGTGTGACTGCAC |
| | | TCCAGGGTTTCACTGCCTGGGGGCAGGAT |
| | | GCAGCATGTGTGAACAGGATTGTAAACAA |
| | | GGTCAAGAACTGACAAAAAAAGGTTGTAA |
| | | AGACTGTTGCTTTGGGACATTTAACGATCA |
| | | GAAACGTGGCATCTGTCGACCCTGGACAA |
| | | ACTGTTCTTTGGATGGAAAGTCTGTGCTTG |
| | | TGAATGGGACGAAGGAGAGGGACGTGGTC |
| | | TGTGGACCATCTCCAGCCGACCTCTCTCCG |
| | | GGAGCATCCTCTGTGACCCCGCCTGCCCCT |
| | | GCGAGAGAGCCAGGACACTCTCCGCAGAT |
| | | CATCTCCTTCTTTCTTGCGCTGACGTCGAC |
| | | TGCGTTGCTCTTCCTGCTGTTCTTCCTCAC |
| | | GCTCCGTTTCTCTGTTGTTAAACGGGGCAG |
| | | AAAGAAACTCCTGTATATATTCAAACAACC |
| | | ATTTATGAGACCAGTACAAACTACTCAAGA |
| | | GGAAGATGGCTGTAGCTGCCGATTTCCAGA |
| | | AGAAGAAGAAGGAGGATGTGAACTG |
| human CD137 AA | SEQ ID NO: 96 | LQDPCSNCPAGTFCDNNRNQICSPCPPNSFSS |
| 24-183 | | AGGQRTCDICRQCKGVFRTRKECSSTSNAEC |
| | | DCTPGFHCLGAGCSMCEQDCKQGQELTKK |
| | | GCKDCCFGTFNDQKRGICRPWTNCSLDGKS |
| | | VLVNGTKERDVVCGPSPADLSPGASSVTPPA |
| | | PAREPGH |
| human CD137 24- | SEQ ID NO: 97 | CTGCAGGATCCTTGTAGTAACTGCCCAGCT |
| 183 DNA | | GGTACATTCTGTGATAATAACAGGAATCAG |
| | | ATTTGCAGTCCCTGTCCTCCAAATAGTTTCT |
| | | CCAGCGCAGGTGGACAAAGGACCTGTGAC |
| | | ATATGCAGGCAGTGTAAAGGTGTTTTCAGG |
| | | ACCAGGAAGGAGTGTTCCTCCACCAGCAA |
| | | TGCAGAGTGTGACTGCACTCCAGGGTTTC |
| | | ACTGCCTGGGGGCAGGATGCAGCATGTGT |
| | | GAACAGGATTGTAAACAAGGTCAAGAACT |
| | | GACAAAAAAAGGTTGTAAAGACTGTTGCT |
| | | TTGGGACATTTAACGATCAGAAACGTGGC |
| | | ATCTGTCGACCCTGGACAAACTGTTCTTTG |
| | | GATGGAAAGTCTGTGCTTGTGAATGGGAC |
| | | GAAGGAGAGGGACGTGGTCTGTGGACCAT |
| | | CTCCAGCCGACCTCTCTCCGGGAGCATCCT |
| | | CTGTGACCCCGCCTGCCCCTGCGAGAGAG |
| | | CCAGGACAC |
| human CD137-his | SEQ ID NO: 98 | LQDPCSNCPAGTFCDNNRNQICSPCPPNSFSS |
| | | AGGQRTCDICRQCKGVFRTRKECSSTSNAEC |
| | | DCTPGFHCLGAGCSMCEQDCKQGQELTKK |
| | | GCKDCCFGTFNDQKRGICRPWTNCSLDGKS |
| | | VLVNGTKERDVVCGPSPADLSPGASSVTPPA |
| | | PAREPGHHHHHHHGGGLNDIFEAQKIEWHE |
| human CD137-his | SEQ ID NO: 99 | CTGCAGGATCCTTGTAGTAACTGCCCAGCT |
| DNA | | GGTACATTCTGTGATAATAACAGGAATCAG |
| | | ATTTGCAGTCCCTGTCCTCCAAATAGTTTCT |
| | | CCAGCGCAGGTGGACAAAGGACCTGTGAC |
| | | ATATGCAGGCAGTGTAAAGGTGTTTTCAGG |
| | | ACCAGGAAGGAGTGTTCCTCCACCAGCAA |
| | | TGCAGAGTGTGACTGCACTCCAGGGTTTC |
| | | ACTGCCTGGGGGCAGGATGCAGCATGTGT |
| | | GAACAGGATTGTAAACAAGGTCAAGAACT |
| | | GACAAAAAAAGGTTGTAAAGACTGTTGCT |
| | | TTGGGACATTTAACGATCAGAAACGTGGC |
| | | ATCTGTCGACCCTGGACAAACTGTTCTTTG |
| | | GATGGAAAGTCTGTGCTTGTGAATGGGAC |
| | | GAAGGAGAGGGACGTGGTCTGTGGACCAT |
| | | CTCCAGCCGACCTCTCTCCGGGAGCATCCT |
| | | CTGTGACCCCGCCTGCCCCTGCGAGAGAG |
| | | CCAGGACACCATCACCATCACCATCACGGA |
| | | GGCGGTCTGAACGACATCTTCGAGGCTCA |
| | | GAAAATCGAATGGCACGAA |
| human CD137 | SEQ ID NO: | LQDPCSNCPAGTFCDNNRNQICSPCPPNSFSS |
| ECD-mIgG2a AA | 100 | AGGQRTCDICRQCKGVFRTRKECSSTSNAEC |
| | | DCTPGFHCLGAGCSMCEQDCKQGQELTKK |
| | | GCKDCCFGTFNDQKRGICRPWTNCSLDGKS |
| | | VLVNGTKERDVVCGPSPADLSPGASSVTPPA |
| | | PAREPGHEPRGPTIKPCPPCKCPAPNLLGGPS |
| | | VFIFPPKIKDVLMISLSPIVTCVVVDVSEDDP |
| | | DVQISWFVNNVEVHTAQTQTHREDYNSTLR |
| | | VVSALPIQHQDWMSGKEFKCKVNNKDLPAP |
| | | IERTISKPKGSVRAPQVYVLPPPEEEMTKKQ |
| | | VTLTCMVTDFMPEDIYVEWTNNGKTELNYK |
| | | NTEPVLDSDGSYFMYSKLRVEKKNWVERNS |
| | | YSCSVVHEGLHNHHTTKSFSRTPGK |
| human CD137 | SEQ ID NO: | CTGCAGGATCCTTGTAGTAACTGCCCAGCT |
| ECD-mIgG2a | 101 | GGTACATTCTGTGATAATAACAGGAATCAG |
| DNA | | ATTTGCAGTCCCTGTCCTCCAAATAGTTTCT |
| | | CCAGCGCAGGTGGACAAAGGACCTGTGAC |
| | | ATATGCAGGCAGTGTAAAGGTGTTTTCAGG |
| | | ACCAGGAAGGAGTGTTCCTCCACCAGCAA |
| | | TGCAGAGTGTGACTGCACTCCAGGGTTTC |
| | | ACTGCCTGGGGGCAGGATGCAGCATGTGT |
| | | GAACAGGATTGTAAACAAGGTCAAGAACT |
| | | GACAAAAAAAGGTTGTAAAGACTGTTGCT |
| | | TTGGGACATTTAACGATCAGAAACGTGGC |
| | | ATCTGTCGACCCTGGACAAACTGTTCTTTG |
| | | GATGGAAAGTCTGTGCTTGTGAATGGGAC |
| | | GAAGGAGAGGGACGTGGTCTGTGGACCAT |
| | | CTCCAGCCGACCTCTCTCCGGGAGCATCCT |
| | | CTGTGACCCCGCCTGCCCCTGCGAGAGAG |
| | | CCAGGACACGAGCCCAGAGGGCCCACAAT |
| | | CAAGCCCTGTCCTCCATGCAAATGCCCAGC |
| | | ACCTAACCTCTTGGGTGGACCATCCGTCTT |
| | | CATCTTCCCTCCAAAGATCAAGGATGTACT |
| | | CATGATCTCCCTGAGCCCCATAGTCACATG |
| | | TGTGGTGGTGGATGTGAGCGAGGATGACC |
| | | CAGATGTCCAGATCAGCTGGTTTGTGAACA |
| | | ACGTGGAAGTACACACAGCTCAGACACAA |
| | | ACCCATAGAGAGGATTACAACAGTACTCTC |
| | | CGGGTGGTCAGTGCCCTCCCCATCCAGCAC |
| | | CAGGACTGGATGAGTGGCAAGGAGTTCAA |
| | | ATGCAAGGTCAACAACAAAGACCTCCCAG |
| | | CGCCCATCGAGAGAACCATCTCAAAACCC |
| | | AAAGGGTCAGTAAGAGCTCCACAGGTATAT |
| | | GTCTTGCCTCCACCAGAAGAAGAGATGAC |
| | | TAAGAAACAGGTCACTCTGACCTGCATGG |
| | | TCACAGACTTCATGCCTGAAGACATTTACG |
| | | TGGAGTGGACCAACAACGGGAAAACAGA |
| | | GCTAAACTACAAGAACACTGAACCAGTCC |
| | | TGGACTCTGATGGTTCTTACTTCATGTACA |
| | | GCAAGCTGAGAGTGGAAAAGAAGAACTG |
| | | GGTGGAAAGAAATAGCTACTCCTGTTCAGT |
| | | GGTCCACGAGGGTCTGCACAATCACCACA |
| | | CGACTAAGAGCTTCTCCCGGACTCCGGGTA |
| | | AA |
| mIgG2a AA | SEQ ID NO: | EPRGPTIKPCPPCKCPAPNLLGGPSVFIFPPKI |
| | 102 | KDVLMISLSPIVTCVVVDVSEDDPDVQISWF |
| | | VNNVEVHTAQTQTHREDYNSTLRVVSALPI |
| | | QHQDWMSGKEFKCKVNNKDLPAPIERTISK |
| | | PKGSVRAPQVYVLPPPEEEMTKKQVTLTCM |
| | | VTDFMPEDIYVEWTNNGKTELNYKNTEPVL |
| | | DSDGSYFMYSKLRVEKKNWVERNSYSCSV |
| | | VHEGLHNHHTTKSFSRTPGK |
| mIgG2a DNA | SEQ ID NO: | GAGCCCAGAGGGCCCACAATCAAGCCCTG |
| | 103 | TCCTCCATGCAAATGCCCAGCACCTAACCT |
| | | CTTGGGTGGACCATCCGTCTTCATCTTCCC |
| | | TCCAAAGATCAAGGATGTACTCATGATCTC |
| | | CCTGAGCCCCATAGTCACATGTGTGGTGGT |
| | | GGATGTGAGCGAGGATGACCCAGATGTCC |
| | | AGATCAGCTGGTTTGTGAACAACGTGGAA |
| | | GTACACACAGCTCAGACACAAACCCATAG |
| | | AGAGGATTACAACAGTACTCTCCGGGTGGT |
| | | CAGTGCCCTCCCCATCCAGCACCAGGACT |
| | | GGATGAGTGGCAAGGAGTTCAAATGCAAG |
| | | GTCAACAACAAAGACCTCCCAGCGCCCAT |
| | | CGAGAGAACCATCTCAAAACCCAAAGGGT |
| | | CAGTAAGAGCTCCACAGGTATATGTCTTGC |
| | | CTCCACCAGAAGAAGAGATGACTAAGAAA |
| | | CAGGTCACTCTGACCTGCATGGTCACAGA |
| | | CTTCATGCCTGAAGACATTTACGTGGAGTG |
| | | GACCAACAACGGGAAAACAGAGCTAAACT |
| | | ACAAGAACACTGAACCAGTCCTGGACTCT |
| | | GATGGTTCTTACTTCATGTACAGCAAGCTG |
| | | AGAGTGGAAAAGAAGAACTGGGTGGAAA |
| | | GAAATAGCTACTCCTGTTCAGTGGTCCACG |
| | | AGGGTCTGCACAATCACCACACGACTAAG |
| | | AGCTTCTCCCGGACTCCGGGTAAA |
| human CD137 | SEQ ID NO: | MEYASDASLDPEAPWPPAPRARACRVLPWA |
| ligand AA | 104 | LVAGLLLLLLLAAACAVFLACPWAVSGARAS |
| | | PGSAASPRLREGPELSPDDPAGLLDLRQGMF |
| | | AQLVAQNVLLIDGPLSWYSDPGLAGVSLTG |
| | | GLSYKEDTKELVVAKAGVYYVFFQLELRRV |
| | | VAGEGSGSVSLALHLQPLRSAAGAAALALT |
| | | VDLPPASSEARNSAFGFQGRLLHLSAGQRLG |
| | | VHLHTEARARHAWQLTQGATVLGLFRVTPEI |
| | | PAGLPSPRSE |
| human CD137 | SEQ ID NO: | ATGGAATACGCCTCTGACGCTTCACTGGAC |
| ligand cDNA | 105 | CCCGAAGCCCCGTGGCCTCCCGCGCCCCG |
| | | CGCTCGCGCCTGCCGCGTACTGCCTTGGGC |
| | | CCTGGTCGCGGGGCTGCTGCTGCTGCTGCT |
| | | GCTCGCTGCCGCCTGCGCCGTCTTCCTCGC |
| | | CTGCCCCTGGGCCGTGTCCGGGGCTCGCG |
| | | CCTCGCCCGGCTCCGCGGCCAGCCCGAGA |
| | | CTCCGCGAGGGTCCCGAGCTTTCGCCCGA |
| | | CGATCCCGCCGGCCTCTTGGACCTGCGGCA |
| | | GGGCATGTTTGCGCAGCTGGTGGCCCAAA |
| | | ATGTTCTGCTGATCGATGGGCCCCTGAGCT |
| | | GGTACAGTGACCCAGGCCTGGCAGGCGTG |
| | | TCCCTGACGGGGGGCCTGAGCTACAAAGA |
| | | GGACACGAAGGAGCTGGTGGTGGCCAAG |
| | | GCTGGAGTCTACTATGTCTTCTTTCAACTA |
| | | GAGCTGCGGCGCGTGGTGGCCGGCGAGGG |
| | | CTCAGGCTCCGTTTCACTTGCGCTGCACCT |
| | | GCAGCCACTGCGCTCTGCTGCTGGGGCCG |
| | | CCGCCCTGGCTTTGACCGTGGACCTGCCAC |
| | | CCGCCTCCTCCGAGGCTCGGAACTCGGCC |
| | | TTCGGTTTCCAGGGCCGCTTGCTGCACCTG |
| | | AGTGCCGGCCAGCGCCTGGGCGTCCATCTT |
| | | CACACTGAGGCCAGGGCACGCCATGCCTG |
| | | GCAGCTTACCCAGGGCGCCACAGTCTTGG |
| | | GACTCTTCCGGGTGACCCCCGAAATCCCA |
| | | GCCGGACTCCCTTCACCGAGGTCGGAATA |
| | | A |
| human CD137 | SEQ ID NO: | REGPELSPDDPAGLLDLRQGMFAQLVAQNV |
| ligand 71-254 AA | 106 | LLIDGPLSWYSDPGLAGVSLTGGLSYKEDTK |
| | | ELVVAKAGVYYVFFQLELRRVVAGEGSGSV |
| | | SLALHLQPLRSAAGAAALALTVDLPPASSEA |
| | | RNSAFGFQGRLLHLSAGQRLGVHLHTEARA |
| | | RHAWQLTQGATVLGLFRVTPEIPAGLPSPRSE |
| human CD137 | SEQ ID NO: | CGCGAGGGTCCCGAGCTTTCGCCCGACGA |
| ligand 71-254 DNA | 107 | TCCCGCCGGCCTCTTGGACCTGCGGCAGG |
| | | GCATGTTTGCGCAGCTGGTGGCCCAAAATG |
| | | TTCTGCTGATCGATGGGCCCCTGAGCTGGT |
| | | ACAGTGACCCAGGCCTGGCAGGCGTGTCC |
| | | CTGACGGGGGGCCTGAGCTACAAAGAGGA |
| | | CACGAAGGAGCTGGTGGTGGCCAAGGCTG |
| | | GAGTCTACTATGTCTTCTTTCAACTAGAGC |
| | | TGCGGCGCGTGGTGGCCGGCGAGGGCTCA |
| | | GGCTCCGTTTCACTTGCGCTGCACCTGCAG |
| | | CCACTGCGCTCTGCTGCTGGGGCCGCCGC |
| | | CCTGGCTTTGACCGTGGACCTGCCACCCGC |
| | | CTCCTCCGAGGCTCGGAACTCGGCCTTCG |
| | | GTTTCCAGGGCCGCTTGCTGCACCTGAGTG |
| | | CCGGCCAGCGCCTGGGCGTCCATCTTCACA |
| | | CTGAGGCCAGGGCACGCCATGCCTGGCAG |
| | | CTTACCCAGGGCGCCACAGTCTTGGGACT |
| | | CTTCCGGGTGACCCCCGAAATCCCAGCCG |
| | | GACTCCCTTCACCGAGGTCGGAA |
| human CD137 | SEQ ID NO: | EPRGPTIKPCPPCKCPAPNLLGGPSVFIFPPKI |
| ligand-mIgG2a AA | 108 | KDVLMISLSPIVTCVVVDVSEDDPDVQISWF |
| | | VNNVEVHTAQTQTHREDYNSTLRVVSALPI |
| | | QHQDWMSGKEFKCKVNNKDLPAPIERTISK |
| | | PKGSVRAPQVYVLPPPEEEMTKKQVTLTCM |
| | | VTDFMPEDIYVEWTNNGKTELNYKNTEPVL |
| | | DSDGSYFMYSKLRVEKKNWVERNSYSCSV |
| | | VHEGLHNHHTTKSFSRTPGKGGGGSREGPE |
| | | LSPDDPAGLLDLRQGMFAQLVAQNVLLIDGP |
| | | LSWYSDPGLAGVSLTGGLSYKEDTKELVVA |
| | | KAGVYYVFFQLELRRVVAGEGSGSVSLALH |
| | | LQPLRSAAGAAALALTVDLPPASSEARNSAF |
| | | GFQGRLLHLSAGQRLGVHLHTEARARHAW |
| | | QLTQGATVLGLFRVTPEIPAGLPSPRSE |
| human CD137 | SEQ ID NO: | GAGCCCAGAGGGCCCACAATCAAGCCCTG |
| ligand-mIgG2a | 109 | TCCTCCATGCAAATGCCCAGCACCTAACCT |
| DNA | | CTTGGGTGGACCATCCGTCTTCATCTTCCC |
| | | TCCAAAGATCAAGGATGTACTCATGATCTC |
| | | CCTGAGCCCCATAGTCACATGTGTGGTGGT |
| | | GGATGTGAGCGAGGATGACCCAGATGTCC |
| | | AGATCAGCTGGTTTGTGAACAACGTGGAA |
| | | GTACACACAGCTCAGACACAAACCCATAG |
| | | AGAGGATTACAACAGTACTCTCCGGGTGGT |
| | | CAGTGCCCTCCCCATCCAGCACCAGGACT |
| | | GGATGAGTGGCAAGGAGTTCAAATGCAAG |
| | | GTCAACAACAAAGACCTCCCAGCGCCCAT |
| | | CGAGAGAACCATCTCAAAACCCAAAGGGT |
| | | CAGTAAGAGCTCCACAGGTATATGTCTTGC |
| | | CTCCACCAGAAGAAGAGATGACTAAGAAA |
| | | CAGGTCACTCTGACCTGCATGGTCACAGA |
| | | CTTCATGCCTGAAGACATTTACGTGGAGTG |
| | | GACCAACAACGGGAAAACAGAGCTAAACT |
| | | ACAAGAACACTGAACCAGTCCTGGACTCT |
| | | GATGGTTCTTACTTCATGTACAGCAAGCTG |
| | | AGAGTGGAAAAGAAGAACTGGGTGGAAA |
| | | GAAATAGCTACTCCTGTTCAGTGGTCCACG |
| | | AGGGTCTGCACAATCACCACACGACTAAG |
| | | AGCTTCTCCCGGACTCCGGGTAAAGGTGG |
| | | AGGCGGTTCACGCGAGGGTCCCGAGCTTT |
| | | CGCCCGACGATCCCGCCGGCCTCTTGGACC |
| | | TGCGGCAGGGCATGTTTGCGCAGCTGGTG |
| | | GCCCAAAATGTTCTGCTGATCGATGGGCCC |
| | | CTGAGCTGGTACAGTGACCCAGGCCTGGC |
| | | AGGCGTGTCCCTGACGGGGGGCCTGAGCT |
| | | ACAAAGAGGACACGAAGGAGCTGGTGGT |
| | | GGCCAAGGCTGGAGTCTACTATGTCTTCTT |
| | | TCAACTAGAGCTGCGGCGCGTGGTGGCCG |
| | | GCGAGGGCTCAGGCTCCGTTTCACTTGCG |
| | | CTGCACCTGCAGCCACTGCGCTCTGCTGCT |
| | | GGGGCCGCCGCCCTGGCTTTGACCGTGGA |
| | | CCTGCCACCCGCCTCCTCCGAGGCTCGGA |
| | | ACTCGGCCTTCGGTTTCCAGGGCCGCTTGC |
| | | TGCACCTGAGTGCCGGCCAGCGCCTGGGC |
| | | GTCCATCTTCACACTGAGGCCAGGGCACG |
| | | CCATGCCTGGCAGCTTACCCAGGGCGCCA |
| | | CAGTCTTGGGACTCTTCCGGGTGACCCCCG |
| | | AAATCCCAGCCGGACTCCCTTCACCGAGG |
| | | TCGGAA |
| cyno CD137 FL | SEQ ID NO: | MGNSCYNIVATLLLVLNFERTRSLQDLCSNC |
| AA | 110 | PAGTFCDNNRSQICSPCPPNSFSSAGGQRTCD |
| | | ICRQCKGVFKTRKECSSTSNAECDCISGYHC |
| | | LGAECSMCEQDCKQGQELTKKGCKDCCFG |
| | | TFNDQKRGICRPWTNCSLDGKSVLVNGTKE |
| | | RDVVCGPSPADLSPGASSATPPAPAREPGHSP |
| | | QIIFFLALTSTVVLFLLFFLVLRFSVVKRSRKK |
| | | LLYIFKQPFMRPVQTTQEEDGCSCRFPEEEE |
| | | GGCEL |
| cyno CD137 FL | SEQ ID NO: | ATGGGAAACAGCTGTTACAACATAGTAGCC |
| DNA | 111 | ACTCTGTTGCTGGTCCTGAACTTTGAGAGG |
| | | ACAAGATCCTTGCAGGATCTGTGTAGTAAC |
| | | TGCCCAGCTGGTACATTCTGTGATAATAAC |
| | | AGGAGTCAGATTTGCAGTCCCTGTCCTCCA |
| | | AATAGTTTCTCCAGCGCAGGTGGACAAAG |
| | | GACCTGTGACATATGCAGGCAGTGTAAAG |
| | | GTGTTTTCAAGACCAGGAAGGAGTGTTCC |
| | | TCCACCAGCAATGCAGAGTGTGACTGCATT |
| | | TCAGGATATCACTGCCTGGGGGCAGAATGC |
| | | AGCATGTGTGAACAGGATTGTAAACAAGG |
| | | TCAAGAATTGACAAAAAAAGGTTGTAAAG |
| | | ACTGTTGCTTTGGGACATTTAATGACCAGA |
| | | AACGTGGCATCTGTCGACCCTGGACAAAC |
| | | TGTTCTTTGGATGGAAAGTCTGTGCTTGTG |
| | | AATGGGACGAAGGAGAGGGACGTGGTCTG |
| | | CGGACCATCTCCAGCCGACCTCTCTCCAGG |
| | | AGCATCCTCTGCGACCCCGCCTGCCCCTGC |
| | | GAGAGAGCCAGGACACTCTCCGCAGATCA |
| | | TCTTCTTTCTTGCGCTGACTTCGACTGTGG |
| | | TACTCTTCCTGCTGTTCTTCCTCGTGCTCCG |
| | | TTTCTCTGTTGTTAAACGGAGCAGAAAGA |
| | | AACTCCTGTATATATTCAAACAACCATTTAT |
| | | GAGACCGGTACAAACCACTCAAGAGGAAG |
| | | ATGGATGTAGCTGCCGATTTCCAGAAGAAG |
| | | AAGAAGGAGGATGTGAACTGTGA |
| cyno CD137 ECD | SEQ ID NO: | LQDLCSNCPAGTFCDNNRSQICSPCPPNSFSS |
| 24-186 AA | 112 | AGGQRTCDICRQCKGVFKTRKECSSTSNAEC |
| | | DCISGYHCLGAECSMCEQDCKQGQELTKKG |
| | | CKDCCFGTFNDQKRGICRPWTNCSLDGKSV |
| | | LVNGTKERDVVCGPSPADLSPGASSATPPAP |
| | | AREPGHSPQ |
| cyno CD137 ECD | SEQ ID NO: | CTGCAGGATCTGTGTAGTAACTGCCCAGCT |
| 24-186 DNA | 113 | GGTACATTCTGTGATAATAACAGGAGTCAG |
| | | ATTTGCAGTCCCTGTCCTCCAAATAGTTTCT |
| | | CCAGCGCAGGTGGACAAAGGACCTGTGAC |
| | | ATATGCAGGCAGTGTAAAGGTGTTTTCAAG |
| | | ACCAGGAAGGAGTGTTCCTCCACCAGCAA |
| | | TGCAGAGTGTGACTGCATTTCAGGATATCA |
| | | CTGCCTGGGGGCAGAATGCAGCATGTGTG |
| | | AACAGGATTGTAAACAAGGTCAAGAATTG |
| | | ACAAAAAAAGGTTGTAAAGACTGTTGCTT |
| | | TGGGACATTTAATGACCAGAAACGTGGCAT |
| | | CTGTCGACCCTGGACAAACTGTTCTTTGGA |
| | | TGGAAAGTCTGTGCTTGTGAATGGGACGA |
| | | AGGAGAGGGACGTGGTCTGCGGACCATCT |
| | | CCAGCCGACCTCTCTCCAGGAGCATCCTCT |
| | | GCGACCCCGCCTGCCCCTGCGAGAGAGCC |
| | | AGGACACTCTCCGCAG |
| cyno CD137 ECD- | SEQ ID NO: | LQDLCSNCPAGTFCDNNRSQICSPCPPNSFSS |
| mIgG2a AA | 114 | AGGQRTCDICRQCKGVFKTRKECSSTSNAEC |
| | | DCISGYHCLGAECSMCEQDCKQGQELTKKG |
| | | CKDCCFGTFNDQKRGICRPWTNCSLDGKSV |
| | | LVNGTKERDVVCGPSPADLSPGASSATPPAP |
| | | AREPGHSPQEPRGPTIKPCPPCKCPAPNLLGG |
| | | PSVFIFPPKIKDVLMISLSPIVTCVVVDVSEDD |
| | | PDVQISWFVNNVEVHTAQTQTHREDYNSTL |
| | | RVVSALPIQHQDWMSGKEFKCKVNNKDLPA |
| | | PIERTISKPKGSVRAPQVYVLPPPEEEMTKKQ |
| | | VTLTCMVTDFMPEDIYVEWTNNGKTELNYK |
| | | NTEPVLDSDGSYFMYSKLRVEKKNWVERNS |
| | | YSCSVVHEGLHNHHTTKSFSRTPGK |
| cyno CD137 ECD- | SEQ ID NO: | CTGCAGGATCTGTGTAGTAACTGCCCAGCT |
| mIgG2a DNA | 115 | GGTACATTCTGTGATAATAACAGGAGTCAG |
| | | ATTTGCAGTCCCTGTCCTCCAAATAGTTTCT |
| | | CCAGCGCAGGTGGACAAAGGACCTGTGAC |
| | | ATATGCAGGCAGTGTAAAGGTGTTTTCAAG |
| | | ACCAGGAAGGAGTGTTCCTCCACCAGCAA |
| | | TGCAGAGTGTGACTGCATTTCAGGATATCA |
| | | CTGCCTGGGGGCAGAATGCAGCATGTGTG |
| | | AACAGGATTGTAAACAAGGTCAAGAATTG |
| | | ACAAAAAAAGGTTGTAAAGACTGTTGCTT |
| | | TGGGACATTTAATGACCAGAAACGTGGCAT |
| | | CTGTCGACCCTGGACAAACTGTTCTTTGGA |
| | | TGGAAAGTCTGTGCTTGTGAATGGGACGA |
| | | AGGAGAGGGACGTGGTCTGCGGACCATCT |
| | | CCAGCCGACCTCTCTCCAGGAGCATCCTCT |
| | | GCGACCCCGCCTGCCCCTGCGAGAGAGCC |
| | | AGGACACTCTCCGCAGGAGCCCAGAGGGC |
| | | CCACAATCAAGCCCTGTCCTCCATGCAAAT |
| | | GCCCAGCACCTAACCTCTTGGGTGGACCAT |
| | | CCGTCTTCATCTTCCCTCCAAAGATCAAGG |
| | | ATGTACTCATGATCTCCCTGAGCCCCATAGT |
| | | CACATGTGTGGTGGTGGATGTGAGCGAGG |
| | | ATGACCCAGATGTCCAGATCAGCTGGTTTG |
| | | TGAACAACGTGGAAGTACACACAGCTCAG |
| | | ACACAAACCCATAGAGAGGATTACAACAG |
| | | TACTCTCCGGGTGGTCAGTGCCCTCCCCAT |
| | | CCAGCACCAGGACTGGATGAGTGGCAAGG |
| | | AGTTCAAATGCAAGGTCAACAACAAAGAC |
| | | CTCCCAGCGCCCATCGAGAGAACCATCTCA |
| | | AAACCCAAAGGGTCAGTAAGAGCTCCACA |
| | | GGTATATGTCTTGCCTCCACCAGAAGAAGA |
| | | GATGACTAAGAAACAGGTCACTCTGACCT |
| | | GCATGGTCACAGACTTCATGCCTGAAGAC |
| | | ATTTACGTGGAGTGGACCAACAACGGGAA |
| | | AACAGAGCTAAACTACAAGAACACTGAAC |
| | | CAGTCCTGGACTCTGATGGTTCTTACTTCA |
| | | TGTACAGCAAGCTGAGAGTGGAAAAGAAG |
| | | AACTGGGTGGAAAGAAATAGCTACTCCTG |
| | | TTCAGTGGTCCACGAGGGTCTGCACAATC |
| | | ACCACACGACTAAGAGCTTCTCCCGGACT |
| | | CCGGGTAAA |
| human CD40 AA | SEQ ID NO: | MVRLPLQCVLWGCLLTAVHPEPPTACREKQ |
| | 116 | YLINSQCCSLCQPGQKLVSDCTEFTETECLPC |
| | | GESEFLDTWNRETHCHQHKYCDPNLGLRVQ |
| | | QKGTSETDTICTCEEGWHCTSEACESCVLHR |
| | | SCSPGFGVKQIATGVSDTICEPCPVGFFSNVS |
| | | SAFEKCHPWTSCETKDLVVQQAGTNKTDVV |
| | | CGPQDRLRALVVIPIIFGILFAILLVLVFIKKVA |
| | | KKPTNKAPHPKQEPQEINFPDDLPGSNTAAP |
| | | VQETLHGCQPVTQEDGKESRISVQERQ |
| human CD40 DNA | SEQ ID NO: | ATGGTTCGTCTGCCTCTGCAGTGCGTCCTC |
| | 117 | TGGGGCTGCTTGCTGACCGCTGTCCATCCA |
| | | GAACCACCCACTGCATGCAGAGAAAAACA |
| | | GTACCTAATAAACAGTCAGTGCTGTTCTTT |
| | | GTGCCAGCCAGGACAGAAACTGGTGAGTG |
| | | ACTGCACAGAGTTCACTGAAACGGAATGC |
| | | CTTCCTTGCGGTGAAAGCGAATTCCTAGAC |
| | | ACCTGGAACAGAGAGACACACTGCCACCA |
| | | GCACAAATACTGCGACCCCAACCTAGGGC |
| | | TTCGGGTCCAGCAGAAGGGCACCTCAGAA |
| | | ACAGACACCATCTGCACCTGTGAAGAAGG |
| | | CTGGCACTGTACGAGTGAGGCCTGTGAGA |
| | | GCTGTGTCCTGCACCGCTCATGCTCGCCCG |
| | | GCTTTGGGGTCAAGCAGATTGCTACAGGG |
| | | GTTTCTGATACCATCTGCGAGCCCTGCCCA |
| | | GTCGGCTTCTTCTCCAATGTGTCATCTGCTT |
| | | TCGAAAAATGTCACCCTTGGACAAGCTGT |
| | | GAGACCAAAGACCTGGTTGTGCAACAGGC |
| | | AGGCACAAACAAGACTGATGTTGTCTGTG |
| | | GTCCCCAGGATCGGCTGAGAGCCCTGGTG |
| | | GTGATCCCCATCATCTTCGGGATCCTGTTTG |
| | | CCATCCTCTTGGTGCTGGTCTTTATCAAAA |
| | | AGGTGGCCAAGAAGCCAACCAATAAGGCC |
| | | CCCCACCCCAAGCAGGAACCCCAGGAGAT |
| | | CAATTTTCCCGACGATCTTCCTGGCTCCAA |
| | | CACTGCTGCTCCAGTGCAGGAGACTTTAC |
| | | ATGGATGCCAACCGGTCACCCAGGAGGAT |
| | | GGCAAAGAGAGTCGCATCTCAGTGCAGGA |
| | | GAGACAGTGA |
| huCD40 ECD AA | SEQ ID NO: | MVRLPLQCVLWGCLLTAVHPEPPTACREKQ |
| (1-194) | 118 | YLINSQCCSLCQPGQKLVSDCTEFTETECLPC |
| | | GESEFLDTWNRETHCHQHKYCDPNLGLRVQ |
| | | QKGTSETDTICTCEEGWHCTSEACESCVLHR |
| | | SCSPGFGVKQIATGVSDTICEPCPVGFFSNVS |
| | | SAFEKCHPWTSCETKDLVVQQAGTNKTDVV |
| | | CGPQDRLRA |
| huCD40 ECD | SEQ ID NO: | ATGGTTCGTCTGCCTCTGCAGTGCGTCCTC |
| DNA (1-194) | 119 | TGGGGCTGCTTGCTGACCGCTGTCCATCCA |
| | | GAACCACCCACTGCATGCAGAGAAAAACA |
| | | GTACCTAATAAACAGTCAGTGCTGTTCTTT |
| | | GTGCCAGCCAGGACAGAAACTGGTGAGTG |
| | | ACTGCACAGAGTTCACTGAAACGGAATGC |
| | | CTTCCTTGCGGTGAAAGCGAATTCCTAGAC |
| | | ACCTGGAACAGAGAGACACACTGCCACCA |
| | | GCACAAATACTGCGACCCCAACCTAGGGC |
| | | TTCGGGTCCAGCAGAAGGGCACCTCAGAA |
| | | ACAGACACCATCTGCACCTGTGAAGAAGG |
| | | CTGGCACTGTACGAGTGAGGCCTGTGAGA |
| | | GCTGTGTCCTGCACCGCTCATGCTCGCCCG |
| | | GCTTTGGGGTCAAGCAGATTGCTACAGGG |
| | | GTTTCTGATACCATCTGCGAGCCCTGCCCA |
| | | GTCGGCTTCTTCTCCAATGTGTCATCTGCTT |
| | | TCGAAAAATGTCACCCTTGGACAAGCTGT |
| | | GAGACCAAAGACCTGGTTGTGCAACAGGC |
| | | AGGCACAAACAAGACTGATGTTGTCTGTG |
| | | GTCCCCAGGATCGGCTGAGAGCC |
| human CD40 ECD- | SEQ ID NO: | MVRLPLQCVLWGCLLTAVHPEPPTACREKQ |
| mIgG2a AA | 120 | YLINSQCCSLCQPGQKLVSDCTEFTETECLPC |
| | | GESEFLDTWNRETHCHQHKYCDPNLGLRVQ |
| | | QKGTSETDTICTCEEGWHCTSEACESCVLHR |
| | | SCSPGFGVKQIATGVSDTICEPCPVGFFSNVS |
| | | SAFEKCHPWTSCETKDLVVQQAGTNKTDVV |
| | | CGPQDRLRASEPRGPTIKPCPPCKCPAPNLLG |
| | | GPSVFIFPPKIKDVLMISLSPMVTCVVVDVSE |
| | | DDPDVQISWFVNNVEVLTAQTQTHREDYNS |
| | | TLRVVSALPIQHQDWMSGKEFKCKVNNKAL |
| | | PAPIERTISKPKGSVRAPQVYVLPPPEEEMTK |
| | | KQVTLTCMVTDFMPEDIYVEWTNNGKTELN |
| | | YKNTEPVLDSDGSYFMYSKLRVEKKNWVE |
| | | RNSYSCSVVHEGLHNHHTTKSFSRTPGK |
| human CD40 ECD- | SEQ ID NO: | ATGGTTCGTCTGCCTCTGCAGTGCGTCCTC |
| mIgG2a DNA | 121 | TGGGGCTGCTTGCTGACCGCTGTCCATCCA |
| | | GAACCACCCACTGCATGCAGAGAAAAACA |
| | | GTACCTAATAAACAGTCAGTGCTGTTCTTT |
| | | GTGCCAGCCAGGACAGAAACTGGTGAGTG |
| | | ACTGCACAGAGTTCACTGAAACGGAATGC |
| | | CTTCCTTGCGGTGAAAGCGAGTTCCTAGAC |
| | | ACCTGGAACAGAGAGACACACTGCCACCA |
| | | GCACAAATACTGCGACCCCAACCTAGGGC |
| | | TTCGGGTCCAGCAGAAGGGCACCTCAGAA |
| | | ACAGACACCATCTGCACCTGTGAAGAAGG |
| | | CTGGCACTGTACGAGTGAGGCCTGTGAGA |
| | | GCTGTGTCCTGCACCGCTCATGCTCGCCCG |
| | | GCTTTGGGGTCAAGCAGATTGCTACAGGG |
| | | GTTTCTGATACCATCTGCGAGCCCTGCCCA |
| | | GTCGGCTTCTTCTCCAATGTGTCATCTGCTT |
| | | TCGAAAAATGTCACCCTTGGACAAGCTGT |
| | | GAGACCAAAGACCTGGTTGTGCAACAGGC |
| | | AGGCACAAACAAGACTGATGTTGTCTGTG |
| | | GTCCCCAGGATCGGCTGAGAGCTAGCGAG |
| | | CCCAGAGGGCCCACAATCAAGCCCTGTCC |
| | | TCCATGCAAATGCCCAGCACCTAACCTCTT |
| | | GGGTGGACCATCCGTCTTCATCTTCCCTCC |
| | | AAAGATCAAGGATGTACTCATGATCTCCCT |
| | | GAGCCCCATGGTCACATGTGTGGTGGTGG |
| | | ATGTGAGCGAGGATGACCCAGATGTCCAG |
| | | ATCAGCTGGTTCGTGAACAACGTGGAAGT |
| | | ACTCACAGCTCAGACACAAACCCATAGAG |
| | | AGGATTACAACAGTACTCTCCGGGTGGTCA |
| | | GTGCCCTCCCCATCCAGCACCAGGACTGG |
| | | ATGAGTGGCAAGGAGTTCAAATGCAAGGT |
| | | CAACAACAAAGCCCTCCCAGCGCCCATCG |
| | | AGAGAACCATCTCAAAACCCAAAGGGTCA |
| | | GTAAGAGCTCCACAGGTATATGTCTTGCCT |
| | | CCACCAGAAGAAGAGATGACTAAGAAACA |
| | | GGTCACTCTGACCTGCATGGTCACAGACTT |
| | | CATGCCTGAAGACATTTACGTGGAGTGGAC |
| | | CAACAACGGGAAAACAGAGCTAAACTACA |
| | | AGAACACTGAACCAGTCCTGGACTCTGAT |
| | | GGTTCTTACTTCATGTACAGCAAGCTGAGA |
| | | GTGGAAAAGAAGAACTGGGTGGAAAGAA |
| | | ATAGCTACTCCTGCTCAGTGGTCCACGAGG |
| | | GTCTGCACAATCACCACACGACTAAGAGC |
| | | TTCTCCCGGACTCCGGGTAAA |
| human OX40 AA | SEQ ID NO: | MCVGARRLGRGPCAALLLLGLGLSTVTGLH |
| | 122 | CVGDTYPSNDRCCHECRPGNGMVSRCSRSQ |
| | | NTVCRPCGPGFYNDVVSSKPCKPCTWCNLR |
| | | SGSERKQLCTATQDTVCRCRAGTQPLDSYKP |
| | | GVDCAPCPPGHFSPGDNQACKPWTNCTLAG |
| | | KHTLQPASNSSDAICEDRDPPATQPQETQGPP |
| | | ARPITVQPTEAWPRTSQGPSTRPVEVPGGRA |
| | | VAAILGLGLVLGLLGPLAILLALYLLRRDQRL |
| | | PPDAHKPPGGGSFRTPIQEEQADAHSTLAKI |
| human OX40 DNA | SEQ ID NO: | ATGTGCGTGGGGGCTCGGCGGCTGGGCCG |
| | 123 | CGGGCCGTGTGCGGCTCTGCTCCTCCTGGG |
| | | CCTGGGGCTGAGCACCGTGACGGGGCTCC |
| | | ACTGTGTCGGGGACACCTACCCCAGCAAC |
| | | GACCGGTGCTGCCACGAGTGCAGGCCAGG |
| | | CAACGGGATGGTGAGCCGCTGCAGCCGCT |
| | | CCCAGAACACGGTGTGCCGTCCGTGCGGG |
| | | CCGGGCTTCTACAACGACGTGGTCAGCTC |
| | | CAAGCCGTGCAAGCCCTGCACGTGGTGTA |
| | | ACCTCAGAAGTGGGAGTGAGCGGAAGCA |
| | | GCTGTGCACGGCCACACAGGACACAGTCT |
| | | GCCGCTGCCGGGCGGGCACCCAGCCCCTG |
| | | GACAGCTACAAGCCTGGAGTTGACTGTGC |
| | | CCCCTGCCCTCCAGGGCACTTCTCCCCAGG |
| | | CGACAACCAGGCCTGCAAGCCCTGGACCA |
| | | ACTGCACCTTGGCTGGGAAGCACACCCTG |
| | | CAGCCGGCCAGCAATAGCTCGGACGCAAT |
| | | CTGTGAGGACAGGGACCCCCCAGCCACGC |
| | | AGCCCCAGGAGACCCAGGGCCCCCCGGCC |
| | | AGGCCCATCACTGTCCAGCCCACTGAAGC |
| | | CTGGCCCAGAACCTCACAGGGACCCTCCA |
| | | CCCGGCCCGTGGAGGTCCCCGGGGGCCGT |
| | | GCGGTTGCCGCCATCCTGGGCCTGGGCCTG |
| | | GTGCTGGGGCTGCTGGGCCCCCTGGCCATC |
| | | CTGCTGGCCCTGTACCTGCTCCGGAGGGA |
| | | CCAGAGGCTGCCCCCCGATGCCCACAAGC |
| | | CCCCTGGGGGAGGCAGTTTCCGGACCCCC |
| | | ATCCAAGAGGAGCAGGCCGACGCCCACTC |
| | | CACCCTGGCCAAGATCTGA |
| human OX40 ECD | SEQ ID NO: | MCVGARRLGRGPCAALLLLGLGLSTVTGLH |
| (1-216) AA | 124 | CVGDTYPSNDRCCHECRPGNGMVSRCSRSQ |
| | | NTVCRPCGPGFYNDVVSSKPCKPCTWCNLR |
| | | SGSERKQLCTATQDTVCRCRAGTQPLDSYKP |
| | | GVDCAPCPPGHFSPGDNQACKPWTNCTLAG |
| | | KHTLQPASNSSDAICEDRDPPATQPQETQGPP |
| | | ARPITVQPTEAWPRTSQGPSTRPVEVPGGRA |
| | | VA |
| human OX40 ECD | SEQ ID NO: | ATGTGCGTGGGGGCTCGGCGGCTGGGCCG |
| (1-216) DNA | 125 | CGGGCCGTGTGCGGCTCTGCTCCTCCTGGG |
| | | CCTGGGGCTGAGCACCGTGACGGGGCTCC |
| | | ACTGTGTCGGGGACACCTACCCCAGCAAC |
| | | GACCGGTGCTGCCACGAGTGCAGGCCAGG |
| | | CAACGGGATGGTGAGCCGCTGCAGCCGCT |
| | | CCCAGAACACGGTGTGCCGTCCGTGCGGG |
| | | CCGGGCTTCTACAACGACGTGGTCAGCTC |
| | | CAAGCCGTGCAAGCCCTGCACGTGGTGTA |
| | | ACCTCAGAAGTGGGAGTGAGCGGAAGCA |
| | | GCTGTGCACGGCCACACAGGACACAGTCT |
| | | GCCGCTGCCGGGCGGGCACCCAGCCCCTG |
| | | GACAGCTACAAGCCTGGAGTTGACTGTGC |
| | | CCCCTGCCCTCCAGGGCACTTCTCCCCAGG |
| | | CGACAACCAGGCCTGCAAGCCCTGGACCA |
| | | ACTGCACCTTGGCTGGGAAGCACACCCTG |
| | | CAGCCGGCCAGCAATAGCTCGGACGCAAT |
| | | CTGTGAGGACAGGGACCCCCCAGCCACGC |
| | | AGCCCCAGGAGACCCAGGGCCCCCCGGCC |
| | | AGGCCCATCACTGTCCAGCCCACTGAAGC |
| | | CTGGCCCAGAACCTCACAGGGACCCTCCA |
| | | CCCGGCCCGTGGAGGTCCCCGGGGGCCGT |
| | | GCGGTTGCC |
| human OX40 ECD- | SEQ ID NO: | MCVGARRLGRGPCAALLLLGLGLSTVTGLH |
| His AA | 126 | CVGDTYPSNDRCCHECRPGNGMVSRCSRSQ |
| | | NTVCRPCGPGFYNDVVSSKPCKPCTWCNLR |
| | | SGSERKQLCTATQDTVCRCRAGTQPLDSYKP |
| | | GVDCAPCPPGHFSPGDNQACKPWTNCTLAG |
| | | KHTLQPASNSSDAICEDRDPPATQPQETQGPP |
| | | ARPITVQPTEAWPRTSQGPSTRPVEVPGGRA |
| | | VAHHHHHH |
| human OX40 ECD- | SEQ ID NO: | ATGTGCGTGGGGGCTCGGCGGCTGGGCCG |
| His DNA | 127 | CGGGCCGTGTGCGGCTCTGCTCCTCCTGGG |
| | | CCTGGGGCTGAGCACCGTGACGGGGCTCC |
| | | ACTGTGTCGGGGACACCTACCCCAGCAAC |
| | | GACCGGTGCTGCCACGAGTGCAGGCCAGG |
| | | CAACGGGATGGTGAGCCGCTGCAGCCGCT |
| | | CCCAGAACACGGTGTGCCGTCCGTGCGGG |
| | | CCGGGCTTCTACAACGACGTGGTCAGCTC |
| | | CAAGCCGTGCAAGCCCTGCACGTGGTGTA |
| | | ACCTCAGAAGTGGGAGTGAGCGGAAGCA |
| | | GCTGTGCACGGCCACACAGGACACAGTCT |
| | | GCCGCTGCCGGGCGGGCACCCAGCCCCTG |
| | | GACAGCTACAAGCCTGGAGTTGACTGTGC |
| | | CCCCTGCCCTCCAGGGCACTTCTCCCCAGG |
| | | CGACAACCAGGCCTGCAAGCCCTGGACCA |
| | | ACTGCACCTTGGCTGGGAAGCACACCCTG |
| | | CAGCCGGCCAGCAATAGCTCGGACGCAAT |
| | | CTGTGAGGACAGGGACCCCCCAGCCACGC |
| | | AGCCCCAGGAGACCCAGGGCCCCCCGGCC |
| | | AGGCCCATCACTGTCCAGCCCACTGAAGC |
| | | CTGGCCCAGAACCTCACAGGGACCCTCCA |
| | | CCCGGCCCGTGGAGGTCCCCGGGGGCCGT |
| | | GCGGTTGCCCATCACCATCACCATCAC |

### Example 2. Generation of Anti-huCD137 VH Domain Antibodies

### Construction of Synthetic Human VH Antibody Repertoires

Synthetic libraries were constructed essentially using the germline 3-23 (SEQ ID NO: 128 and 129). Randomization of heavy chain CDRs (HCDRs) was carried out by combinatorial mutagenesis using degenerate oligonucleotides. Randomization of the HCDR1 and HCDR2 regions was carried out via multiple site-specific mutations by polymerase chain reaction as described by Meetei (Meetei et al., (1998) Anal. Biochem, 264, 288-91; Meetei et al., (2002) Methods Mol Biol, 182, 95-102). For CDR3 regions, different lengths from 8 to 14 (Kabat definition) of degenerate oligonucleotides were synthesized (Invitrogen), and diversity was introduced by splice-overlap extension PCR. The PCR products after the mutagenesis steps, were double-digested by NcoI/NotI and ligated into the phagemid vector pCANTAB-5E. Repertoires were then transformed into Escherichia coli TG1 bacteria and validated by DNA Sanger sequencing of random clones (>96 clones analyzed). Phages were purified by two precipitations with PEG/NaCl directly from the culture supernatant after a rescue step using KM13 helper phage. A library with a total size of 1.38×10¹¹ was obtained after transformation into E. coli bacteria.

TABLE 3
Germline used for library construction

| | | |
|---|---|---|
| 3-23 germline | SEQ ID NO: 128 | EVQLLESGGGLVQPGGSLRLSCAASGFTFSSY |
| AA | | AISWVRQAPGKGLEWVSAISGSGGSTYYADS |
| | | VKGRFTISRDNSKNTLYLQMNSLRAEDTAVY |
| | | YCAR |
| 3-23 germline | SEQ ID NO: 129 | GAAGTGCAGCTGCTGGAAAGCGGCGGCGG |
| DNA | | CCTGGTGCAGCCGGGCGGCAGCCTGCGCCT |
| | | GAGCTGCGCGGCGAGCGGCTTTACCTTTAG |
| | | CAGCTATGCGATTAGCTGGGTGCGCCAGGC |
| | | GCCGGGCAAAGGCCTGGAATGGGTGAGCG |
| | | CGATTAGCGGCAGCGGCGGTAGCACCTATT |
| | | ATGCGGATAGCGTGAAAGGCCGCTTTACCA |
| | | TTAGCCGCGATAACAGCAAAAACACCCTGT |
| | | ATCTGCAGATGAACAGCCTGCGCGCGGAAG |
| | | ATACCGCCGTGTATTATTGCGCGCGT |

### Phage Display Panning and Screening

Phage display selection was carried out by phage display using standard protocols (Silacci et al., (2005) Proteomics, 5, 2340-50; Zhao et al., (2014) PLOS One, 9, e111339). In brief, 10 μg/ml of immobilized human CD137 ECD-mIgG2a in immunotubes (Cat. 470319, ThermoFisher) was utilized in round 1 and 2. Hut78/huCD137 cells were used for selection in round 3 and 4. Immunotubes were blocked with 5% milk powder (w/v) in PBS supplemented with 1% Tween 20 (MPBST) for 1 h. After washes with PBST (PBS buffer supplemented with 0.05% Tween 20), 5×10¹² (round 1) or 5×10¹¹ (round 2) phages from each sub library were depleted by human CD40 ECD-mIgG2a in MPBST for 1 hour and then incubated with the antigen for 1 hour. For the third and fourth rounds of selections, cell panning was carried out using Hut78/huCD137 cells (round 3) with HEK293(ATCC, CRL-1573) cells as depletion cells. After washes with PBST, bound phages were eluted with 100 mM triethylamine (Sigma-Aldrich). Eluted phages were used to infect mid-log phase E. coli TG1 bacteria and plated onto TYE-agar plates supplemented with 2% glucose and 100 μg/ml ampicillin. After four rounds of selections, individual clones were picked up and phage containing supernatants were prepared using standard protocols. Phage ELISA and FACS were used to screen anti-huCD137 VH domain antibodies.

For phage ELISA, a Maxisorp™ immunoplate was coated with antigens and blocked with 5% milk powder (w/v) in PBS buffer. Phage supernatant was blocked with MPBST for 30 min and added to wells of the ELISA plate for 1 hour. After washes with PBST, bound phage was detected using HRP-conjugated anti-M13 antibody (GE Healthcare) and 3,3′,5,5′-tetramethylbenzidine substrate (Cat.: 00-4201-56, eBioscience, USA). The ELISA-positive clones were further verified by flow cytometry using Hut78/huCD137 cells. CD137-expressing cells (10⁵ cells/well) were incubated with ELISA-positive phage supernatants, followed by binding with Alexa Fluro-647-labeled anti-M13 antibody (GE Healthcare). Cell fluorescence was quantified using a flow cytometer (Guava easyCyte™ 8HT, Merck-Millipore, USA).

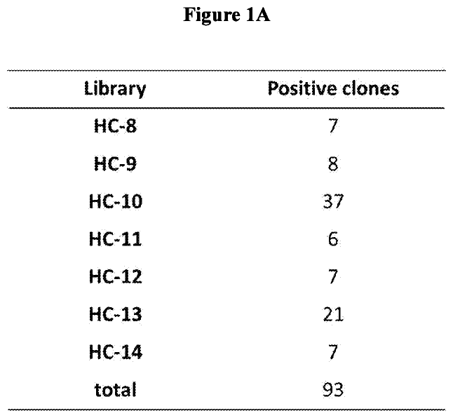

The clones that showed positive signals in FACS screening, and binding to both huCD137 and cynoCD137 but not huOX40 and huCD40, were picked up and sequenced. Approximately 76 unique sequences from 93 positive clones were identified (FIG. 1A-1B).

### Expression and Purification of Fc Fusion VH Antibodies

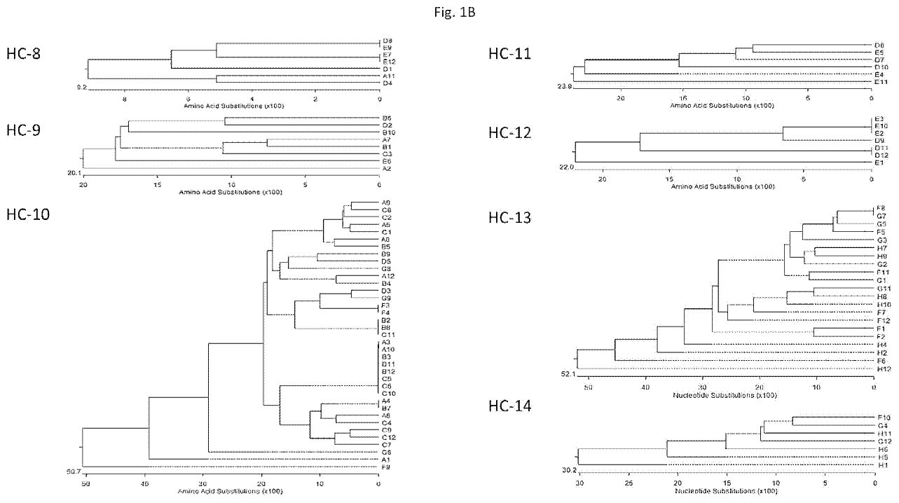

The VH sequences were analyzed by comparing sequence homology and grouped based on sequence similarity. Complementary determining regions (CDRs) were defined based on the Kabat (Wu and Kabat (1970) J. Exp. Med. 132:211-250) and IMGT (Lefranc (1999) Nucleic Acids Research 27:209-212) system by sequence annotation and by internet-based sequence analysis. The amino acid and DNA sequences of two representative top clones BGA-7207 and BGA-4712 are listed in Table 4 below. After the sequence checking and analysis of binding curve by SPR, anti-huCD137 VH domain antibodies were then constructed as human Fc fusion VH antibody format (VH-Fc) using in-house developed expression vectors. As shown in FIG. 2A, VH domain antibodies were fused at the N terminal of human Fc with a G4S (SEQ ID NO: 23) linker in between. A Fc-null version (an inert Fc without FcγR-binding) of human IgG1 (SEQ ID NO: 134) was used. Expression and preparation of Fc fusion VH antibodies were achieved by transfection into 293G cells and by purification using a Protein A column (Cat. No. 17543802, GE Life Sciences). The purified antibodies were concentrated to 0.5-5 mg/mL in PBS and stored in aliquots in a −80° C. freezer.

TABLE 4
Amino acid and DNA sequences of two selected anti-huCD137 VH domain
antibodies

| Antibody | SEQ ID NO | | SEQUENCE |
|---|---|---|---|
| BGA-7207 | SEQ ID NO: 60 | VH | EVQLLESGGGLVQPGGSLRLSCAASGFR |
| | | | LDTTEVGWVRQAPGKGLEWVSTIMGIS |
| | | | GSTYYADSVKGRFTISRDNSKNTLYLQM |
| | | | NSLRAEDTAVYYCARVVDSLFDDSAVFD |
| | | | YWGQGTLVTVSS |
| | SEQ ID NO: 61 | VH DNA | GAGGTCCAGTTACTTGAGAGTGGTGG |
| | | | AGGTCTGGTCCAACCAGGAGGTTCGC |
| | | | TGCGTTTATCCTGCGCCGCGTCTGGATT |
| | | | CAGATTGGACACCACCGAAGTAGGTT |
| | | | GGGTGCGTCAAGCGCCGGGGAAAGGA |
| | | | CTGGAATGGGTCTCCACCATCATGGGT |
| | | | ATTAGTGGTTCGACATACTATGCGGAC |
| | | | AGTGTCAAAGGGCGCTTTACGATCTCG |
| | | | CGCGATAACTCAAAAAATACTCTTTAC |
| | | | CTTCAAATGAATAGCCTTCGTGCTGAG |
| | | | GACACTGCGGTGTACTACTGCGCCCGC |
| | | | GTCGTCGATTCCCTCTTTGATGACAGC |
| | | | GCCGTTTTTGATTACTGGGGACAGGGC |
| | | | ACCTTAGTTACAGTCTCATCG |
| | SEQ ID NO: | VH-Fc | EVQLLESGGGLVQPGGSLRLSCAASGFR |
| | 130 | | LDTTEVGWVRQAPGKGLEWVSTIMGIS |
| | | | GSTYYADSVKGRFTISRDNSKNTLYLQM |
| | | | NSLRAEDTAVYYCARVVDSLFDDSAVFD |
| | | | YWGQGTLVTVSSGGGGSDKTHTCPPCP |
| | | | APPAAGPSVFLFPPKPKDTLMISRTPEVT |
| | | | CVVVDVSHEDPEVKFNWYVDGVEVHN |
| | | | AKTKPREEQYNSTYRVVSVLTVLHQDW |
| | | | LNGKEYKCKVSNKALAAPIEKTISKAKG |
| | | | QPREPQVYTLPPSREEMTKNQVSLTCLV |
| | | | KGFYPSDIAVEWESNGQPENNYKTTPPV |
| | | | LDSDGSFFLYSKLTVDKSRWQQGNVFSC |
| | | | SVMHEALHNHYTQKSLSLSPGK |
| | SEQ ID NO: | VH-Fc DNA | GAGGTCCAGTTACTTGAGAGTGGTGG |
| | 131 | | AGGTCTGGTCCAACCAGGAGGTTCGC |
| | | | TGCGTTTATCCTGCGCCGCGTCTGGATT |
| | | | CAGATTGGACACCACCGAAGTAGGTT |
| | | | GGGTGCGTCAAGCGCCGGGGAAAGGA |
| | | | CTGGAATGGGTCTCCACCATCATGGGT |
| | | | ATTAGTGGTTCGACATACTATGCGGAC |
| | | | AGTGTCAAAGGGCGCTTTACGATCTCG |
| | | | CGCGATAACTCAAAAAATACTCTTTAC |
| | | | CTTCAAATGAATAGCCTTCGTGCTGAG |
| | | | GACACTGCGGTGTACTACTGCGCCCGC |
| | | | GTCGTCGATTCCCTCTTTGATGACAGC |
| | | | GCCGTTTTTGATTACTGGGGACAGGGC |
| | | | ACCTTAGTTACAGTCTCATCGGGCGGC |
| | | | GGAGGGTCTGACAAGACCCACACATG |
| | | | CCCCCCTTGTCCTGCTCCACCAGCTGC |
| | | | AGGACCAAGCGTGTTCCTGTTTCCACC |
| | | | CAAGCCCAAGGATACCCTGATGATCTC |
| | | | TCGGACCCCAGAGGTGACATGCGTGG |
| | | | TGGTGGATGTGAGCCACGAGGACCCC |
| | | | GAGGTGAAGTTCAACTGGTATGTGGAC |
| | | | GGCGTGGAGGTGCACAATGCTAAGAC |
| | | | CAAGCCCAGGGAGGAGCAGTACAACT |
| | | | CCACCTATAGAGTGGTGTCTGTGCTGA |
| | | | CAGTGCTGCACCAGGATTGGCTGAAC |
| | | | GGCAAGGAGTATAAGTGCAAGGTGTC |
| | | | CAATAAGGCCCTGGCCGCTCCTATCGA |
| | | | GAAGACCATCTCTAAGGCCAAGGGCC |
| | | | AGCCCAGAGAGCCTCAGGTGTACACA |
| | | | CTGCCTCCATCCCGGGAAGAGATGACC |
| | | | AAGAACCAGGTGTCTCTGACATGTCTG |
| | | | GTCAAGGGCTTCTATCCCTCTGACATC |
| | | | GCCGTGGAGTGGGAGAGCAATGGCCA |
| | | | GCCTGAGAACAATTACAAGACCACAC |
| | | | CCCCTGTGCTGGATTCCGACGGCTCTT |
| | | | TCTTTCTGTATAGCAAGCTGACCGTGG |
| | | | ACAAGTCCCGGTGGCAGCAGGGCAAC |
| | | | GTGTTCAGCTGTTCCGTGATGCACGAA |
| | | | GCTCTGCATAATCACTATACTCAGAAAT |
| | | | CCCTGTCACTGTCACCTGGTAAA |
| BGA-4712 | SEQ ID NO: 70 | VH | EVQLLESGGGLVQPGGSLRLSCAASGF |
| | | | MLSAEDVGWVRQAPGKGLEWVSAILD |
| | | | FGGSTYYADSVKGRFTISRDNSKNTLYL |
| | | | QMNSLRAEDTAVYYCARVVYHAGGGV |
| | | | TFDYWGQGTLVTVSS |
| | SEQ ID NO: 71 | VH DNA | GAGGTCCAGTTACTTGAGAGTGGTGG |
| | | | AGGTCTGGTCCAACCAGGAGGTTCGC |
| | | | TGCGTTTATCCTGCGCCGCGTCTGGATT |
| | | | CATGTTGTCCGCCGAAGACGTGGGTTG |
| | | | GGTGCGTCAAGCGCCGGGGAAAGGAC |
| | | | TGGAATGGGTCTCCGCCATCTTGGATT |
| | | | TTGGTGGTTCGACATACTATGCGGACA |
| | | | GTGTCAAAGGGCGCTTTACGATCTCGC |
| | | | GCGATAACTCAAAAAATACTCTTTACC |
| | | | TTCAAATGAATAGCCTTCGTGCTGAGG |
| | | | ACACTGCGGTGTACTACTGCGCCCGCG |
| | | | TCGTCTACCATGCTGGTGGTGGCGTCA |
| | | | CCTTTGATTACTGGGGACAGGGCACCT |
| | | | TAGTTACAGTCTCATCG |
| | SEQ ID NO: | VH-Fc | EVQLLESGGGLVQPGGSLRLSCAASGF |
| | 132 | | MLSAEDVGWVRQAPGKGLEWVSAILD |
| | | | FGGSTYYADSVKGRFTISRDNSKNTLYL |
| | | | QMNSLRAEDTAVYYCARVVYHAGGGV |
| | | | TFDYWGQGTLVTVSSGGGGSDKTHTCP |
| | | | PCPAPPAAGPSVFLFPPKPKDTLMISRTP |
| | | | EVTCVVVDVSHEDPEVKFNWYVDGVE |
| | | | VHNAKTKPREEQYNSTYRVVSVLTVLH |
| | | | QDWLNGKEYKCKVSNKALAAPIEKTIS |
| | | | KAKGQPREPQVYTLPPSREEMTKNQVS |
| | | | LTCLVKGFYPSDIAVEWESNGQPENNYK |
| Inert Fc | SEQ ID NO: | VH-Fc DNA | TTPPVLDSDGSFFLYSKLTVDKSRWQQG |
| | 133 | | NVFSCSVMHEALHNHYTQKSLSLSPGK |
| | | | GAGGTCCAGTTACTTGAGAGTGGTGG |
| | | | AGGTCTGGTCCAACCAGGAGGTTCGC |
| | | | TGCGTTTATCCTGCGCCGCGTCTGGATT |
| | | | CATGTTGTCCGCCGAAGACGTGGGTTG |
| | | | GGTGCGTCAAGCGCCGGGGAAAGGAC |
| | | | TGGAATGGGTCTCCGCCATCTTGGATT |
| | | | TTGGTGGTTCGACATACTATGCGGACA |
| | | | GTGTCAAAGGGCGCTTTACGATCTCGC |
| | | | GCGATAACTCAAAAAATACTCTTTACC |
| | | | TTCAAATGAATAGCCTTCGTGCTGAGG |
| | | | ACACTGCGGTGTACTACTGCGCCCGCG |
| | | | TCGTCTACCATGCTGGTGGTGGCGTCA |
| | | | CCTTTGATTACTGGGGACAGGGCACCT |
| | | | TAGTTACAGTCTCATCGGGCGGCGGAG |
| | | | GGTCTGACAAGACCCACACATGCCCC |
| | | | CCTTGTCCTGCTCCACCAGCTGCAGGA |
| | | | CCAAGCGTGTTCCTGTTTCCACCCAAG |
| | | | CCCAAGGATACCCTGATGATCTCTCGG |
| | | | ACCCCAGAGGTGACATGCGTGGTGGT |
| | | | GGATGTGAGCCACGAGGACCCCGAGG |
| | | | TGAAGTTCAACTGGTATGTGGACGGCG |
| | | | TGGAGGTGCACAATGCTAAGACCAAG |
| | | | CCCAGGGAGGAGCAGTACAACTCCAC |
| | | | CTATAGAGTGGTGTCTGTGCTGACAGT |
| | | | GCTGCACCAGGATTGGCTGAACGGCA |
| | | | AGGAGTATAAGTGCAAGGTGTCCAATA |
| | | | AGGCCCTGGCCGCTCCTATCGAGAAGA |
| | | | CCATCTCTAAGGCCAAGGGCCAGCCCA |
| | | | GAGAGCCTCAGGTGTACACACTGCCTC |
| | | | CATCCCGGGAAGAGATGACCAAGAAC |
| | | | CAGGTGTCTCTGACATGTCTGGTCAAG |
| | | | GGCTTCTATCCCTCTGACATCGCCGTG |
| | | | GAGTGGGAGAGCAATGGCCAGCCTGA |
| | | | GAACAATTACAAGACCACACCCCCTGT |
| | | | GCTGGATTCCGACGGCTCTTTCTTTCT |
| | | | GTATAGCAAGCTGACCGTGGACAAGTC |
| | | | CCGGTGGCAGCAGGGCAACGTGTTCA |
| | | | GCTGTTCCGTGATGCACGAAGCTCTGC |
| | | | ATAATCACTATACTCAGAAATCCCTGTC |
| | | | ACTGTCACCTGGTAAA |
| | SEQ ID NO: | A Fc-null | DKTHTCPPCPAPPAAGPSVFLFPPKPKDT |
| | 134 | version | LMISRTPEVTCVVVDVSHEDPEVKFNW |
| | | IgG1 | YVDGVEVHNAKTKPREEQYNSTYRVVS |
| | | | VLTVLHQDWLNGKEYKCKVSNKALAA |
| | | | PIEKTISKAKGQPREPQVYTLPPSREEMT |
| | | | KNQVSLTCLVKGFYPSDIAVEWESNGQP |
| | | | ENNYKTTPPVLDSDGSFFLYSKLTVDKS |
| | | | RWQQGNVFSCSVMHEALHNHYTQKSL |
| | | | SLSPGK |
| | SEQ ID NO: | A Fc-null | GACAAGACCCACACATGCCCCCCTTGT |
| | 135 | version | CCTGCTCCACCAGCTGCAGGACCAAG |
| | | IgG1 DNA | CGTGTTCCTGTTTCCACCCAAGCCCAA |
| | | | GGATACCCTGATGATCTCTCGGACCCC |
| | | | AGAGGTGACATGCGTGGTGGTGGATG |
| | | | TGAGCCACGAGGACCCCGAGGTGAAG |
| | | | TTCAACTGGTATGTGGACGGCGTGGAG |
| | | | GTGCACAATGCTAAGACCAAGCCCAG |
| | | | GGAGGAGCAGTACAACTCCACCTATAG |
| | | | AGTGGTGTCTGTGCTGACAGTGCTGCA |
| | | | CCAGGATTGGCTGAACGGCAAGGAGT |
| | | | ATAAGTGCAAGGTGTCCAATAAGGCCC |
| | | | TGGCCGCTCCTATCGAGAAGACCATCT |
| | | | CTAAGGCCAAGGGCCAGCCCAGAGAG |
| | | | CCTCAGGTGTACACACTGCCTCCATCC |
| | | | CGGGAAGAGATGACCAAGAACCAGGT |
| | | | GTCTCTGACATGTCTGGTCAAGGGCTT |
| | | | CTATCCCTCTGACATCGCCGTGGAGTG |
| | | | GGAGAGCAATGGCCAGCCTGAGAACA |
| | | | ATTACAAGACCACACCCCCTGTGCTGG |
| | | | ATTCCGACGGCTCTTTCTTTCTGTATAG |
| | | | CAAGCTGACCGTGGACAAGTCCCGGT |
| | | | GGCAGCAGGGCAACGTGTTCAGCTGT |
| | | | TCCGTGATGCACGAAGCTCTGCATAAT |
| | | | CACTATACTCAGAAATCCCTGTCACTG |
| | | | TCACCTGGTAAA |

### Example 3. Functional Screening of Anti-huCD137 VH Domain Antibodies

Functional screening was applied to selected anti-huCD137 VH domain antibodies with strong agonism using supernatant containing VH-Fc proteins. In brief, the 96-well white/clear bottom plates (Thermo Fisher) were pre-incubated with 3 μg/ml anti-hu CD3 (Invitrogen, Cat. No. 16-0037-85) at 50 μl/well for 5 min and then washed away by PBS buffer. Next, Hut78/huCD137 cells were resuspended at 5×10⁵ cells/ml, and directly plated into the pre-coated plates at 50 μl/well (25,000 well per well). Supernatants containing various VH-Fc proteins were mixed with the cells. Alternatively, for purified VH domain antibodies with Fc fusion, a dose titration of purified VH-Fc protein preparations was added in duplicate at 25, 5, 1, 0.2, 0.04, 0.008 or 0.0016 μg/ml at 50 μl/well. As a crosslinker, goat anti-hu IgG(H&L) polystyrene particles (6.46 um) (Cat. No. HUP-60-5, Spherotech) were added. Assay plates were incubated overnight at 37° C., and the concentrations of IL-2 were measured after 24 hours. Data was plotted as IL-2 fold increase compared with the concentration in the well with media only. FIG. 2B shows a representative screening result using supernatants containing VH-Fc proteins, and one of the clones, BGA-4712 has been shown to be capable to stimulate IL-2 production in Hut78/huCD137 cells in a dose dependent manner (FIG. 2C).

### Example 4. Characterization of Purified Anti-huCD137 VH Domain Antibodies

### Characterization of Purified Antibodies Via ELISA

For antigen ELISA, a Maxisorp immunoplate was coated with antigens and blocked with 3% BSA (w/v) in PBS buffer (blocking buffer). Monoclonal VH domain antibodies were blocked with blocking buffer for 30 min and added to wells of the ELISA plate for 1 h. After washes with PBST, bound antibodies were detected using HRP-conjugated anti-human IgG antibody (Sigma, A0170) and 3,3′,5,5′-tetramethylbenzidine substrate (Cat.: 00-4201-56, eBioscience, USA). All selected clones were shown to cross-react with cynoCD137 with no binding to human OX40 ECD and human CD40 ECD.

### Characterization of Purified Antibodies Via SPR Analysis

Characterization of anti-huCD137 VH domain antibodies were made by SPR assays using BIAcore™ T-200 (GE Life Sciences). Briefly, anti-human IgG (Fc) antibody was immobilized on an activated CM5 biosensor chip (Cat.: BR100839, GE Life Sciences). Anti-huCD137 domain antibodies were flowed over the chip surface and captured by anti-human IgG (Fc) antibody. Then a serial dilution (6.0 nM to 2150 nM) of human CD137 ECD-mIgG2a was flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ.

### Characterization of Purified Antibodies Via Flow Cytometry

For flow cytometry, human CD137 expressing cells (10⁵ cells/well) were incubated with various concentrations of purified VH domain antibodies, followed by binding with Alexa Fluro-647-labeled anti-hu IgG Fc antibody (Cat.: 409320, BioLegend, USA). Cell fluorescence was quantified using a flow cytometer (Guava easyCyte™ 8HT, Merck-Millipore, USA). Ligand competition was also applied in a flow cytometry based assay. In brief, Hut78/huCD137 was incubated with Fc fusion VH domain antibodies (VH-Fc) in the presence of serially diluted human CD137 ligand-mIgG2a, followed by detection with Alexa Fluro-647-labeled anti-hu IgG Fc antibody (Cat.: 409320, BioLegend, USA).

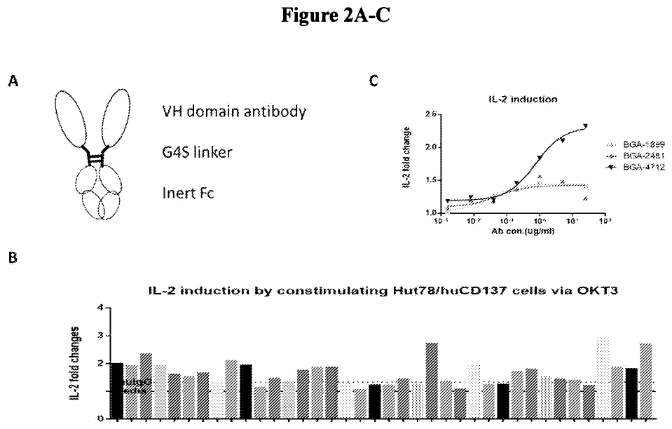

Selected VH domain antibodies were then characterized for affinity, cell binding and ligand competition. The SPR study, FACS analysis and the ligand competition result of one representative top clone BGA-4712 are shown in FIG. 3A-3B.

### Example 5. Engineering and Affinity Maturation

### Engineering

Engineering was performed for the selected clone BGA-4712 to improve biochemical and biophysical properties. The considerations include amino acid compositions, heat stability (Tm), surface hydrophobicity, removal of post-translational modification (PTM) sites and isoelectronic points (pIs) while maintaining functional activities. Substitutions were made mainly in HCDRs and framework regions based on the BGA-4712 sequence. The substitutions included amino acid changes F28R, M29T, V35M, V37F or Y, G44E, L45R or G or Y, and W47G or S or F or L or R or Y, D62E, S75A, N84S, W103R (Kabat definition). The variants were expressed in Fc fusion VH. The substitutions without significant affinity reduction were identified (Table 5). Combination of mutations were made. The sequences of BGA-4712-M3 and BGA-7556 are disclosed in Table 6 and 7.

### Affinity Maturation

To further explore potential effective CD137 based mechanisms of action (MOAs), we aimed to generate affinity matured BGA-4712-M3 variants with improved drug-developability by phage display. The library construction was described as before. In brief, a phagemid vector pCANTAB 5E (GE Healthcare) was used by standard molecular biology techniques to construct a phagemid designed to display CH3-G4S (linker)-BGA-4712-M3 (Table 8) on the surface of M13 bacteriophage as a fusion with the N-terminus of a fragment of the gene-3 minor coat protein. Generation of affinity-matured BGA-4712 variants was carried out by phage display using standard protocols (Silacci et al., (2005) Proteomics, 5, 2340-50; Zhao et al., (2014) PLOS One, 9, e111339). The phagemid was used as the template to construct phage-displayed libraries containing 2.0×10⁸ unique members. All three CDRs were randomized but each CDR had a maximum of one mutation in each clone except HCDR3, which could have two simultaneous mutations. Each position was randomized with an NNK codon (IUPAC code) encoding any amino acid or an amber stop codon.

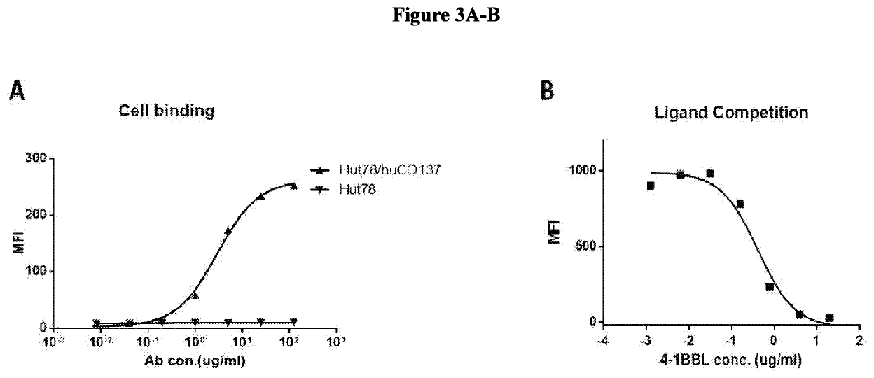

The frequency of mutations in each HCDR after four rounds of selection was relatively high. FIG. 4 shows the sequences of HCDR regions after four rounds of selections. All mutations were introduced in BGA-7556 (SEQ ID NO: 86) to make affinity-matured variants except for BGA-3386, of which the mutations were introduced in BGA-4712-M3 (SEQ ID NO: 75). All variants were expressed as both monoclonal antibodies (VH-Fc). The purified antibodies were concentrated to 0.5-10 mg/mL in PBS and stored in aliquots in a −80° C. freezer.

Affinity comparison on CD137 affinity matured variants was made by SPR assays using BIAcore™ T-200 (GE Life Sciences) and flow cytometry as described. The sequence information is shown in Table 10 and the results of SPR-determined binding profiles of anti-huCD137 antibodies were summarized in Table 9.

TABLE 5
Comparison of CD137 binding affinities

| | | | | EC50 on |
|---|---|---|---|---|
| | | | | Hut78/ |
| | | SEC- | | huCD137 |
| | | HPLC | KD | cells |
| Antibody | Format | (%) | (M) | (ug/ml) |
| BGA-4712-M3 | Fc fusion VH | 97.98 | 1.69E−07 | 2.37 |
| BGA-7556 | Fc fusion VH | 98.36 | 2.00E−07 | 5.71 |

TABLE 6
Sequence information of BGA-4712-M3

| Antibody | SEQ ID NO | | SEQUENCE |
|---|---|---|---|
| BGA-4712-M3 | SEQ ID NO: 75 | VH | EVQLLESGGGLVQPGGSLRLSCAASGF |
| | | | TLSAEDVGWVRQAPGKGLEWVSAILD |
| | | | FGGSTYYAESVKGRFTISRDNAKNTLY |
| | | | LQMSSLRAEDTAVYYCARVVYHAGGG |
| | | | VTEDYWGQGTLVTVSS |
| | SEQ ID NO: 76 | VH DNA | GAGGTCCAGTTACTTGAGAGTGGTGG |
| | | | AGGTCTGGTCCAACCAGGAGGTTCG |
| | | | CTGCGTTTATCCTGCGCCGCGTCTGG |
| | | | ATTCACGTTGTCCGCCGAAGACGTGG |
| | | | GTTGGGTGCGTCAAGCGCCGGGGAA |
| | | | AGGACTGGAATGGGTCTCCGCCATCT |
| | | | TGGATTTTGGTGGTTCGACATACTATG |
| | | | CGGAAAGTGTCAAAGGGCGCTTTAC |
| | | | GATCTCGCGCGATAACGCAAAAAATA |
| | | | CTCTTTACCTTCAAATGTCTAGCCTTC |
| | | | GTGCTGAGGACACTGCGGTGTACTAC |
| | | | TGCGCCCGCGTCGTCTACCATGCTGG |
| | | | TGGTGGCGTCACCTTTGATTACTGGG |
| | | | GACAGGGCACCTTAGTTACAGTCTCA |
| | | | TCG |
| BGA-4712-M3- | SEQ ID NO: 136 | AA | EVQLLESGGGLVQPGGSLRLSCAASGF |
| | | | TLSAEDVGWVRQAPGKGLEWVSAILD |
| | | | FGGSTYYAESVKGRFTISRDNAKNTLY |
| | | | LQMSSLRAEDTAVYYCARVVYHAGGG |
| | | | VTFDYWGQGTLVTVSSGGGGSDKTHT |
| | | | CPPCPAPPAAGPSVFLFPPKPKDTLMIS |
| | | | RTPEVTCVVVDVSHEDPEVKFNWYVD |
| | | | GVEVHNAKTKPREEQYNSTYRVVSVL |
| | | | TVLHQDWLNGKEYKCKVSNKALAAPI |
| | | | EKTISKAKGQPREPQVYTLPPSREEMT |
| | | | KNQVSLTCLVKGFYPSDIAVEWESNGQ |
| | | | PENNYKTTPPVLDSDGSFFLYSKLTVD |
| | | | KSRWQQGNVFSCSVMHEALHNHYTQ |
| | | | KSLSLSPGK |
| mutFc | SEQ ID NO: 137 | DNA | GAGGTCCAGTTACTTGAGAGTGGTGG |
| | | | AGGTCTGGTCCAACCAGGAGGTTCG |
| | | | CTGCGTTTATCCTGCGCCGCGTCTGG |
| | | | ATTCACGTTGTCCGCCGAAGACGTGG |
| | | | GTTGGGTGCGTCAAGCGCCGGGGAA |
| | | | AGGACTGGAATGGGTCTCCGCCATCT |
| | | | TGGATTTTGGTGGTTCGACATACTATG |
| | | | CGGAAAGTGTCAAAGGGCGCTTTAC |
| | | | GATCTCGCGCGATAACGCAAAAAATA |
| | | | CTCTTTACCTTCAAATGTCTAGCCTTC |
| | | | GTGCTGAGGACACTGCGGTGTACTAC |
| | | | TGCGCCCGCGTCGTCTACCATGCTGG |
| | | | TGGTGGCGTCACCTTTGATTACTGGG |
| | | | GACAGGGCACCTTAGTTACAGTCTCA |
| | | | TCGGGCGGCGGAGGGTCTGACAAGA |
| | | | CCCACACATGCCCCCCTTGTCCTGCT |
| | | | CCACCAGCTGCAGGACCAAGCGTGT |
| | | | TCCTGTTTCCACCCAAGCCCAAGGAT |
| | | | ACCCTGATGATCTCTCGGACCCCAGA |
| | | | GGTGACATGCGTGGTGGTGGATGTGA |
| | | | GCCACGAGGACCCCGAGGTGAAGTT |
| | | | CAACTGGTATGTGGACGGCGTGGAGG |
| | | | TGCACAATGCTAAGACCAAGCCCAGG |
| | | | GAGGAGCAGTACAACTCCACCTATAG |
| | | | AGTGGTGTCTGTGCTGACAGTGCTGC |
| | | | ACCAGGATTGGCTGAACGGCAAGGA |
| | | | GTATAAGTGCAAGGTGTCCAATAAGG |
| | | | CCCTGGCCGCTCCTATCGAGAAGACC |
| | | | ATCTCTAAGGCCAAGGGCCAGCCCAG |
| | | | AGAGCCTCAGGTGTACACACTGCCTC |
| | | | CATCCCGGGAAGAGATGACCAAGAA |
| | | | CCAGGTGTCTCTGACATGTCTGGTCA |
| | | | AGGGCTTCTATCCCTCTGACATCGCC |
| | | | GTGGAGTGGGAGAGCAATGGCCAGC |
| | | | CTGAGAACAATTACAAGACCACACCC |
| | | | CCTGTGCTGGATTCCGACGGCTCTTT |
| | | | CTTTCTGTATAGCAAGCTGACCGTGG |
| | | | ACAAGTCCCGGTGGCAGCAGGGCAA |
| | | | CGTGTTCAGCTGTTCCGTGATGCACG |
| | | | AAGCTCTGCATAATCACTATACTCAGA |
| | | | AATCCCTGTCACTGTCACCTGGTAAA |

TABLE 7
Sequence information of BGA-7556 in Fc fusion VH

| Antibody | SEQ ID NO | | SEQUENCE |
|---|---|---|---|
| BGA-7556 | SEQ ID NO: 86 | VH | EVQLLESGGGLVQPGGSLRLSCAASG |
| | | | FTLSAEDVGWVRQAPGKGLEWVSAI |
| | | | LDFGGSTYYAESVKGRFTISRDNAKN |
| | | | TLYLQMSSLRAEDTAVYYCARVVYH |
| | | | AGGGVTFDYRGQGTQVTVSS |
| | SEQ ID NO: 87 | VH DNA | GAGGTCCAGTTACTTGAGAGTGGT |
| | | | GGAGGTCTGGTCCAACCAGGAGGT |
| | | | TCGCTGCGTTTATCCTGCGCCGCGT |
| | | | CTGGATTCACGTTGTCCGCCGAAGA |
| | | | CGTGGGTTGGGTGCGTCAAGCGCC |
| | | | GGGGAAAGGACTGGAATGGGTCTC |
| | | | CGCCATCTTGGATTTTGGTGGTTCG |
| | | | ACATACTATGCGGAAAGTGTCAAAG |
| | | | GGCGCTTTACGATCTCGCGCGATAA |
| | | | CGCAAAAAATACTCTTTACCTTCAA |
| | | | ATGTCTAGCCTTCGTGCTGAGGACA |
| | | | CTGCGGTGTACTACTGCGCCCGCGT |
| | | | CGTCTACCATGCTGGTGGTGGCGTC |
| | | | ACCTTTGATTACCGGGGACAGGGCA |
| | | | CCCAAGTTACAGTCTCATCG |
| BGA-7556- | SEQ ID NO: 138 | AA | EVQLLESGGGLVQPGGSLRLSCAASG |
| mutFc | | | FTLSAEDVGWVRQAPGKGLEWVSAI |
| | | | LDFGGSTYYAESVKGRFTISRDNAKN |
| | | | TLYLQMSSLRAEDTAVYYCARVVYH |
| | | | AGGGVTFDYRGQGTQVTVSSGGGGS |
| | | | DKTHTCPPCPAPPAAGPSVFLFPPKPK |
| | | | DTLMISRTPEVTCVVVDVSHEDPEVK |
| | | | FNWYVDGVEVHNAKTKPREEQYNS |
| | | | TYRVVSVLTVLHQDWLNGKEYKCK |
| | | | VSNKALAAPIEKTISKAKGQPREPQV |
| | | | YTLPPSREEMTKNQVSLTCLVKGFYP |
| | | | SDIAVEWESNGQPENNYKTTPPVLDS |
| | | | DGSFFLYSKLTVDKSRWQQGNVFSCS |
| | | | VMHEALHNHYTQKSLSLSPGK |
| | SEQ ID NO: 139 | DNA | GAGGTCCAGTTACTTGAGAGTGGT |
| | | | GGAGGTCTGGTCCAACCAGGAGGT |
| | | | TCGCTGCGTTTATCCTGCGCCGCGT |
| | | | CTGGATTCACGTTGTCCGCCGAAGA |
| | | | CGTGGGTTGGGTGCGTCAAGCGCC |
| | | | GGGGAAAGGACTGGAATGGGTCTC |
| | | | CGCCATCTTGGATTTTGGTGGTTCG |
| | | | ACATACTATGCGGAAAGTGTCAAAG |
| | | | GGCGCTTTACGATCTCGCGCGATAA |
| | | | CGCAAAAAATACTCTTTACCTTCAA |
| | | | ATGTCTAGCCTTCGTGCTGAGGACA |
| | | | CTGCGGTGTACTACTGCGCCCGCGT |
| | | | CGTCTACCATGCTGGTGGTGGCGTC |
| | | | ACCTTTGATTACCGGGGACAGGGCA |
| | | | CCCAAGTTACAGTCTCATCGGGCGG |
| | | | CGGAGGGTCTGACAAGACCCACAC |
| | | | ATGCCCCCCTTGTCCTGCTCCACCA |
| | | | GCTGCAGGACCAAGCGTGTTCCTGT |
| | | | TTCCACCCAAGCCCAAGGATACCCT |
| | | | GATGATCTCTCGGACCCCAGAGGTG |
| | | | ACATGCGTGGTGGTGGATGTGAGCC |
| | | | ACGAGGACCCCGAGGTGAAGTTCA |
| | | | ACTGGTATGTGGACGGCGTGGAGG |
| | | | TGCACAATGCTAAGACCAAGCCCA |
| | | | GGGAGGAGCAGTACAACTCCACCT |
| | | | ATAGAGTGGTGTCTGTGCTGACAGT |
| | | | GCTGCACCAGGATTGGCTGAACGG |
| | | | CAAGGAGTATAAGTGCAAGGTGTCC |
| | | | AATAAGGCCCTGGCCGCTCCTATCG |
| | | | AGAAGACCATCTCTAAGGCCAAGG |
| | | | GCCAGCCCAGAGAGCCTCAGGTGT |
| | | | ACACACTGCCTCCATCCCGGGAAG |
| | | | AGATGACCAAGAACCAGGTGTCTC |
| | | | TGACATGTCTGGTCAAGGGCTTCTA |
| | | | TCCCTCTGACATCGCCGTGGAGTGG |
| | | | GAGAGCAATGGCCAGCCTGAGAAC |
| | | | AATTACAAGACCACACCCCCTGTGC |
| | | | TGGATTCCGACGGCTCTTTCTTTCT |
| | | | GTATAGCAAGCTGACCGTGGACAA |
| | | | GTCCCGGTGGCAGCAGGGCAACGT |
| | | | GTTCAGCTGTTCCGTGATGCACGAA |
| | | | GCTCTGCATAATCACTATACTCAGA |
| | | | AATCCCTGTCACTGTCACCTGGTAA |
| | | | A |

TABLE 8
Sequence information

| CONSTRUCT | SEQ ID NO | | SEQUENCE |
|---|---|---|---|
| CH3 | SEQ ID NO: | AA | GQPREPQVYTLPPSREEMTKNQVSL |
| | 140 | | TCLVKGFYPSDIAVEWESNGQPENN |
| | | | YKTTPPVLDSDGSFFLYSKLTVDKSR |
| | | | WQQGNVFSCSVMHEALHNHYTQK |
| | | | SLSLSPGK |
| | SEQ ID NO: | DNA | GGCCAGCCCAGAGAGCCTCAGGT |
| | 141 | | GTACACACTGCCTCCATCCCGGGA |
| | | | AGAGATGACCAAGAACCAGGTGT |
| | | | CTCTGACATGTCTGGTCAAGGGCT |
| | | | TCTATCCCTCTGACATCGCCGTGGA |
| | | | GTGGGAGAGCAATGGCCAGCCTG |
| | | | AGAACAATTACAAGACCACACCCC |
| | | | CTGTGCTGGATTCCGACGGCTCTTT |
| | | | CTTTCTGTATAGCAAGCTGACCGTG |
| | | | GACAAGTCCCGGTGGCAGCAGGG |
| | | | CAACGTGTTCAGCTGTTCCGTGAT |
| | | | GCACGAAGCTCTGCATAATCACTAT |
| | | | ACTCAGAAATCCCTGTCACTGTCA |
| | | | CCTGGTAAA |
| CH3-BGA-4712- | SEQ ID NO: | AA | GQPREPQVYTLPPSREEMTKNQVSL |
| M3 | 142 | | TCLVKGFYPSDIAVEWESNGQPENN |
| | | | YKTTPPVLDSDGSFFLYSKLTVDKSR |
| | | | WQQGNVFSCSVMHEALHNHYTQK |
| | | | SLSLSPGKGGGGSEVQLLESGGGLV |
| | | | QPGGSLRLSCAASGFTLSAEDVGW |
| | | | VRQAPGKGLEWVSAILDFGGSTYY |
| | | | AESVKGRFTISRDNAKNTLYLQMSS |
| | | | LRAEDTAVYYCARVVYHAGGGVTF |
| | | | DYWGQGTLVTVSS |
| | SEQ ID NO: | DNA | GGCCAGCCCAGAGAGCCTCAGGT |
| | 143 | | GTACACACTGCCTCCATCCCGGGA |
| | | | AGAGATGACCAAGAACCAGGTGT |
| | | | CTCTGACATGTCTGGTCAAGGGCT |
| | | | TCTATCCCTCTGACATCGCCGTGGA |
| | | | GTGGGAGAGCAATGGCCAGCCTG |
| | | | AGAACAATTACAAGACCACACCCC |
| | | | CTGTGCTGGATTCCGACGGCTCTTT |
| | | | CTTTCTGTATAGCAAGCTGACCGTG |
| | | | GACAAGTCCCGGTGGCAGCAGGG |
| | | | CAACGTGTTCAGCTGTTCCGTGAT |
| | | | GCACGAAGCTCTGCATAATCACTAT |
| | | | ACTCAGAAATCCCTGTCACTGTCA |
| | | | CCTGGTAAAGGTGGAGGCGGTTCA |
| | | | GAGGTCCAGTTACTTGAGAGTGGT |
| | | | GGAGGTCTGGTCCAACCAGGAGG |
| | | | TTCGCTGCGTTTATCCTGCGCCGCG |
| | | | TCTGGATTCACGTTGTCCGCCGAA |
| | | | GACGTGGGTTGGGTGCGTCAAGCG |
| | | | CCGGGGAAAGGACTGGAATGGGT |
| | | | CTCCGCCATCTTGGATTTTGGTGGT |
| | | | TCGACATACTATGCGGAAAGTGTC |
| | | | AAAGGGCGCTTTACGATCTCGCGC |
| | | | GATAACGCAAAAAATACTCTTTACC |
| | | | TTCAAATGTCTAGCCTTCGTGCTGA |
| | | | GGACACTGCGGTGTACTACTGCGC |
| | | | CCGCGTCGTCTACCATGCTGGTGG |
| | | | TGGCGTCACCTTTGATTACTGGGG |
| | | | ACAGGGCACCTTAGTTACAGTCTC |
| | | | ATCG |

TABLE 9
Affinity comparison of affinity matured
BGA-4712 variants as Fc fusion antibody

| Clone # | Format | SEC-HPLC (%) | KD (M) |
|---|---|---|---|
| BGA-5623 | Fc-fusion VH | 97.62 | 5.03E−08 |
| BGA-2690 | | 97.82 | 6.75E−05 |
| BGA-3849 | | 98.75 | 7.63E−08 |
| BGA-7916 | | 97.57 | 8.77E−08 |
| BGA-4988 | | 97.71 | 1.03E−06 |
| BGA-5164 | | 99.07 | 1.38E−05 |
| BGA-3953 | | 97.29 | 4.83E−08 |
| BGA-5385 | | 97.86 | 1.00E−07 |
| BGA-9468 | | 97.56 | 1.90E−07 |
| BGA-3285 | | 97.83 | 1.86E−07 |
| BGA-9442 | | 98.6 | 1.51E−07 |
| BGA-7746 | | 97.68 | 1.93E−07 |
| BGA-8425 | | 98.2 | 9.34E−08 |
| BGA-6468 | | 92.00 | 1.12E−08 |
| BGA-4712-M3 | | 97.98 | 1.69E−07 |
| BGA-7556 | | 98.36 | 2.00E−07 |

TABLE 10
Sequence information for affinity matured BGA-4712 variants

| | | |
|---|---|---|
| SEQ ID NO: | BGA-5623 | EVQLLESGGGLVQPGGSLRLSCAASGFTVSAEDVGWV |
| 84 | AA | RQAPGKGLEWVSAILDKGGSTYYAESVKGRFTISRDN |
| | | AKNTLYLQMSSLRAEDTAVYYCARIVYHAGGGVTFD |
| | | TRGQGTQVTVSS |
| SEQ ID NO: | BGA-5623 | GAGGTCCAGTTACTTGAGAGTGGTGGAGGTCTGGTC |
| 85 | DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATAAGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCATTGTCTACCATGCTGGTGGTGGCGTCAC |
| | | CTTTGATACTCGGGGACAGGGCACCCAAGTTACAGT |
| | | CTCATCG |
| SEQ ID NO: | BGA-2690 | EVQLLESGGGLVQPGGSLRLSCAASGFTLSREDVGWV |
| 144 | AA | RQAPGKGLEWVSAILDFGSSTYYAESVKGRFTISRDNA |
| | | KNTLYLQMSSLRAEDTAVYYCARVVYHAGGGVTYD |
| | | YRGQGTQVTVSS |
| SEQ ID NO: | BGA-2690 | GAGGTCCAGTTACTTGAGAGTGGTGGAGGTCTGGTC |
| 145 | DNA | CAACCAGGAGGTTCGCTGCGTTTATCCTGCGCCGCG |
| | | TCTGGATTCACGCTTTCCAGAGAAGACGTGGGTTGG |
| | | GTGCGTCAAGCGCCGGGGAAAGGACTGGAATGGGT |
| | | CTCCGCCATCTTGGATTTCGGTTCTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCGTTGTCTACCATGCTGGTGGTGGCGTCACC |
| | | TATGATTATCGGGGACAGGGCACCCAAGTTACAGTC |
| | | TCATCG |
| SEQ ID NO: | BGA-3849 | EVQLLESGGGLVQPGGSLRLSCAASGFTLSHEDVGWV |
| 146 | AA | RQAPGKGLEWVSAILDSGGSTYYAESVKGRFTISRDN |
| | | AKNTLYLQMSSLRAEDTAVYYCARYVYHAGGGVTFD |
| | | TRGQGTQVTVSS |
| SEQ ID NO: | BGA-3849 | GAGGTCCAGTTACTTGAGAGTGGTGGAGGTCTGGTC |
| 147 | DNA | CAACCAGGAGGTTCGCTGCGTTTATCCTGCGCCGCG |
| | | TCTGGATTCACGCTTTCCCATGAAGACGTGGGTTGG |
| | | GTGCGTCAAGCGCCGGGGAAAGGACTGGAATGGGT |
| | | CTCCGCCATCTTGGATTCCGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCTATGTCTACCATGCTGGTGGTGGCGTCACC |
| | | TTTGATACTCGGGGACAGGGCACCCAAGTTACAGTC |
| | | TCATCG |
| SEQ ID NO: | BGA-7916 | EVQLLESGGGLVQPGGSLRLSCAASGFTVSAEDVGWV |
| 148 | AA | RQAPGKGLEWVSAILDSGGSTYYAESVKGRFTISRDN |
| | | AKNTLYLQMSSLRAEDTAVYYCARVVYHAGGGVTY |
| | | DYRGQGTQVTVSS |
| SEQ ID NO: | BGA-7916 | GAGGTCCAGTTACTTGAGAGTGGTGGAGGTCTGGTC |
| 149 | DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATTCGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTGTCTACCATGCTGGTGGTGGCGTCAC |
| | | CTATGATACTCGGGGACAGGGCACCCAAGTTACAGT |
| | | CTCATCG |
| SEQ ID NO: | BGA-4988 | EVQLLESGGGLVQPGGSLRLSCAASGFTVSAEDVGWV |
| 150 | AA | RQAPGKGLEWVSAILDRGGSTYYAESVKGRFTISRDN |
| | | AKNTLYLQMSSLRAEDTAVYYCARVVYHAGGGVTY |
| | | DYRGQGTQVTVSS |
| SEQ ID NO: | BGA-4988 | GAGGTCCAGTTACTTGAGAGTGGTGGAGGTCTGGTC |
| 151 | DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATCGGGGGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTGTCTACCATGCTGGTGGTGGCGTCAC |
| | | CTATGATACTCGGGGACAGGGCACCCAAGTTACAGT |
| | | CTCATCG |
| SEQ ID NO: | BGA-5164 | EVQLLESGGGLVQPGGSLRLSCAASGFTLSAEDVGWV |
| 152 | AA | RQAPGKGLEWVSAILDHGGSTYYAESVKGRFTISRDN |
| | | AKNTLYLQMSSLRAEDTAVYYCARVVYHAGGGVTY |
| | | DYRGQGTQVTVSS |
| SEQ ID NO: | BGA-5164 | GAGGTCCAGTTACTTGAGAGTGGTGGAGGTCTGGTC |
| 153 | DNA | CAACCAGGAGGTTCGCTGCGTTTATCCTGCGCCGCG |
| | | TCTGGATTCACGCTTTCCGCCGAAGACGTGGGTTGG |
| | | GTGCGTCAAGCGCCGGGGAAAGGACTGGAATGGGT |
| | | CTCCGCCATCTTGGATCATGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTGTCTACCATGCTGGTGGTGGCGTCAC |
| | | CTATGATTATCGGGGACAGGGCACCCAAGTTACAGT |
| | | CTCATCG |
| SEQ ID NO: | BGA-3953 | EVQLLESGGGLVQPGGSLRLSCAASGFTVSAEDVGWV |
| 154 | AA | RQAPGKGLEWVSAILDSGGSTYYAESVKGRFTISRDN |
| | | AKNTLYLQMSSLRAEDTAVYYCARIVYHAGGGVTFD |
| | | TRGQGTQVTVSS |
| SEQ ID NO: | BGA-3953 | GAGGTCCAGTTACTTGAGAGTGGTGGAGGTCTGGTC |
| 155 | DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATtcGGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCATTGTCTACCATGCTGGTGGTGGCGTCACC |
| | | TTTGATACTCGGGGACAGGGCACCCAAGTTACAGTC |
| | | TCATCG |
| SEQ ID NO: | BGA-5385 | EVQLLESGGGLVQPGGSLRLSCAASGFTLSAEDVGWV |
| 156 | AA | RQAPGKGLEWVSAILDHGGSTYYAESVKGRFTISRDN |
| | | AKNTLYLQMSSLRAEDTAVYYCARVIYHAGGGVTFD |
| | | TRGQGTQVTVSS |
| SEQ ID NO: | BGA-5385 | GAGGTCCAGTTACTTGAGAGTGGTGGAGGTCTGGTC |
| 157 | DNA | CAACCAGGAGGTTCGCTGCGTTTATCCTGCGCCGCG |
| | | TCTGGATTCACGCTTTCCGCCGAAGACGTGGGTTGG |
| | | GTGCGTCAAGCGCCGGGGAAAGGACTGGAATGGGT |
| | | CTCCGCCATCTTGGATCATGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTATCTACCATGCTGGTGGTGGCGTCAC |
| | | CTTTGATACTCGGGGACAGGGCACCCAAGTTACAGT |
| | | CTCATCG |
| SEQ ID NO: | BGA-9468 | EVQLLESGGGLVQPGGSLRLSCAASGFTVSAEDVGWV |
| 158 | AA | RQAPGKGLEWVSAILDSGGSTYYAESVKGRFTISRDN |
| | | AKNTLYLQMSSLRAEDTAVYYCARVVYHSGGGVTFD |
| | | LRGQGTQVTVSS |
| SEQ ID NO: | BGA-9468 | GAGGTCCAGTTACTTGAGAGTGGTGGAGGTCTGGTC |
| 159 | DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATtcGGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCGTTGTCTACCATTCTGGTGGTGGCGTCACC |
| | | TTTGATCTTCGGGGACAGGGCACCCAAGTTACAGTC |
| | | TCATCG |
| SEQ ID NO: | BGA-3285 | EVQLLESGGGLVQPGGSLRLSCAASGFTISAEDVGWV |
| 160 | AA | RQAPGKGLEWVSAILDFGGSTYYAESVKGRFTISRDN |
| | | AKNTLYLQMSSLRAEDTAVYYCARVVYHAGGGVTFD |
| | | TRGQGTQVTVSS |
| SEQ ID NO: | BGA-3285 | GAGGTCCAGTTACTTGAGAGTGGTGGAGGTCTGGTC |
| 161 | DNA | CAACCAGGAGGTTCGCTGCGTTTATCCTGCGCCGCG |
| | | TCTGGATTCACGATTTCCGCCGAAGACGTGGGTTGG |
| | | GTGCGTCAAGCGCCGGGGAAAGGACTGGAATGGGT |
| | | CTCCGCCATCTTGGATTTTGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCGTTGTCTACCATGCTGGTGGTGGCGTCACC |
| | | TTTGATACTCGGGGACAGGGCACCCAAGTTACAGTC |
| | | TCATCG |
| SEQ ID NO: | BGA-9442 | EVQLLESGGGLVQPGGSLRLSCAASGFTISAEDVGWV |
| 162 | AA | RQAPGKGLEWVSAILDSGGSTYYAESVKGRFTISRDN |
| | | AKNTLYLQMSSLRAEDTAVYYCARYVYHAGGGVTFD |
| | | LRGQGTQVTVSS |
| SEQ ID NO: | BGA-9442 | GAGGTCCAGTTACTTGAGAGTGGTGGAGGTCTGGTC |
| 163 | DNA | CAACCAGGAGGTTCGCTGCGTTTATCCTGCGCCGCG |
| | | TCTGGATTCACGATTTCCGCCGAAGACGTGGGTTGG |
| | | GTGCGTCAAGCGCCGGGGAAAGGACTGGAATGGGT |
| | | CTCCGCCATCTTGGATTCTGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCTATGTCTACCATGCTGGTGGTGGCGTCACC |
| | | TTTGATCTTCGGGGACAGGGCACCCAAGTTACAGTC |
| | | TCATCG |
| SEQ ID NO: | BGA-7746 | EVQLLESGGGLVQPGGSLRLSCAASGFTVSAEDVGWV |
| 164 | AA | RQAPGKGLEWVSAILDFGGSTYYAESVKGRFTISRDN |
| | | AKNTLYLQMSSLRAEDTAVYYCARVVYHAGGGVTFD |
| | | TRGQGTQVTVSS |
| SEQ ID NO: | BGA-7746 | GAGGTCCAGTTACTTGAGAGTGGTGGAGGTCTGGTC |
| 165 | DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATTTTGGTGGTTCGACATACTAT |
| | | GCAGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCGTCGTCTACCATGCTGGTGGTGGCGTCACC |
| | | TTTGATACGCGGGGACAGGGCACCCAAGTTACAGTC |
| | | TCATCG |
| SEQ ID NO: | BGA-8425 | EVQLLESGGGLVQPGGSLRLSCAASGFTLSAEDVGWV |
| 166 | AA | RQAPGKGLEWVSAILDSGGSTYYAESVKGRFTISRDN |
| | | AKNTLYLQMSSLRAEDTAVYYCARVVYHAGGGVTY |
| | | DYRGQGTQVTVSS |
| SEQ ID NO: | BGA-8425 | GAGGTCCAGTTACTTGAGAGTGGTGGAGGTCTGGTC |
| 167 | DNA | CAACCAGGAGGTTCGCTGCGTTTATCCTGCGCCGCG |
| | | TCTGGATTCACGCTGTCCGCCGAAGACGTGGGTTGG |
| | | GTGCGTCAAGCGCCGGGGAAAGGACTGGAATGGGT |
| | | CTCCGCCATCTTGGATTCGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTCGTCTACCATGCTGGTGGTGGCGTCAC |
| | | CTATGATTACCGGGGACAGGGCACCCAAGTTACAGT |
| | | CTCATCG |
| SEQ ID NO: | BGA-3386 | EVQLLESGGGLVQPGGSLRLSCAASGFTVSAEDVGWV |
| 168 | AA | RQAPGKGLEWVSAILDKGGSTYYAESVKGRFTISRDN |
| | | AKNTLYLQMSSLRAEDTAVYYCARIVYHAGGGVTFD |
| | | TWGQGTLVTVSS |
| SEQ ID NO: | BGA-3386 | GAGGTCCAGTTACTTGAGAGTGGTGGAGGTCTGGTC |
| 169 | DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATAAGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCATTGTCTACCATGCTGGTGGTGGCGTCAC |
| | | CTTTGATACTTGGGGACAGGGCACCTTAGTTACAGT |
| | | CTCATCG |
| SEQ ID NO: | BGA-5623- | EVQLLESGGGLVQPGGSLRLSCAASGFTVSAEDVGWV |
| 170 | mutFc-AA | RQAPGKGLEWVSAILDKGGSTYYAESVKGRFTISRDN |
| | | AKNTLYLQMSSLRAEDTAVYYCARIVYHAGGGVTFD |
| | | TRGQGTQVTVSSGGGGSDKTHTCPPCPAPPAAGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALAAPIEKTISKAKGQPREPQVYTLPP |
| | | SREEMTKNQVSLTCLVKGFYPSDIAVEWESNGQPENN |
| | | YKTTPPVLDSDGSFFLYSKLTVDKSRWQQGNVFSCSV |
| | | MHEALHNHYTQKSLSLSPGK |
| SEQ ID NO: | BGA-5623- | GAGGTCCAGTTACTTGAGAGTGGTGGAGGTCTGGTC |
| 171 | mutFc DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATAAGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCATTGTCTACCATGCTGGTGGTGGCGTCAC |
| | | CTTTGATACTCGGGGACAGGGCACCCAAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |
| SEQ ID NO: | BGA-2690- | EVQLLESGGGLVQPGGSLRLSCAASGFTLSREDVGWV |
| 172 | mutFc AA | RQAPGKGLEWVSAILDFGSSTYYAESVKGRFTISRDNA |
| | | KNTLYLQMSSLRAEDTAVYYCARVVYHAGGGVTYD |
| | | YRGQGTQVTVSSGGGGSDKTHTCPPCPAPPAAGPSVF |
| | | LFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALAAPIEKTISKAKGQPREPQVYTLP |
| | | PSREEMTKNQVSLTCLVKGFYPSDIAVEWESNGQPEN |
| | | NYKTTPPVLDSDGSFFLYSKLTVDKSRWQQGNVFSCS |
| | | VMHEALHNHYTQKSLSLSPGK |
| SEQ ID NO: | BGA-2690 | GAGGTCCAGTTACTTGAGAGTGGTGGAGGTCTGGTC |
| 173 | mutFc DNA | CAACCAGGAGGTTCGCTGCGTTTATCCTGCGCCGCG |
| | | TCTGGATTCACGCTTTCCAGAGAAGACGTGGGTTGG |
| | | GTGCGTCAAGCGCCGGGGAAAGGACTGGAATGGGT |
| | | CTCCGCCATCTTGGATTTCGGTTCTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCGTTGTCTACCATGCTGGTGGTGGCGTCACC |
| | | TATGATTATCGGGGACAGGGCACCCAAGTTACAGTC |
| | | TCATCGGGCGGCGGAGGGTCTGACAAGACCCACAC |
| | | ATGCCCCCCTTGTCCTGCTCCACCAGCTGCAGGACC |
| | | AAGCGTGTTCCTGTTTCCACCCAAGCCCAAGGATAC |
| | | CCTGATGATCTCTCGGACCCCAGAGGTGACATGCGT |
| | | GGTGGTGGATGTGAGCCACGAGGACCCCGAGGTGA |
| | | AGTTCAACTGGTATGTGGACGGCGTGGAGGTGCACA |
| | | ATGCTAAGACCAAGCCCAGGGAGGAGCAGTACAAC |
| | | TCCACCTATAGAGTGGTGTCTGTGCTGACAGTGCTG |
| | | CACCAGGATTGGCTGAACGGCAAGGAGTATAAGTG |
| | | CAAGGTGTCCAATAAGGCCCTGGCCGCTCCTATCGA |
| | | GAAGACCATCTCTAAGGCCAAGGGCCAGCCCAGAG |
| | | AGCCTCAGGTGTACACACTGCCTCCATCCCGGGAAG |
| | | AGATGACCAAGAACCAGGTGTCTCTGACATGTCTGG |
| | | TCAAGGGCTTCTATCCCTCTGACATCGCCGTGGAGT |
| | | GGGAGAGCAATGGCCAGCCTGAGAACAATTACAAG |
| | | ACCACACCCCCTGTGCTGGATTCCGACGGCTCTTTCT |
| | | TTCTGTATAGCAAGCTGACCGTGGACAAGTCCCGGT |
| | | GGCAGCAGGGCAACGTGTTCAGCTGTTCCGTGATGC |
| | | ACGAAGCTCTGCATAATCACTATACTCAGAAATCCC |
| | | TGTCACTGTCACCTGGTAAA |
| SEQ ID NO: | BGA-3849- | EVQLLESGGGLVQPGGSLRLSCAASGFTLSHEDVGWV |
| 174 | mutFc | RQAPGKGLEWVSAILDSGGSTYYAESVKGRFTISRDN |
| | AA | AKNTLYLQMSSLRAEDTAVYYCARYVYHAGGGVTFD |
| | | TRGQGTQVTVSSGGGGSDKTHTCPPCPAPPAAGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALAAPIEKTISKAKGQPREPQVYTLPP |
| | | SREEMTKNQVSLTCLVKGFYPSDIAVEWESNGQPENN |
| | | YKTTPPVLDSDGSFFLYSKLTVDKSRWQQGNVFSCSV |
| | | MHEALHNHYTQKSLSLSPGK |
| SEQ ID NO: | BGA-3849 | GAGGTCCAGTTACTTGAGAGTGGTGGAGGTCTGGTC |
| 175 | mutFc DNA | CAACCAGGAGGTTCGCTGCGTTTATCCTGCGCCGCG |
| | | TCTGGATTCACGCTTTCCCATGAAGACGTGGGTTGG |
| | | GTGCGTCAAGCGCCGGGGAAAGGACTGGAATGGGT |
| | | CTCCGCCATCTTGGATTCCGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCTATGTCTACCATGCTGGTGGTGGCGTCACC |
| | | TTTGATACTCGGGGACAGGGCACCCAAGTTACAGTC |
| | | TCATCGGGCGGCGGAGGGTCTGACAAGACCCACAC |
| | | ATGCCCCCCTTGTCCTGCTCCACCAGCTGCAGGACC |
| | | AAGCGTGTTCCTGTTTCCACCCAAGCCCAAGGATAC |
| | | CCTGATGATCTCTCGGACCCCAGAGGTGACATGCGT |
| | | GGTGGTGGATGTGAGCCACGAGGACCCCGAGGTGA |
| | | AGTTCAACTGGTATGTGGACGGCGTGGAGGTGCACA |
| | | ATGCTAAGACCAAGCCCAGGGAGGAGCAGTACAAC |
| | | TCCACCTATAGAGTGGTGTCTGTGCTGACAGTGCTG |
| | | CACCAGGATTGGCTGAACGGCAAGGAGTATAAGTG |
| | | CAAGGTGTCCAATAAGGCCCTGGCCGCTCCTATCGA |
| | | GAAGACCATCTCTAAGGCCAAGGGCCAGCCCAGAG |
| | | AGCCTCAGGTGTACACACTGCCTCCATCCCGGGAAG |
| | | AGATGACCAAGAACCAGGTGTCTCTGACATGTCTGG |
| | | TCAAGGGCTTCTATCCCTCTGACATCGCCGTGGAGT |
| | | GGGAGAGCAATGGCCAGCCTGAGAACAATTACAAG |
| | | ACCACACCCCCTGTGCTGGATTCCGACGGCTCTTTCT |
| | | TTCTGTATAGCAAGCTGACCGTGGACAAGTCCCGGT |
| | | GGCAGCAGGGCAACGTGTTCAGCTGTTCCGTGATGC |
| | | ACGAAGCTCTGCATAATCACTATACTCAGAAATCCC |
| | | TGTCACTGTCACCTGGTAAA |
| SEQ ID NO: | BGA-7916- | EVQLLESGGGLVQPGGSLRLSCAASGFTVSAEDVGWV |
| 176 | mutFc AA | RQAPGKGLEWVSAILDSGGSTYYAESVKGRFTISRDN |
| | | AKNTLYLQMSSLRAEDTAVYYCARVVYHAGGGVTY |
| | | DTRGQGTQVTVSSGGGGSDKTHTCPPCPAPPAAGPSV |
| | | FLFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALAAPIEKTISKAKGQPREPQVYTLP |
| | | PSREEMTKNQVSLTCLVKGFYPSDIAVEWESNGQPEN |
| | | NYKTTPPVLDSDGSFFLYSKLTVDKSRWQQGNVFSCS |
| | | VMHEALHNHYTQKSLSLSPGK |
| SEQ ID NO: | BGA-7916- | GAGGTCCAGTTACTTGAGAGTGGTGGAGGTCTGGTC |
| 177 | mutFc DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATTCGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTGTCTACCATGCTGGTGGTGGCGTCAC |
| | | CTATGATACTCGGGGACAGGGCACCCAAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |
| SEQ ID NO: | BGA-4988- | EVQLLESGGGLVQPGGSLRLSCAASGFTVSAEDVGWV |
| 178 | mutFc AA | RQAPGKGLEWVSAILDRGGSTYYAESVKGRFTISRDN |
| | | AKNTLYLQMSSLRAEDTAVYYCARVVYHAGGGVTY |
| | | DTRGQGTQVTVSSGGGGSDKTHTCPPCPAPPAAGPSV |
| | | FLFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALAAPIEKTISKAKGQPREPQVYTLP |
| | | PSREEMTKNQVSLTCLVKGFYPSDIAVEWESNGQPEN |
| | | NYKTTPPVLDSDGSFFLYSKLTVDKSRWQQGNVFSCS |
| | | VMHEALHNHYTQKSLSLSPGK |
| SEQ ID NO: | BGA-4988- | GAGGTCCAGTTACTTGAGAGTGGTGGAGGTCTGGTC |
| 179 | mutFc DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATCGGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTGTCTACCATGCTGGTGGTGGCGTCAC |
| | | CTATGATACTCGGGGACAGGGCACCCAAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |
| SEQ ID NO: | BGA-5164- | EVQLLESGGGLVQPGGSLRLSCAASGFTLSAEDVGWV |
| 180 | mutFc AA | RQAPGKGLEWVSAILDHGGSTYYAESVKGRFTISRDN |
| | | AKNTLYLQMSSLRAEDTAVYYCARVVYHAGGGVTY |
| | | DYRGQGTQVTVSSGGGGSDKTHTCPPCPAPPAAGPSV |
| | | FLFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALAAPIEKTISKAKGQPREPQVYTLP |
| | | PSREEMTKNQVSLTCLVKGFYPSDIAVEWESNGQPEN |
| | | NYKTTPPVLDSDGSFFLYSKLTVDKSRWQQGNVFSCS |
| | | VMHEALHNHYTQKSLSLSPGK |
| SEQ ID NO: | BGA-5164- | GAGGTCCAGTTACTTGAGAGTGGTGGAGGTCTGGTC |
| 181 | mutFc | CAACCAGGAGGTTCGCTGCGTTTATCCTGCGCCGCG |
| | DNA | TCTGGATTCACGCTTTCCGCCGAAGACGTGGGTTGG |
| | | GTGCGTCAAGCGCCGGGGAAAGGACTGGAATGGGT |
| | | CTCCGCCATCTTGGATCATGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTGTCTACCATGCTGGTGGTGGCGTCAC |
| | | CTATGATTATCGGGGACAGGGCACCCAAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |
| SEQ ID NO: | BGA-3953- | EVQLLESGGGLVQPGGSLRLSCAASGFTVSAEDVGWV |
| 182 | mutFc AA | RQAPGKGLEWVSAILDSGGSTYYAESVKGRFTISRDN |
| | | AKNTLYLQMSSLRAEDTAVYYCARIVYHAGGGVTFD |
| | | TRGQGTQVTVSSGGGGSDKTHTCPPCPAPPAAGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALAAPIEKTISKAKGQPREPQVYTLPP |
| | | SREEMTKNQVSLTCLVKGFYPSDIAVEWESNGQPENN |
| | | YKTTPPVLDSDGSFFLYSKLTVDKSRWQQGNVFSCSV |
| | | MHEALHNHYTQKSLSLSPGK |
| SEQ ID NO: | BGA-3953- | GAGGTCCAGTTACTTGAGAGTGGTGGAGGTCTGGTC |
| 183 | mutFc DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATtcGGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCATTGTCTACCATGCTGGTGGTGGCGTCACC |
| | | TTTGATACTCGGGGACAGGGCACCCAAGTTACAGTC |
| | | TCATCGGGCGGCGGAGGGTCTGACAAGACCCACAC |
| | | ATGCCCCCCTTGTCCTGCTCCACCAGCTGCAGGACC |
| | | AAGCGTGTTCCTGTTTCCACCCAAGCCCAAGGATAC |
| | | CCTGATGATCTCTCGGACCCCAGAGGTGACATGCGT |
| | | GGTGGTGGATGTGAGCCACGAGGACCCCGAGGTGA |
| | | AGTTCAACTGGTATGTGGACGGCGTGGAGGTGCACA |
| | | ATGCTAAGACCAAGCCCAGGGAGGAGCAGTACAAC |
| | | TCCACCTATAGAGTGGTGTCTGTGCTGACAGTGCTG |
| | | CACCAGGATTGGCTGAACGGCAAGGAGTATAAGTG |
| | | CAAGGTGTCCAATAAGGCCCTGGCCGCTCCTATCGA |
| | | GAAGACCATCTCTAAGGCCAAGGGCCAGCCCAGAG |
| | | AGCCTCAGGTGTACACACTGCCTCCATCCCGGGAAG |
| | | AGATGACCAAGAACCAGGTGTCTCTGACATGTCTGG |
| | | TCAAGGGCTTCTATCCCTCTGACATCGCCGTGGAGT |
| | | GGGAGAGCAATGGCCAGCCTGAGAACAATTACAAG |
| | | ACCACACCCCCTGTGCTGGATTCCGACGGCTCTTTCT |
| | | TTCTGTATAGCAAGCTGACCGTGGACAAGTCCCGGT |
| | | GGCAGCAGGGCAACGTGTTCAGCTGTTCCGTGATGC |
| | | ACGAAGCTCTGCATAATCACTATACTCAGAAATCCC |
| | | TGTCACTGTCACCTGGTAAA |
| SEQ ID NO: | BGA-5385- | EVQLLESGGGLVQPGGSLRLSCAASGFTLSAEDVGWV |
| 184 | mutFc AA | RQAPGKGLEWVSAILDHGGSTYYAESVKGRFTISRDN |
| | | AKNTLYLQMSSLRAEDTAVYYCARVIYHAGGGVTYD |
| | | YRGQGTQVTVSSGGGGSDKTHTCPPCPAPPAAGPSVF |
| | | LFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALAAPIEKTISKAKGQPREPQVYTLP |
| | | PSREEMTKNQVSLTCLVKGFYPSDIAVEWESNGQPEN |
| | | NYKTTPPVLDSDGSFFLYSKLTVDKSRWQQGNVFSCS |
| | | VMHEALHNHYTQKSLSLSPGK |
| SEQ ID NO: | BGA-5385- | GAGGTCCAGTTACTTGAGAGTGGTGGAGGTCTGGTC |
| 185 | mutFc DNA | CAACCAGGAGGTTCGCTGCGTTTATCCTGCGCCGCG |
| | | TCTGGATTCACGcTTTCCGCCGAAGACGTGGGTTGG |
| | | GTGCGTCAAGCGCCGGGGAAAGGACTGGAATGGGT |
| | | CTCCGCCATCTTGGATCATGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTATCTACCATGCTGGTGGTGGCGTCAC |
| | | CTATGATTATCGGGGACAGGGCACCCAAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |
| SEQ ID NO: | BGA-9468- | EVQLLESGGGLVQPGGSLRLSCAASGFTVSAEDVGWV |
| 186 | mutFc AA | RQAPGKGLEWVSAILDSGGSTYYAESVKGRFTISRDN |
| | | AKNTLYLQMSSLRAEDTAVYYCARVVYHSGGGVTFD |
| | | LRGQGTQVTVSSGGGGSDKTHTCPPCPAPPAAGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALAAPIEKTISKAKGQPREPQVYTLPP |
| | | SREEMTKNQVSLTCLVKGFYPSDIAVEWESNGQPENN |
| | | YKTTPPVLDSDGSFFLYSKLTVDKSRWQQGNVFSCSV |
| | | MHEALHNHYTQKSLSLSPGK |
| SEQ ID NO: | BGA-9468- | GAGGTCCAGTTACTTGAGAGTGGTGGAGGTCTGGTC |
| 187 | mutFc DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATTCGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTTGTCTACCATTCTGGTGGTGGCGTCAC |
| | | CTTTGATCTTCGGGGACAGGGCACCCAAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |
| SEQ ID NO: | BGA-3285- | EVQLLESGGGLVQPGGSLRLSCAASGFTISAEDVGWV |
| 188 | mutFc | RQAPGKGLEWVSAILDFGGSTYYAESVKGRFTISRDN |
| | AA | AKNTLYLQMSSLRAEDTAVYYCARVVYHAGGGVTFD |
| | | TRGQGTQVTVSSGGGGSDKTHTCPPCPAPPAAGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALAAPIEKTISKAKGQPREPQVYTLPP |
| | | SREEMTKNQVSLTCLVKGFYPSDIAVEWESNGQPENN |
| | | YKTTPPVLDSDGSFFLYSKLTVDKSRWQQGNVFSCSV |
| | | MHEALHNHYTQKSLSLSPGK |
| SEQ ID NO: | BGA-3285- | GAGGTCCAGTTACTTGAGAGTGGTGGAGGTCTGGTC |
| 189 | mutFc DNA | CAACCAGGAGGTTCGCTGCGTTTATCCTGCGCCGCG |
| | | TCTGGATTCACGATTTCCGCCGAAGACGTGGGTTGG |
| | | GTGCGTCAAGCGCCGGGGAAAGGACTGGAATGGGT |
| | | CTCCGCCATCTTGGATTTTGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCGTTGTCTACCATGCTGGTGGTGGCGTCACC |
| | | TTTGATACTCGGGGACAGGGCACCCAAGTTACAGTC |
| | | TCATCGGGCGGCGGAGGGTCTGACAAGACCCACAC |
| | | ATGCCCCCCTTGTCCTGCTCCACCAGCTGCAGGACC |
| | | AAGCGTGTTCCTGTTTCCACCCAAGCCCAAGGATAC |
| | | CCTGATGATCTCTCGGACCCCAGAGGTGACATGCGT |
| | | GGTGGTGGATGTGAGCCACGAGGACCCCGAGGTGA |
| | | AGTTCAACTGGTATGTGGACGGCGTGGAGGTGCACA |
| | | ATGCTAAGACCAAGCCCAGGGAGGAGCAGTACAAC |
| | | TCCACCTATAGAGTGGTGTCTGTGCTGACAGTGCTG |
| | | CACCAGGATTGGCTGAACGGCAAGGAGTATAAGTG |
| | | CAAGGTGTCCAATAAGGCCCTGGCCGCTCCTATCGA |
| | | GAAGACCATCTCTAAGGCCAAGGGCCAGCCCAGAG |
| | | AGCCTCAGGTGTACACACTGCCTCCATCCCGGGAAG |
| | | AGATGACCAAGAACCAGGTGTCTCTGACATGTCTGG |
| | | TCAAGGGCTTCTATCCCTCTGACATCGCCGTGGAGT |
| | | GGGAGAGCAATGGCCAGCCTGAGAACAATTACAAG |
| | | ACCACACCCCCTGTGCTGGATTCCGACGGCTCTTTCT |
| | | TTCTGTATAGCAAGCTGACCGTGGACAAGTCCCGGT |
| | | GGCAGCAGGGCAACGTGTTCAGCTGTTCCGTGATGC |
| | | ACGAAGCTCTGCATAATCACTATACTCAGAAATCCC |
| | | TGTCACTGTCACCTGGTAAA |
| SEQ ID NO: | BGA-9442- | EVQLLESGGGLVQPGGSLRLSCAASGFTISAEDVGWV |
| 190 | mutFc AA | RQAPGKGLEWVSAILDSGGSTYYAESVKGRFTISRDN |
| | | AKNTLYLQMSSLRAEDTAVYYCARYVYHAGGGVTFD |
| | | LRGQGTQVTVSSGGGGSDKTHTCPPCPAPPAAGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALAAPIEKTISKAKGQPREPQVYTLPP |
| | | SREEMTKNQVSLTCLVKGFYPSDIAVEWESNGQPENN |
| | | YKTTPPVLDSDGSFFLYSKLTVDKSRWQQGNVFSCSV |
| | | MHEALHNHYTQKSLSLSPGK |
| SEQ ID NO: | BGA-9442- | GAGGTCCAGTTACTTGAGAGTGGTGGAGGTCTGGTC |
| 191 | mutFc DNA | CAACCAGGAGGTTCGCTGCGTTTATCCTGCGCCGCG |
| | | TCTGGATTCACGATTTCCGCCGAAGACGTGGGTTGG |
| | | GTGCGTCAAGCGCCGGGGAAAGGACTGGAATGGGT |
| | | CTCCGCCATCTTGGATTCTGGTGGTTCGACATACTAT |
| | | GCGGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCTATGTCTACCATGCTGGTGGTGGCGTCACC |
| | | TTTGATCTTCGGGGACAGGGCACCCAAGTTACAGTC |
| | | TCATCGGGCGGCGGAGGGTCTGACAAGACCCACAC |
| | | ATGCCCCCCTTGTCCTGCTCCACCAGCTGCAGGACC |
| | | AAGCGTGTTCCTGTTTCCACCCAAGCCCAAGGATAC |
| | | CCTGATGATCTCTCGGACCCCAGAGGTGACATGCGT |
| | | GGTGGTGGATGTGAGCCACGAGGACCCCGAGGTGA |
| | | AGTTCAACTGGTATGTGGACGGCGTGGAGGTGCACA |
| | | ATGCTAAGACCAAGCCCAGGGAGGAGCAGTACAAC |
| | | TCCACCTATAGAGTGGTGTCTGTGCTGACAGTGCTG |
| | | CACCAGGATTGGCTGAACGGCAAGGAGTATAAGTG |
| | | CAAGGTGTCCAATAAGGCCCTGGCCGCTCCTATCGA |
| | | GAAGACCATCTCTAAGGCCAAGGGCCAGCCCAGAG |
| | | AGCCTCAGGTGTACACACTGCCTCCATCCCGGGAAG |
| | | AGATGACCAAGAACCAGGTGTCTCTGACATGTCTGG |
| | | TCAAGGGCTTCTATCCCTCTGACATCGCCGTGGAGT |
| | | GGGAGAGCAATGGCCAGCCTGAGAACAATTACAAG |
| | | ACCACACCCCCTGTGCTGGATTCCGACGGCTCTTTCT |
| | | TTCTGTATAGCAAGCTGACCGTGGACAAGTCCCGGT |
| | | GGCAGCAGGGCAACGTGTTCAGCTGTTCCGTGATGC |
| | | ACGAAGCTCTGCATAATCACTATACTCAGAAATCCC |
| | | TGTCACTGTCACCTGGTAAA |
| SEQ ID NO: | BGA-7746- | EVQLLESGGGLVQPGGSLRLSCAASGFTVSAEDVGWV |
| 192 | mutFc AA | RQAPGKGLEWVSAILDFGGSTYYAESVKGRFTISRDN |
| | | AKNTLYLQMSSLRAEDTAVYYCARVVYHAGGGVTFD |
| | | TRGQGTQVTVSSGGGGSDKTHTCPPCPAPPAAGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALAAPIEKTISKAKGQPREPQVYTLPP |
| | | SREEMTKNQVSLTCLVKGFYPSDIAVEWESNGQPENN |
| | | YKTTPPVLDSDGSFFLYSKLTVDKSRWQQGNVFSCSV |
| | | MHEALHNHYTQKSLSLSPGK |
| SEQ ID NO: | BGA-7746- | GAGGTCCAGTTACTTGAGAGTGGTGGAGGTCTGGTC |
| 193 | mutFc DNA | CAACCAGGAGGTTCGCTGCGTTTATCCTGCGCCGCG |
| | | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATTTTGGTGGTTCGACATACTAT |
| | | GCAGAAAGTGTCAAAGGGCGCTTTACGATCTCGCGC |
| | | GATAACGCAAAAAATACTCTTTACCTTCAAATGTCT |
| | | AGCCTTCGTGCTGAGGACACTGCGGTGTACTACTGC |
| | | GCCCGCGTCGTCTACCATGCTGGTGGTGGCGTCACC |
| | | TTTGATACGCGGGGACAGGGCACCCAAGTTACAGTC |
| | | TCATCGGGCGGCGGAGGGTCTGACAAGACCCACAC |
| | | ATGCCCCCCTTGTCCTGCTCCACCAGCTGCAGGACC |
| | | AAGCGTGTTCCTGTTTCCACCCAAGCCCAAGGATAC |
| | | CCTGATGATCTCTCGGACCCCAGAGGTGACATGCGT |
| | | GGTGGTGGATGTGAGCCACGAGGACCCCGAGGTGA |
| | | AGTTCAACTGGTATGTGGACGGCGTGGAGGTGCACA |
| | | ATGCTAAGACCAAGCCCAGGGAGGAGCAGTACAAC |
| | | TCCACCTATAGAGTGGTGTCTGTGCTGACAGTGCTG |
| | | CACCAGGATTGGCTGAACGGCAAGGAGTATAAGTG |
| | | CAAGGTGTCCAATAAGGCCCTGGCCGCTCCTATCGA |
| | | GAAGACCATCTCTAAGGCCAAGGGCCAGCCCAGAG |
| | | AGCCTCAGGTGTACACACTGCCTCCATCCCGGGAAG |
| | | AGATGACCAAGAACCAGGTGTCTCTGACATGTCTGG |
| | | TCAAGGGCTTCTATCCCTCTGACATCGCCGTGGAGT |
| | | GGGAGAGCAATGGCCAGCCTGAGAACAATTACAAG |
| | | ACCACACCCCCTGTGCTGGATTCCGACGGCTCTTTCT |
| | | TTCTGTATAGCAAGCTGACCGTGGACAAGTCCCGGT |
| | | GGCAGCAGGGCAACGTGTTCAGCTGTTCCGTGATGC |
| | | ACGAAGCTCTGCATAATCACTATACTCAGAAATCCC |
| | | TGTCACTGTCACCTGGTAAA |
| SEQ ID NO: | BGA-8425- | EVQLLESGGGLVQPGGSLRLSCAASGFTLSAEDVGWV |
| 194 | mutFc AA | RQAPGKGLEWVSAILDSGGSTYYAESVKGRFTISRDN |
| | | AKNTLYLQMSSLRAEDTAVYYCARVVYHAGGGVTY |
| | | DYRGQGTQVTVSSGGGGSDKTHTCPPCPAPPAAGPSV |
| | | FLFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALAAPIEKTISKAKGQPREPQVYTLP |
| | | PSREEMTKNQVSLTCLVKGFYPSDIAVEWESNGQPEN |
| | | NYKTTPPVLDSDGSFFLYSKLTVDKSRWQQGNVFSCS |
| | | VMHEALHNHYTQKSLSLSPGK |
| SEQ ID NO: | BGA-8425- | GAGGTCCAGTTACTTGAGAGTGGTGGAGGTCTGGTC |
| 195 | mutFc DNA | CAACCAGGAGGTTCGCTGCGTTTATCCTGCGCCGCG |
| | | TCTGGATTCACGCTGTCCGCCGAAGACGTGGGTTGG |
| | | GTGCGTCAAGCGCCGGGGAAAGGACTGGAATGGGT |
| | | CTCCGCCATCTTGGATTCGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCGTCGTCTACCATGCTGGTGGTGGCGTCAC |
| | | CTATGATTACCGGGGACAGGGCACCCAAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |
| SEQ ID NO: | BGA-3386- | EVQLLESGGGLVQPGGSLRLSCAASGFTVSAEDVGWV |
| 196 | mutFc AA | RQAPGKGLEWVSAILDKGGSTYYAESVKGRFTISRDN |
| | | AKNTLYLQMSSLRAEDTAVYYCARIVYHAGGGVTFD |
| | | TWGQGTLVTVSSGGGGSDKTHTCPPCPAPPAAGPSVF |
| | | LFPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALAAPIEKTISKAKGQPREPQVYTLP |
| | | PSREEMTKNQVSLTCLVKGFYPSDIAVEWESNGQPEN |
| | | NYKTTPPVLDSDGSFFLYSKLTVDKSRWQQGNVFSCS |
| | | VMHEALHNHYTQKSLSLSPGK |
| SEQ ID NO: | BGA-3386- | GAGGTCCAGTTACTTGAGAGTGGTGGAGGTCTGGTC |
| 197 | mutFc | CAACCAGGAGGTTCGCTGCGTTTATCCTGCGCCGCG |
| | DNA | TCTGGATTCACGGTTTCCGCCGAAGACGTGGGTTGG |
| | | GTGCGTCAAGCGCCGGGGAAAGGACTGGAATGGGT |
| | | CTCCGCCATCTTGGATAAGGGTGGTTCGACATACTA |
| | | TGCGGAAAGTGTCAAAGGGCGCTTTACGATCTCGCG |
| | | CGATAACGCAAAAAATACTCTTTACCTTCAAATGTC |
| | | TAGCCTTCGTGCTGAGGACACTGCGGTGTACTACTG |
| | | CGCCCGCATTGTCTACCATGCTGGTGGTGGCGTCAC |
| | | CTTTGATACTTGGGGACAGGGCACCTTAGTTACAGT |
| | | CTCATCGGGCGGCGGAGGGTCTGACAAGACCCACA |
| | | CATGCCCCCCTTGTCCTGCTCCACCAGCTGCAGGAC |
| | | CAAGCGTGTTCCTGTTTCCACCCAAGCCCAAGGATA |
| | | CCCTGATGATCTCTCGGACCCCAGAGGTGACATGCG |
| | | TGGTGGTGGATGTGAGCCACGAGGACCCCGAGGTG |
| | | AAGTTCAACTGGTATGTGGACGGCGTGGAGGTGCAC |
| | | AATGCTAAGACCAAGCCCAGGGAGGAGCAGTACAA |
| | | CTCCACCTATAGAGTGGTGTCTGTGCTGACAGTGCT |
| | | GCACCAGGATTGGCTGAACGGCAAGGAGTATAAGT |
| | | GCAAGGTGTCCAATAAGGCCCTGGCCGCTCCTATCG |
| | | AGAAGACCATCTCTAAGGCCAAGGGCCAGCCCAGA |
| | | GAGCCTCAGGTGTACACACTGCCTCCATCCCGGGAA |
| | | GAGATGACCAAGAACCAGGTGTCTCTGACATGTCTG |
| | | GTCAAGGGCTTCTATCCCTCTGACATCGCCGTGGAG |
| | | TGGGAGAGCAATGGCCAGCCTGAGAACAATTACAA |
| | | GACCACACCCCCTGTGCTGGATTCCGACGGCTCTTT |
| | | CTTTCTGTATAGCAAGCTGACCGTGGACAAGTCCCG |
| | | GTGGCAGCAGGGCAACGTGTTCAGCTGTTCCGTGAT |
| | | GCACGAAGCTCTGCATAATCACTATACTCAGAAATC |
| | | CCTGTCACTGTCACCTGGTAAA |

### Example 6. Binding Profiles of Anti-CD137 Antibody BGA-5623

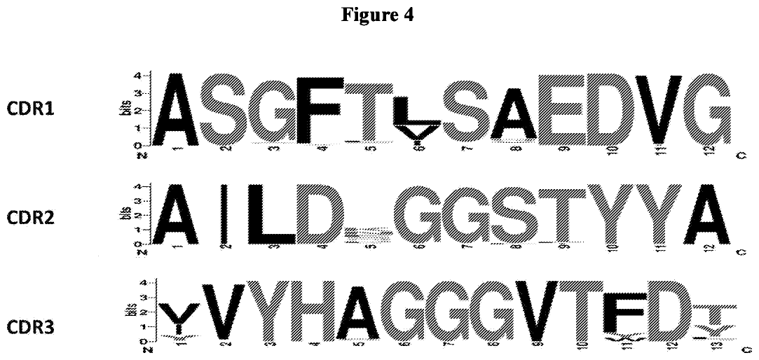

BGA-5623 was generated with human IgG1 Fc fusion and characterized for their binding kinetics by SPR assays using BIAcore™ T-200 (GE Life Sciences). Briefly, anti-human IgG (Fc) antibody was immobilized on an activated CM5 biosensor chip (Cat.: BR100839, GE Life Sciences). The anti-huCD137 domain antibody was flowed through the chip surface and captured by anti-human IgG (Fc) antibody. Then a serial dilution (6.0 nM to 2150 nM) of human CD137 ECD-mIgG2a or cyno CD137 ECD-mIgG2a were flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The result demonstrated that BGA-5623 has higher affinity for cynoCD137 than huCD137, as shown in Table 11 below. To evaluate the binding activity of the anti-huCD137 VH domain antibody to native huCD137 on living cells, Hut78 cells were transfected to over-express human CD137. Live Hut78/huCD137 expressing cells were seeded in 96-well plates and were incubated with a serial dilution of anti-huCD137 VH domain antibodies. Goat anti-Human IgG was used as secondary antibody to detect antibody binding to the cell surface. EC₅₀ values for dose-dependent binding to human native CD137 were determined by fitting the dose-response data to the four-parameter logistic model with GraphPad Prism™. As shown in FIG. 5, BGA-5623 demonstrated specific binding to native CD137 on living cells in a dose-responsive manner with EC50 of 2.97 μg/ml.

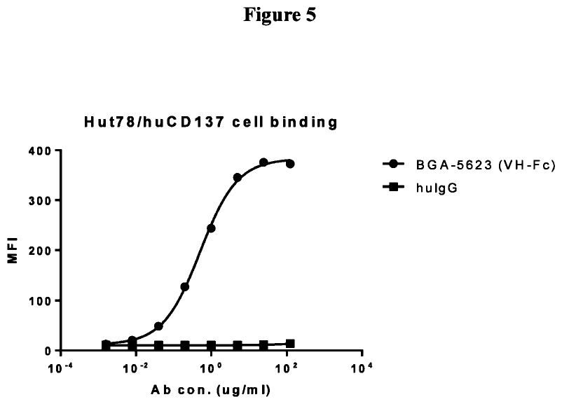

The off-target specificity of BGA-5623 was evaluated via ELISA. TNF receptor family members such as TNFRSF1A(CD120a) (Cat. No. 10872-H08H, Sino Biological, China), TNFRSF1B(CD120b) (Cat. No. 10417-H08H1, Sino Biological, China), TNFRSF4(OX40) (SEQ ID NO: 126), TNFRSF5(CD40) (SEQ ID NO: 120), TNFRSF7(CD27) (Cat. No. 10039-H08B1, Sino Biological, China), TNFRSF9(CD137) (SEQ ID NO: 94) and TNFRSF18(GITR) (Cat. No. 13643-H08H, Sino Biological, China) were coated in 96-well plates at a concentration of 10 μg/ml overnight at 4° C. BGA-5623 fused with wild type IgG1 Fc (SEQ ID NO: 198) was added. As shown in FIG. 6, no binding to other TNF receptor family members was observed.

TABLE 11
Affinity by SPR

| Sample | Species | Ka(1/Ms) | Kd(1/s) | KD(M) |
|---|---|---|---|---|
| BGA-5623 | huCD137 | 2.12E+05 | 5.03E−03 | 2.38E−08 |
| | cynoCD137 | 1.30E+05 | 2.54E−03 | 1.96E−08 |

TABLE 12
Amino acid and DNA sequences

| | SEQ ID | | |
|---|---|---|---|
| Antibody | NO | | SEQUENCE |
| BGA-5623 | SEQ ID | VH | EVQLLESGGGLVQPGGSLRLSCAASGFTVSAEDVGWVR |
| | NO: 84 | | QAPGKGLEWVSAILDKGGSTYYAESVKGRFTISRDNAK |
| | | | NTLYLQMSSLRAEDTAVYYCARIVYHAGGGVTFDTRGQ |
| | | | GTQVTVSS |
| | SEQ ID | VH | GAGGTCCAGTTACTTGAGAGTGGTGGAGGTCTGGTCC |
| | NO: 85 | DNA | AACCAGGAGGTTCGCTGCGTTTATCCTGCGCCGCGTCT |
| | | | GGATTCACGGTTTCCGCCGAAGACGTGGGTTGGGTGC |
| | | | GTCAAGCGCCGGGGAAAGGACTGGAATGGGTCTCCGC |
| | | | CATCTTGGATAAGGGTGGTTCGACATACTATGCGGAAA |
| | | | GTGTCAAAGGGCGCTTTACGATCTCGCGCGATAACGC |
| | | | AAAAAATACTCTTTACCTTCAAATGTCTAGCCTTCGTG |
| | | | CTGAGGACACTGCGGTGTACTACTGCGCCCGCATTGTC |
| | | | TACCATGCTGGTGGTGGCGTCACCTTTGATACTCGGGG |
| | | | ACAGGGCACCCAAGTTACAGTCTCATCG |
| | SEQ ID | IgG1 | EVQLLESGGGLVQPGGSLRLSCAASGFTVSAEDVGWVR |
| | NO: 198 | AA | QAPGKGLEWVSAILDKGGSTYYAESVKGRFTISRDNAK |
| | | | NTLYLQMSSLRAEDTAVYYCARIVYHAGGGVTFDTRGQ |
| | | | GTQVTVSSEPKSSDKTHTCPPCPAPELLGGPSVFLFPPKP |
| | | | KDTLMISRTPEVTCVVVDVSHEDPEVKFNWYVDGVEVH |
| | | | NAKTKPREEQYNSTYRVVSVLTVLHQDWLNGKEYKCK |
| | | | VSNKALPAPIEKTISKAKGQPREPQVYTLPPSRDELTKNQ |
| | | | VSLTCLVKGFYPSDIAVEWESNGQPENNYKTTPPVLDSD |
| | | | GSFFLYSKLTVDKSRWQQGNVFSCSVMHEALHNHYTQK |
| | | | SLSLSPGK |
| | SEQ ID | IgG1 | GAGGTCCAGTTACTTGAGAGTGGTGGAGGTCTGGTCC |
| | NO: 199 | DNA | AACCAGGAGGTTCGCTGCGTTTATCCTGCGCCGCGTCT |
| | | | GGATTCACGGTTTCCGCCGAAGACGTGGGTTGGGTGC |
| | | | GTCAAGCGCCGGGGAAAGGACTGGAATGGGTCTCCGC |
| | | | CATCTTGGATAAGGGTGGTTCGACATACTATGCGGAAA |
| | | | GTGTCAAAGGGCGCTTTACGATCTCGCGCGATAACGC |
| | | | AAAAAATACTCTTTACCTTCAAATGTCTAGCCTTCGTG |
| | | | CTGAGGACACTGCGGTGTACTACTGCGCCCGCATTGTC |
| | | | TACCATGCTGGTGGTGGCGTCACCTTTGATACTCGGGG |
| | | | ACAGGGCACCCAAGTTACAGTCTCATCGGAGCCCAAA |
| | | | TCTTCTGACAAAACTCACACATGCCCACCGTGCCCAG |
| | | | CACCTGAACTCCTGGGGGGACCGTCAGTCTTCCTCTTC |
| | | | CCCCCAAAACCCAAGGACACCCTCATGATCTCCCGGA |
| | | | CCCCTGAGGTCACATGCGTGGTGGTGGACGTGAGCCA |
| | | | CGAAGACCCTGAGGTCAAGTTCAACTGGTACGTGGAC |
| | | | GGCGTGGAGGTGCATAATGCCAAGACAAAGCCGCGGG |
| | | | AGGAGCAGTACAACAGCACGTACCGTGTGGTCAGCGT |
| | | | CCTCACCGTCCTGCACCAGGACTGGCTGAATGGCAAG |
| | | | GAGTACAAGTGCAAGGTCTCCAACAAAGCCCTCCCAG |
| | | | CCCCCATCGAGAAAACCATCTCCAAAGCCAAAGGGCA |
| | | | GCCCCGAGAACCACAGGTGTACACCCTGCCCCCATCC |
| | | | CGGGATGAGCTGACCAAGAACCAGGTCAGCCTGACCT |
| | | | GCCTGGTCAAAGGCTTCTATCCCAGCGACATCGCCGTG |
| | | | GAGTGGGAGAGCAATGGGCAGCCGGAGAACAACTAC |
| | | | AAGACCACGCCTCCCGTGCTGGACTCCGACGGCTCCT |
| | | | TCTTCCTCTACAGCAAGCTCACCGTGGACAAGAGCAG |
| | | | GTGGCAGCAGGGGAACGTCTTCTCATGCTCCGTGATG |
| | | | CATGAGGCTCTGCACAACCACTACACGCAGAAGAGCC |
| | | | TCTCCCTGTCTCCGGGTAAA |
| | SEQ ID | IgG4 | EVQLLESGGGLVQPGGSLRLSCAASGFTVSAEDVGWVR |
| | NO: 200 | AA | QAPGKGLEWVSAILDKGGSTYYAESVKGRFTISRDNAK |
| | | | NTLYLQMSSLRAEDTAVYYCARIVYHAGGGVTFDTRGQ |
| | | | GTQVTVSSESKYGPPCPPCPAPEFLGGPSVFLFPPKPKDT |
| | | | LMISRTPEVTCVVVDVSQEDPEVQFNWYVDGVEVHNA |
| | | | KTKPREEQFNSTYRWSVLTVLHQDWLNGKEYKCKVSN |
| | | | KGLPSSIEKTISKAKGQPREPQVYTLPPSQEEMTKNQVSL |
| | | | TCLVKGFYPSDIAVEWESNGQPENNYKTTPPVLDSDGSF |
| | | | FLYSRLTVDKSRWQEGNVFSCSVMHEALHNHYTQKSLS |
| | | | LSLGK |
| | SEQ ID | IgG4 | GAGGTCCAGTTACTTGAGAGTGGTGGAGGTCTGGTCC |
| | NO: 201 | DNA | AACCAGGAGGTTCGCTGCGTTTATCCTGCGCCGCGTCT |
| | | | GGATTCACGGTTTCCGCCGAAGACGTGGGTTGGGTGC |
| | | | GTCAAGCGCCGGGGAAAGGACTGGAATGGGTCTCCGC |
| | | | CATCTTGGATAAGGGTGGTTCGACATACTATGCGGAAA |
| | | | GTGTCAAAGGGCGCTTTACGATCTCGCGCGATAACGC |
| | | | AAAAAATACTCTTTACCTTCAAATGTCTAGCCTTCGTG |
| | | | CTGAGGACACTGCGGTGTACTACTGCGCCCGCATTGTC |
| | | | TACCATGCTGGTGGTGGCGTCACCTTTGATACTCGGGG |
| | | | ACAGGGCACCCAAGTTACAGTCTCATCGGAATCCAAG |
| | | | TATGGTCCGCCATGTCCACCGTGCCCGGCACCCGAGTT |
| | | | TTTGGGAGGGCCTAGTGTGTTTTTGTTTCCTCCTAAGC |
| | | | CAAAGGACACGCTTATGATTAGTCGCACACCCGAGGT |
| | | | TACCTGTGTAGTGGTGGACGTATCACAAGAGGATCCGG |
| | | | AAGTCCAATTTAATTGGTACGTCGACGGAGTGGAAGT |
| | | | CCACAATGCAAAGACCAAGCCTAGGGAAGAACAGTTC |
| | | | AATTCTACCTATCGCTGGTCTGTACTCACTGTACTCCAC |
| | | | CAGGATTGGCTCAATGGAAAGGAATACAAATGCAAGG |
| | | | TTTCCAACAAAGGGCTCCCCTCTTCCATTGAGAAAACT |
| | | | ATTAGCAAGGCTAAAGGCCAACCCAGAGAACCACAGG |
| | | | TTTATACCCTTCCACCTTCCCAGGAGGAGATGACGAAG |
| | | | AATCAGGTTAGTCTTACGTGCCTCGTTAAAGGATTCTA |
| | | | CCCTTCAGATATCGCAGTTGAATGGGAGTCTAACGGGC |
| | | | AACCCGAGAACAACTACAAGACCACCCCGCCGGTGCT |
| | | | GGACTCAGATGGTAGTTTCTTTCTTTACAGTCGGCTTA |
| | | | CCGTCGACAAGTCACGCTGGCAGGAGGGAAATGTATT |
| | | | CTCCTGCAGCGTAATGCACGAAGCTCTGCACAATCACT |
| | | | ATACCCAGAAGTCACTGAGCCTTTCTCTGGGTAAG |

### Example 7. Epitope Mapping of BGA-5623 by Alanine Scanning

To characterize the binding epitope of BGA-5623, 17 amino acid residues of human CD137 were mutated to alanine individually to generate 17 single-mutation huCD137 variants based upon the information from the crystal structure of CD137 reported previously (Bitra et al., (2018) J Biol Chem, 293, 9958-9969; Chin et al., (2018) Nat. Commun. 9, 4679).

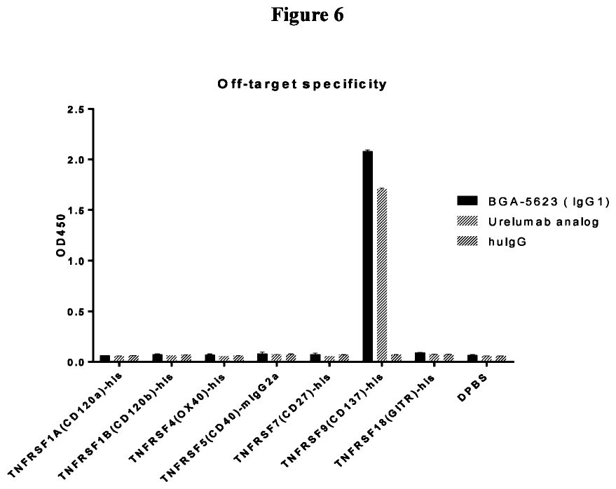

The CD137 mutants along with the wild-type CD137 were transiently expressed in HEK293 cells (ATCC CRL-1573). Their recognition and binding by BGA-5623 was analyzed by flow cytometry. An Urelumab analog (SEQ ID NOs: 202-205) that was generated in house by using the publicly available sequences of Urelumab, was used in the same assay to monitor the expression of CD137 mutants. In this assay, human CD137 or human CD137 mutant expressing cells (10⁵ cells/well) were incubated with 2 μg/ml of purified BGA-5623-mutFc (Fc fusion VH Ab) or Urelumab analog, followed by binding with Alexa Fluro-647-labeled anti-hu IgG Fc antibody (Cat.: 409320, BioLegend, USA). Cell fluorescence was quantified using a flow cytometer (Guava easyCyte™ 8HT, Merck-Millipore, USA). All results were normalized using the mean values of the fluorescence reading of wild type CD137 binding signal as the standard. To simplify data analysis, if an antibody's FACS binding signal for a specific mutant CD137 dropped to or below 25%, then the amino acid at that site was considered critical to the epitope. As shown in the FIG. 7A, the epitope of BGA-5623 has important residues for binding at amino acids F36, I44, P47, P49 and S52 of CD137.

In order to further explore the BGA-5623 epitope, human CD137 ECD mutants with single-AA substitution were expressed and purified to prepare for ELISA. In addition, a Utomilumab analog antibody (SEQ ID NOs: 206-209) was created in house by using the publicly available sequences of Utomilumab. The CD137 mutants along with the wild-type CD137 were analyzed for binding by BGA-5623 by direct ELISA. In brief, 50 ng each of wild-type or mutant CD137 was coated in an ELISA plate. After blocking, 100 μl of BGA-5623-mutFc, Urelumab analog or Utomilumab analog antibody at a concentration of 2 μg/ml was added to the plate and the binding signal of each antibody was detected by HRP-linked secondary antibody. In the ELISA binding assay using wild-type or mutant huCD137, amino acids F36A, P47A and P49A significantly impaired the binding of CD137 and BGA-5623 (FIGS. 7A-7B). Changes at amino acid F36A only slightly reduced the binding of the Urelumab or Utomilumab analogs, which indicates F36A plays a critical role in the conformation integrity of CD137. In contrast, neither changes at amino acids P47A or P49A disrupted the binding of the Urelumab or Utomilumab analog to CD137, indicating that BGA-5623, Urelumab analog or Utomilumab have different epitopes. This data indicated that amino acids F36A, P47A and P49A are critical residues in the epitope for antibody BGA-5623.

TABLE 13
Amino acid and DNA sequences

| | SEQ ID | | |
|---|---|---|---|
| Antibody | NO | | SEQUENCE |
| Urelumab | SEQ ID | Heavy | QVQLQQWGAGLLKPSETLSLTCAVYGGSFSGYYWS |
| analog | NO: 202 | chain | WIRQSPEKGLEWIGEINHGGYVTYNPSLESRVTISVD |
| | | AA | TSKNQFSLKLSSVTAADTAVYYCARDYGPGNYDWY |
| | | | FDLWGRGTLVTVSSASTKGPSVFPLAPCSRSTSESTA |
| | | | ALGCLVKDYFPEPVTVSWNSGALTSGVHTFPAVLQS |
| | | | SGLYSLSSVVTVPSSSLGTKTYTCNVDHKPSNTKVD |
| | | | KRVESKYGPPCPPCPAPEFLGGPSVFLFPPKPKDTLM |
| | | | ISRTPEVTCVVVDVSQEDPEVQFNWYVDGVEVHNA |
| | | | KTKPREEQFNSTYRWSVLTVLHQDWLNGKEYKCK |
| | | | VSNKGLPSSIEKTISKAKGQPREPQVYTLPPSQEEMT |
| | | | KNQVSLTCLVKGFYPSDIAVEWESNGQPENNYKTTP |
| | | | PVLDSDGSFFLYSRLTVDKSRWQEGNVFSCSVMHE |
| | | | ALHNHYTQKSLSLSLGK |
| | SEQ ID | Heavy | CAAGTTCAACTTCAGCAGTGGGGAGCGGGTCTCT |
| | NO: 203 | chain | TGAAGCCTAGTGAGACGCTGAGCCTGACCTGTGC |
| | | DNA | AGTCTACGGGGGTTCATTTTCTGGGTACTACTGGT |
| | | | CCTGGATACGCCAAAGCCCAGAGAAAGGCCTGGA |
| | | | ATGGATTGGGGAGATAAATCACGGGGGTTATGTGA |
| | | | CATATAATCCTTCTCTTGAGAGCCGGGTCACGATAT |
| | | | CTGTTGACACAAGTAAAAATCAGTTCTCCCTCAA |
| | | | ACTGAGCTCCGTTACCGCAGCAGACACTGCCGTT |
| | | | TACTACTGTGCGCGGGACTATGGGCCAGGTAATTA |
| | | | CGATTGGTACTTCGACCTGTGGGGTAGAGGAACG |
| | | | CTGGTCACTGTAAGCAGTGCATCTACAAAGGGCC |
| | | | CCAGCGTGTTTCCCCTTGCACCATGTTCACGGAGC |
| | | | ACTAGTGAAAGCACTGCCGCGCTCGGGTGTCTCG |
| | | | TTAAGGATTATTTTCCGGAGCCTGTCACTGTCTCTT |
| | | | GGAACTCTGGTGCACTGACATCAGGCGTTCACAC |
| | | | GTTCCCTGCAGTACTCCAGAGCTCAGGATTGTACA |
| | | | GCCTGTCCAGTGTAGTTACAGTGCCTTCCTCCTCC |
| | | | CTTGGAACAAAGACCTACACCTGCAACGTGGACC |
| | | | ATAAGCCCAGCAATACGAAAGTAGACAAACGAGT |
| | | | CGAATCCAAGTATGGTCCGCCATGTCCACCGTGCC |
| | | | CGGCACCCGAGTTTTTGGGAGGGCCTAGTGTGTT |
| | | | TTTGTTTCCTCCTAAGCCAAAGGACACGCTTATGA |
| | | | TTAGTCGCACACCCGAGGTTACCTGTGTAGTGGTG |
| | | | GACGTATCACAAGAGGATCCGGAAGTCCAATTTA |
| | | | ATTGGTACGTCGACGGAGTGGAAGTCCACAATGC |
| | | | AAAGACCAAGCCTAGGGAAGAACAGTTCAATTCT |
| | | | ACCTATCGCTGGTCTGTACTCACTGTACTCCACCA |
| | | | GGATTGGCTCAATGGAAAGGAATACAAATGCAAG |
| | | | GTTTCCAACAAAGGGCTCCCCTCTTCCATTGAGA |
| | | | AAACTATTAGCAAGGCTAAAGGCCAACCCAGAGA |
| | | | ACCACAGGTTTATACCCTTCCACCTTCCCAGGAGG |
| | | | AGATGACGAAGAATCAGGTTAGTCTTACGTGCCTC |
| | | | GTTAAAGGATTCTACCCTTCAGATATCGCAGTTGA |
| | | | ATGGGAGTCTAACGGGCAACCCGAGAACAACTAC |
| | | | AAGACCACCCCGCCGGTGCTGGACTCAGATGGTA |
| | | | GTTTCTTTCTTTACAGTCGGCTTACCGTCGACAAG |
| | | | TCACGCTGGCAGGAGGGAAATGTATTCTCCTGCA |
| | | | GCGTAATGCACGAAGCTCTGCACAATCACTATACC |
| | | | CAGAAGTCACTGAGCCTTTCTCTGGGTAAG |
| | SEQ ID | Light | EIVLTQSPATLSLSPGERATLSCRASQSVSSYLAWYQ |
| | NO: 204 | chain | QKPGQAPRLLIYDASNRATGIPARFSGSGSGTDFTLTI |
| | | AA | SSLEPEDFAVYYCQQRSNWPPALTFGGGTKVEIKRT |
| | | | VAAPSVFIFPPSDEQLKSGTASVVCLLNNFYPREAKV |
| | | | QWKVDNALQSGNSQESVTEQDSKDSTYSLSSTLTLS |
| | | | KADYEKHKVYACEVTHQGLSSPVTKSFNRGEC |
| | SEQ ID | Light | GAAATCGTGCTGACCCAATCACCTGCGACCCTCTC |
| | NO: 205 | chain | CCTTAGCCCTGGAGAACGAGCAACTCTCAGTTGC |
| | | DNA | AGAGCAAGCCAGAGCGTCAGCTCATATCTCGCAT |
| | | | GGTATCAACAAAAACCAGGCCAAGCTCCTAGACT |
| | | | GTTGATTTATGATGCCTCTAACCGGGCGACTGGCA |
| | | | TACCAGCCCGCTTTAGCGGGTCTGGGAGTGGCAC |
| | | | TGATTTTACGCTCACGATTTCATCTTTGGAGCCAG |
| | | | AAGATTTCGCTGTATACTACTGTCAACAAAGGTCA |
| | | | AACTGGCCGCCTGCACTTACGTTCgGCGGGGGTAC |
| | | | CAAGGTGGAGATTAAGAGGACAGTTGCGGCGCCA |
| | | | TCAGTTTTTATATTCCCACCCTCCGATGAGCAGTTG |
| | | | AAAAGTGGCACGGCATCAGTTGTCTGTCTGCTGA |
| | | | ACAATTTCTATCCACGGGAGGCGAAAGTCCAATG |
| | | | GAAAGTGGACAATGCGCTTCAGAGCGGGAACTCT |
| | | | CAGGAGAGTGTCACTGAACAGGATTCAAAGGACT |
| | | | CCACGTACTCACTCTCATCCACTCTCACCCTTTCA |
| | | | AAGGCGGATTACGAAAAGCATAAGGTCTACGCTT |
| | | | GTGAGGTAACACATCAGGGGCTTTCCAGTCCTGT |
| | | | AACCAAGAGCTTCAATCGCGGCGAATGC |
| Utomilumab | SEQ ID | Heavy | EVQLVQSGAEVKKPGESLRISCKGSGYSFSTYWISW |
| analog | NO: 206 | chain | VRQMPGKGLEWMGKIYPGDSYTNYSPSFQGQVTIS |
| | | AA | ADKSISTAYLQWSSLKASDTAMYYCARGYGIFDYW |
| | | | GQGTLVTVSSASTKGPSVFPLAPCSRSTSESTAALGC |
| | | | LVKDYFPEPVTVSWNSGALTSGVHTFPAVLQSSGLY |
| | | | SLSSVVTVPSSNFGTQTYTCNVDHKPSNTKVDKTV |
| | | | ERKCCVECPPCPAPPVAGPSVFLFPPKPKDTLMISRT |
| | | | PEVTCVVVDVSHEDPEVQFNWYVDGVEVHNAKTK |
| | | | PREEQFNSTFRVVSVLTVVHQDWLNGKEYKCKVSN |
| | | | KGLPAPIEKTISKTKGQPREPQVYTLPPSREEMTKNQ |
| | | | VSLTCLVKGFYPSDIAVEWESNGQPENNYKTTPPML |
| | | | DSDGSFFLYSKLTVDKSRWQQGNVFSCSVMHEALH |
| | | | NHYTQKSLSLSPGK |
| | SEQ ID | Heavy | GAAGTTCAACTGGTACAGTCCGGTGCTGAAGTTA |
| | NO: 207 | chain | AAAAACCCGGAGAGAGTCTGAGAATCTCTTGCAA |
| | | DNA | GGGGTCTGGGTACTCTTTTAGTACATATTGGATATC |
| | | | TTGGGTGAGACAAATGCCGGGGAAAGGCCTCGA |
| | | | ATGGATGGGCAAGATTTATCCCGGAGACAGCTATA |
| | | | CAAATTACTCACCIAGTTTTCAAGGTCAGGTAACT |
| | | | ATCAGCGCGGACAAATCCATAAGTACGGCCTATCT |
| | | | GCAATGGTCTTCCTTGAAGGCATCCGACACGGCG |
| | | | ATGTATTACTGCGCAAGAGGGTATGGTATATTCGAT |
| | | | TACTGGGGGCAGGGCACTCTCGTTACCGTGAGCA |
| | | | GCGCTTCAACAAAAGGCCCATCTGTCTTCCCTCTC |
| | | | GCGCCTTGCTCCCGCTCTACTTCAGAGTCAACAGC |
| | | | TGCTCTGGGGTGCTTGGTGAAGGATTACTTTCCTG |
| | | | AACCGGTTACTGTCAGCTGGAATTCTGGGGCTTTG |
| | | | ACGAGCGGAGTTCACACTTTCCCAGCTGTACTGC |
| | | | AAAGTTCTGGACTTTACAGCTTGAGCTCCGTGGT |
| | | | GACAGTGCCGAGCTCAAACTTCGGTACGCAGACA |
| | | | TATACATGCAATGTGGATCATAAACCCAGCAATAC |
| | | | GAAAGTAGATAAGACAGTCGAAAGGAAGTGTTGT |
| | | | GTAGAGTGCCCACCGTGCCCCGCCCCTCCGGTTG |
| | | | CAGGCCCATCAGTGTTTCTCTTCCCGCCCAAACCT |
| | | | AAGGACACGCTGATGATTTCACGAACGCCCGAGG |
| | | | TCACCTGTGTCGTAGTCGATGTGTCCCATGAGGAC |
| | | | CCAGAAGTTCAGTTCAATTGGTATGTAGACGGCGT |
| | | | GGAGGTACACAATGCAAAAACGAAACCACGCGA |
| | | | GGAACAGTTCAATAGTACGTTCCGGGTCGTGAGT |
| | | | GTGCTCACGGTGGTTCACCAAGACTGGCTGAACG |
| | | | GGAAAGAATATAAGTGCAAAGTATCCAATAAAGG |
| | | | CCTGCCCGCACCCATCGAAAAAACTATAAGTAAA |
| | | | ACTAAAGGGCAACCGCGAGAGCCTCAGGTCTATA |
| | | | CTTTGCCACCGTCACGCGAAGAGATGACCAAGAA |
| | | | CCAAGTAAGTCTTACTTGCTTGGTCAAGGGGTTCT |
| | | | ACCCCTCAGATATAGCGGTGGAGTGGGAATCCAAT |
| | | | GGCCAACCCGAGAACAATTACAAGACAACACCAC |
| | | | CAATGCTGGACTCTGACGGGAGTTTCTTTCTGTAT |
| | | | AGTAAACTTACCGTCGATAAATCACGCTGGCAGCA |
| | | | AGGCAACGTATTCTCATGCTCAGTAATGCATGAAG |
| | | | CCTTGCATAATCACTACACTCAGAAAAGCCTGTCC |
| | | | CTCTCTCCAGGTAAG |
| | SEQ ID | Light | SYELTQPPSVSVSPGQTASITCSGDNIGDQYAHWYQ |
| | NO: 208 | chain | QKPGQSPVLVIYQDKNRPSGIPERFSGSNSGNTATLTI |
| | | AA | SGTQAMDEADYYCATYTGFGSLAVFGGGTKLTVLG |
| | | | QPKAAPSVTLFPPSSEELQANKATLVCLISDFYPGAV |
| | | | TVAWKADSSPVKAGVETTTPSKQSNNKYAASSYLS |
| | | | LTPEQWKSHRSYSCQVTHEGSTVEKTVAPTECS |
| | SEQ ID | Light | AGCTACGAGTTGACTCAGCCACCATCCGTCAGTG |
| | NO: 209 | chain | TTAGCCCCGGACAGACGGCGTCAATTACTTGTAGT |
| | | DNA | GGAGACAACATTGGCGATCAGTACGCCCATTGGT |
| | | | ATCAGCAAAAACCGGGTCAATCACCAGTTCTGGT |
| | | | GATATATCAGGATAAGAATCGACCATCAGGGATTC |
| | | | CAGAGAGGTTCTCAGGTAGTAACTCAGGTAACAC |
| | | | AGCTACTCTGACCATAAGTGGTACCCAGGCTATGG |
| | | | ACGAAGCAGACTACTACTGCGCCACCTACACAGG |
| | | | CTTCGGATCACTTGCGGTTTTCGGGGGCGGAACC |
| | | | AAACTAACCGTTCTTGGGCAACCTAAGGCTGCCC |
| | | | CGTCCGTGACTCTTTTTCCTCCGTCTTCCGAAGAG |
| | | | CTTCAAGCCAATAAAGCAACCTTGGTATGTTTGAT |
| | | | TTCCGATTTTTACCCTGGAGCGGTGACGGTGGCAT |
| | | | GGAAAGCTGATTCAAGCCCTGTGAAAGCTGGGGT |
| | | | GGAAACTACTACGCCGTCCAAACAGAGCAATAAT |
| | | | AAGTACGCGGCCTCCAGCTATTTGTCACTTACCCC |
| | | | GGAGCAATGGAAGTCACACCGAAGTTACAGTTGT |
| | | | CAAGTTACTCACGAAGGCAGCACTGTTGAAAAGA |
| | | | CCGTAGCACCCACCGAATGCTCA |

### Example 8. Ligand Competition

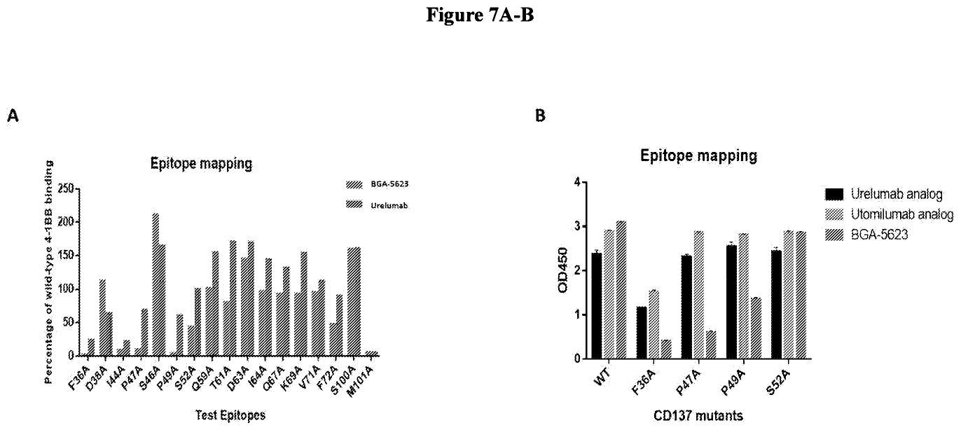

Human CD137 binds to its major ligand human CD137 ligand (CD137L) with weak affinity at an approximate Kd of three-digit M (Chin et al., (2018) Nat Commun 9, 4679). The epitope mapping results in Example 7 above, shows that amino acid residues F36A, P47A and P49A of CD137 are critical amino acid residues that make up part of the epitope for the BGA-5623 antibody. In addition, the ligand binds CD137 along the entire length of receptor CRD-2 and the A2 motif of CRD-3, and the interface between the receptor and ligand is primarily mediated by hydrogen bonds and van der Waals interactions (Bitra et al., (2018) J Biol Chem, 293, 9958-9969). Based on this data, it was hypothesized that the BGA-5623 antibody can block CD137/CD137 ligand interaction. BGA-5623 was generated with a human IgG4 Fc fusion. For CD137 ligand competition ELISA, a Maxisorp immunoplate was coated with human CD137 ECD-mIgG2a and blocked with 3% BSA (w/v) in PBS buffer (blocking buffer). VH domain antibody BGA-5623 was blocked with blocking buffer for 30 minutes and added to wells of the ELISA plate for 1 hour in the presence of serially diluted human CD137 ligand ECD-mIgG2a. After washes with PBST, bound antibodies were detected using HRP-conjugated anti-human IgG antibody (Sigma, A0170) and 3,3′,5,5′-tetramethylbenzidine substrate (Cat.: 00-4201-56, eBioscience, USA) (FIG. 8). As shown in FIG. 8, BGA-5623 competes with CD137 ligand and reduces CD137/CD137 ligand interaction.

### Example 9. Structural and Functional CD137 Epitope Mapping

To better understand how the anti-CD137 single domain antibody arm is capable of high affinity binding for CD137, and robust agonist of CD137/CD137L interaction, the crystal structure of VH (BGA-5623) in complex with CD137 was determined.

### A. CD137 and VH (BGA-5623) Expression, Purification, and Crystallization

Human CD137 ectodomain containing four CRDs (1-4; amino acids 24-162 of SEQ ID NO: 94 (human CD137 FL)) harboring C121S, N138D, and N149Q mutations was expressed in HEK293G cells. The cDNA coding CD137 was cloned into in house expression vector with an N-terminal secretion sequence and a C-terminal TEV cleavage site followed by an Fc tag. The culture supernatant containing the secreted CD137-Fc fusion protein was mixed with Mab Select Sure™ resin (GE Healthcare Life Sciences) for 3 hours at 4° C. The protein was washed with buffer containing 20 mM Tris-HCl pH 8.0, 150 mM NaCl, then eluted with 50 mM acetic acid (adjust pH value to 3.5 with 5 M NaOH), and finally neutralized with 1/10 CV 1.0M Tris-HCl pH8.0. The eluted protein was mixed with TEV proteases (10:1 molar ratio) and dialyzed against buffer (20 mM Tris-HCl, pH 8.0, 100 mM NaCl) at 4° C. overnight. The mixture was loaded onto a Ni-NTA column (Qiagen) and Mab Select Sure™ resin to remove the TEV proteases and Fc tag, and then the flow-through was further purified by size-exclusion chromatography in buffer (20 mM Tris pH 8.0, 100 mM NaCl) using a HiLoad 16/600 Superdex™ 75 pg column (GE Healthcare Life Sciences).

DNA sequence encoding VH (BGA-5623) was cloned into a PET21a vector with N-terminal HIS-MBP tag followed by TEV protease site. Protein expression in Shuffle T7 was induced at OD600 of 0.6-1.0 with 1 mM IPTG at 18° C. for 16 h. The cells were harvested by centrifugation at 7,000 g, 10 min. The cell pellets were re-suspended in lysis buffer (50 mM Na₃PO₄ pH 7.0, 300 mM NaCl) and lysed under sonication on ice. The lysate then was centrifuged at 48,000 g at 4° C. for 30 min. The supernatant was mixed with Talon resin and batched at 4° C. for 3 hours. The resin was washed with lysis buffer containing 5 mM imidazole, the protein was eluted in lysis buffer with additional 100 mM imidazole. The eluate was mixed with TEV proteases (10:1 molar ratio) and dialyzed against buffer (20 mM Tris-HCl, pH 8.0, 100 mM NaCl) at 4° C. overnight. The mixture was loaded onto a Talon column to remove the TEV proteases and HIS-MBP tag, and then the flow-through was further purified by size-exclusion chromatography in buffer (20 mM Tris pH 8.0, 100 mM NaCl) using a HiLoad 16/600 Superdex™ 75 pg column (GE Healthcare Life Sciences).

Purified CD137 was mixed with an excess of purified VH (BGA-5623) (1:1.5 molar ratio) to generate the CD137/VH (BGA-5623) complex. The complex was then further purified by gel filtration in buffer (20 mM Tris pH 8.0, 100 mM NaCl) using a HiLoad 16/600 Superdex™ 75 pg column (GE Healthcare Life Sciences). The CD137/VH (BGA-5623) complex (10 mg/ml) was crystallized in 0.6 M Li₂SO₄, 0.01 M NiCl₂, 0.1 M Tris pH 9.0. Crystals cryoprotected with stepwise 5% D-(+)-Sucrose to a final 20% concentration were flash frozen in liquid nitrogen. Besides, the apoVH (BGA-5623) was crystallized in 1.2 M (NH₄)₂SO₄, 0.1 M Citric Acid pH 5.0. Crystal was cryoprotected with 7% glycerol and flash frozen in liquid nitrogen. The X-ray diffraction data was collected at beamline BL45XU at Spring-8 synchrotron radiation facility (Hyogo, Japan).

### B. Data Collection and Structure Solution

The X-ray diffraction data was collected under cryo cooled conditions at 100 Kelvin at beamline BL45XU equipped with ZOO (Hirata, K., et al., Acta Crystallogr D Struct Biol, 2019. 75(Pt 2): 138-150) automated data collection system in Spring-8 synchrotron radiation facility (Hyogo, Japan). Diffraction images were processed with the integrated data processing software KAMO (Yamashita, et al., Acta Crystallogr D Struct Biol, 2018. 74 (Pt 5): 441-449) employing XDS (Kabsch W., Acta Crystallogr D Biol Crystallogr, 2010. 66 (Pt 2): 125-32). The structure of human CD137 (PDB: 6MGP) and VHH model (PDB:4U3X) were used as search models. The initial solution was found with molecular replacement program PHASER (McCoy et al., Phaser crystallographic software. J Appl Crystallogr, 2007. 40(Pt 4): 658-674). Then this model was iterative manually built with program COOT (Emsley et al., Acta Crystallogr D Biol Crystallogr, 2004. 60(Pt 12 Pt 1): 2126-32) and refinement using PHENIX (Adams et al., Acta Crystallogr D Biol Crystallogr, 2010. 66(Pt 2): 213-21). The final model was refined to acceptable R and R free values and Ramachandran statistics (calculated by Molprobity). Data processing and refinement statistics can be found in Table 14.

### C. The Structure of VH (BGA-5623) Bound to Human CD137

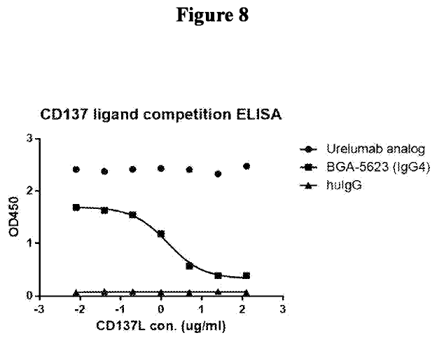

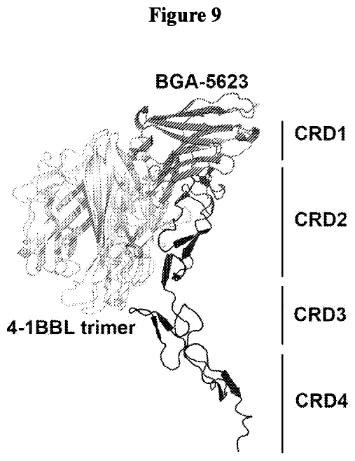

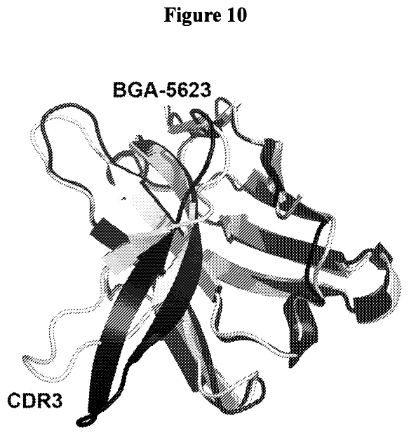

The VH (BGA-5623) in complex with CD137 crystallized in the 141 space group, with one complex in the asymmetric unit, and diffracted to 2.58 Å. The structure of VH (BGA-5623) bound to human CD137 shows that VH (BGA-5623) partially sterically interfaces with CD137L binding (FIG. 9). The buried surface area between VH (BGA-5623) and CD137 is approximately 571 Å2. VH (BGA-5623) interactions are clustered around CD137 CRD2 domain. These interactions are primarily mediated by VH (BGA-5623) CDR2 and CDR3 and make more extensive contact with CD137. VH (BGA-5623) CDR1 does not directly contact CD137 while CDR3 undergoes a dramatic conformational change from unstructured loop to ß-sheet upon CD137 binding (FIG. 10). VH (BGA-5623) CDR2 Leu52, Tyr58 contact CD137 residues Pro50, Asn51. VH (BGA-5623) CDR3 residues Gly 100A, Gly 100B, Val100C, Thr100D, Phe100E contact CD137 residues Phe36, Pro47, Pro49, Arg60, Cys62, Ile64. Besides, FR2 Leu45 and Trp47 contact CD137 residues Pro47, Cys48, Pro49, Pro50 which contribute significantly to CD137 binding. VH (BGA-5623) interacts with CD137 using a combination of hydrogen bonds and hydrophobic interactions. For example, FR2 Trp47 forms strong hydrophobic contacts with CD137 residues Pro47, Cys48, Pro49 and Pro50. CDR3 residue Phe100E forms hydrophobic interactions with CD137 residues Phe36 and Pro47. FR2 residue Trp47 and CDR3 residue Gly 100A form one hydrogen bond with CD137 residues Pro47 and Ile64, respectively. CDR3 residue Val100C forms two hydrogen bonds with CD137 residue Cys62 (FIG. 11).

Based on the crystal structure of the VH (BGA-5623)/CD137 complex, the residues of CD137 that are contacted by VH (BGA-5623) (i.e., the epitopic residues of CD137 bound by VH) and the residues of VH (BGA-5623) that are contacted by CD137 (i.e. the paratopic residues of VH contacted by CD137) were determined. Table 15, below, show the residues of CD137 and VH (BGA-5623) to which they contact, as assessed using a contact distance stringency of 3.7 Å, a point at which van der Waals (non-polar) interaction forces are highest. The epitope mapping analysis based on the crystal structure also rationalize the previous alanine scanning results which identified several epitopic residues of CD137 by VH (BGA-5623).

TABLE 14
Data collection and refinement statistics

| | | | VH (BGA-5623)/CD137 | |
|---|---|---|---|---|
| Crystals | apoVH (BGA-5623) | | complex | |
| Data collection | | | | |
| X-ray source | BL45XU | | BL45XU | |
| Wavelength (Å) | 1.0 | | 1.0 | |
| Space group | P4₁2₁2 | | I4₁ | |
| Cell Dimensions | | | | |
| a (Å) | 68.342 | | 123.943 | |
| b (Å) | 68.342 | | 123.943 | |
| c (Å) | 102.952 | | 57.877 | |
| α (°) | 90 | | 90 | |
| β (°) | 90 | | 90 | |
| γ (°) | 90 | | 90 | |
| Resolution (Å) | 56.94-1.70 | (1.80-1.70)^{a} | 87.64-2.58 | (2.73-2.58) |
| Rsym (%) | 18.0 | (145.8) | 21.4 | (209.3) |
| <I/σ> | 14.3 | (1.6) | 12.3 | (1.2) |
| CC1/2 | 99.8 | (56.1) | 99.7 | (44.4) |
| Redundancy | 11.6 | (5.9) | 6.8 | (6.6) |
| Completeness (%) | 97.3 | (84.3) | 99.9 | (99.4) |

| | | |
|---|---|---|
| Refinement | | |
| Resolution (Å) | 48.33-1.70 | 43.82-2.58 |
| No. reflections | 26791 | 14038 |
| Rwork/Rfree ^{b}(%) | 19.84/23.46 | 22.8/28.9 |
| r.m.s.d. ^{c}bonds/ | 0.006/0.892 | 0.010/1.314 |
| angles | | |
| Number of atoms | | |
| Protein | 1787 | 1959 |
| Water | 204 | 5 |
| B factors | | |
| Protein | 27.37 | 83.97 |
| Water | 36.42 | 66.79 |
| Ramachandran | | |
| Favored (%) | 98.71 | 94.21 |
| Allowed (%) | 1.29 | 3.47 |
| Outlier (%) | 0 | 2.32 |

^{a}Values in parentheses are those of the highest resolution shell.
^{b}Calculated from about 5% of the reflection set aside during refinement
^{c}r.m.s.d., root mean square deviation

TABLE 15
Epitopic residues of CD137 and their corresponding
paratopic residues of VH(BGA-5623)

| CD137 | | VH (BGA-5623) | |
|---|---|---|---|
| Phe | 36 | Thr | 100D |
| | | Phe | 100E |
| Pro | 47 | Leu | 45 |
| | | Trp | 47 |
| | | Phe | 100E |
| Cys | 48 | Trp | 47 |
| Pro | 49 | Trp | 47 |
| | | Val | 100C |
| | | Thr | 100D |
| Pro | 50 | Trp | 47 |
| | | Tyr | 58 |
| Asn | 51 | Leu | 52 |
| Arg | 60 | Thr | 100D |
| Cys | 62 | Gly | 100B |
| | | Val | 100C |
| Ile | 64 | Gly | 100A |

The VH (BGA-5623) residues are numbered in Kabat nomenclature.

### Example 10. GPC3×CD137 Agonists

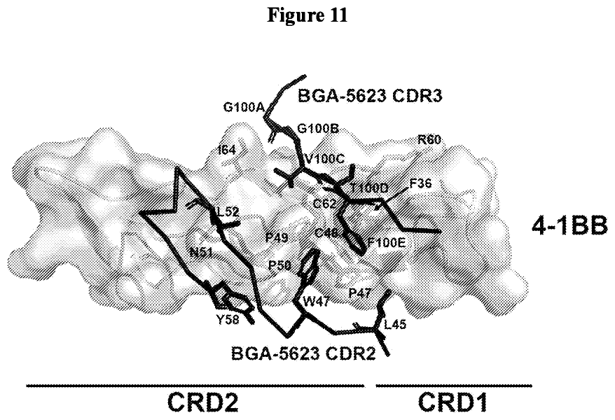

Agonistic anti-huCD137 antibodies have demonstrated toxicity in the clinical setting, which may indicate that systemic FcγR cross-linking is not ideal for CD137 activation. The aim was to achieve potent CD137 stimulation specifically at the tumor site without systemic CD137 activation for a broad range of cancers. To overcome the dependency of FcγR cross-linking, we generated a GPC3×CD137 multispecific antibody with the following features as shown in FIG. 12. This specific construct included an IgG-fusion like multispecific antibody format with a module ratio of 2:2, a bivalent F(ab′)₂ fragment that binds to GPC3, VH domain fragments with a fusion at the C terminal of CH3, which bind huCD137, and a Fc null version of huIgG1, which has no FcγR binding but retains FcRn binding. The sequence information is shown in Table 16.

TABLE 16
Amino acid and DNA sequences of GPC3 x CD137 (BE-830)

| | | |
|---|---|---|
| BE-830 | SEQ ID | QVQLVQSGAEVKKPGASVKVSCKASGYTFTDYEMHWVRQAPGQGLEWM |
| heavy | NO: 1 | GALDPKTGDTAYSQKFKGRVTLTADKSTSTAYMELSSLTSEDTAVYYCTRFY |
| chain AA | | SYTYWGQGTLVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHK |
| | | PSNTKVDKKVEPKSCDKTHTCPPCPAPPAAGPSVFLFPPKPKDTLMISRTPE |
| | | VTCVVVDVSHEDPEVKFNWYVDGVEVHNAKTKPREEQYNSTYRVVSVLT |
| | | VLHQDWLNGKEYKCKVSNKALAAPIEKTISKAKGQPREPQVYTLPPSREE |
| | | MTKNQVSLTCLVKGFYPSDIAVEWESNGQPENNYKTTPPVLDSDGSFFLYS |
| | | KLTVDKSRWQQGNVFSCSVMHEALHNHYTQKSLSLSPGKGGGGSEVQLLE |
| | | SGGGLVQPGGSLRLSCAASGFTVSAEDVGWVRQAPGKGLEWVSAILDKGG |
| | | STYYAESVKGRFTISRDNAKNTLYLQMSSLRAEDTAVYYCARIVYHAGGGV |
| | | TFDTRGQGTQVTVSS |
| BE-830 | SEQ ID | CAGGTGCAGCTGGTGCAGTCTGGAGCAGAGGTGAAGAAGCCAGGAGCC |
| heavy | NO: 2 | AGCGTGAAGGTGTCCTGCAAGGCCTCTGGCTATACCTTCACAGACTACG |
| chain DNA | | AGATGCACTGGGTGCGGCAGGCACCAGGACAGGGACTGGAGTGGATGG |
| | | GCGCCCTGGACCCTAAGACCGGCGATACAGCCTATAGCCAGAAGTTTAA |
| | | GGGCAGAGTGACCCTGACAGCCGACAAGTCCACCTCTACAGCCTACATG |
| | | GAGCTGAGCTCCCTGACCAGCGAGGATACAGCCGTGTACTATTGTACCC |
| | | GGTTCTACTCCTATACATACTGGGGCCAGGGCACCCTGGTGACAGTGTCT |
| | | AGCGCTAGCACCAAGGGGCCCTCGGTCTTCCCCCTGGCACCCTCCTCCA |
| | | AGAGTACTTCTGGGGGCACAGCGGCCCTGGGCTGCCTGGTCAAGGACTA |
| | | CTTCCCCGAACCGGTGACGGTGTCGTGGAACTCAGGCGCCCTGACCAGC |
| | | GGCGTGCACACCTTCCCGGCTGTCCTACAGTCCTCAGGACTCTACTCCCT |
| | | CAGCAGCGTGGTGACCGTGCCCTCCAGCAGCTTGGGCACCCAGACCTAC |
| | | ATCTGCAACGTGAATCACAAGCCCAGCAACACCAAGGTGGACAAGAAA |
| | | GTTGAGCCAAAGTCCTGTGACAAGACCCACACATGCCCCCCTTGTCCTG |
| | | CTCCACCAGCTGCAGGACCAAGCGTGTTCCTGTTTCCACCCAAGCCCAA |
| | | GGATACCCTGATGATCTCTCGGACCCCAGAGGTGACATGCGTGGTGGTG |
| | | GATGTGAGCCACGAGGACCCCGAGGTGAAGTTCAACTGGTATGTGGACG |
| | | GCGTGGAGGTGCACAATGCTAAGACCAAGCCCAGGGAGGAGCAGTACA |
| | | ACTCCACCTATAGAGTGGTGTCTGTGCTGACAGTGCTGCACCAGGATTG |
| | | GCTGAACGGCAAGGAGTATAAGTGCAAGGTGTCCAATAAGGCCCTGGCC |
| | | GCTCCTATCGAGAAGACCATCTCTAAGGCCAAGGGCCAGCCCAGAGAGC |
| | | CTCAGGTGTACACACTGCCTCCATCCCGGGAAGAGATGACCAAGAACCA |
| | | GGTGTCTCTGACATGTCTGGTCAAGGGCTTCTATCCCTCTGACATCGCCG |
| | | TGGAGTGGGAGAGCAATGGCCAGCCTGAGAACAATTACAAGACCACAC |
| | | CCCCTGTGCTGGATTCCGACGGCTCTTTCTTTCTGTATAGCAAGCTGACC |
| | | GTGGACAAGTCCCGGTGGCAGCAGGGCAACGTGTTCAGCTGTTCCGTG |
| | | ATGCACGAAGCTCTGCATAATCACTATACTCAGAAATCCCTGTCACTGTC |
| | | ACCTGGTAAAGGTGGAGGCGGTTCAGAGGTCCAGTTACTTGAGAGTGGT |
| | | GGAGGTCTGGTCCAACCAGGAGGTTCGCTGCGTTTATCCTGCGCCGCGT |
| | | CTGGATTCACGGTTTCCGCCGAAGACGTGGGTTGGGTGCGTCAAGCGCC |
| | | GGGGAAAGGACTGGAATGGGTCTCCGCCATCTTGGATAAGGGTGGTTCG |
| | | ACATACTATGCGGAAAGTGTCAAAGGGCGCTTTACGATCTCGCGCGATAA |
| | | CGCAAAAAATACTCTTTACCTTCAAATGTCTAGCCTTCGTGCTGAGGACA |
| | | CTGCGGTGTACTACTGCGCCCGCATTGTCTACCATGCTGGTGGTGGCGTC |
| | | ACCTTTGATACTCGGGGACAGGGCACCCAAGTTACAGTCTCATCGTGA |
| BE-830 | SEQ ID | DVVMTQSPLSLPVTPGEPASISCRSSQSLVHSNRNTYLHWYLQKPGQSPQLL |
| light chain | NO: 3 | IYKVSNRFSGVPDRFSGSGSGTDFTLKISRVEAEDVGVYYCSQNTHVPPTFG |
| AA | | QGTKLEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQGL |
| | | SSPVTKSFNRGEC |
| BE-830 | SEQ ID | GACGTGGTCATGACACAGTCCCCTCTGTCTCTGCCAGTGACCCCAGGAG |
| light chain | NO: 4 | AGCCTGCCTCTATCAGCTGCAGGAGCTCCCAGAGCCTGGTGCACTCCAA |
| DNA | | CCGCAATACATACCTGCACTGGTATCTGCAGAAGCCAGGCCAGTCCCCCC |
| | | AGCTGCTGATCTACAAGGTGTCTAACCGGTTCAGCGGCGTGCCCGACAG |
| | | ATTTTCCGGCTCTGGCAGCGGCACCGATTTCACACTGAAGATCTCCCGGG |
| | | TGGAGGCAGAGGATGTGGGCGTGTACTATTGTTCTCAGAATACCCACGT |
| | | GCCCCCTACATTTGGCCAGGGCACCAAGCTGGAGATCAAGCGTACGGTG |
| | | GCGGCGCCATCTGTCTTCATCTTCCCGCCATCTGATGAGCAGTTGAAATC |
| | | TGGTACCGCTAGCGTTGTGTGCCTGCTGAATAACTTCTATCCCAGAGAGG |
| | | CCAAAGTACAGTGGAAGGTGGATAACGCCCTCCAATCGGGTAACTCCCA |
| | | GGAGAGTGTCACAGAGCAGGACAGCAAGGACAGCACCTACAGCCTCAG |
| | | CAGCACCCTGACGCTGAGCAAAGCAGACTACGAGAAACACAAAGTCTA |
| | | CGCCTGCGAAGTCACCCATCAGGGCCTGAGCTCGCCCGTCACAAAGAGC |
| | | TTCAACAGGGGAGAGTGT |
| GPC3 | SEQ ID | QVQLVQSGAEVKKPGASVKVSCKASGYTFTDYEMHWVRQAPGQGLEWM |
| VH in BE- | NO: 5 | GALDPKTGDTAYSQKFKGRVTLTADKSTSTAYMELSSLTSEDTAVYYCTRFY |
| 830 | | SYTYWGQGTLVTVSS |
| AA | | |
| GPC3 | SEQ ID | CAGGTGCAGCTGGTGCAGTCTGGAGCAGAGGTGAAGAAGCCAGGAGCC |
| VH in BE- | NO: 6 | AGCGTGAAGGTGTCCTGCAAGGCCTCTGGCTATACCTTCACAGACTACG |
| 830 | | AGATGCACTGGGTGCGGCAGGCACCAGGACAGGGACTGGAGTGGATGG |
| DNA | | GCGCCCTGGACCCTAAGACCGGCGATACAGCCTATAGCCAGAAGTTTAA |
| | | GGGCAGAGTGACCCTGACAGCCGACAAGTCCACCTCTACAGCCTACATG |
| | | GAGCTGAGCTCCCTGACCAGCGAGGATACAGCCGTGTACTATTGTACCC |
| | | GGTTCTACTCCTATACATACTGGGGCCAGGGCACCCTGGTGACAGTGTCT |
| | | AGC |
| GPC3 | SEQ ID | DVVMTQSPLSLPVTPGEPASISCRSSQSLVHSNRNTYLHWYLQKPGQSPQLL |
| VL in BE- | NO: 7 | IYKVSNRFSGVPDRFSGSGSGTDFTLKISRVEAEDVGVYYCSQNTHVPPTFG |
| 830 | | QGTKLEIK |
| AA | | |
| GPC3 | SEQ ID | GACGTGGTCATGACACAGTCCCCTCTGTCTCTGCCAGTGACCCCAGGAG |
| VL in BE- | NO: 8 | AGCCTGCCTCTATCAGCTGCAGGAGCTCCCAGAGCCTGGTGCACTCCAA |
| 830 | | CCGCAATACATACCTGCACTGGTATCTGCAGAAGCCAGGCCAGTCCCCCC |
| DNA | | AGCTGCTGATCTACAAGGTGTCTAACCGGTTCAGCGGCGTGCCCGACAG |
| | | ATTTTCCGGCTCTGGCAGCGGCACCGATTTCACACTGAAGATCTCCCGGG |
| | | TGGAGGCAGAGGATGTGGGCGTGTACTATTGTTCTCAGAATACCCACGT |
| | | GCCCCCTACATTTGGCCAGGGCACCAAGCTGGAGATCAAG |
| Fc in BE- | SEQ ID | EPKSCDKTHTCPPCPAPPAAGPSVFLFPPKPKDTLMISRTPEVTCVVVDVSH |
| 830 | NO: 9 | EDPEVKFNWYVDGVEVHNAKTKPREEQYNSTYRVVSVLTVLHQDWLNGK |
| AA | | EYKCKVSNKALAAPIEKTISKAKGQPREPQVYTLPPSREEMTKNQVSLTCL |
| | | VKGFYPSDIAVEWESNGQPENNYKTTPPVLDSDGSFFLYSKLTVDKSRWQQ |
| | | GNVFSCSVMHEALHNHYTQKSLSLSPGK |
| Anti-GPC3 | SEQ ID | DYEMH |
| antibody | NO: 10 | |
| HCDR1 | | |
| (Kabat) | | |
| Anti-GPC3 | SEQ ID | ALDPKTGDTAYSQKFKG |
| antibody | NO: 11 | |
| HCDR2 | | |
| (Kabat) | | |
| Anti-GPC3 | SEQ ID | FYSYTY |
| antibody | NO: 12 | |
| HCDR3 | | |
| (Kabat) | | |
| Anti-GPC3 | SEQ ID | RSSQSLVHSNRNTYLH |
| antibody | NO: 13 | |
| LCDR1 | | |
| (Kabat) | | |
| Anti-GPC3 | SEQ ID | KVSNRFS |
| antibody | NO: 14 | |
| LCDR2 | | |
| (Kabat) | | |
| Anti-GPC3 | SEQ ID | SQNTHVPPT |
| antibody | NO: 15 | |
| LCDR3 | | |
| (Kabat) | | |

### Example 11. Target-Binding Activity of GPC3×CD137

### Binding to Recombinant CD137 and GPC3

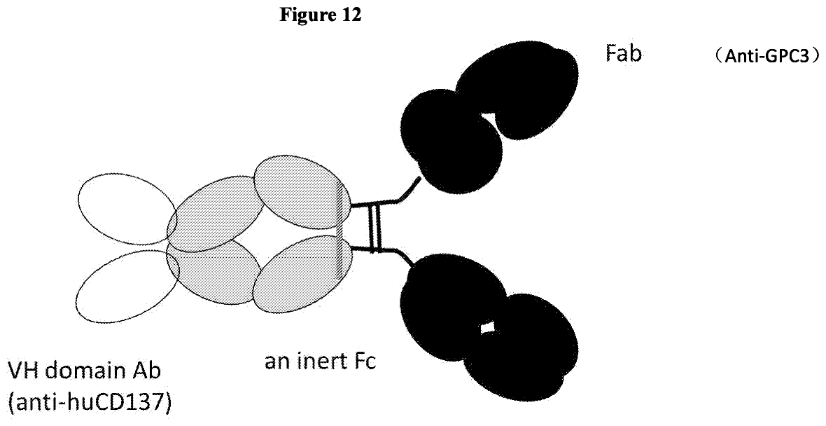

The dual binding ELISA results demonstrated that BE-830 is capable of binding both targets (CD137/GPC3) simultaneously, while the 2 negative controls, human IgG and DS (drug substance) buffer, had no detectable binding to GPC3 and CD137 (FIG. 13).

The binding kinetics of the BE-830 were measured using surface plasmon resonance (SPR). We used SPR to measure the on-rate constant (kₐ) and off-rate constant (k_{d}) of the antibodies to recombinant proteins of CD137 and GPC3, and then determined the affinity constant (K_{D}). The results showed that BE-830 had a high binding affinity to human CD137 and human GPC3.

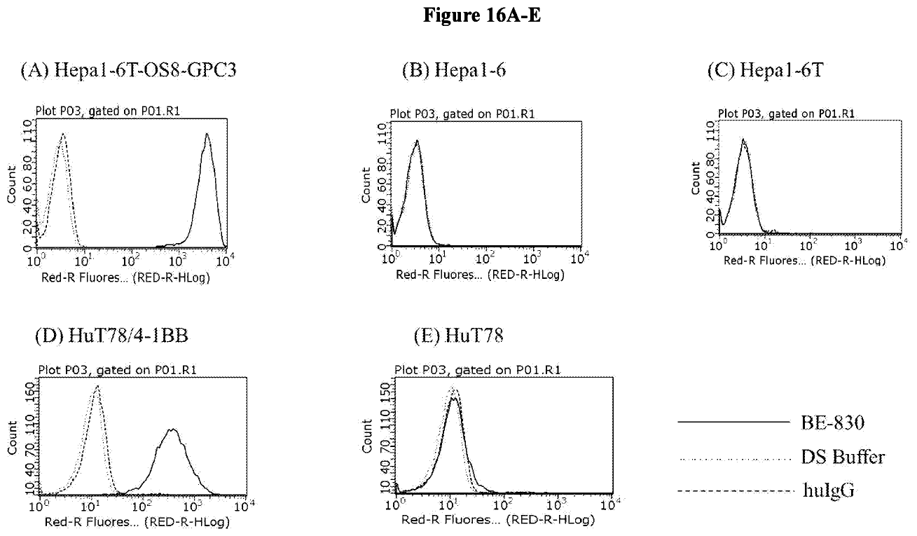

Human CD137 protein has low sequence homology to murine CD137, with only 61.0% sequence identity. In contrast, CD137 is highly homologous to cynomolgus monkey CD137, with 95% sequence identity. To test the species specificity of BE-830 binding function, SPR binding studies were performed using human, cynomolgus monkey, and mouse CD137 as binding proteins (FIG. 17A-C) BE-830 displayed a high binding affinity to human CD137 with a K_{D} of about 40.1 nM (FIG. 17A). In comparison, the binding affinity of BE-830 to cynomolgus monkey CD137 with a similar K_{D} of about 19.2 nM (FIG. 17B). BE-830 had no detectable binding signaling to mouse CD137 in SPR assay as shown in FIG. 17C. This is summarized in Table 17.

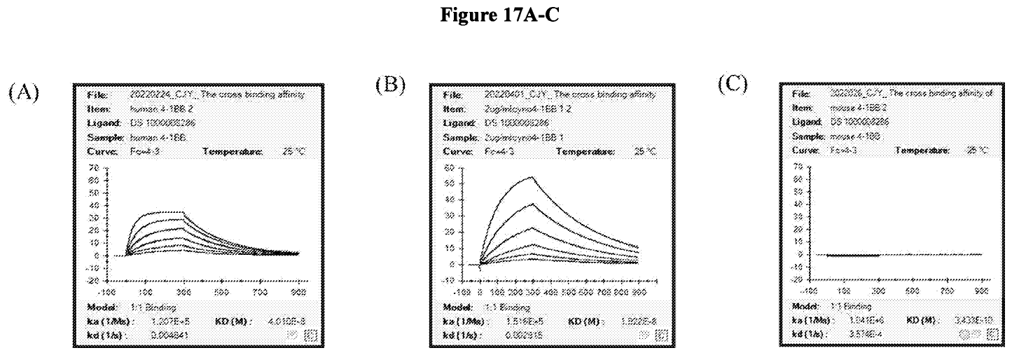

The binding affinity tests of GPC3s between human and cynomolgous monkey species indicated that BE-830 displayed a similar binding affinity to human GPC3 (K_{D}: about 0.56 nM, FIG. 18A) and monkey GPC3 (FIG. 18B) (Cat #GP3-C5225, Acrobiosystem) (K_{D}: about 0.64 nM). BE-830 had no detectable binding signaling to mouse GPC3 in SPR assay (FIG. 18C). This data is summarized in Table 18 below.

### Binding to Native CD137 and GPC3

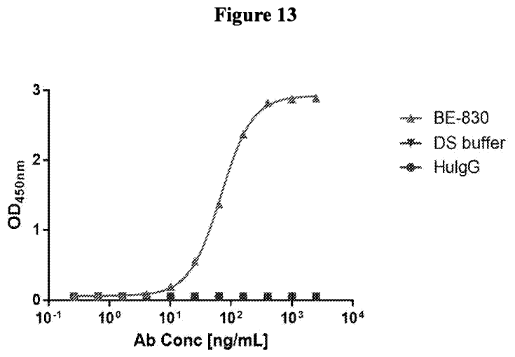

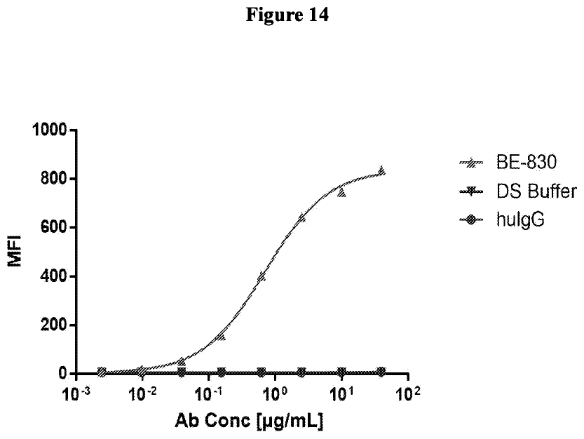

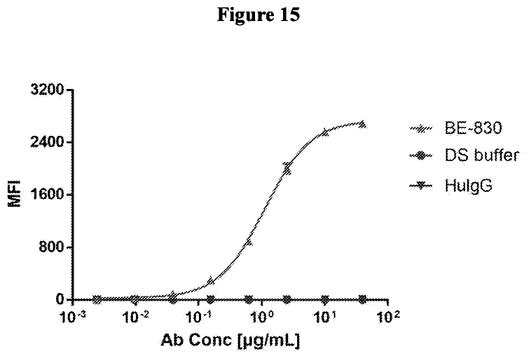

The FACS results further confirmed the binding activity of BE-830 to CD137 expressed on HuT78/CD137 cell surface. BE-830 showed strong binding activities to CD137 in a dose-responsive manner with EC₅₀ of 0.7124 μg/ml (4.09 nM); whereas the negative control human antibodies (hIgG) had no binding to HuT78/CD137 as expected (FIG. 14 and FIG. 16A-C). Similarly, BE-830 showed strong binding activities to GPC3 in a dose-responsive manner with EC₅₀ of 1.115 μg/ml (6.41 nM); whereas the negative control human antibodies (hIgG) had no binding to Hepa1-6T-OS8-GPC3 as expected (FIG. 15 and FIG. 16D-E).

TABLE 17
Comparative analysis of SPR determined kinetic
parameters to different species of CD137

| | | Kₒₙ | K_{off} | K_{D} |
|---|---|---|---|---|
| Antigen | Antibody | (1/Ms) | (1/s) | (M)^{a, b} |
| Human CD137 | BE-830 | 1.21E+05 | 4.84E−03 | 4.01E−08 |
| Monkey CD137 | | 1.52E+05 | 2.91E−03 | 1.92E−08 |
| Mouse CD137 | | ND^{c} | ND | ND |

Abbreviations: K_{D}, affinity constant; K_{off}, rate constant of dissociation; Kₒₙ, rate constant of association; ND, not determinable; SPR, surface plasmon resonance.
^{a}K_{D} values are calculated from the ratio of the kinetic constants as K_{D} = K_{off}/Kₒₙ.
^{b}K_{D} values are determined by the analyte concentration at which half of the ligands are occupied at equilibrium.
^{c}ND: Affinity is too weak for determination.

TABLE 18
Comparative analysis of SPR determined kinetic
parameters to different species of GPC3

| | | Kₒₙ | K_{off} | K_{D} |
|---|---|---|---|---|
| Antigen | Antibody | (1/Ms) | (1/s) | (M)^{a, b} |
| Human GPC3 | BE-830 | 5.59E+05 | 3.12E−04 | 5.59E−10 |
| Monkey GPC3 | | 4.25E+05 | 2.73E−04 | 6.43E−10 |
| Mouse GPC3 | | ND^{c} | ND | ND |

Abbreviations: GPC3, Glypican 3; K_{D}, affinity constant; K_{off}, rate constant of dissociation; Kₒₙ, rate constant of association; ND, not determinable; SPR, surface plasmon resonance.
^{a}K_{D} values are calculated from the ratio of the kinetic constants as K_{D} = K_{off}/Kₒₙ.
^{b}K_{D} values are determined by the analyte concentration at which half of the ligands are occupied at equilibrium.
^{c}ND: Affinity is too weak for determination.

### Example 12. Critical Binding Epitopes

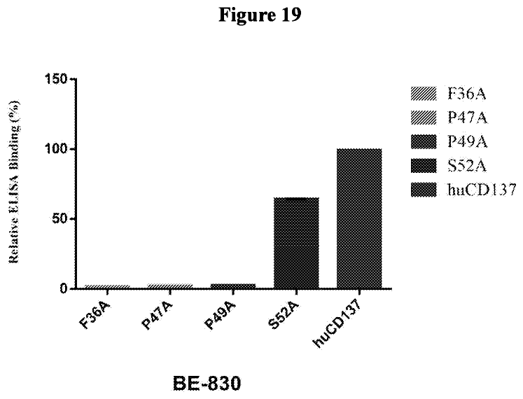

To study the binding epitopes of BE-830, single point mutations were introduced into CD137 on critical interface that are required for ligand-binding, and we also identified the GPC3 binding domain involved in BE-830 binding. ELISA assay was performed to compare BE-830 binding to wild type and mutant CD137 proteins, meanwhile, we also prepared two fusion proteins of C-terminal peptide of GPC3 (GK28b and DS50b) to determine the binding domain for GPC3 arm of BE-830. GK28b (human GPC3 AA 537-563) and DS50b (human GPC3 AA 511-560) were constructed to the N terminal of biotin, generating GK28b-KLH and DS50b-KLH (FIG. 20A-B). Human GPC3 AA 25-559 was constructed with His tag, generating huGPC3-His (FIG. 20C). The sequence thereof are shown in Table 19.

TABLE 19
Sequences of C-terminal peptides of GPC3

| CONSTRUCT | SEQ ID NO | SEQUENCE |
|---|---|---|
| human GPC3 FL | SEQ ID NO: 212 | MAGTVRTACLVVAMLLSLDFPGQAQPPPPPPDATCHQV |
| AA | | RSFFQRLQPGLKWVPETPVPGSDLQVCLPKGPTCCSRKM |
| | | EEKYQLTARLNMEQLLQSASMELKFLIIQNAAVFQEAFEI |
| | | VVRHAKNYTNAMFKNNYPSLTPQAFEFVGEFFTDVSLYI |
| | | LGSDINVDDMVNELFDSLFPVIYTQLMNPGLPDSALDINE |
| | | CLRGARRDLKVFGNFPKLIMTQVSKSLQVTRIFLQALNL |
| | | GIEVINTTDHLKFSKDCGRMLTRMWYCSYCQGLMMVKP |
| | | CGGYCNVVMQGCMAGVVEIDKYWREYILSLEELVNGM |
| | | YRIYDMENVLLGLFSTIHDSIQYVQKNAGKLTTTIGKLCA |
| | | HSQQRQYRSAYYPEDLFIDKKVLKVAHVEHEETLSSRRR |
| | | ELIQKLKSFISFYSALPGYICSHSPVAENDTLCWNGQELV |
| | | ERYSQKAARNGMKNQFNLHELKMKGPEPVVSQIIDKLK |
| | | HINQLLRTMSMPKGRVLDKNLDEEGFESGDCGDDEDECI |
| | | GGSGDGMIKVKNQLRFLAELAYDLDVDDAPGNSQQATP |
| | | KDNEISTFHNLGNVHSPLKLLTSMAISVVCFFFLVH |
| human GPC3 FL | SEQ ID NO: 213 | ATGGCCGGGACCGTGCGCACCGCGTGCTTGGTGGTGG |
| DNA | | CGATGCTGCTCAGCTTGGACTTCCCGGGACAGGCGCA |
| | | GCCCCCGCCGCCGCCGCCGGACGCCACCTGTCACCAA |
| | | GTCCGCTCCTTCTTCCAGAGACTGCAGCCCGGACTCAA |
| | | GTGGGTGCCAGAAACTCCCGTGCCAGGATCAGATTTG |
| | | CAAGTATGTCTCCCTAAGGGCCCAACATGCTGCTCAAG |
| | | AAAGATGGAAGAAAAATACCAACTAACAGCACGATTG |
| | | AACATGGAACAGCTGCTTCAGTCTGCAAGTATGGAGC |
| | | TCAAGTTCTTAATTATTCAGAATGCTGCGGTTTTCCAA |
| | | GAGGCCTTTGAAATTGTTGTTCGCCATGCCAAGAACTA |
| | | CACCAATGCCATGTTCAAGAACAACTACCCAAGCCTG |
| | | ACTCCACAAGCTTTTGAGTTTGTGGGTGAATTTTTCAC |
| | | AGATGTGTCTCTCTACATCTTGGGTTCTGACATCAATG |
| | | TAGATGACATGGTCAATGAATTGTTTGACAGCCTGTTT |
| | | CCAGTCATCTATACCCAGCTAATGAACCCAGGCCTGCC |
| | | TGATTCAGCCTTGGACATCAATGAGTGCCTCCGAGGA |
| | | GCAAGACGTGACCTGAAAGTATTTGGGAATTTCCCCA |
| | | AGCTTATTATGACCCAGGTTTCCAAGTCACTGCAAGTC |
| | | ACTAGGATCTTCCTTCAGGCTCTGAATCTTGGAATTGA |
| | | AGTGATCAACACAACTGATCACCTGAAGTTCAGTAAG |
| | | GACTGTGGCCGAATGCTCACCAGAATGTGGTACTGCTC |
| | | TTACTGCCAGGGACTGATGATGGTTAAACCCTGTGGCG |
| | | GTTACTGCAATGTGGTCATGCAAGGCTGTATGGCAGGT |
| | | GTGGTGGAGATTGACAAGTACTGGAGAGAATACATTC |
| | | TGTCCCTTGAAGAACTTGTGAATGGCATGTACAGAATC |
| | | TATGACATGGAGAACGTACTGCTTGGTCTCTTTTCAAC |
| | | AATCCATGATTCTATCCAGTATGTCCAGAAGAATGCAG |
| | | GAAAGCTGACCACCACTATTGGCAAGTTATGTGCCCAT |
| | | TCTCAACAACGCCAATATAGATCTGCTTATTATCCTGA |
| | | AGATCTCTTTATTGACAAGAAAGTATTAAAAGTTGCTC |
| | | ATGTAGAACATGAAGAAACCTTATCCAGCCGAAGAAG |
| | | GGAACTAATTCAGAAGTTGAAGTCTTTCATCAGCTTCT |
| | | ATAGTGCTTTGCCTGGCTACATCTGCAGCCATAGCCCT |
| | | GTGGCGGAAAACGACACCCTTTGCTGGAATGGACAAG |
| | | AACTCGTGGAGAGATACAGCCAAAAGGCAGCAAGGA |
| | | ATGGAATGAAAAACCAGTTCAATCTCCATGAGCTGAA |
| | | AATGAAGGGCCCTGAGCCAGTGGTCAGTCAAATTATT |
| | | GACAAACTGAAGCACATTAACCAGCTCCTGAGAACCA |
| | | TGTCTATGCCCAAAGGTAGAGTTCTGGATAAAAACCT |
| | | GGATGAGGAAGGGTTTGAAAGTGGAGACTGCGGTGAT |
| | | GATGAAGATGAGTGCATTGGAGGCTCTGGTGATGGAA |
| | | TGATAAAAGTGAAGAATCAGCTCCGCTTCCTTGCAGA |
| | | ACTGGCCTATGATCTGGATGTGGATGATGCGCCTGGA |
| | | AACAGTCAGCAGGCAACTCCGAAGGACAACGAGATAA |
| | | GCACCTTTCACAACCTCGGGAACGTTCATTCCCCGCTG |
| | | AAGCTTCTCACCAGCATGGCCATCTCGGTGGTGTGCTT |
| | | CTTCTTCCTGGTGCACTGA |
| human GPC3 AA | SEQ ID NO: 214 | GNSQQATPKDNEISTFHNLGNVHSPLK |
| 537-563 | | |
| (GK28b) | | |
| human GPC3 AA | SEQ ID NO: 215 | DGMIKVKNQLRFLAELAYDLDVDDAPGNSQQATPKDNE |
| 511-560 | | ISTFHNLGNVHS |
| (DS50b) | | |
| human GPC3 AA | SEQ ID NO: 216 | QPPPPPPDATCHQVRSFFQRLQPGLKWVPETPVPGSDLQ |
| 25-559 | | VCLPKGPTCCSRKMEEKYQLTARLNMEQLLQSASMELK |
| | | FLIIQNAAVFQEAFEIVVRHAKNYTNAMFKNNYPSLTPQ |
| | | AFEFVGEFFTDVSLYILGSDINVDDMVNELFDSLFPVIYT |
| | | QLMNPGLPDSALDINECLRGARRDLKVFGNFPKLIMTQV |
| | | SKSLQVTRIFLQALNLGIEVINTTDHLKFSKDCGRMLTRM |
| | | WYCSYCQGLMMVKPCGGYCNVVMQGCMAGVVEIDKY |
| | | WREYILSLEELVNGMYRIYDMENVLLGLFSTIHDSIQYVQ |
| | | KNAGKLTTTIGKLCAHSQQRQYRSAYYPEDLFIDKKVLK |
| | | VAHVEHEETLSSRRRELIQKLKSFISFYSALPGYICSHSPV |
| | | AENDTLCWNGQELVERYSQKAARNGMKNQFNLHELKM |
| | | KGPEPVVSQIIDKLKHINQLLRTMSMPKGRVLDKNLDEE |
| | | GFESGDCGDDEDECIGGSGDGMIKVKNQLRFLAELAYDL |
| | | DVDDAPGNSQQATPKDNEISTFHNLGNVH |

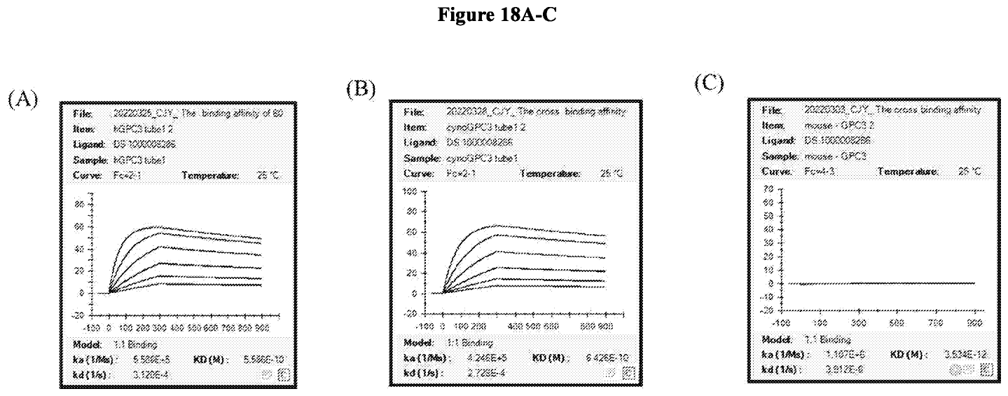

The results showed that mutations of CD137 at amino acids: F36A, P47A, P49A led to a significant loss of BE-830 binding activity (>75% decrease, respectively) whereas S52A mutation had less effect on the BE-830 binding activity than the other three mutations (FIG. 19). Thus, the residues F36, P47, P49 in all of the selected amino acid residues in CD137, were significant contact residues for BE-830 binding, similar to what was seen for the anti-CD137 only constructs. On the other hand, BE-830 displayed a binding to DS50b, GK28b and huGPC3-His in a dose-dependent manner. Given that DS50b and GK28b bound to BE-830 more strongly, comparing with overlapping amino acids sequence of the three peptides (FIG. 20A-C), it is suggested that the epitope for GPC3 arm is located within the sequence of GK28b at the C-terminus of GPC3.

### Example 13. BE-830 Enhances T Cell Activation

A cell-based bioluminescent assay was developed and used to measure the activity of BE-830 which target and stimulate an inducible costimulatory receptor CD137 and enhances T cell activation.

Two genetically modified cell lines, JK-NFκB-CD137 and Hepa1-6T-OS8-GPC3, were used as effector cells and target cells respectively in this assay. JK-NFκB-CD137 was developed from the Jurkat cell line, clone E6-1 (ATCC, TIB-152) by stably transfecting a human CD137 gene vector and a luciferase construct with a NFκB response element that can respond to both T cell receptor (TCR) activation and CD137 co-stimulation. Hepa1-6T-OS8-GPC3 cell line was generated from Hepa1-6T cells by ectopically expressing a human GPC3 and the T cell engager OS8 (a membrane-bound form of anti-CD3 antibody). When the two cell lines are co-cultured, addition of the bispecific antibody BE-830 would interact with both CD137 expressing on the effector cells and GPC3 expressing on the target cells and initiate the GPC3-dependent CD137 co-stimulation and activation of luciferase gene promoter in a dose dependent manner. JK-NFκB-CD137 (5×10+ cells/well) and Hepa1-6T-OS8-GPC3 (1×10⁴ cells/well) were co-cultured for 5-6 hours in the presence of serially diluted BE-830. As a negative control, human IgG (hIgG) and a buffer containing no antibody was used.

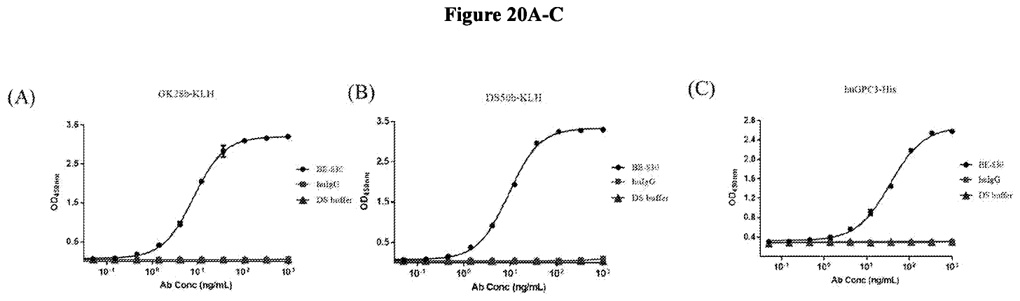

BE-830 showed agonistic functional activity in a dose-responsive manner. This experiment was performed in duplicate and the EC₅₀ for BE-830 was 0.1489 μg/ml (0.86 nM) as shown in FIG. 21. The buffer and human IgG controls had no activity.

### Example 14. Effector Receptor Binding and Effector Functions

BE-830 uses an engineered human IgG1 Fc moiety, which has diminished binding activities to effector function receptors. ELISA assays demonstrated that BE-830 has reduced binding activities to FcγRI, FcγRIIAH131, FcγRIIAR131, FcγRIIB, FcγRIIIAV158, FcγRIIIAF158, FcγRIIIB and C1q when BE-830 was compared to human IgGs (huIgG). As FcγRs and C1q are the key receptors mediating immune complex-induced effector functions, BE-830 has undetectable effector functions, such as antibody dependent cellular cytotoxicity (ADCC), antibody dependent cellular phagocytosis (ADCP) and complement-dependent cytotoxicity (CDC).

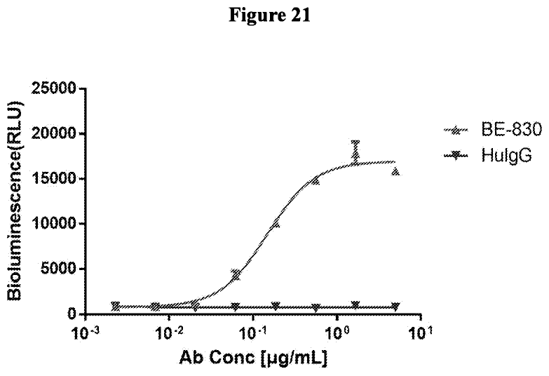

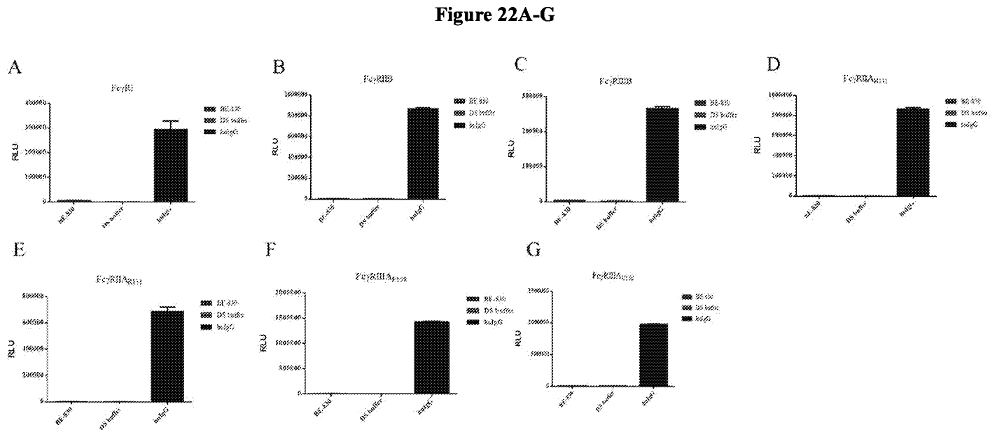

FcγR binding activities were assessed by ELISA. BE-830 did not exhibit any significant binding activity to FcγRI, FcγRIIAH131, FcγRIIAR131, FcγRIIB, FcγRIIIAV158, FcγRIIIAF158, FcγRIIIB, which were comparable to negative control. In contrast, the positive control human IgG produced a strong binding signal to any of the FcγRs in the assay (FIG. 22A-G). In addition, the modified Fc region of BE-830 also has a weak binding to C1q, and the negative control showed no binding to C1q (FIG. 23).

### Example 15. GPC3×CD137 Induces T Cell Activation Cocultured with GPC3 Positive Tumor Cells

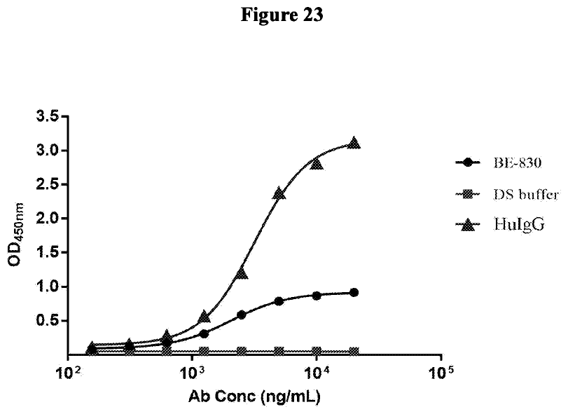

The functional activity of GPC3×CD137 bispecific antibody BE-830 was assessed in in vitro co-culture experiments using human peripheral blood mononuclear cell (PBMC) and OS8-expressing hepatocellular carcinoma (HCC) cell lines (FIG. 24A). OS8 is a single chain variable fragment (scFv) of an anti-human CD3 mAb OKT3 fused to the C-terminal domain (aa 113-220) of mouse CD8a which includes hinge, transmembrane and cytoplasmic domains. When expressed on target cells, OS8 could provide a first signal for T cell activation. Three HCC cell lines, HepG2, Huh7 and Hep3B, with high to low GPC3 expression based on the FACS analysis (FIG. 24B) were chosen to evaluate the effect of GPC3 levels on the functional activity of BE-830. No GPC3 expressing SK-HEP-1 and SK-OV-3 were used as negative control cell lines.

Frozen human PBMC (AllCells) were thawed in RPMI 1640 medium and incubated at 37° C. overnight. OS8-expressing target cells were seeded into 384-well plates and left to attach for 16 hours. The next day, PBMC were added into the 384-well plates with the effector to target cell ratio (E:T) of 2:1. Then co-cultured cells were treated with a series dilution of BE-830 for 48 hours at 37° C. Culture supernatant was collected for subsequent measurement of IFN-γ and IL-2 concentration by a TR-FRET-based method (Degorce et al., Current chemical genomics. 2009, 3: 22) as described by the manufacturer manual (Cisbio). The results showed that BE-830 induced dose-dependent cytokine release in PBMC cocultured with GPC3 expressing cells but not with GPC3 negative cells (FIG. 24C). Expression level of GPC3 on target cells did not significantly impact the potency of BE-830, as indicated by similar cytokine production EC₅₀s in PBMC cocultured with cell lines with low-to-high GPC3 levels (FIG. 24B-24C). PBMCs from three donors were tested and results were shown as mean±SD of triplicates.

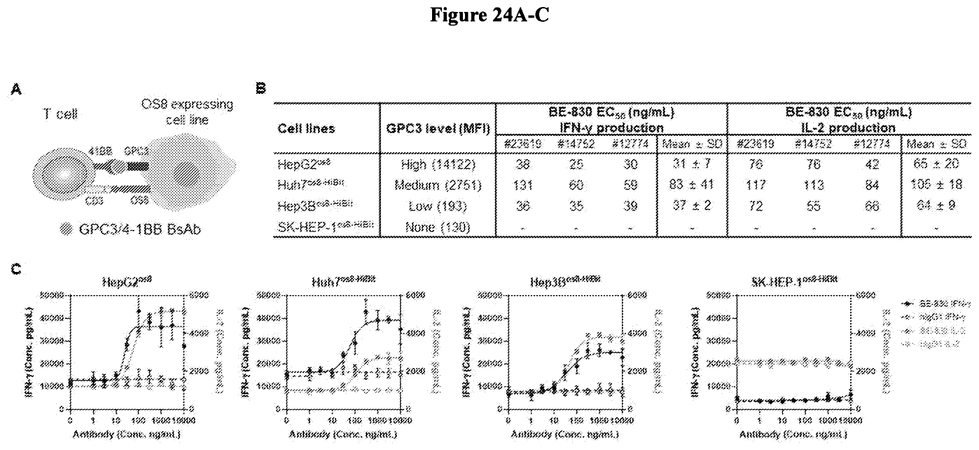

BE-830 regulated T-cell killing activity was assessed in co-culture experiments with impedance measurements using an xCELLigence™ RTCA MP instrument (Agilent Technologies). Frozen human PBMC (AllCells) were thawed in RPMI 1640 medium and incubated at 37° C. overnight. Target cells were seeded into 96-well-E plates (Agilent Technologies) and left to attach for 16 hours. The next day, PBMC were added into the 96-well-E plates with the effector to target cell ratio (E:T) of 5:1. Then co-cultured cells were treated with a series dilution of BE-830 in combination with an EpCAM/CD3 bispecific T-cell engager (BiTE) which provides a first signal for T cell activation (FIG. 25A). The experiment was allowed to proceed for 4 days to measure electrode impedance by live adherent target cells. The results showed that BE-830 dose-dependently enhanced T cell killing activity to GPC3 expressing cells but not to GPC3 negative cells (FIG. 25C). Consistent with cytokine production assay, BE-830 showed similar potency in enhancing T cell mediated killing effect to target cells with different GPC3 expression levels (FIG. 25B-25C). PBMCs from three donors were tested and results were shown as mean±SD of triplicates.

### Example 16. Combination with Anti-PD-1 Antibody BGB-A317 (Tislelizumab) Further Promotes Immune Cell Activation

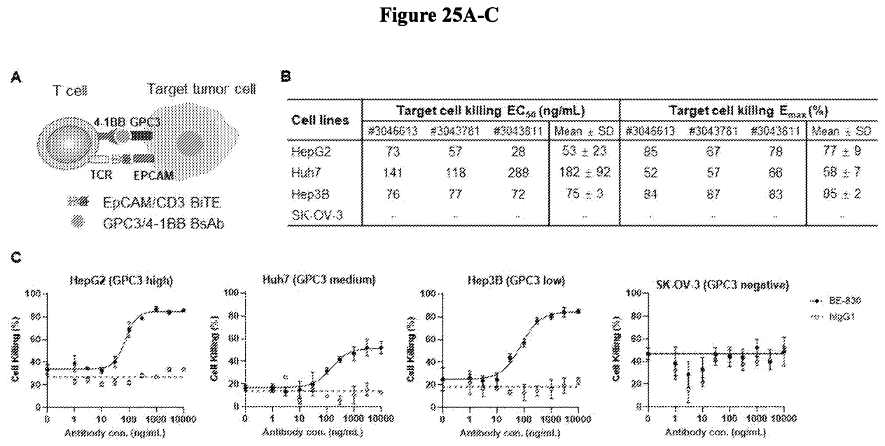

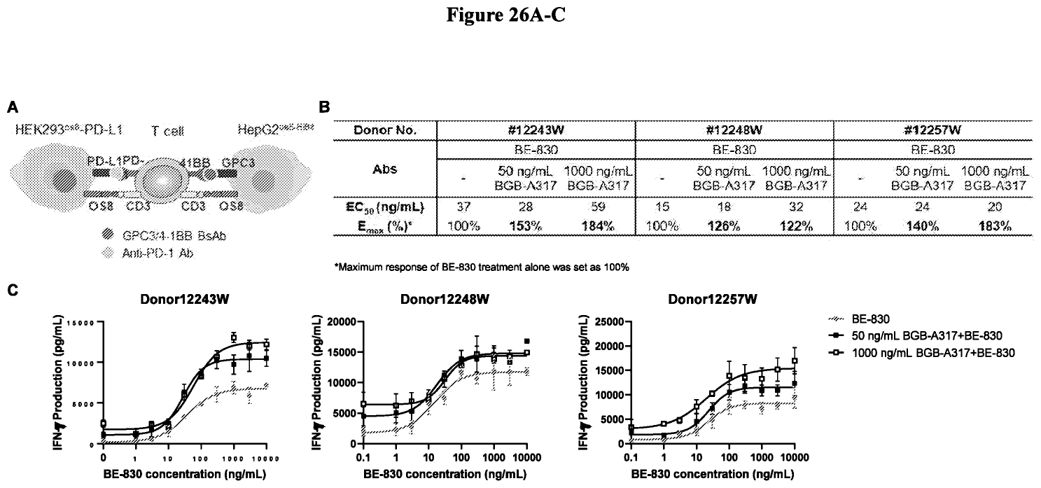

Costimulatory receptor CD137 can induce T-cell activation intracellular signals, but the signals were usually repressed by immune-checkpoint ligation, such as PD-1/PD-L1. Therefore, PD-1 blockade antibody BGB-A317 (Tislelizumab) and BE-830 may have a combinatorial effect in enhancing T cell activation. To determine the functional activity of BE-830 in combination with the anti-PD-1 antibody BGB-A317, human PBMCs were co-cultured with GPC3 and PD-L1 expressing target cells (FIG. 26A), and the IFN-γ release was measured as functional readout (FIG. 26B). Frozen human PBMC (SailyBio) were thawed in RPMI 1640 medium and incubated at 37° C. overnight. High GPC3 expressed HepG2-OS8 cells mixed with HEK293/OS8-PD-L1 cells, which were engineered to express PD-L1 and T-cell engaging OS8, were seeded with the ratio of 1:1 into 96-well plates and left to attach for 16 hours. The next day, PBMC were added into the 96-well plates with the effector to target cell ratio (E:T) of 2:1. Then co-cultured cells were treated with a series dilution of BE-830 in combination with 50 or 1000 ng/mL BGB-A317 for 48 hours at 37° C. Culture supernatant was collected for subsequent measurement of IFN-γ concentration by a AlphaLISA-based method (Bielefeld S. M., Assay and drug development technologies. 2009, 7(1), 90-92) as described by the manufacturer manual (PerkinElmer). The results showed that in three cell co-culture system, combination of BE-830 and BGB-A317 could further enhance IFN-γ production in T cells in contrast to treatment with BE-830 and BGB-A317 as a single agent, indicating the BE-830 and BGB-A317 have an additive effect on human T cell activation (FIG. 26B-26C).

BGB-A317 (Tislelizumab) is disclosed in U.S. Pat. No. 8,735,553 and the VH/VL sequences are shown in Table 20 below.

TABLE 20
Tislelizumab sequence table

| | |
|---|---|
| Tislelizumab | QVQLQESGPGLVKPSETLSLTCTVSGFSLTSYGVHWIRQPPGKGLE |
| VH | WIGVIYADGSTNYNPSLKSRVTISKDTSKNQVSLKLSSVTAADTAV |
| | YYCARAYGNYWYIDVWGQGTTVTVSS |
| | (SEQ ID NO: 210) |
| Tislelizumab | DIVMTQSPDSLAVSLGERATINCKSSESVSNDVAWYQQKPGQPPK |
| VL | LLINYAFHRFTGVPDRFSGSGYGTDFTLTISSLQAEDVAVYYCHQA |
| | YSSPYTFGQGTKLEIK |
| | (SEQ ID NO: 211) |

### Example 17. Efficacy of BE-830 Monotherapy in Hepa1-6/hGPC3 Model in Humanized CD137 Knock-In Mice

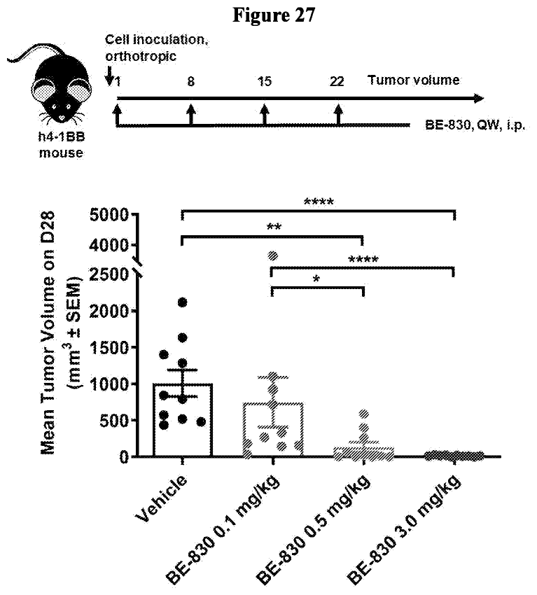

The in vivo efficacy of BE-830 was examined in the Hepa1-6/hGPC3 mouse hepatocellular carcinoma model in humanized CD137 knock-in mice. Hepa1-6/hGPC3 cells were orthotopically implanted into the left liver lobe of the recipient mice, and the mice were randomized into 4 groups according to body weight. BE-830 was intraperitoneally administrated on Day 1 and were administrated weekly for 4 weeks. BE-830 (0.1, 0.5, and 3.0 mg/kg, once weekly) effectively inhibited tumor growth. The tumor volume at the primary inoculation site was significantly decreased at study endpoint (D28). In addition, the ratios of tumor free in 0.1, 0.5 and 3.0 mg/kg groups was 0%, 40%, and 20% on Day 28, respectively (FIG. 27 and Table 21). Pharmacokinetic (PK) profiles of BE-830 at the three dosage levels (0.1, 0.5, and 3.0 mg/kg) were characterized after the first dosing. Drug exposure of BE-830 (AUC₀₋₁₆₈ₕ and Cₘₐₓ) were increased proportionally (Table 21). There was no significant impact on animal body weight in any of the treatment group throughout the study.

TABLE 21
Efficacy and PK Parameters of BE-830 in the Hepa1-6/hGPC3
Syngeneic Tumor Model in Humanized CD137 Knock-in Mice

| | | | Mean tumor | | Ratio | PK parameter (single dose) | |
|---|---|---|---|---|---|---|---|
| | | | volume on | P-value vs | of tumor | | AUC0- |
| | | Dose | Day 28 | vehicle on | free on | Cmax (μg/mL) | 168 h (h*μg/mL) |
| BE-830 | n | (mg/kg) | (mm3 ± SEM) | Day 28 | Day 28 | (Mean ± SEM) | (Mean ± SEM) |
| Vehicle | 10 | NA | 1008.4 | NA | 0% | NA | NA |
| (control) | | | (±181.4) | | (0/10) | | |
| BE-830 | 10 | 0.1, QW | 749.3 | 0.2466 | 0% | 0.48 | 34.07 |
| | | | (±340.8) | | (0/10) | (±0.15) | (±5.60) |
| BE-830 | 10 | 0.5, QW | 134.8 | 0.0016 | 40% | 3.90 | 195.58 |
| | | | (±66.3) | | (4/10) | (±0.63) | (±26.66) |
| BE-830 | 10 | 3.0, QW | 13.2 | 0.0000 | 20% | 17.97 | 1699.26 |
| | | | (±2.8) | | (2/10) | (±3.30) | (±302.88) |

Abbreviations: AUC_{0-168 h}, area under the serum concentration-time curve from 0 to 168 hours; Cₘₐₓ, maximum concentration; hGPC3, human Glypican 3; n, number of animals; NA, not applicable; PK, pharmacokinetics; QW, once weekly; SEM, standard error of the mean.

### Example 18. Efficacy of the Combination of BE-830 and Anti-PD-1 Antibody in H22/hGPC3 Model in Humanized CD137 Knock-In Mice

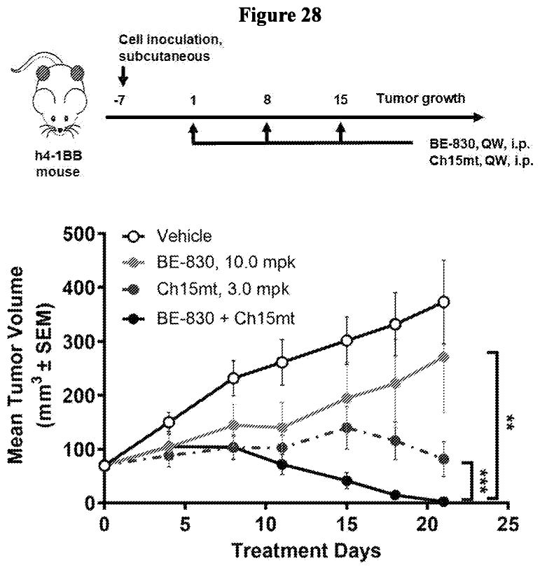

The antitumor activity of the combination of BE-830 and anti-mouse PD-1 antibody was investigated in the H22/hGPC3 syngeneic model in humanized CD137 knock-in mice. H22/hGPC3 cells were implanted into female mice. On Day 7 after cell inoculation, the mice were randomized into 4 groups according to tumor volume. Mice receiving the combination treatment of BE-830 (10.0 mg/kg, once weekly) and anti-mouse PD-1 antibody Ch15mt (3.0 mg/kg, once weekly) exhibited synergistic effects. The tumor growth inhibition rate in the combination group was 122% on Day 21, which was significantly higher than that in the group treated with BE-830 (34%) or Ch15mt (96%) alone (FIG. 28 and Table 22). The ratio of tumor free in the combination group was 80% on Day 21, which was higher than that in the group treated with BE-830 (40%) or Ch15mt (10%) alone (Table 22). No significant impact on animal body weight was observed in any treatment group throughout the study.

TABLE 22
Antitumor Effect of BE-830 and anti-mouse PD-1 antibody Ch15mt
in H22/hGPC3 Syngeneic Model in Humanized CD137 Knock-in Mice

| | | | | Mean tumor | P value vs. BE- | Ratio of |
|---|---|---|---|---|---|---|
| | | Dosage | TGI (%) | volume on Day 21 | 830 + Ch15mt | tumor free |
| Antibody | n | (mg/kg) | on Day 21 | (mm³ ± SEM) | group on Day 21 | on Day 21 |
| Vehicle | 10 | NA | NA | 373.4 ± 77.6 | NA | 0% |
| (control) | | | | | | (0/10) |
| BE-830 | 10 | 10.0 QW | 34 | 271.4 ± 103.7 | 0.0017 | 40% |
| | | | | | | (4/10) |
| Ch15mt | 10 | 3.0 QW | 96 | 81.5 ± 32.7 | 0.0007 | 10% |
| | | | | | | (1/10) |
| BE-830 + | 10 | 10.0 + | 122 | 2.6 ± 2.3 | NA | 80% |
| Ch15mt | | 3.0 QW | | | | (8/10) |

Abbreviations: hGPC3, human Glypican 3; n, number of animals; NA, not applicable; QW, once weekly; SEM, standard error of the mean; TGI, tumor growth inhibition.
Note:
TGI rate was calculated according to the following formula: % TGI = [1 − (treated Tt − treated T0)/(vehicle Tt − vehicle T0)] × 100%. This table shows the TGI on Day 21. Treated Tt = mean tumor volume of a dosing group on Day t; treated T0 = mean tumor volume of a dosing group on Day 0; vehicle Tt = mean tumor volume of vehicle group on Day t; and vehicle T0 = mean tumor volume of vehicle group on Day 0.

### Example 19. Liver Toxicity of BE-830 in Humanized CD137 Knock-In Mice

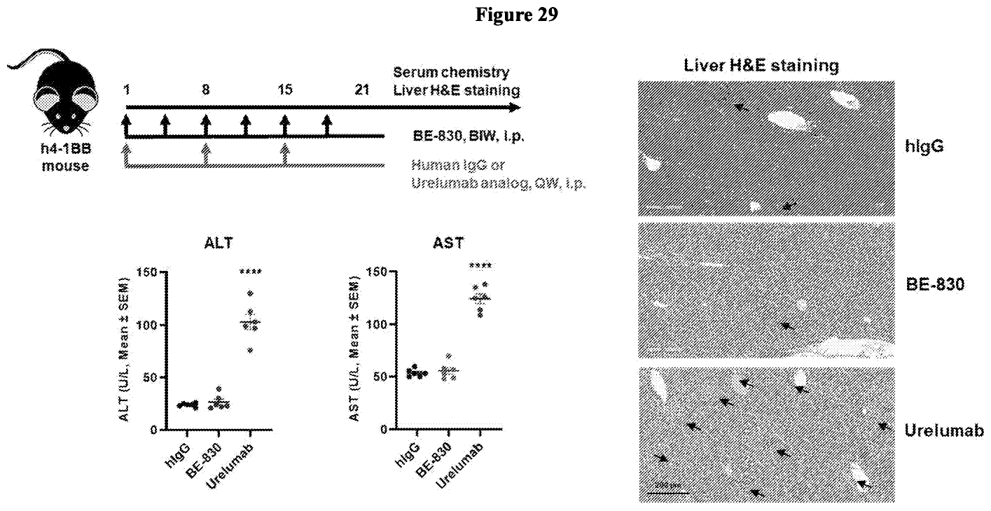

BE-830 (30 mg/kg, twice weekly) or the Urelumab analog antibody (30 mg/kg, once weekly) were intraperitoneally injected into humanized CD137 mice. The blood and liver tissues of the mice were collected on Day 21 after the antibodies treatment, and serum chemistry and liver tissue histopathology were tested. The Urelumab analog antibody, but not BE-830, induced significant alanine transaminase (ALT) and aspartate aminotransferase (AST) elevation in the serum, which indicated liver toxicity of the Uremulab analog. In addition, pathological changes were observed in the liver tissues from the Urelumab analog antibody treated group, shown as increased inflammatory cells infiltration. The pathological changes were not observed in the BE-830 treated group (FIG. 29).

### REFERENCES

Filmus J, Selleck S B. Glypicans: proteoglycans with a surprise. The Journal of clinical investigation. 2001; 108: 497-501.
Li N, Gao W, Zhang Y F, Ho M. Glypicans as Cancer Therapeutic Targets. Trends in cancer. 2018; 4: 741-54.
Ho M, Kim H. Glypican-3: a new target for cancer immunotherapy. European journal of cancer. 2011; 47: 333-8.
Zhou F, Shang W, Yu X, Tian J. Glypican-3: A promising biomarker for hepatocellular carcinoma diagnosis and treatment. Medicinal research reviews. 2018; 38: 741-67.
Kirsten L Moek, Rudolf S N Fehrmann, Bert van der Vegt, Elisabeth G E de Vries, Derk J A de Groot. Glypican 3 Overexpression across a Broad Spectrum of Tumor Types Discovered with Functional Genomic mRNA Profiling of a Large Cancer Database. 2018; 188(9): 1973-1981.
Arnon, T. I., H. Achdout, O. Levi, G. Markel, N. Saleh, G. Katz, R. Gazit, T. Gonen-Gross, J. Hanna, E. Nahari, A. Porgador, A. Honigman, B. Plachter, D. Mevorach, D. G. Wolf & O. Mandelboim (2005) Inhibition of the CD137 activating receptor by pp65 of human cytomegalovirus. Nat Immunol, 6, 515-23.
Augugliaro, R., S. Parolini, R. Castriconi, E. Marcenaro, C. Cantoni, M. Nanni, L. Moretta, A. Moretta & C. Bottino (2003) Selective cross-talk among natural cytotoxicity receptors in human natural killer cells. Eur J Immunol, 33, 1235-41.
Barrow, A. D., C. J. Martin & M. Colonna (2019) The Natural Cytotoxicity Receptors in Health and Disease. Front Immunol, 10, 909.
Beck, A., T. Wurch, C. Bailly & N. Corvaia (2010) Strategies and challenges for the next generation of therapeutic antibodies. Nat Rev Immunol, 10, 345-52.
Biron, C. A., K. B. Nguyen, G. C. Pien, L. P. Cousens & T. P. Salazar-Mather (1999) Natural killer cells in antiviral defense: function and regulation by innate cytokines. Annu Rev Immunol, 17, 189-220.
Bozzano, F., A. Picciotto, P. Costa, F. Marras, V. Fazio, I. Hirsch, D. Olive, L. Moretta & A. De Maria (2011) Activating NK cell receptor expression/function (CD137, NKp46, DNAM-1) during chronic viraemic HCV infection is associated with the outcome of combined treatment. Eur J Immunol, 41, 2905-14.
Brinkmann, U. & R. E. Kontermann (2017) The making of bispecific antibodies. MAbs, 9, 182-212.
Brocks, B., P. Garin-Chesa, E. Behrle, J. E. Park, W. J. Rettig, K. Pfizenmaier & D. Moosmayer (2001) Species-crossreactive scFv against the tumor stroma marker “fibroblast activation protein” selected by phage display from an immunized FAP−/− knock-out mouse. Molecular medicine (Cambridge, Mass.), 7, 461-469.
Castriconi, R., C. Cantoni, M. Della Chiesa, M. Vitale, E. Marcenaro, R. Conte, R. Biassoni, C. Bottino, L. Moretta & A. Moretta (2003) Transforming growth factor beta 1 inhibits expression of CD137 and NKG2D receptors: consequences for the NK-mediated killing of dendritic cells. Proc Natl Acad Sci USA, 100, 4120-5.
Correia, M. P., A. Stojanovic, K. Bauer, D. Juraeva, L. O. Tykocinski, H. M. Lorenz, B. Brors & A. Cerwenka (2018) Distinct human circulating CD137(+)FcepsilonRIgamma(+)CD8(+) T cell population exhibiting high natural killer-like antitumor potential. Proc Natl Acad Sci USA, 115, E5980-E5989.
Dahlen, E., N. Veitonmaki & P. Norlen (2018) Bispecific antibodies in cancer immunotherapy. Ther Adv Vaccines Immunother, 6, 3-17.
Delahaye, N. F., S. Rusakiewicz, I. Martins, C. Menard, S. Roux, L. Lyonnet, P. Paul, M. Sarabi, N. Chaput, M. Semeraro, V. Minard-Colin, V. Poirier-Colame, K. Chaba, C. Flament, V. Baud, H. Authier, S. Kerdine-Romer, M. Pallardy, I. Cremer, L. Peaudecerf, B. Rocha, D. Valteau-Couanet, J. C. Gutierrez, J. A. Nunes, F. Commo, S. Bonvalot, N. Ibrahim, P. Terrier, P. Opolon, C. Bottino, A. Moretta, J. Tavernier, P. Rihet, J. M. Coindre, J. Y. Blay, N. Isambert, J. F. Emile, E. Vivier, A. Lecesne, G. Kroemer & L. Zitvogel (2011) Alternatively spliced CD137 isoforms affect the prognosis of gastrointestinal stromal tumors. Nat Med, 17, 700-7.
Fischer, E., K. Chaitanya, T. Wuest, A. Wadle, A. M. Scott, M. van den Broek, R. Schibli, S. Bauer & C. Renner (2012) Radioimmunotherapy of fibroblast activation protein positive tumors by rapidly internalizing antibodies. Clin Cancer Res, 18, 6208-18.
Gauthier, L., A. Morel, N. Anceriz, B. Rossi, A. Blanchard-Alvarez, G. Grondin, S. Trichard, C. Cesari, M. Sapet, F. Bosco, H. Rispaud-Blanc, F. Guillot, S. Cornen, A. Roussel, B. Amigues, G. Habif, F. Caraguel, S. Arrufat, R. Remark, F. Romagne, Y. Morel, E. Narni-Mancinelli & E. Vivier (2019) Multifunctional Natural Killer Cell Engagers Targeting NKp46 Trigger Protective Tumor Immunity. Cell, 177, 1701-1713 e16.
Gold, P. & S. O. Freedman (1965) Demonstration of tumor-specific antigens in human colonic carcinomata by immunological tolerance and absorption techniques. J Exp Med, 121, 439-62.
Hezareh, M., A. J. Hessell, R. C. Jensen, J. G. van de Winkel & P. W. Parren (2001) Effector function activities of a panel of mutants of a broadly neutralizing antibody against human immunodeficiency virus type 1. J Virol, 75, 12161-8.
Hollyoake, M., R. D. Campbell & B. Aguado (2005) CD137 (NCR3) is a pseudogene in 12 inbred and wild mouse strains, but an expressed gene in Mus caroli. Mol Biol Evol, 22, 1661-72.
Hsieh, C. L., K. Nagasaki, O. M. Martinez & S. M. Krams (2006) CD137 is a functional activation receptor on a subset of rat natural killer cells. Eur J Immunol, 36, 2170-80.
Hudspeth, K., M. Fogli, D. V. Correia, J. Mikulak, A. Roberto, S. Della Bella, B. Silva-Santos & D. Mavilio (2012) Engagement of CD137 on Vdelta1 T cells induces the production of CCL3, CCL4, and CCL5 and suppresses HIV-1 replication. Blood, 119, 4013-6.
Hudspeth, K., B. Silva-Santos & D. Mavilio (2013) Natural cytotoxicity receptors: broader expression patterns and functions in innate and adaptive immune cells. Front Immunol, 4, 69.
Hurwitz, E., I. Stancovski, M. Sela & Y. Yarden (1995) Suppression and promotion of tumor growth by monoclonal antibodies to ErbB-2 differentially correlate with cellular uptake. Proc Natl Acad Sci USA, 92, 3353-7.
Igawa, T., H. Tsunoda, Y. Kikuchi, M. Yoshida, M. Tanaka, A. Koga, Y. Sekimori, T. Orita, Y. Aso, K. Hattori & M. Tsuchiya (2010) VH/VL interface engineering to promote selective expression and inhibit conformational isomerization of thrombopoietin receptor agonist single-chain diabody. Protein Eng Des Sel, 23, 667-77.
Joyce, M. G., P. Tran, M. A. Zhuravleva, J. Jaw, M. Colonna & P. D. Sun (2011) Crystal structure of human natural cytotoxicity receptor CD137 and identification of its ligand binding site. Proc Natl Acad Sci USA, 108, 6223-8.
Koch, J., A. Steinle, C. Watzl & O. Mandelboim (2013) Activating natural cytotoxicity receptors of natural killer cells in cancer and infection. Trends Immunol, 34, 182-91.
Kruse, P. H., J. Matta, S. Ugolini & E. Vivier (2014) Natural cytotoxicity receptors and their ligands. Immunol Cell Biol, 92, 221-9.
Lanier, L. L. (2015) NKG2D Receptor and Its Ligands in Host Defense. Cancer Immunol Res, 3, 575-82.
Lefranc, M. P., V. Giudicelli, C. Ginestoux, J. Bodmer, W. Muller, R. Bontrop, M. Lemaitre,
A. Malik, V. Barbie & D. Chaume (1999) IMGT, the international ImMunoGeneTics database. Nucleic Acids Res, 27, 209-12.
Li, Y., Q. Wang & R. A. Mariuzza (2011) Structure of the human activating natural cytotoxicity receptor CD137 bound to its tumor cell ligand B7-H6. J Exp Med, 208, 703-14.
Liu, L., W. Zeng, M. A. Wortinger, S. B. Yan, P. Cornwell, V. L. Peek, J. R. Stephens, J. W. Tetreault, J. Xia, J. R. Manro, K. M. Credille, D. W. Ballard, P. Brown-Augsburger, V. Wacheck, C. K. Chow, L. Huang, Y. Wang, I. Denning, J. Davies, Y. Tang, P. Vaillancourt & J. Lu (2014) LY2875358, a neutralizing and internalizing anti-MET bivalent antibody, inhibits HGF-dependent and HGF-independent MET activation and tumor growth. Clin Cancer Res, 20, 6059-70.
Mechetner, E. (2007) Development and characterization of mouse hybridomas. Methods Mol Biol, 378, 1-13.
Muller, D. & R. E. Kontermann (2010) Bispecific antibodies for cancer immunotherapy: Current perspectives. BioDrugs, 24, 89-98.
Pegram, H. J., D. M. Andrews, M. J. Smyth, P. K. Darcy & M. H. Kershaw (2011) Activating and inhibitory receptors of natural killer cells. Immunol Cell Biol, 89, 216-24.
Pende, D., S. Parolini, A. Pessino, S. Sivori, R. Augugliaro, L. Morelli, E. Marcenaro, L. Accame, A. Malaspina, R. Biassoni, C. Bottino, L. Moretta & A. Moretta (1999) Identification and molecular characterization of CD137, a novel triggering receptor involved in natural cytotoxicity mediated by human natural killer cells. J Exp Med, 190, 1505-16.
Pesce, S., G. Tabellini, C. Cantoni, O. Patrizi, D. Coltrini, F. Rampinelli, J. Matta, E. Vivier, A. Moretta, S. Parolini & E. Marcenaro (2015) B7-H6-mediated downregulation of CD137 in NK cells contributes to ovarian carcinoma immune escape. Oncoimmunology, 4, e1001224.
Pogge von Strandmann, E., V. R. Simhadri, B. von Tresckow, S. Sasse, K. S. Reiners, H. P. Hansen, A. Rothe, B. Boll, V. L. Simhadri, P. Borchmann, P. J. McKinnon, M. Hallek & A. Engert (2007) Human leukocyte antigen-B-associated transcript 3 is released from tumor cells and engages the CD137 receptor on natural killer cells. Immunity, 27, 965-74.
Poul, M. A., B. Becerril, U. B. Nielsen, P. Morisson & J. D. Marks (2000) Selection of tumor-specific internalizing human antibodies from phage libraries. J Mol Biol, 301, 1149-61.
Richman, P. I. & W. F. Bodmer (1987) Monoclonal antibodies to human colorectal epithelium: markers for differentiation and tumour characterization. Int J Cancer, 39, 317-28.
Ridgway, J. B., L. G. Presta & P. Carter (1996) ‘Knobs-into-holes’ engineering of antibody CH3 domains for heavy chain heterodimerization. Protein Eng, 9, 617-21.
Silacci, M., S. Brack, G. Schirru, J. Marlind, A. Ettorre, A. Merlo, F. Viti & D. Neri (2005) Design, construction, and characterization of a large synthetic human antibody phage display library. Proteomics, 5, 2340-50.
Takahashi, T., S. Minami, Y. Tsuchiya, K. Tajima, N. Sakai, K. Suga, S. I. Hisanaga, N. Ohbayashi, M. Fukuda & H. Kawahara (2019) Cytoplasmic control of Rab family small GTPases through BAG6. EMBO Rep, 20.
Von Kreudenstein, T. S., E. Escobar-Carbrera, P. I. Lario, I. D'Angelo, K. Brault, J. Kelly, Y. Durocher, J. Baardsnes, R. J. Woods, M. H. Xie, P. A. Girod, M. D. Suits, M. J. Boulanger, D. K. Poon, G. Y. Ng & S. B. Dixit (2013) Improving biophysical properties of a bispecific antibody scaffold to aid developability: quality by molecular design. MAbs, 5, 646-54.
Wu, J., Y. Song, A. B. Bakker, S. Bauer, T. Spies, L. L. Lanier & J. H. Phillips (1999) An activating immunoreceptor complex formed by NKG2D and DAP10. Science, 285, 730-2.
Wu, T. T. & E. A. Kabat (1970) An analysis of the sequences of the variable regions of Bence Jones proteins and myeloma light chains and their implications for antibody complementarity. J Exp Med, 132, 211-50.
Zhang, T., B. A. Lemoi & C. L. Sentman (2005) Chimeric NK-receptor-bearing T cells mediate antitumor immunotherapy. Blood, 106, 1544-1551.
Zhang, T., X. Song, L. Xu, J. Ma, Y. Zhang, W. Gong, Y. Zhang, X. Zhou, Z. Wang, Y. Wang, Y. Shi, H. Bai, N. Liu, X. Yang, X. Cui, Y. Cao, Q. Liu, J. Song, Y. Li, Z. Tang, M. Guo, L. Wang & K. Li (2018) The binding of an anti-PD-1 antibody to FcgammaRIota has a profound impact on its biological functions. Cancer Immunol Immunother, 67, 1079-1090.
Zhang, T., M. R. Wu & C. L. Sentman (2012) An CD137-based chimeric antigen receptor promotes T cell effector functions and antitumor efficacy in vivo. J Immunol, 189, 2290-9.
Zhao, L., L. Qu, J. Zhou, Z. Sun, H. Zou, Y. Y. Chen, J. D. Marks & Y. Zhou (2014) High throughput identification of monoclonal antibodies to membrane bound and secreted proteins using yeast and phage display. PLOS One, 9, e111339.

1. A multispecific antibody or antigen-binding fragment thereof, comprising a first antigen binding domain that specifically binds to human Glypican 3 (GPC3) and a second antigen binding domain that specifically binds to human CD137, wherein the second antigen binding domain that specifically binds to human CD137 comprises:
(i) a heavy chain variable region that comprises (a) a HCDR1 of SEQ ID NO: 65, (b) a HCDR2 of SEQ ID NO: 80, and (c) a HCDR3 of SEQ ID NO: 81;
(ii) a heavy chain variable region that comprises (a) a HCDR1 of SEQ ID NO: 65, (b) a HCDR2 of SEQ ID NO: 73, and (c) a HCDR3 of SEQ ID NO: 67;
(iii) a heavy chain variable region that comprises (a) a HCDR1 of SEQ ID NO: 65, (b) a HCDR2 of SEQ ID NO: 66, and (c) a HCDR3 of SEQ ID NO: 67; or
(iv) a heavy chain variable region that comprises (a) a HCDR1 of SEQ ID NO: 55, (b) a HCDR2 of SEQ ID NO: 56, and (c) a HCDR3 of SEQ ID NO: 57.

2. (canceled)

3. The multispecific antibody or antigen-binding fragment thereof of claim 1, wherein the second antigen binding domain that specifically binds to human CD137 comprises:
(i) a heavy chain variable region comprising SEQ ID NO: 84 or an amino acid sequence at least 90% identical to SEQ ID NO: 84;
(ii) a heavy chain variable region comprising SEQ ID NO: 86 or an amino acid sequence at least 90% identical to SEQ ID NO: 86;
(iii) a heavy chain variable region comprising SEQ ID NO: 75 or an amino acid sequence at least 90% identical to SEQ ID NO: 75;
(iv) a heavy chain variable region comprising SEQ ID NO: 70 or an amino acid sequence at least 90% identical to SEQ ID NO: 70; or
(v) a heavy chain variable region comprising SEQ ID NO: 60 or an amino acid sequence at least 90% identical to SEQ ID NO: 60.

4-5. (canceled)

6. The multispecific antibody or antigen-binding fragment thereof of claim 1, wherein the second antigen binding domain that specifically binds to human CD137 binds to an epitope of human CD137 comprising amino acids F36, P47 and P49.

7. The multispecific antibody or antigen-binding fragment thereof of claim 1, wherein the first antigen binding domain that specifically binds to human GPC3 comprises:
a heavy chain variable region that comprises (a) a HCDR1 of SEQ ID NO: 10, (b) a HCDR2 of SEQ ID NO: 11, and (c) a HCDR3 of SEQ ID NO: 12; and
a light chain variable region that comprises (d) a LCDR1 of SEQ ID NO: 13, (e) a LCDR2 of SEQ ID NO: 14, and (f) a LCDR3 of SEQ ID NO: 15.

8. The multispecific antibody or antigen-binding fragment thereof of claim 1, wherein the first antigen binding domain that specifically binds to human GPC3 comprises: a heavy chain variable region comprising SEQ ID NO: 5 or an amino acid sequence at least 90% identical to SEQ ID NO: 5, and a light chain variable region comprising SEQ ID NO: 7 or an amino acid sequence at least 90% identical to SEQ ID NO: 7.

9-10. (canceled)

11. The multispecific antibody or antigen-binding fragment thereof of claim 1, wherein:
(a) the first antigen binding domain that specifically binds to human GPC3 comprises: a heavy chain variable region that comprises (a) a HCDR1 of SEQ ID NO: 10, (b) a HCDR2 of SEQ ID NO: 11, and (c) a HCDR3 of SEQ ID NO: 12; and a light chain variable region that comprises (d) a LCDR1 of SEQ ID NO: 13, (e) a LCDR2 of SEQ ID NO: 14, and (f) a LCDR3 of SEQ ID NO: 15, and
(b) the second antigen binding domain that specifically binds to human CD137 comprises:
(i) a heavy chain variable region that comprises (a) a HCDR1 of SEQ ID NO: 65, (b) a HCDR2 of SEQ ID NO: 80, (c) a HCDR3 of SEQ ID NO: 81;
(ii) a heavy chain variable region that comprises (a) a HCDR1 of SEQ ID NO: 65, (b) a HCDR2 of SEQ ID NO: 73, (c) a HCDR3 of SEQ ID NO: 67;
(iii) a heavy chain variable region that comprises (a) a HCDR1 of SEQ ID NO: 65, (b) a HCDR2 of SEQ ID NO: 66, (c) a HCDR3 of SEQ ID NO: 67; or
(iv) a heavy chain variable region that comprises (a) a HCDR1 of SEQ ID NO: 55, (b) a HCDR2 of SEQ ID NO: 56, (c) a HCDR3 of SEQ ID NO: 57.

12. The multispecific antibody or antigen-binding fragment thereof of claim 1, wherein;
(a) the first antigen binding domain that specifically binds to human GPC3 comprises: a heavy chain variable region that comprises SEQ ID NO: 5, and a light chain variable region that comprises SEQ ID NO: 7; and
(b) the second antigen binding domain that specifically binds to human CD137 comprises:
(i) a heavy chain variable region that comprises SEQ ID NO: 84;
(ii) a heavy chain variable region that comprises SEQ ID NO: 86;
(iii) a heavy chain variable region that comprises SEQ ID NO: 75;
(iv) a heavy chain variable region that comprises SEQ ID NO: 70; or
(v) a heavy chain variable region that comprises SEQ ID NO: 60.

13. The multispecific antibody or antigen-binding fragment thereof of claim 1, wherein the multispecific antibody or antigen-binding fragment is a monoclonal antibody, a chimeric antibody, a humanized antibody, a human engineered antibody, a single chain antibody (scFv), a Fab fragment, a Fab′ fragment, or a F(ab′)₂ fragment; or
wherein the first antigen binding domain that specifically binds to human GPC3 is a monoclonal antibody, a chimeric antibody, a humanized antibody, a human engineered antibody, a single chain antibody (scFv), a single domain antibody, a Fab fragment, a Fab′ fragment, or a F(ab′)₂ fragment, and the second antigen binding domain that specifically binds to human CD137 is a monoclonal antibody, a chimeric antibody, a humanized antibody, a human engineered antibody, a single chain antibody (scFv), a single domain antibody, a Fab fragment, a Fab′ fragment, or a F(ab′)₂ fragment; or
wherein the multispecific antibody or antigen-binding fragment thereof is a bispecific antibody.

14-15. (canceled)

16. The multispecific antibody or antigen-binding fragment thereof of claim 1, wherein the multispecific antibody or antigen-binding fragment comprises a linker selected from SEQ ID NO:16 to SEQ ID NO: 51 and SEQ ID NO: 88 to SEQ ID NO: 93.

17-18. (canceled)

19. The multispecific antibody or antigen-binding fragment thereof of claim 1, wherein the multispecific antibody or antigen-binding fragment comprises a heavy chain constant region of the subclass of IgG1, IgG2, IgG3, or IgG4, and/or a light chain constant region of the type of kappa or lambda, and
wherein the heavy chain constant region comprises a CH1 and/or an Fc domain.

20. The multispecific antibody or antigen-binding fragment thereof of claim 1, wherein the multispecific antibody or antigen-binding fragment thereof has one or more of the following: antibody dependent cellular cytotoxicity (ADCC), complement dependent cytotoxicity (CDC), reduced glycosylation, no glycosylation, hypofucosylation, or increased bisecting GlcNac structures.

21-22. (canceled)

23. The multispecific antibody or antigen-binding fragment thereof of claim 1, wherein the multispecific antibody or antigen-binding fragment thereof comprises a Fc domain, and wherein the Fc domain is an IgG1 with reduced effector function or an IgG4, optionally the Fc domain comprises an amino acid sequence of SEQ ID NO: 9.

24. (canceled)

25. The multispecific antibody or antigen-binding fragment thereof of claim 1, wherein:
a) a heavy chain variable region of the first antigen binding domain that specifically binds to human GPC3, a CH1 domain, an Fc domain, and the heavy chain variable region of the second antigen binding domain that specifically binds to human CD137 are arranged in a first polypeptide in the direction of N terminus to C terminus;
optionally, C terminus of the Fc domain is linked to N terminus of the heavy chain variable region of the second antigen binding domain via a linker; and
b) a light chain variable region of the first antigen binding domain that specifically binds to human GPC3 and a first light chain constant region are arranged in a second polypeptide in the direction of N terminus to C terminus.

26. The multispecific antibody or antigen-binding fragment thereof of claim 1, wherein the multispecific antibody or antigen-binding fragment comprises a first polypeptide of SEQ ID NO: 1 and a second polypeptide of SEQ ID NO: 3.

27. A pharmaceutical composition comprising the multispecific antibody or antigen-binding fragment thereof of claim 1 and a pharmaceutically acceptable carrier.

28. A method of treating a cancer expressing GPC3 comprising administering to a patient in need an effective amount of the multispecific antibody or antigen-binding fragment thereof of claim 1.

29-39. (canceled)

40. An isolated nucleic acid that encodes the multispecific antibody or antigen-binding fragment thereof of claim 1.

41. A vector comprising the nucleic acid of claim 40.

42. A host cell comprising the nucleic acid of claim 40.

43. A process for producing a multispecific antibody or antigen-binding fragment thereof comprising cultivating the host cell of claim 42 and recovering the antibody or antigen-binding fragment thereof from the culture.
